SEQUENCE LISTING (1 sequence)

SEQ ID NO: 1 - protein, 448 aa, Homo sp.
MKTIIALSYIFCLVFADYKDDDDGAPLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPATIMVITYFLTIKSLQKEAADLEDNWETLNDNLKVIEKADNAAQVKDALTKMRAAALDAQKATPPKLEDKSPDSPEMKDFRHGFDILVGQIDDALKLANEGKVKEAQAAAEQLKTTRNAYIQKYLTMQSISNEQKACKVLGIVFFLFVVMWCPFFITNIMAVICKESCNEDVIGALLNVFVWIGYASSAVNPLVYTLFNKTYRSAFSRYIQCQYKENKKPGRPLEVLFQGPHHHHHHHHHH

(19) United States
(12) Patent Application Publication
(10) Pub. No.: US 2025/0382266 A1
(43) Pub. Date: Dec. 18, 2025

(54) 5-HT2A RECEPTOR LIGANDS

(71) Applicant: Psilera Inc., Tampa, FL (US)

(72) Inventors: Elena Bray, Temple Terrance, FL (US); Daniel Santiago, Largo, FL (US); Jeanine Yacoub, Lakeland, FL (US); Christopher Witowski, Tampa, FL (US); Jacqueline Salm, Tampa, FL (US)

(21) Appl. No.: 19/116,549

(22) PCT Filed: Oct. 19, 2023
(86) PCT No.: PCT/US2023/077243

§ 371 Date: Mar. 28, 2025

Related U.S. Application Data
(60) Provisional application No. 63/417,732, filed on Oct. 20, 2022.

(51) Int. Cl. C07D 209/16; C07D 471/04; C07D 519/00

(52) U.S. Cl. CPC C07D 209/16; C07D 471/04; C07D 519/00

(57) ABSTRACT

5-HT2A receptor ligands and receptor-ligand complexes are synthesized. These ligands, which may link two indole containing moieties through an amine, may be used in therapeutically effective amounts, to treat mood disorders and neurologic disorders.

### CROSS REFERENCE TO RELATED APPLICATION

This patent application is a national stage application of PCT/US2023/077243, filed Oct. 19, 2023, which claims the benefit of the filing date of U.S. provisional patent application Ser. No. 63/417,732, filed Oct. 20, 2022, the entire disclosures of which are incorporated by reference.

### FIELD OF THE INVENTION

The present invention relates to 5-HT2A receptor ligands.

### BACKGROUND OF THE INVENTION

Indoles are heterocyclic organic compounds that comprise a benzene ring that is a fused to a pyrrole ring. They are part of a diverse class of compounds that have broad biomedical potential. Among the uses of these compounds is the targeting of cancers, as well as the treatment of cardiovascular, gastrointestinal, and a wide range of neurological disorders. (Kaushik, Kaushik et al. 2013)

During in vivo biosynthesis of indoles, often the amino acid tryptophan, which is a precursor of serotonin, has been the scaffold of choice. Tryptophan is an understandable starting point because serotonin (5-HT) supports many important bodily functions including mood, sleep, appetite, intestinal motility, and sexual health. The serotonergic system consists of a class of G-coupled protein receptors, 5-HT1 through 5-HT7, as well as their subtypes (1A, 2A, 2B, etc.), which modulate many biological pathways. (Pithadia and Jain 2009)

Most serotonergic targeting therapeutics are antidepressants that act as: selective reuptake inhibitors (collectively SSRIs); direct 5-HT modulators (atypical); or in combination with norepinephrine inhibitors (SNRIs). While still not fully understood, the general mechanism of action of many approved therapeutics relies on increasing the concentration of the monoamines, 5-HT, and norepinephrine, in the postsynaptic receptors (Artigas, Nutt et al. 2002) to restore synaptic balance. However, these medications generally lack efficacy, being only 20-30% effective over placebo (Penn and Tracy 2012), have considerable side effects, and have a delayed period of buildup to reach maximum effectiveness of weeks to months. (Artigas, Nutt et al. 2002)

Approvals of new antidepressants by the FDA and other regulatory bodies around the world have lessened in recent decades despite the increased prevalence of depression. According to the National Institute of Mental Health, 17.3 million adults suffered from at least one depressive episode in 2017. One medication that the FDA has approved in the dissociative esketamine (also known as-ketamine or S-ketamine, which is the S enantiomer of ketamine), which was approved as a nasal spray for treatment-resistant depression. It has proven beneficial in large part due to its quick mechanism of action when paired with an oral antidepressant. (Daly, Singh et al. 2018) The success of this medication has spurred clinical research into psilocybin, a 5-HT2A agonist commonly derived from Psilocybe spp. mushrooms, which has rapid action similar to esketamine but appears to have a longer effectiveness despite fewer dosing regimens. (Griffiths, Johnson et al. 2016) While there is growing interest in these therapies, during treatment, there still needs to be extensive supervision and psychotherapy because of the hallucinogenic effects of these drugs. Consequently, there is a need to find and to develop other exogenous serotonergic modulators, e.g., for the treatment of mood disorders.

One avenue that researchers have explored is the use of triptans, which are a class of migraine medications that have substitutions at the 3- and 5-positions of the indole ring. To date, seven triptans have been approved by the FDA as 5-HT1B/1D agonists. These drugs were ultimately developed after early ergot alkaloids showed promise as vasoconstrictors for reduced postpartum bleeding and for the treatment of headaches. (Schiff 2006) The most famous ergot alkaloid, lysergic acid diethylamide (LSD), has promiscuous pharmacology throughout the central nervous system but is best described as a 5-HT1/2 partial agonist with potent hallucinogenic effects afforded by 5-HT2A agonism. (Passie, Halpern et al. 2008) The 2-brominated LSD derivative (BOL-148) inhibits serotonin receptors, and interestingly, is devoid of pyschoactivity.

BOL-148 (Karst, Halpern et al. 2010) is also being clinically evaluated to treat cluster headaches but could hold promise as an antidepressant as with other 5-HT2A antagonists like trazadone and nefazodone.

Despite the aforementioned therapeutics and agonists, researchers have only begun to explore the essentially limitless number of indole and indole derived compounds and their potential uses.

### SUMMARY OF THE INVENTION

The present invention is directed to 5-hydroxytryptamine receptor 2A (“5H2A”) ligands, methods of making these compounds, complexes formed between these (as well as other ligands) and receptors, formulations containing these compounds and uses of these compounds. In various embodiments, the compounds of the present invention link two indole containing moieties through a tertiary amine on an alkyl chain at the 3-position of the indole. In various embodiments, these compounds are dimerized compounds that have potent, and even selective, 5-HT2A receptor modulators.

Linking these tryptamines as both homodimers and heterodimers renders various compounds active at 5-HT2A, and various modifications of the indole scaffold may be used to improve pharmaceutical properties such as lipophilicity and stability. For this reason, various substitutions of nitrogen groups within the ring structure, referred to as azaindoles, are advantageous for 5-HT2A modulation with better drug-like properties.

According to a first embodiment, the present invention provides a compound of any of formulas I-VI:

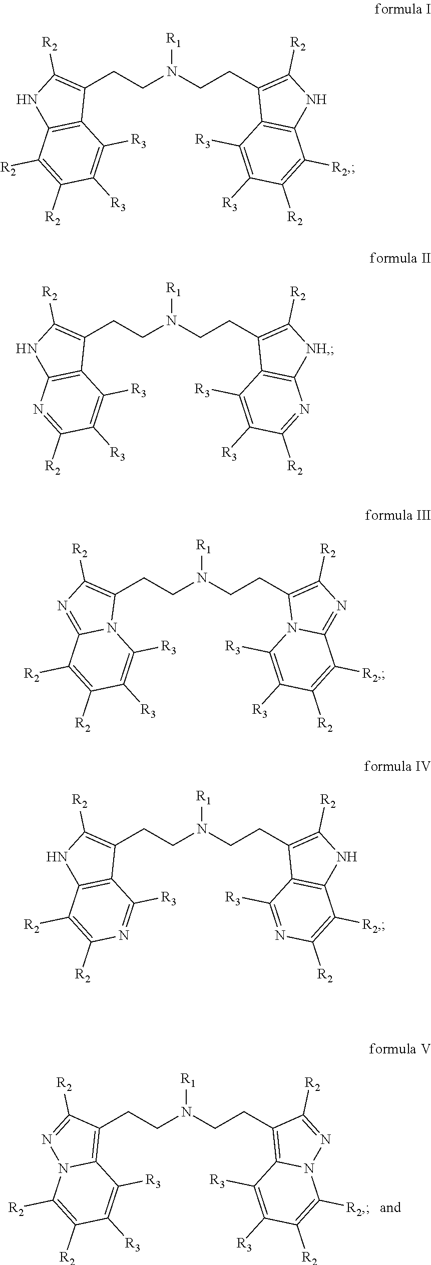

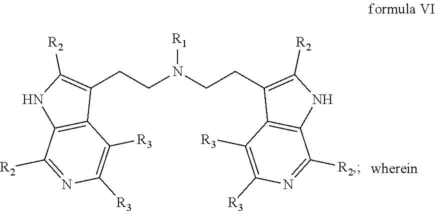

R₁ is H, D or an aliphatic substituent with a primary, secondary, tertiary, or quaternary amine;
R₂ is H, D, CH₃, CF₃ or halogen; and
R₃ is H, D, or an oxidized substituent with an alcohol, ester, ether, aldehyde, acid, amide, thiol, sulfone, sulfonamide, reversed sulfonamide or halogen.

According to a second embodiment, the present invention provides a pharmaceutical composition comprising a compound of any of formulas I to VI, wherein the compound is present in an amount that is an effective dose.

According to a third embodiment, the present invention provides a method of treating a neurological disorder or condition comprising administering a pharmaceutical composition of the present invention to a subject.

According to a fourth embodiment, the present invention provides a method of treating a mood disorder comprising administering a pharmaceutical composition of the present invention to a subject.

According to a fifth embodiment, the present invention provides a method of treating a substance abuse disorder comprising administering a pharmaceutical composition of the present invention to a subject.

According to a sixth embodiment, the present invention provides a medicament comprising a compound of any of formulas I to VI.

According to a seventh embodiment, the present invention provides use of a compound of any of formulas I to VI for treatment of a neurological disorder, a mood disorder or a substance abuse disorder.

According to an eighth embodiment, the present invention provides a ligand-receptor complex comprising: (a) a serotonin 2A receptor, wherein the serotonin 2A receptor comprises SEQ ID NO: 1 or a sequence that is at least 80% similar to SEQ ID NO: 1; and (b) a ligand, wherein the ligand and the serotonin 2A receptor have either or both of Van der Waals interactions and hydrogen bonds at either or both of ASP 155 and SER 242 of the serotonin 2A receptor, and further wherein as compared to an (+)-LSD-a serotonin 2A receptor complex in a normalized differential, within the ligand-receptor complex: (i) at one or more of THR 139, THR 160, and ASN 343 of the serotonin 2A receptor, the ligand forms hydrogen bonds that are at least 60% more of the normalized differential; or (ii) at one or more of ILE 135, TYR 139, GLY 238, SER 239 of the serotonin 2A receptor, the ligand forms Van der Waals interactions that are at least 60% more of the normalized differential; or (iii) at one or more of SER 131, LEU 229, GLY 238, SER 239, SER 242, ASN 363, TYR 370 of the serotonin 2A receptor, the ligand forms hydrogen bonds that are at least 60% less of the normalized distribution; or (iv) at one or more of ILE 152, CYS 227, LEU 228, LEU 229, PHE 243, LEU 362 of the serotonin 2A receptor, the ligand forms Van der Waals interactions that are at least 60% less of the normalized distribution.

### BRIEF DESCRIPTION OF THE FIGURES

FIG. 1 is a synthetic pathway for the production of a compound of the present invention. In FIG. 1 as well as in FIG. 2 and FIG. 3, the following acronyms are used: tert-butyldimethylsilyl chloride (TBSCl), dimethylformamide (DMF), room temperature (rt), diethyl ether (Et₂O), tetrahydrofuran (THF), 2-methyl tetrahydrofuran (2-Me-THF), N-methyltryptamine (NMT), lithium aluminum hydride (LAH), N-ethylcarbodiimide (EDC), 1-hydroxybenzotriazole (HOBt), sodium cyanoborohydride (NaBH₃CN), diisopropylethylamine (DIPEA), triethylamine (TEA), tert-butoxycarbonyl (Boc), and tetra-n-butylammonium fluoride (TBAF).

FIG. 2 is a synthetic pathway for the production of another compound of the present invention.

FIG. 3 is a synthetic pathway for the production of another compound of the present invention.

FIG. 4 is a bar graph that depicts normalized atomic Van der Waals interactions between sDMT (gray) and (+)-LSD (black) modeled in the active site of a Serotonin 2A Receptor.

FIG. 5 is a bar graph that depicts normalized hydrogen bond contacts between sDMT (gray) and (+)-LSD (black) modeled in the active site of a Serotonin 2A Receptor.

### DETAILED DESCRIPTION OF THE INVENTION

Reference will now be made in detail to various embodiments of the present invention, examples of which are illustrated in the accompanying figures. In the following description, numerous specific details are set forth in order to provide a thorough understanding of the present invention. However, unless otherwise indicated or implicit from context, the details are intended to be examples and should not be deemed to limit the scope of the invention in any way. Additionally, features described in connection with the various or specific embodiments are not to be construed as not appropriate for use in connection with other embodiments disclosed herein unless such exclusivity is explicitly stated or implicit from context.

Headers are provided herein for the convenience of the reader and do not limit the scope of any of the embodiments disclosed herein.

### Definitions

Unless otherwise stated or implicit from context the following terms and phrases have the meanings provided below.

The indefinite articles “a” and “an” and the definite article “the” include plural as well as singular referents, unless the context clearly dictates otherwise.

The terms “about” and “approximately” mean an acceptable error for a particular value as determined by one of ordinary skill in the art, which depends in part on how the value is measured or determined. In certain embodiments, the term “about” or “approximately” means within 1, 2, 3 or 4 standard deviations. In certain embodiments, the term “about” or “approximately” means with 30%, 20%, 15%, 10%, 9%, 8%, 7%, 6%, 5%, 4%, 3%, 2%, 1%, 0.5%, 0.1%, or 0.05%, of a given value or range.

The phrase “abuse disorder” refers to a disorder or disease that affects a person's brain and behavior and leads to an inability to control the use of a legal or illegal drug or medication. Prescription medicines, non-prescription medicines, and non-approved drugs may all be abused drugs, the use of which may lead to an abuse disorder. Drugs and medications may also include substances such as amphetamines, opioids, cocaine, barbiturates, alcohol, marijuana, and nicotine.

The phrase “effective amount” means the amount necessary to create a desirable result in a subject.

The term “hydrogen bond” refers to the electrostatic force of attraction between a hydrogen atom that is covalently bound to a more electronegative “donor” atom or group, and another electronegative atom bearing a lone pair of electrons—the hydrogen bond acceptor. Hydrogen bonds may, for example, have a length of about 2.7-3.3 Angstroms.

The terms “manage,” “managing,” and “management” refer to preventing or slowing the progression, spread or worsening of a disease or disorder, or of one or more symptoms thereof. Often, the beneficial effects that a subject derives from a prophylactic and/or therapeutic agent do not result in a cure of the disease or disorder.

In this regard, the term “managing” encompasses treating a subject who has suffered from the particular disease in an attempt to prevent or minimize the recurrence of the disease.

A “mood disorder” refers to a group of conditions in which a disturbance in the person's mood is the underlying feature. Mood disorders may be groups of mania (elevated mood disorders) or hypomania (depression). The classification is in the Diagnostic and Statistical Manual of Mental Disorders (DSM) and the International Classification of Diseases (ICD).

A “neurological disorder” refers to conditions and diseases of the central and peripheral nervous system e.g., the brain, spinal cord, cranial nerves, peripheral nerves, nerve roots, autonomic nervous system, neuromuscular junction, and muscles. These disorders include, but are not limited to, epilepsy, Alzheimer's disease and other dementias, cerebrovascular diseases including stroke, migraine, cluster headaches and other headache disorders, multiple sclerosis, Parkinson's disease, neuro-infections, brain tumors, traumatic disorders of the nervous system due to head trauma, and traumatic disorders due to traumatic or terrifying experiences (Posttraumatic Stress Disorder e.g., PTSD) and neurological disorders as a result of malnutrition and substance abuse. The substance abused may be any number of addictive substances, especially alcohol and drugs and combinations thereof. Additionally, many bacterial (e.g., Mycobacterial tuberculosis, Neisseria meningitides), viral (e.g., Human Immunodeficiency Virus (HIV), Lyme Disease, Enteroviruses, West Nile Virus, Zika), fungal (e.g., Cryptococcus, Aspergillus), amoebic (e.g. Naegleria, Acanthamoeba), and parasitic (e.g., malaria, Chagas) infections can affect the nervous system and lead to neurological disorders. Neurological symptoms that accompany these disorders may occur because of an infection itself, and/or an immune response.

A “pharmaceutically acceptable salt” is a salt that is of sufficient purity and quality for use in a formulation of a composition or medicament of the present invention. Both human use (clinical and over-the-counter) and veterinary use are included within the scope of the present invention. A formulation of the present invention includes a composition or medicament for either human or veterinary use. Pharmaceutically acceptable salts, include but are not limited to acid addition salts that have been formed with the free amino groups of a protein. Unless otherwise specified or apparent from context, any recitation of a compound includes its pharmaceutically acceptable salts.

The terms “prevent,” “preventing,” and “prevention” refer to the prevention of the onset, recurrence or spread of a disease or disorder or of one or more symptoms thereof. In certain embodiments, the terms refer to the treatment with or administration of a compounder dosage, with or without one or more other additional active agent(s), prior to the onset of symptoms, particularly to a subject at risk of diseases or disorders provided herein. The terms encompass the inhibition or reduction of a symptom of the particular disease. Subjects with familial history of a disease in particular are candidates for preventive regimes in certain embodiments. In addition, subjects who have a history of recurring symptoms are also potential candidates for prevention. In this regard, the term “prevention” may be interchangeably used with the term “prophylactic treatment.”

A “prophylactically effective amount” of a compound is an amount sufficient to prevent a disease or disorder or prevent its recurrence. A prophylactically effective amount of a compound means an amount of therapeutic agent, alone or in combination with one or more other agent(s), that provides a prophylactic benefit in the prevention of the disease. The term “prophylactically effective amount” can encompass an amount that improves overall prophylaxis or enhances the prophylactic efficacy of another prophylactic agent.

The term “subject” is defined herein to include animals such as mammals, including, but not limited to, primates (e.g., humans), cows, sheep, goats, horses, dogs, cats, rabbits, rats, mice, and the like. In certain embodiments, the subject is a human.

As used herein, and unless otherwise specified, the terms “therapeutically effective amount” and “effective amount” mean an amount sufficient to provide a therapeutic benefit in the treatment or management of a disease or disorder, or to delay or minimize one or more symptoms associated with the disease or disorder. The terms “therapeutically effective amount” and “effective amount” can encompass an amount that improves overall therapy, reduces or avoids symptoms or causes of disease or disorder, or enhances the therapeutic efficacy of another therapeutic agent.

The terms “treat,” “treating,” and “treatment” refer to the eradication or amelioration of a disease or disorder, or of one or more symptoms associated with the disease or disorder. In certain embodiments, the terms refer to minimizing the spread or worsening of the disease or disorder by administering of one or more prophylactic or therapeutic agents to a subject with such disease or disorder. In some embodiments the terms refer to the administration of a compound or dosage form provided herein, with or without one or more additional active agent(s), after the onset of symptoms of a particular disease.

The phrase “Van der Waals interaction” refers to an association between two atoms due to a force (the Van der Waals force) in which an atom such as oxygen or nitrogen draws an electron cloud toward itself through the covalent bond from its less electronegative neighbor atom. Van der Waals interactions are weaker than hydrogen bonds and may, for example, have a length of about 3.4-4.0 Angstroms.

### Compounds

Various embodiments, of the present invention are directed to different compounds within any of formulas I-VI:

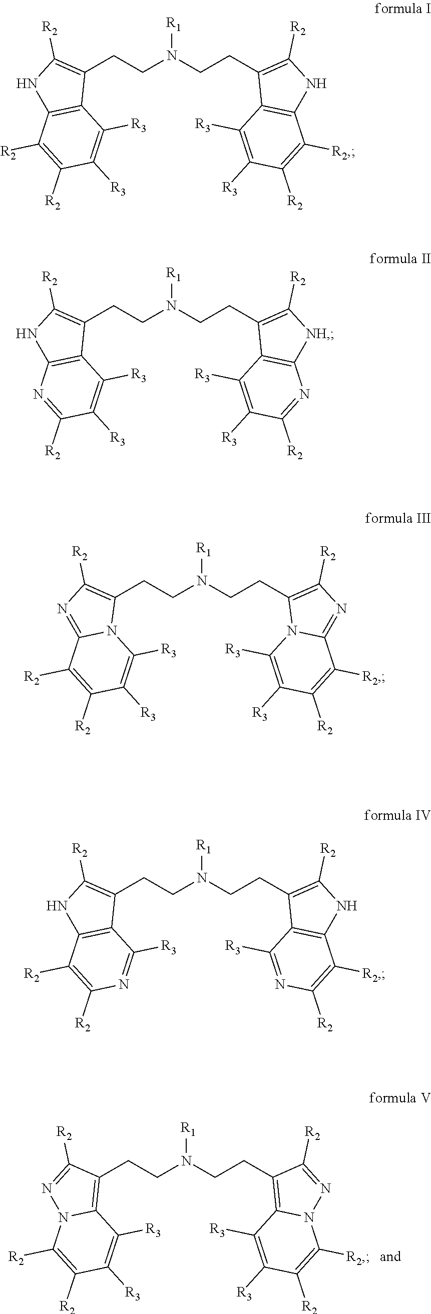

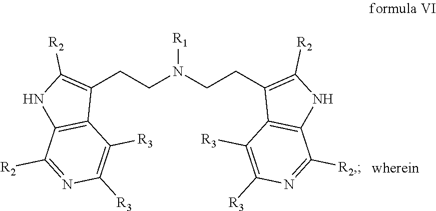

R₁ is H, D or an aliphatic substituent with a primary, secondary, tertiary, or quaternary amine;
R₂ is H, D, CH₃, CF₃ or halogen; and
R₃ is H, D, or an oxidized substituent with an alcohol, ester, ether, aldehyde, acid, amide, thiol, sulfone, sulfonamide, reversed sulfonamide, urea or halogen.

Within any compound and unless otherwise specified each R₂ may be different, a plurality but fewer than all of the R₂ moieties may be different, or all of the R₂ moieties may be the same. Similarly, within any compound and unless otherwise specified each R₃ may be different, a plurality but fewer than all of the R₃ moieties may be different, or all of the R₂ moieties may be the same.

In some embodiments, the compound is within formula I. For example, the compound may be:

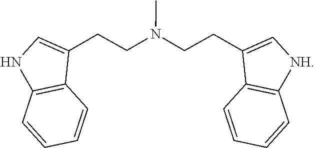 [SMILES: CN(CCc1c[nH]c2ccccc12)CCc1c[nH]c2ccccc12]

In some embodiments, the compound is within formula II. For example, the compound may be:

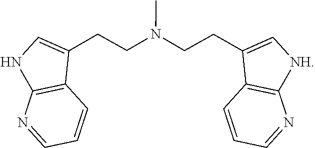 [SMILES: CN(CCc1c[nH]c2ncccc12)CCc1c[nH]c2ncccc12]

In some embodiments, the compound is within formula III. For example, the compound may be:

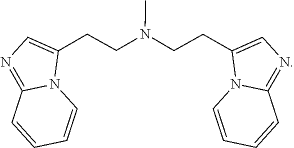 [SMILES: CN(CCc1cnc2ccccn12)CCc1cnc2ccccn12]

In some embodiments, the compound is within formula IV. For example, the compound may be:

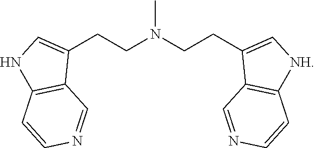 [SMILES: CN(CCc1c[nH]c2ccncc12)CCc1c[nH]c2ccncc12]

In some embodiments, the compound is within formula V.

In some embodiments, the compound is within formula VI.

In some embodiments, R₁, every R₂, and every R₃ are H. In some embodiments, R₁, at least one R₂, and at least one R₃ are H. In some embodiments, R₁, every R₂, and every R₃ are D. In some embodiments, R₁, at least one R₂, and at least one R₃ are D. In some embodiments, R₁ and every R₃ are H, while every R₂ is a methyl group. In some embodiments, R₁ and every R₃ are H, while every R₂ is a halogen, e.g., F or Br.

In some embodiments, the compound is selected from the group consisting of the compounds listed in Table 1, which appears at the end of this specification before the examples section.

### Synthesis

By way of a non-limiting example, compounds of formula I can be synthesized as follows: Indole-3-acetic acid can be coupled to tryptamine via a peptide coupling reagent. The amide can be reduced with a reducing agent (e.g., lithium aluminum hydride) in refluxing solvent. Finally, the N-methyl group(s) can be installed via a reductive amination with the appropriate aldehyde. An example of a compound that can be synthesized according to this protocol is N-(2-(1H-indol-3-yl)ethyl)-2-(1H-indol-3-yl)-N-methylethan-1-amine.

By way of further example of methods for synthesizing compounds of the present invention, azaindole (at the 1a, 3a, 5, 6, or 7 position) can be treated with methyl chlorooxoacetate and a Lewis acid in an appropriate solvent. The methyl ester can be saponified to form the carboxylic acid intermediate. The acid chloride, formed in the presence of a chlorinating reagent, can be treated with methylamine to create an alpha ketoamide. The carboxylic acid intermediate can then be converted to the acid chloride and treated with a methyl amide to form an imide, which can then be reduced with a strong reducing agent (e.g., lithium aluminum hydride) under refluxing conditions to form the final product as the azatryptamine dimer of choice. Finally, the R alkyl group can be installed via a reductive amination with the appropriate aldehyde.

### Ligand-Receptor Complexes

In some embodiments, the present invention provides a ligand-receptor complex. A ligand-receptor complex contains both a ligand and a receptor in which association between a ligand and a receptor is maintained by hydrogen bonds and or Van der Waals interactions. In some embodiments, the receptor is a serotonin 2A receptor. The serotonin 2A receptor may comprise, consist essentially of, or consist of a protein that has the amino acid sequence of SEQ ID NO: 1 or a sequence that is at least 80%, at least 85%, at least 90%, or at least at least 95% similar to SEQ ID NO: 1. SEQ ID NO: 1 is

MKTIIALSYIFCLVFADYKDDDDGAPLSLLHLQEKNWSALLTAVV
IILTIAGNILVIMAVSLEKKLQNATNYFLMSLAIADMLLGFLVMP
VSMLTILYGYRWPLPSKLCAVWIYLDVLFSTASIMHLCAISLDRY
VAIQNPIHHSRFNSRTKAFLKIIAVWTISVGISMPIPVFGLQDDS
KVFKEGSCLLADDNFVLIGSFVSFFIPATIMVITYFLTIKSLQKE
AADLEDNWETLNDNLKVIEKADNAAQVKDALTKMRAAALDAQKAT
PPKLEDKSPDSPEMKDFRHGFDILVGQIDDALKLANEGKVKEAQA
AAEQLKTTRNAYIQKYLTMOSISNEQKACKVLGIVFFLFVVMWCP
FFITNIMAVICKESCNEDVIGALLNVFVWIGYASSAVNPLVYTLE
NKTYRSAFSRYIQCQYKENKKPGRPLEVLFQGPHHHHHHHHHH.

When the receptor is not identical to SEQ ID NO: 1, this may be due to one or more deletions, one or more insertions, and/or one or more substitutions of amino acids. As persons of ordinary skill in the art will recognize, when there is a deletion or insertion of one or more amino acids, the numbering of the amino acids after the deletion will change by the number of amino acids deleted or inserted. As used herein, the position number of amino acids refers to the positions in SEQ ID NO: 1, and it is implicit that such numbering includes any higher or lower position at which that amino acid would be located due the addition and or deletion of other amino acids. Thus, the amino acid with which the ligand forms the hydrogen bond or Van der Waals force would not change, but it location might in a truncated or elongated variant of SEQ ID NO: 1.

A ligand is a molecule that is capable of forming Van der Waals interactions and hydrogen bonds with the receptor, and e.g., the ligand many be a molecule within any of formula I, formula II, formula III, formula IV, formula V or formula VI, or any other molecule (e.g., other dimers of derivatives of indole) that is capable of interacting with the receptor as described herein.

The Van der Waals interactions and hydrogen bonds within the ligand-receptor complexes of the present invention are described relative to and distinct from those within an (+)-LSD-a serotonin 2A receptor complex. Techniques for measuring Van der Waals interactions and hydrogen bonds include but are not limited to solvent, all-atom molecular dynamics, which are, for example, described in Guvench O, MacKerell A D Jr (2009) Computational Fragment-Based Binding Site Identification by Ligand Competitive Saturation. PLOS Comput Biol 5 (7): e1000435, doi:10.1371/journal.pcbi.1000435, the entire disclosure of which is incorporated by reference as if set forth herein, as well as more cumbersome but more well-known methods such as NMR and X-ray crystallography.

In some embodiments, within the ligand-receptor complex of the present invention the ligand and the serotonin 2A receptor have either or both of Van der Waals interactions and hydrogen bonds at either or both of ASP 155 and SER 242 of the serotonin 2A receptor.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at one or more of THR 139, THR 160, and ASN 343 of the serotonin 2A receptor, the ligand forms hydrogen bonds that are at least 60% more of the normalized differential; or
at one or more of ILE 135, TYR 139, GLY 238, and SER 239 of the serotonin 2A receptor, the ligand forms Van der Waals interactions that are at least 60% more of the normalized differential; or
at one or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at one or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at two or more of THR 139, THR 160, and ASN 343 of the serotonin 2A receptor, the ligand forms hydrogen bonds that are at least 60% more of the normalized differential; or
at two or more of ILE 135, TYR 139, GLY 238, and SER 239 of the serotonin 2A receptor, the ligand forms Van der Waals interactions that are at least 60% more of the normalized differential; or
at two or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at two or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at each of THR 139, THR 160, and ASN 343 of the serotonin 2A receptor, the ligand forms hydrogen bonds that are at least 60% more of the normalized differential; or
at three or more of ILE 135, TYR 139, GLY 238, and SER 239 of the serotonin 2A receptor, the ligand forms Van der Waals interactions that are at least 60% more of the normalized differential; or
at three or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at three or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at each of THR 139, THR 160, and ASN 343 of the serotonin 2A receptor, the ligand forms hydrogen bonds that are at least 60% more of the normalized differential; or
at each of ILE 135, TYR 139, GLY 238, and SER 239 of the serotonin 2A receptor, the ligand forms Van der Waals interactions that are at least 60% more of the normalized differential; or.
at four or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at four or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at each of THR 139, THR 160, and ASN 343 of the serotonin 2A receptor, the ligand forms hydrogen bonds that are at least 60% more of the normalized differential; or
at each of ILE 135, TYR 139, GLY 238, and SER 239 of the serotonin 2A receptor, the ligand forms Van der Waals interactions that are at least 60% more of the normalized differential; or
at five or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at five or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, where as compared to an (+)-LSD-a serotonin 2A receptor complex in a normalized differential, within the ligand-receptor complex at least one of the following conditions exists:
at each of THR 139, THR 160, and ASN 343 of the serotonin 2A receptor, the ligand forms hydrogen bonds that are at least 60% more of the normalized differential; or
at each of ILE 135, TYR 139, GLY 238, and SER 239 of the serotonin 2A receptor, the ligand forms Van der Waals interactions that are at least 60% more of the normalized differential; or
at six or more of SER 131, LEU 229, GLY 238, SER 239, SER 242, ASN 363, and TYR 370 of the serotonin 2A receptor, the ligand forms hydrogen bonds that are at least 60% less of the normalized distribution; or
at each of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex at least one of the following conditions exists:
at each of THR 139, THR 160, and ASN 343 of the serotonin 2A receptor, the ligand forms hydrogen bonds that are at least 60% more of the normalized differential; or
at each of ILE 135, TYR 139, GLY 238, and SER 239 of the serotonin 2A receptor, the ligand forms Van der Waals interactions that are at least 60% more of the normalized differential; or
at each of SER 131, LEU 229, GLY 238, SER 239, SER 242, ASN 363, and TYR 370 of the serotonin 2A receptor, the ligand forms hydrogen bonds that are at least 60% less of the normalized distribution; or
at each of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at one or more of THR 139, THR 160, and ASN 343 of the serotonin 2A receptor, the ligand forms hydrogen bonds that are at least 60% more of the normalized differential; and
at one or more of ILE 135, TYR 139, GLY 238, and SER 239 of the serotonin 2A receptor, the ligand forms Van der Waals interactions that are at least 60% more of the normalized differential; and
at one or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at one or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at two or more of THR 139, THR 160, and ASN 343 of the serotonin 2A receptor, the ligand forms hydrogen bonds that are at least 60% more of the normalized differential; and
at two or more of ILE 135, TYR 139, GLY 238, and SER 239 of the serotonin 2A receptor, the ligand forms Van der Waals interactions that are at least 60% more of the normalized differential; and
at two or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at two or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at each of THR 139, THR 160, and ASN 343 of the serotonin 2A receptor, the ligand forms hydrogen bonds that are at least 60% more of the normalized differential; and
at three or more of ILE 135, TYR 139, GLY 238, and SER 239 of the serotonin 2A receptor, the ligand forms Van der Waals interactions that are at least 60% more of the normalized differential; and
at three or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at three or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at each of THR 139, THR 160, and ASN 343 of the serotonin 2A receptor, the ligand forms hydrogen bonds that are at least 60% more of the normalized differential; and
at each of ILE 135, TYR 139, GLY 238, and SER 239 of the serotonin 2A receptor, the ligand forms Van der Waals interactions that are at least 60% more of the normalized differential; and
at four or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at four or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at each of THR 139, THR 160, and ASN 343 of the serotonin 2A receptor, the ligand forms hydrogen bonds that are at least 60% more of the normalized differential; and
at each of ILE 135, TYR 139, GLY 238, and SER 239 of the serotonin 2A receptor, the ligand forms Van der Waals interactions that are at least 60% more of the normalized differential; and
at five or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at five or more of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, where as compared to an (+)-LSD-a serotonin 2A receptor complex in a normalized differential, within the ligand-receptor complex the following conditions exist:
at each of THR 139, THR 160, and ASN 343 of the serotonin 2A receptor, the ligand forms hydrogen bonds that are at least 60% more of the normalized differential; and
at each of ILE 135, TYR 139, GLY 238, and SER 239 of the serotonin 2A receptor, the ligand forms Van der Waals interactions that are at least 60% more of the normalized differential; and
at six or more of SER 131, LEU 229, GLY 238, SER 239, SER 242, ASN 363, and TYR 370 of the serotonin 2A receptor, the ligand forms hydrogen bonds that are at least 60% less of the normalized distribution; and
at each of ILE 152, CYS 227, LEU 228, LEU 229, PHE 243, and LEU 362 of the serotonin 2A receptor, the ligand forms Van der Waals interactions that are at least 60% less of the normalized distribution.

In some embodiments, in addition to the aforementioned Van der Waals interactions and hydrogen bonds at ASP 155 and SER 242 of the serotonin 2A receptor, as compared to an (+)-LSD-a serotonin 2A receptor complex in a normalized differential, within the ligand-receptor complex the following conditions exist:
at each of THR 139, THR 160, and ASN 343 of the serotonin 2A receptor, the ligand forms hydrogen bonds that are at least 60% more of the normalized differential; and
at each of ILE 135, TYR 139, GLY 238, and SER 239 of the serotonin 2A receptor, the ligand forms Van der Waals interactions that are at least 60% more of the normalized differential; and
at each of SER 131, LEU 229, GLY 238, SER 239, SER 242, ASN 363, and TYR 370 of the serotonin 2A receptor, the ligand forms hydrogen bonds that are at least 60% less of the normalized distribution; and
at each of ILE 152, CYS 227, LEU 228, LEU 229, PHE 243, and LEU 362 of the serotonin 2A receptor, the ligand forms Van der Waals interactions that are at least 60% less of the normalized distribution.

The aforementioned percentages are all recited as being at least 60% more or at least 60% less of the normalized distribution. In some embodiments, one, more than one, or each of those percentages is at least 65% more or at least 65% less; at least 70% more or at least 70% less; at least 75% more or at least 75% less; at least 80% more or at least 80% less; at least 85% more or at least 85% less; or at least 90% more or at least 90% less.

The aforementioned ligand-receptor complexes may, for example, exist in vivo, in vitro, or ex vivo. In some embodiments, the complex forms while the receptor is part of a cell that may be located within or outside of a subject, e.g., a mammal. The mammal may, for example, be a primate such as a human, or a non-human primate or an organism other than a primate.

### Methods of Use

The compounds of the present invention may be the active ingredient in, or one of a plurality of active ingredients in, formulations or medicaments that are designed to deliver these compounds. The formulations or medicaments may, for example, be applied transdermally and be in the form of sprayable liquids, gels, creams, lotions, ointments, or transdermal patches.

Alternatively they may be delivered orally. Oral formulations may be manufactured in the form of tablets, capsules, soft gels, strips, and oral patches.

Alternatively they may be delivered, intranasally. Intranasal administration may, for example, be in the form of a mist that is delivered to the nasal cavity.

In some embodiments, a compound of any of formulas I-VI is co-administered with one or more therapeutic agents. Co-administration may be simultaneously or sequentially. The co-administered agent may, for example, be a monoamine oxidase inhibitor (“MAOI”). MAOIs include, but are not limited to, harmala alkaloids, harmine, harmane, harmaline, hydrazine, iproniazid, isocarboxazid, nialamide, phenelzine, hydracarbazine, tranylcypromine, bifemelane, moclobemide, pirlindole, toloxatone, rasagiline, selegiline, safinamide, and other reversible inhibitors of monoamine oxidase A (RIMAs).

In another embodiment, a compound from another class of neurologically active agents is co-administered with a compound of any of formulas I-VI, thereby providing for a synergistic therapeutic effect. Other neurologically active agents include, but are not limited to, those compounds that fall into the following classes: antipsychotics, antidepressants, anxiolytics, stimulants, reuptake inhibitors (SSRI or SSNRI), cognitive-enhancing agents, tricyclic antidepressants, mood stabilizers, NMDA antagonists and 5-HT antagonists.

In some embodiments, a compound of any of formulas I-VI is co-administered with one or more therapeutic agents to reduce substance abuse. For the treatment of opioid addiction, other co-administered compounds can include, but are not limited to: methadone, buprenorphine, naloxone, naltrexone, and the like. For the treatment of alcoholism, other co-administered compounds can include, but are not limited to: ethyl alcohol, disulfiram, naltrexone, acamprosate, benzodiazepines, and the like. For the treatment of nicotine addiction, other co-administered compounds can include, but are not limited to: low dose nicotine, Bupropion, and Varenicline.

The formulations of the present invention may also comprise one or more excipients. Thus, in some embodiments, the present invention is directed to a composition or medicant that comprises a compound of any of formulas I-VI and one or more excipients. These excipients may, for example, act as one or more of diluents, binders, disintegrants, lubricants, coatings or films, and coloring agents. The formulations that contain these excipients may be designed for immediate release, controlled release or extended release.

TABLE 1
Compounds

N-(2-(1H-indol-3-yl)ethyl)-2-(1H-indol-3-yl)-N-methylethan-1-amine
N-(2-(1H-indol-3-yl)ethyl)-N-ethyl-2-(1H-indol-3-yl)ethan-1-amine
N,N-bis(2-(1H-indol-3-yl)ethyl)propan-2-amine
2(2-(4-D-1H-3a-azaindol-3-yl)-N-(-N-(2-(4-D-1H-3a-azaindol-3-yl)ethyl)-N-ethyl)-N-methylethan-1-
amine
2(2-(5-OH-1H-3a-azaindol-3-yl)-N-(-N-(2-(4-D-1H-3a-azaindol-3-yl)ethyl)-N-ethyl)-N-methylethan-1-
amine
2(2-(6-CF3-1H-3a-azaindol-3-yl)-N-(-N-(2-(4-D-1H-3a-azaindol-3-yl)ethyl)-N-ethyl)-N-methylethan-1-
amine
2(2-(7-F-1H-3a-azaindol-3-yl)-N-(-N-(2-(4-D-1H-3a-azaindol-3-yl)ethyl)-N-ethyl)-N-methylethan-1-
amine
2(2-(4-D-1H-3a-azaindol-3-yl)-N-(-N-(2-(5-OH-1H-3a-azaindol-3-yl)ethyl)-N-ethyl)-N-methylethan-1-
amine
2(2-(5-OH-1H-3a-azaindol-3-yl)-N-(-N-(2-(5-OH-1H-3a-azaindol-3-yl)ethyl)-N-ethyl)-N-methylethan-1-
amine
2(2-(6-CF3-1H-3a-azaindol-3-yl)-N-(-N-(2-(5-OH-1H-3a-azaindol-3-yl)ethyl)-N-ethyl)-N-methylethan-1-
amine
2(2-(7-F-1H-3a-azaindol-3-yl)-N-(-N-(2-(5-OH-1H-3a-azaindol-3-yl)ethyl)-N-ethyl)-N-methylethan-1-
amine
2(2-(4-D-1H-3a-azaindol-3-yl)-N-(-N-(2-(6-CF3-1H-3a-azaindol-3-yl)ethyl)-N-ethyl)-N-methylethan-1-
amine
2(2-(5-OH-1H-3a-azaindol-3-yl)-N-(-N-(2-(6-CF3-1H-3a-azaindol-3-yl)ethyl)-N-ethyl)-N-methylethan-1-
amine
2(2-(6-CF3-1H-3a-azaindol-3-yl)-N-(-N-(2-(6-CF3-1H-3a-azaindol-3-yl)ethyl)-N-ethyl)-N-methylethan-1-
amine
2(2-(7-F-1H-3a-azaindol-3-yl)-N-(-N-(2-(6-CF3-1H-3a-azaindol-3-yl)ethyl)-N-ethyl)-N-methylethan-1-
amine
2(2-(4-D-1H-3a-azaindol-3-yl)-N-(-N-(2-(7-F-1H-3a-azaindol-3-yl)ethyl)-N-ethyl)-N-methylethan-1-
amine
2(2-(5-OH-1H-3a-azaindol-3-yl)-N-(-N-(2-(7-F-1H-3a-azaindol-3-yl)ethyl)-N-ethyl)-N-methylethan-1-
amine
2(2-(6-CF3-1H-3a-azaindol-3-yl)-N-(-N-(2-(7-F-1H-3a-azaindol-3-yl)ethyl)-N-ethyl)-N-methylethan-1-
amine
2(2-(7-F-1H-3a-azaindol-3-yl)-N-(-N-(2-(7-F-1H-3a-azaindol-3-yl)ethyl)-N-ethyl)-N-methylethan-1-
amine
2(2-(4-D-1H-3a-azaindol-3-yl)-N-(-N-(2-(4-D-1H-3a-azaindol-3-yl)ethyl)-N-ethyl)-N-ethylethan-1-amine
2(2-(5-OH-1H-3a-azaindol-3-yl)-N-(-N-(2-(4-D-1H-3a-azaindol-3-yl)ethyl)-N-ethyl)-N-ethylethan-1-
amine
2(2-(6-CF3-1H-3a-azaindol-3-yl)-N-(-N-(2-(4-D-1H-3a-azaindol-3-yl)ethyl)-N-ethyl)-N-ethylethan-1-
amine
2(2-(7-F-1H-3a-azaindol-3-yl)-N-(-N-(2-(4-D-1H-3a-azaindol-3-yl)ethyl)-N-ethyl)-N-ethylethan-1-amine
2(2-(4-D-1H-3a-azaindol-3-yl)-N-(-N-(2-(5-OH-1H-3a-azaindol-3-yl)ethyl)-N-ethyl)-N-ethylethan-1-
amine
2(2-(5-OH-1H-3a-azaindol-3-yl)-N-(-N-(2-(5-OH-1H-3a-azaindol-3-yl)ethyl)-N-ethyl)-N-ethylethan-1-
amine
2(2-(6-CF3-1H-3a-azaindol-3-yl)-N-(-N-(2-(5-OH-1H-3a-azaindol-3-yl)ethyl)-N-ethyl)-N-ethylethan-1-
amine
2(2-(7-F-1H-3a-azaindol-3-yl)-N-(-N-(2-(5-OH-1H-3a-azaindol-3-yl)ethyl)-N-ethyl)-N-ethylethan-1-
amine
2(2-(4-D-1H-3a-azaindol-3-yl)-N-(-N-(2-(6-CF3-1H-3a-azaindol-3-yl)ethyl)-N-ethyl)-N-ethylethan-1-
amine
2(2-(5-OH-1H-3a-azaindol-3-yl)-N-(-N-(2-(6-CF3-1H-3a-azaindol-3-yl)ethyl)-N-ethyl)-N-ethylethan-1-
amine
2(2-(6-CF3-1H-3a-azaindol-3-yl)-N-(-N-(2-(6-CF3-1H-3a-azaindol-3-yl)ethyl)-N-ethyl)-N-ethylethan-1-
amine
2(2-(7-F-1H-3a-azaindol-3-yl)-N-(-N-(2-(6-CF3-1H-3a-azaindol-3-yl)ethyl)-N-ethyl)-N-ethylethan-1-
amine
2(2-(4-D-1H-3a-azaindol-3-yl)-N-(-N-(2-(7-F-1H-3a-azaindol-3-yl)ethyl)-N-ethyl)-N-ethylethan-1-amine
2(2-(5-OH-1H-3a-azaindol-3-yl)-N-(-N-(2-(7-F-1H-3a-azaindol-3-yl)ethyl)-N-ethyl)-N-ethylethan-1-
amine
2(2-(6-CF3-1H-3a-azaindol-3-yl)-N-(-N-(2-(7-F-1H-3a-azaindol-3-yl)ethyl)-N-ethyl)-N-ethylethan-1-
amine
2(2-(7-F-1H-3a-azaindol-3-yl)-N-(-N-(2-(7-F-1H-3a-azaindol-3-yl)ethyl)-N-ethyl)-N-ethylethan-1-amine
2(2-(4-D-1H-3a-azaindol-3-yl)-N-(-N-(2-(4-D-1H-3a-azaindol-3-yl)ethyl)-N-ethyl)-N-isopropylethan-1-
amine
2(2-(5-OH-1H-3a-azaindol-3-yl)-N-(-N-(2-(4-D-1H-3a-azaindol-3-yl)ethyl)-N-ethyl)-N-isopropylethan-1-
amine
2(2-(6-CF3-1H-3a-azaindol-3-yl)-N-(-N-(2-(4-D-1H-3a-azaindol-3-yl)ethyl)-N-ethyl)-N-isopropylethan-1-
amine
2(2-(7-F-1H-3a-azaindol-3-yl)-N-(-N-(2-(4-D-1H-3a-azaindol-3-yl)ethyl)-N-ethyl)-N-isopropylethan-1-
amine
2(2-(4-D-1H-3a-azaindol-3-yl)-N-(-N-(2-(5-OH-1H-3a-azaindol-3-yl)ethyl)-N-ethyl)-N-isopropylethan-1-
amine
2(2-(5-OH-1H-3a-azaindol-3-yl)-N-(-N-(2-(5-OH-1H-3a-azaindol-3-yl)ethyl)-N-ethyl)-N-isopropylethan-
1-amine
2(2-(6-CF3-1H-3a-azaindol-3-yl)-N-(-N-(2-(5-OH-1H-3a-azaindol-3-yl)ethyl)-N-ethyl)-N-isopropylethan-
1-amine
2(2-(7-F-1H-3a-azaindol-3-yl)-N-(-N-(2-(5-OH-1H-3a-azaindol-3-yl)ethyl)-N-ethyl)-N-isopropylethan-1-
amine
2(2-(4-D-1H-3a-azaindol-3-yl)-N-(-N-(2-(6-CF3-1H-3a-azaindol-3-yl)ethyl)-N-ethyl)-N-isopropylethan-1-
amine
2(2-(5-OH-1H-3a-azaindol-3-yl)-N-(-N-(2-(6-CF3-1H-3a-azaindol-3-yl)ethyl)-N-ethyl)-N-isopropylethan-
1-amine
2(2-(6-CF3-1H-3a-azaindol-3-yl)-N-(-N-(2-(6-CF3-1H-3a-azaindol-3-yl)ethyl)-N-ethyl)-N-isopropylethan-
1-amine
2(2-(7-F-1H-3a-azaindol-3-yl)-N-(-N-(2-(6-CF3-1H-3a-azaindol-3-yl)ethyl)-N-ethyl)-N-isopropylethan-1-
amine
2(2-(4-D-1H-3a-azaindol-3-yl)-N-(-N-(2-(7-F-1H-3a-azaindol-3-yl)ethyl)-N-ethyl)-N-isopropylethan-1-
amine
2(2-(5-OH-1H-3a-azaindol-3-yl)-N-(-N-(2-(7-F-1H-3a-azaindol-3-yl)ethyl)-N-ethyl)-N-isopropylethan-1-
amine
2(2-(6-CF3-1H-3a-azaindol-3-yl)-N-(-N-(2-(7-F-1H-3a-azaindol-3-yl)ethyl)-N-ethyl)-N-isopropylethan-1-
amine
2(2-(7-F-1H-3a-azaindol-3-yl)-N-(-N-(2-(7-F-1H-3a-azaindol-3-yl)ethyl)-N-ethyl)-N-isopropylethan-1-
amine
2(2-(4-D-1H-3a-azaindol-3-yl)-N-(-N-(2-(4-D-1H-3a-azaindol-3-yl)ethyl)-N-ethyl)-N-allylethan-1-amine
2(2-(5-OH-1H-3a-azaindol-3-yl)-N-(-N-(2-(4-D-1H-3a-azaindol-3-yl)ethyl)-N-ethyl)-N-allylethan-1-
amine
2(2-(6-CF3-1H-3a-azaindol-3-yl)-N-(-N-(2-(4-D-1H-3a-azaindol-3-yl)ethyl)-N-ethyl)-N-allylethan-1-
amine
2(2-(7-F-1H-3a-azaindol-3-yl)-N-(-N-(2-(4-D-1H-3a-azaindol-3-yl)ethyl)-N-ethyl)-N-allylethan-1-amine
2(2-(4-D-1H-3a-azaindol-3-yl)-N-(-N-(2-(5-OH-1H-3a-azaindol-3-yl)ethyl)-N-ethyl)-N-allylethan-1-
amine
2(2-(5-OH-1H-3a-azaindol-3-yl)-N-(-N-(2-(5-OH-1H-3a-azaindol-3-yl)ethyl)-N-ethyl)-N-allylethan-1-
amine
2(2-(6-CF3-1H-3a-azaindol-3-yl)-N-(-N-(2-(5-OH-1H-3a-azaindol-3-yl)ethyl)-N-ethyl)-N-allylethan-1-
amine
2(2-(7-F-1H-3a-azaindol-3-yl)-N-(-N-(2-(5-OH-1H-3a-azaindol-3-yl)ethyl)-N-ethyl)-N-allylethan-1-amine
2(2-(4-D-1H-3a-azaindol-3-yl)-N-(-N-(2-(6-CF3-1H-3a-azaindol-3-yl)ethyl)-N-ethyl)-N-allylethan-1-
amine
2(2-(5-OH-1H-3a-azaindol-3-yl)-N-(-N-(2-(6-CF3-1H-3a-azaindol-3-yl)ethyl)-N-ethyl)-N-allylethan-1-
amine
2(2-(6-CF3-1H-3a-azaindol-3-yl)-N-(-N-(2-(6-CF3-1H-3a-azaindol-3-yl)ethyl)-N-ethyl)-N-allylethan-1-
amine
2(2-(7-F-1H-3a-azaindol-3-yl)-N-(-N-(2-(6-CF3-1H-3a-azaindol-3-yl)ethyl)-N-ethyl)-N-allylethan-1-
amine
2(2-(4-D-1H-3a-azaindol-3-yl)-N-(-N-(2-(7-F-1H-3a-azaindol-3-yl)ethyl)-N-ethyl)-N-allylethan-1-amine
2(2-(5-OH-1H-3a-azaindol-3-yl)-N-(-N-(2-(7-F-1H-3a-azaindol-3-yl)ethyl)-N-ethyl)-N-allylethan-1-amine
2(2-(6-CF3-1H-3a-azaindol-3-yl)-N-(-N-(2-(7-F-1H-3a-azaindol-3-yl)ethyl)-N-ethyl)-N-allylethan-1-
amine
2(2-(7-F-1H-3a-azaindol-3-yl)-N-(-N-(2-(7-F-1H-3a-azaindol-3-yl)ethyl)-N-ethyl)-N-allylethan-1-amine
2(2-(4-D-1H-3a-azaindol-3-yl)-N-(-N-(2-(4-D-1H-3a-azaindol-3-yl)ethyl)-N-ethyl)-N-hydroethan-1-
amine
2(2-(5-OH-1H-3a-azaindol-3-yl)-N-(-N-(2-(4-D-1H-3a-azaindol-3-yl)ethyl)-N-ethyl)-N-hydroethan-1-
amine
2(2-(6-CF3-1H-3a-azaindol-3-yl)-N-(-N-(2-(4-D-1H-3a-azaindol-3-yl)ethyl)-N-ethyl)-N-hydroethan-1-
amine
2(2-(7-F-1H-3a-azaindol-3-yl)-N-(-N-(2-(4-D-1H-3a-azaindol-3-yl)ethyl)-N-ethyl)-N-hydroethan-1-amine
2(2-(4-D-1H-3a-azaindol-3-yl)-N-(-N-(2-(5-OH-1H-3a-azaindol-3-yl)ethyl)-N-ethyl)-N-hydroethan-1-
amine
2(2-(5-OH-1H-3a-azaindol-3-yl)-N-(-N-(2-(5-OH-1H-3a-azaindol-3-yl)ethyl)-N-ethyl)-N-hydroethan-1-
amine
2(2-(6-CF3-1H-3a-azaindol-3-yl)-N-(-N-(2-(5-OH-1H-3a-azaindol-3-yl)ethyl)-N-ethyl)-N-hydroethan-1-
amine
2(2-(7-F-1H-3a-azaindol-3-yl)-N-(-N-(2-(5-OH-1H-3a-azaindol-3-yl)ethyl)-N-ethyl)-N-hydroethan-1-
amine
2(2-(4-D-1H-3a-azaindol-3-yl)-N-(-N-(2-(6-CF3-1H-3a-azaindol-3-yl)ethyl)-N-ethyl)-N-hydroethan-1-
amine
2(2-(5-OH-1H-3a-azaindol-3-yl)-N-(-N-(2-(6-CF3-1H-3a-azaindol-3-yl)ethyl)-N-ethyl)-N-hydroethan-1-
amine
2(2-(6-CF3-1H-3a-azaindol-3-yl)-N-(-N-(2-(6-CF3-1H-3a-azaindol-3-yl)ethyl)-N-ethyl)-N-hydroethan-1-
amine
2(2-(7-F-1H-3a-azaindol-3-yl)-N-(-N-(2-(6-CF3-1H-3a-azaindol-3-yl)ethyl)-N-ethyl)-N-hydroethan-1-
amine
2(2-(4-D-1H-3a-azaindol-3-yl)-N-(-N-(2-(7-F-1H-3a-azaindol-3-yl)ethyl)-N-ethyl)-N-hydroethan-1-amine
2(2-(5-OH-1H-3a-azaindol-3-yl)-N-(-N-(2-(7-F-1H-3a-azaindol-3-yl)ethyl)-N-ethyl)-N-hydroethan-1-
amine
2(2-(6-CF3-1H-3a-azaindol-3-yl)-N-(-N-(2-(7-F-1H-3a-azaindol-3-yl)ethyl)-N-ethyl)-N-hydroethan-1-
amine
2(2-(7-F-1H-3a-azaindol-3-yl)-N-(-N-(2-(7-F-1H-3a-azaindol-3-yl)ethyl)-N-ethyl)-N-hydroethan-1-amine
2(2-(4-D-1H-3a-azaindol-3-yl)-N-(-N-(2-(4-D-1H-3a-azaindol-3-yl)ethyl)-N-ethyl)-N-(D)ethan-1-amine
2(2-(5-OH-1H-3a-azaindol-3-yl)-N-(-N-(2-(4-D-1H-3a-azaindol-3-yl)ethyl)-N-ethyl)-N-(D)ethan-1-amine
2(2-(6-CF3-1H-3a-azaindol-3-yl)-N-(-N-(2-(4-D-1H-3a-azaindol-3-yl)ethyl)-N-ethyl)-N-(D)ethan-1-amine
2(2-(7-F-1H-3a-azaindol-3-yl)-N-(-N-(2-(4-D-1H-3a-azaindol-3-yl)ethyl)-N-ethyl)-N-(D)ethan-1-amine
2(2-(4-D-1H-3a-azaindol-3-yl)-N-(-N-(2-(5-OH-1H-3a-azaindol-3-yl)ethyl)-N-ethyl)-N-(D)ethan-1-amine
2(2-(5-OH-1H-3a-azaindol-3-yl)-N-(-N-(2-(5-OH-1H-3a-azaindol-3-yl)ethyl)-N-ethyl)-N-(D)ethan-1-
amine
2(2-(6-CF3-1H-3a-azaindol-3-yl)-N-(-N-(2-(5-OH-1H-3a-azaindol-3-yl)ethyl)-N-ethyl)-N-(D)ethan-1-
amine
2(2-(7-F-1H-3a-azaindol-3-yl)-N-(-N-(2-(5-OH-1H-3a-azaindol-3-yl)ethyl)-N-ethyl)-N-(D)ethan-1-amine
2(2-(4-D-1H-3a-azaindol-3-yl)-N-(-N-(2-(6-CF3-1H-3a-azaindol-3-yl)ethyl)-N-ethyl)-N-(D)ethan-1-amine
2(2-(5-OH-1H-3a-azaindol-3-yl)-N-(-N-(2-(6-CF3-1H-3a-azaindol-3-yl)ethyl)-N-ethyl)-N-(D)ethan-1-
amine
2(2-(6-CF3-1H-3a-azaindol-3-yl)-N-(-N-(2-(6-CF3-1H-3a-azaindol-3-yl)ethyl)-N-ethyl)-N-(D)ethan-1-
amine
2(2-(7-F-1H-3a-azaindol-3-yl)-N-(-N-(2-(6-CF3-1H-3a-azaindol-3-yl)ethyl)-N-ethyl)-N-(D)ethan-1-amine
2(2-(4-D-1H-3a-azaindol-3-yl)-N-(-N-(2-(7-F-1H-3a-azaindol-3-yl)ethyl)-N-ethyl)-N-(D)ethan-1-amine
2(2-(5-OH-1H-3a-azaindol-3-yl)-N-(-N-(2-(7-F-1H-3a-azaindol-3-yl)ethyl)-N-ethyl)-N-(D)ethan-1-amine
2(2-(6-CF3-1H-3a-azaindol-3-yl)-N-(-N-(2-(7-F-1H-3a-azaindol-3-yl)ethyl)-N-ethyl)-N-(D)ethan-1-amine
2(2-(7-F-1H-3a-azaindol-3-yl)-N-(-N-(2-(7-F-1H-3a-azaindol-3-yl)ethyl)-N-ethyl)-N-(D)ethan-1-amine
2(2-(4-D-1H-3a-azaindol-3-yl)-N-(-N-(2-(4-D-1H-3a-azaindol-3-yl)ethyl)-N-ethyl)-N-propylethan-1-
amine
2(2-(5-OH-1H-3a-azaindol-3-yl)-N-(-N-(2-(4-D-1H-3a-azaindol-3-yl)ethyl)-N-ethyl)-N-propylethan-1-
amine
2(2-(6-CF3-1H-3a-azaindol-3-yl)-N-(-N-(2-(4-D-1H-3a-azaindol-3-yl)ethyl)-N-ethyl)-N-propylethan-1-
amine
2(2-(7-F-1H-3a-azaindol-3-yl)-N-(-N-(2-(4-D-1H-3a-azaindol-3-yl)ethyl)-N-ethyl)-N-propylethan-1-
amine
2(2-(4-D-1H-3a-azaindol-3-yl)-N-(-N-(2-(5-OH-1H-3a-azaindol-3-yl)ethyl)-N-ethyl)-N-propylethan-1-
amine
2(2-(5-OH-1H-3a-azaindol-3-yl)-N-(-N-(2-(5-OH-1H-3a-azaindol-3-yl)ethyl)-N-ethyl)-N-propylethan-1-
amine
2(2-(6-CF3-1H-3a-azaindol-3-yl)-N-(-N-(2-(5-OH-1H-3a-azaindol-3-yl)ethyl)-N-ethyl)-N-propylethan-1-
amine
2(2-(7-F-1H-3a-azaindol-3-yl)-N-(-N-(2-(5-OH-1H-3a-azaindol-3-yl)ethyl)-N-ethyl)-N-propylethan-1-
amine
2(2-(4-D-1H-3a-azaindol-3-yl)-N-(-N-(2-(6-CF3-1H-3a-azaindol-3-yl)ethyl)-N-ethyl)-N-propylethan-1-
amine
2(2-(5-OH-1H-3a-azaindol-3-yl)-N-(-N-(2-(6-CF3-1H-3a-azaindol-3-yl)ethyl)-N-ethyl)-N-propylethan-1-
amine
2(2-(6-CF3-1H-3a-azaindol-3-yl)-N-(-N-(2-(6-CF3-1H-3a-azaindol-3-yl)ethyl)-N-ethyl)-N-propylethan-1-
amine
2(2-(7-F-1H-3a-azaindol-3-yl)-N-(-N-(2-(6-CF3-1H-3a-azaindol-3-yl)ethyl)-N-ethyl)-N-propylethan-1-
amine
2(2-(4-D-1H-3a-azaindol-3-yl)-N-(-N-(2-(7-F-1H-3a-azaindol-3-yl)ethyl)-N-ethyl)-N-propylethan-1-
amine
2(2-(5-OH-1H-3a-azaindol-3-yl)-N-(-N-(2-(7-F-1H-3a-azaindol-3-yl)ethyl)-N-ethyl)-N-propylethan-1-
amine
2(2-(6-CF3-1H-3a-azaindol-3-yl)-N-(-N-(2-(7-F-1H-3a-azaindol-3-yl)ethyl)-N-ethyl)-N-propylethan-1-
amine
2(2-(7-F-1H-3a-azaindol-3-yl)-N-(-N-(2-(7-F-1H-3a-azaindol-3-yl)ethyl)-N-ethyl)-N-propylethan-1-
amine
2(2-(4-D-1H-3a-azaindol-3-yl)-N-(-N-(2-(4-D-1H-5-azaindol-3-yl)ethyl)-N-ethyl)-N-methylethan-1-
amine
2(2-(5-OH-1H-3a-azaindol-3-yl)-N-(-N-(2-(4-D-1H-5-azaindol-3-yl)ethyl)-N-ethyl)-N-methylethan-1-
amine
2(2-(6-CF3-1H-3a-azaindol-3-yl)-N-(-N-(2-(4-D-1H-5-azaindol-3-yl)ethyl)-N-ethyl)-N-methylethan-1-
amine
2(2-(7-F-1H-3a-azaindol-3-yl)-N-(-N-(2-(4-D-1H-5-azaindol-3-yl)ethyl)-N-ethyl)-N-methylethan-1-amine
2(2-(4-D-1H-3a-azaindol-3-yl)-N-(-N-(2-(6-Me-1H-5-azaindol-3-yl)ethyl)-N-ethyl)-N-methylethan-1-
amine
2(2-(5-OH-1H-3a-azaindol-3-yl)-N-(-N-(2-(6-Me-1H-5-azaindol-3-yl)ethyl)-N-ethyl)-N-methylethan-1-
amine
2(2-(6-CF3-1H-3a-azaindol-3-yl)-N-(-N-(2-(6-Me-1H-5-azaindol-3-yl)ethyl)-N-ethyl)-N-methylethan-1-
amine
2(2-(7-F-1H-3a-azaindol-3-yl)-N-(-N-(2-(6-Me-1H-5-azaindol-3-yl)ethyl)-N-ethyl)-N-methylethan-1-
amine
2(2-(4-D-1H-3a-azaindol-3-yl)-N-(-N-(2-(7-CF3-1H-5-azaindol-3-yl)ethyl)-N-ethyl)-N-methylethan-1-
amine
2(2-(5-OH-1H-3a-azaindol-3-yl)-N-(-N-(2-(7-CF3-1H-5-azaindol-3-yl)ethyl)-N-ethyl)-N-methylethan-1-
amine
2(2-(6-CF3-1H-3a-azaindol-3-yl)-N-(-N-(2-(7-CF3-1H-5-azaindol-3-yl)ethyl)-N-ethyl)-N-methylethan-1-
amine
2(2-(7-F-1H-3a-azaindol-3-yl)-N-(-N-(2-(7-CF3-1H-5-azaindol-3-yl)ethyl)-N-ethyl)-N-methylethan-1-
amine
2(2-(4-D-1H-3a-azaindol-3-yl)-N-(-N-(2-(4-D-1H-5-azaindol-3-yl)ethyl)-N-ethyl)-N-ethylethan-1-amine
2(2-(5-OH-1H-3a-azaindol-3-yl)-N-(-N-(2-(4-D-1H-5-azaindol-3-yl)ethyl)-N-ethyl)-N-ethylethan-1-amine
2(2-(6-CF3-1H-3a-azaindol-3-yl)-N-(-N-(2-(4-D-1H-5-azaindol-3-yl)ethyl)-N-ethyl)-N-ethylethan-1-
amine
2(2-(7-F-1H-3a-azaindol-3-yl)-N-(-N-(2-(4-D-1H-5-azaindol-3-yl)ethyl)-N-ethyl)-N-ethylethan-1-amine
2(2-(4-D-1H-3a-azaindol-3-yl)-N-(-N-(2-(6-Me-1H-5-azaindol-3-yl)ethyl)-N-ethyl)-N-ethylethan-1-
amine
2(2-(5-OH-1H-3a-azaindol-3-yl)-N-(-N-(2-(6-Me-1H-5-azaindol-3-yl)ethyl)-N-ethyl)-N-ethylethan-1-
amine
2(2-(6-CF3-1H-3a-azaindol-3-yl)-N-(-N-(2-(6-Me-1H-5-azaindol-3-yl)ethyl)-N-ethyl)-N-ethylethan-1-
amine
2(2-(7-F-1H-3a-azaindol-3-yl)-N-(-N-(2-(6-Me-1H-5-azaindol-3-yl)ethyl)-N-ethyl)-N-ethylethan-1-amine
2(2-(4-D-1H-3a-azaindol-3-yl)-N-(-N-(2-(7-CF3-1H-5-azaindol-3-yl)ethyl)-N-ethyl)-N-ethylethan-1-
amine
2(2-(5-OH-1H-3a-azaindol-3-yl)-N-(-N-(2-(7-CF3-1H-5-azaindol-3-yl)ethyl)-N-ethyl)-N-ethylethan-1-
amine
2(2-(6-CF3-1H-3a-azaindol-3-yl)-N-(-N-(2-(7-CF3-1H-5-azaindol-3-yl)ethyl)-N-ethyl)-N-ethylethan-1-
amine
2(2-(7-F-1H-3a-azaindol-3-yl)-N-(-N-(2-(7-CF3-1H-5-azaindol-3-yl)ethyl)-N-ethyl)-N-ethylethan-1-
amine
2(2-(4-D-1H-3a-azaindol-3-yl)-N-(-N-(2-(4-D-1H-5-azaindol-3-yl)ethyl)-N-ethyl)-N-isopropylethan-1-
amine
2(2-(5-OH-1H-3a-azaindol-3-yl)-N-(-N-(2-(4-D-1H-5-azaindol-3-yl)ethyl)-N-ethyl)-N-isopropylethan-1-
amine
2(2-(6-CF3-1H-3a-azaindol-3-yl)-N-(-N-(2-(4-D-1H-5-azaindol-3-yl)ethyl)-N-ethyl)-N-isopropylethan-1-
amine
2(2-(7-F-1H-3a-azaindol-3-yl)-N-(-N-(2-(4-D-1H-5-azaindol-3-yl)ethyl)-N-ethyl)-N-isopropylethan-1-
amine
2(2-(4-D-1H-3a-azaindol-3-yl)-N-(-N-(2-(6-Me-1H-5-azaindol-3-yl)ethyl)-N-ethyl)-N-isopropylethan-1-
amine
2(2-(5-OH-1H-3a-azaindol-3-yl)-N-(-N-(2-(6-Me-1H-5-azaindol-3-yl)ethyl)-N-ethyl)-N-isopropylethan-1-
amine
2(2-(6-CF3-1H-3a-azaindol-3-yl)-N-(-N-(2-(6-Me-1H-5-azaindol-3-yl)ethyl)-N-ethyl)-N-isopropylethan-
1-amine
2(2-(7-F-1H-3a-azaindol-3-yl)-N-(-N-(2-(6-Me-1H-5-azaindol-3-yl)ethyl)-N-ethyl)-N-isopropylethan-1-
amine
2(2-(4-D-1H-3a-azaindol-3-yl)-N-(-N-(2-(7-CF3-1H-5-azaindol-3-yl)ethyl)-N-ethyl)-N-isopropylethan-1-
amine
2(2-(5-OH-1H-3a-azaindol-3-yl)-N-(-N-(2-(7-CF3-1H-5-azaindol-3-yl)ethyl)-N-ethyl)-N-isopropylethan-
1-amine
2(2-(6-CF3-1H-3a-azaindol-3-yl)-N-(-N-(2-(7-CF3-1H-5-azaindol-3-yl)ethyl)-N-ethyl)-N-isopropylethan-
1-amine
2(2-(7-F-1H-3a-azaindol-3-yl)-N-(-N-(2-(7-CF3-1H-5-azaindol-3-yl)ethyl)-N-ethyl)-N-isopropylethan-1-
amine
2(2-(4-D-1H-3a-azaindol-3-yl)-N-(-N-(2-(4-D-1H-5-azaindol-3-yl)ethyl)-N-ethyl)-N-allylethan-1-amine
2(2-(5-OH-1H-3a-azaindol-3-yl)-N-(-N-(2-(4-D-1H-5-azaindol-3-yl)ethyl)-N-ethyl)-N-allylethan-1-amine
2(2-(6-CF3-1H-3a-azaindol-3-yl)-N-(-N-(2-(4-D-1H-5-azaindol-3-yl)ethyl)-N-ethyl)-N-allylethan-1-amine
2(2-(7-F-1H-3a-azaindol-3-yl)-N-(-N-(2-(4-D-1H-5-azaindol-3-yl)ethyl)-N-ethyl)-N-allylethan-1-amine
2(2-(4-D-1H-3a-azaindol-3-yl)-N-(-N-(2-(6-Me-1H-5-azaindol-3-yl)ethyl)-N-ethyl)-N-allylethan-1-amine
2(2-(5-OH-1H-3a-azaindol-3-yl)-N-(-N-(2-(6-Me-1H-5-azaindol-3-yl)ethyl)-N-ethyl)-N-allylethan-1-
amine
2(2-(6-CF3-1H-3a-azaindol-3-yl)-N-(-N-(2-(6-Me-1H-5-azaindol-3-yl)ethyl)-N-ethyl)-N-allylethan-1-
amine
2(2-(7-F-1H-3a-azaindol-3-yl)-N-(-N-(2-(6-Me-1H-5-azaindol-3-yl)ethyl)-N-ethyl)-N-allylethan-1-amine
2(2-(4-D-1H-3a-azaindol-3-yl)-N-(-N-(2-(7-CF3-1H-5-azaindol-3-yl)ethyl)-N-ethyl)-N-allylethan-1-amine
2(2-(5-OH-1H-3a-azaindol-3-yl)-N-(-N-(2-(7-CF3-1H-5-azaindol-3-yl)ethyl)-N-ethyl)-N-allylethan-1-
amine
2(2-(6-CF3-1H-3a-azaindol-3-yl)-N-(-N-(2-(7-CF3-1H-5-azaindol-3-yl)ethyl)-N-ethyl)-N-allylethan-1-
amine
2(2-(7-F-1H-3a-azaindol-3-yl)-N-(-N-(2-(7-CF3-1H-5-azaindol-3-yl)ethyl)-N-ethyl)-N-allylethan-1-amine
2(2-(4-D-1H-3a-azaindol-3-yl)-N-(-N-(2-(4-D-1H-5-azaindol-3-yl)ethyl)-N-ethyl)-N-hydroethan-1-amine
2(2-(5-OH-1H-3a-azaindol-3-yl)-N-(-N-(2-(4-D-1H-5-azaindol-3-yl)ethyl)-N-ethyl)-N-hydroethan-1-
amine
2(2-(6-CF3-1H-3a-azaindol-3-yl)-N-(-N-(2-(4-D-1H-5-azaindol-3-yl)ethyl)-N-ethyl)-N-hydroethan-1-
amine
2(2-(7-F-1H-3a-azaindol-3-yl)-N-(-N-(2-(4-D-1H-5-azaindol-3-yl)ethyl)-N-ethyl)-N-hydroethan-1-amine
2(2-(4-D-1H-3a-azaindol-3-yl)-N-(-N-(2-(6-Me-1H-5-azaindol-3-yl)ethyl)-N-ethyl)-N-hydroethan-1-
amine
2(2-(5-OH-1H-3a-azaindol-3-yl)-N-(-N-(2-(6-Me-1H-5-azaindol-3-yl)ethyl)-N-ethyl)-N-hydroethan-1-
amine
2(2-(6-CF3-1H-3a-azaindol-3-yl)-N-(-N-(2-(6-Me-1H-5-azaindol-3-yl)ethyl)-N-ethyl)-N-hydroethan-1-
amine
2(2-(7-F-1H-3a-azaindol-3-yl)-N-(-N-(2-(6-Me-1H-5-azaindol-3-yl)ethyl)-N-ethyl)-N-hydroethan-1-
amine
2(2-(4-D-1H-3a-azaindol-3-yl)-N-(-N-(2-(7-CF3-1H-5-azaindol-3-yl)ethyl)-N-ethyl)-N-hydroethan-1-
amine
2(2-(5-OH-1H-3a-azaindol-3-yl)-N-(-N-(2-(7-CF3-1H-5-azaindol-3-yl)ethyl)-N-ethyl)-N-hydroethan-1-
amine
2(2-(6-CF3-1H-3a-azaindol-3-yl)-N-(-N-(2-(7-CF3-1H-5-azaindol-3-yl)ethyl)-N-ethyl)-N-hydroethan-1-
amine
2(2-(7-F-1H-3a-azaindol-3-yl)-N-(-N-(2-(7-CF3-1H-5-azaindol-3-yl)ethyl)-N-ethyl)-N-hydroethan-1-
amine
2(2-(4-D-1H-3a-azaindol-3-yl)-N-(-N-(2-(4-D-1H-5-azaindol-3-yl)ethyl)-N-ethyl)-N-(D)ethan-1-amine
2(2-(5-OH-1H-3a-azaindol-3-yl)-N-(-N-(2-(4-D-1H-5-azaindol-3-yl)ethyl)-N-ethyl)-N-(D)ethan-1-amine
2(2-(6-CF3-1H-3a-azaindol-3-yl)-N-(-N-(2-(4-D-1H-5-azaindol-3-yl)ethyl)-N-ethyl)-N-(D)ethan-1-amine
2(2-(7-F-1H-3a-azaindol-3-yl)-N-(-N-(2-(4-D-1H-5-azaindol-3-yl)ethyl)-N-ethyl)-N-(D)ethan-1-amine
2(2-(4-D-1H-3a-azaindol-3-yl)-N-(-N-(2-(6-Me-1H-5-azaindol-3-yl)ethyl)-N-ethyl)-N-(D)ethan-1-amine
2(2-(5-OH-1H-3a-azaindol-3-yl)-N-(-N-(2-(6-Me-1H-5-azaindol-3-yl)ethyl)-N-ethyl)-N-(D)ethan-1-amine
2(2-(6-CF3-1H-3a-azaindol-3-yl)-N-(-N-(2-(6-Me-1H-5-azaindol-3-yl)ethyl)-N-ethyl)-N-(D)ethan-1-
amine
2(2-(7-F-1H-3a-azaindol-3-yl)-N-(-N-(2-(6-Me-1H-5-azaindol-3-yl)ethyl)-N-ethyl)-N-(D)ethan-1-amine
2(2-(4-D-1H-3a-azaindol-3-yl)-N-(-N-(2-(7-CF3-1H-5-azaindol-3-yl)ethyl)-N-ethyl)-N-(D)ethan-1-amine
2(2-(5-OH-1H-3a-azaindol-3-yl)-N-(-N-(2-(7-CF3-1H-5-azaindol-3-yl)ethyl)-N-ethyl)-N-(D)ethan-1-
amine
2(2-(6-CF3-1H-3a-azaindol-3-yl)-N-(-N-(2-(7-CF3-1H-5-azaindol-3-yl)ethyl)-N-ethyl)-N-(D)ethan-1-
amine
2(2-(7-F-1H-3a-azaindol-3-yl)-N-(-N-(2-(7-CF3-1H-5-azaindol-3-yl)ethyl)-N-ethyl)-N-(D)ethan-1-amine
2(2-(4-D-1H-3a-azaindol-3-yl)-N-(-N-(2-(4-D-1H-5-azaindol-3-yl)ethyl)-N-ethyl)-N-propylethan-1-amine
2(2-(5-OH-1H-3a-azaindol-3-yl)-N-(-N-(2-(4-D-1H-5-azaindol-3-yl)ethyl)-N-ethyl)-N-propylethan-1-
amine
2(2-(6-CF3-1H-3a-azaindol-3-yl)-N-(-N-(2-(4-D-1H-5-azaindol-3-yl)ethyl)-N-ethyl)-N-propylethan-1-
amine
2(2-(7-F-1H-3a-azaindol-3-yl)-N-(-N-(2-(4-D-1H-5-azaindol-3-yl)ethyl)-N-ethyl)-N-propylethan-1-amine
2(2-(4-D-1H-3a-azaindol-3-yl)-N-(-N-(2-(6-Me-1H-5-azaindol-3-yl)ethyl)-N-ethyl)-N-propylethan-1-
amine
2(2-(5-OH-1H-3a-azaindol-3-yl)-N-(-N-(2-(6-Me-1H-5-azaindol-3-yl)ethyl)-N-ethyl)-N-propylethan-1-
amine
2(2-(6-CF3-1H-3a-azaindol-3-yl)-N-(-N-(2-(6-Me-1H-5-azaindol-3-yl)ethyl)-N-ethyl)-N-propylethan-1-
amine
2(2-(7-F-1H-3a-azaindol-3-yl)-N-(-N-(2-(6-Me-1H-5-azaindol-3-yl)ethyl)-N-ethyl)-N-propylethan-1-
amine
2(2-(4-D-1H-3a-azaindol-3-yl)-N-(-N-(2-(7-CF3-1H-5-azaindol-3-yl)ethyl)-N-ethyl)-N-propylethan-1-
amine
2(2-(5-OH-1H-3a-azaindol-3-yl)-N-(-N-(2-(7-CF3-1H-5-azaindol-3-yl)ethyl)-N-ethyl)-N-propylethan-1-
amine
2(2-(6-CF3-1H-3a-azaindol-3-yl)-N-(-N-(2-(7-CF3-1H-5-azaindol-3-yl)ethyl)-N-ethyl)-N-propylethan-1-
amine
2(2-(7-F-1H-3a-azaindol-3-yl)-N-(-N-(2-(7-CF3-1H-5-azaindol-3-yl)ethyl)-N-ethyl)-N-propylethan-1-
amine
2(2-(4-D-1H-3a-azaindol-3-yl)-N-(-N-(2-(4-D-1H-6-azaindol-3-yl)ethyl)-N-ethyl)-N-methylethan-1-
amine
2(2-(5-OH-1H-3a-azaindol-3-yl)-N-(-N-(2-(4-D-1H-6-azaindol-3-yl)ethyl)-N-ethyl)-N-methylethan-1-
amine
2(2-(6-CF3-1H-3a-azaindol-3-yl)-N-(-N-(2-(4-D-1H-6-azaindol-3-yl)ethyl)-N-ethyl)-N-methylethan-1-
amine
2(2-(7-F-1H-3a-azaindol-3-yl)-N-(-N-(2-(4-D-1H-6-azaindol-3-yl)ethyl)-N-ethyl)-N-methylethan-1-amine
2(2-(4-D-1H-3a-azaindol-3-yl)-N-(-N-(2-(5-OH-1H-6-azaindol-3-yl)ethyl)-N-ethyl)-N-methylethan-1-
amine
2(2-(5-OH-1H-3a-azaindol-3-yl)-N-(-N-(2-(5-OH-1H-6-azaindol-3-yl)ethyl)-N-ethyl)-N-methylethan-1-
amine
2(2-(6-CF3-1H-3a-azaindol-3-yl)-N-(-N-(2-(5-OH-1H-6-azaindol-3-yl)ethyl)-N-ethyl)-N-methylethan-1-
amine
2(2-(7-F-1H-3a-azaindol-3-yl)-N-(-N-(2-(5-OH-1H-6-azaindol-3-yl)ethyl)-N-ethyl)-N-methylethan-1-
amine
2(2-(4-D-1H-3a-azaindol-3-yl)-N-(-N-(2-(7-CF3-1H-6-azaindol-3-yl)ethyl)-N-ethyl)-N-methylethan-1-
amine
2(2-(5-OH-1H-3a-azaindol-3-yl)-N-(-N-(2-(7-CF3-1H-6-azaindol-3-yl)ethyl)-N-ethyl)-N-methylethan-1-
amine
2(2-(6-CF3-1H-3a-azaindol-3-yl)-N-(-N-(2-(7-CF3-1H-6-azaindol-3-yl)ethyl)-N-ethyl)-N-methylethan-1-
amine
2(2-(7-F-1H-3a-azaindol-3-yl)-N-(-N-(2-(7-CF3-1H-6-azaindol-3-yl)ethyl)-N-ethyl)-N-methylethan-1-
amine
2(2-(4-D-1H-3a-azaindol-3-yl)-N-(-N-(2-(4-D-1H-6-azaindol-3-yl)ethyl)-N-ethyl)-N-ethylethan-1-amine
2(2-(5-OH-1H-3a-azaindol-3-yl)-N-(-N-(2-(4-D-1H-6-azaindol-3-yl)ethyl)-N-ethyl)-N-ethylethan-1-amine
2(2-(6-CF3-1H-3a-azaindol-3-yl)-N-(-N-(2-(4-D-1H-6-azaindol-3-yl)ethyl)-N-ethyl)-N-ethylethan-1-
amine
2(2-(7-F-1H-3a-azaindol-3-yl)-N-(-N-(2-(4-D-1H-6-azaindol-3-yl)ethyl)-N-ethyl)-N-ethylethan-1-amine
2(2-(4-D-1H-3a-azaindol-3-yl)-N-(-N-(2-(5-OH-1H-6-azaindol-3-yl)ethyl)-N-ethyl)-N-ethylethan-1-amine
2(2-(5-OH-1H-3a-azaindol-3-yl)-N-(-N-(2-(5-OH-1H-6-azaindol-3-yl)ethyl)-N-ethyl)-N-ethylethan-1-
amine
2(2-(6-CF3-1H-3a-azaindol-3-yl)-N-(-N-(2-(5-OH-1H-6-azaindol-3-yl)ethyl)-N-ethyl)-N-ethylethan-1-
amine
2(2-(7-F-1H-3a-azaindol-3-yl)-N-(-N-(2-(5-OH-1H-6-azaindol-3-yl)ethyl)-N-ethyl)-N-ethylethan-1-amine
2(2-(4-D-1H-3a-azaindol-3-yl)-N-(-N-(2-(7-CF3-1H-6-azaindol-3-yl)ethyl)-N-ethyl)-N-ethylethan-1-
amine
2(2-(5-OH-1H-3a-azaindol-3-yl)-N-(-N-(2-(7-CF3-1H-6-azaindol-3-yl)ethyl)-N-ethyl)-N-ethylethan-1-
amine
2(2-(6-CF3-1H-3a-azaindol-3-yl)-N-(-N-(2-(7-CF3-1H-6-azaindol-3-yl)ethyl)-N-ethyl)-N-ethylethan-1-
amine
2(2-(7-F-1H-3a-azaindol-3-yl)-N-(-N-(2-(7-CF3-1H-6-azaindol-3-yl)ethyl)-N-ethyl)-N-ethylethan-1-
amine
2(2-(4-D-1H-3a-azaindol-3-yl)-N-(-N-(2-(4-D-1H-6-azaindol-3-yl)ethyl)-N-ethyl)-N-isopropylethan-1-
amine
2(2-(5-OH-1H-3a-azaindol-3-yl)-N-(-N-(2-(4-D-1H-6-azaindol-3-yl)ethyl)-N-ethyl)-N-isopropylethan-1-
amine
2(2-(6-CF3-1H-3a-azaindol-3-yl)-N-(-N-(2-(4-D-1H-6-azaindol-3-yl)ethyl)-N-ethyl)-N-isopropylethan-1-
amine
2(2-(7-F-1H-3a-azaindol-3-yl)-N-(-N-(2-(4-D-1H-6-azaindol-3-yl)ethyl)-N-ethyl)-N-isopropylethan-1-
amine
2(2-(4-D-1H-3a-azaindol-3-yl)-N-(-N-(2-(5-OH-1H-6-azaindol-3-yl)ethyl)-N-ethyl)-N-isopropylethan-1-
amine
2(2-(5-OH-1H-3a-azaindol-3-yl)-N-(-N-(2-(5-OH-1H-6-azaindol-3-yl)ethyl)-N-ethyl)-N-isopropylethan-1-
amine
2(2-(6-CF3-1H-3a-azaindol-3-yl)-N-(-N-(2-(5-OH-1H-6-azaindol-3-yl)ethyl)-N-ethyl)-N-isopropylethan-
1-amine
2(2-(7-F-1H-3a-azaindol-3-yl)-N-(-N-(2-(5-OH-1H-6-azaindol-3-yl)ethyl)-N-ethyl)-N-isopropylethan-1-
amine
2(2-(4-D-1H-3a-azaindol-3-yl)-N-(-N-(2-(7-CF3-1H-6-azaindol-3-yl)ethyl)-N-ethyl)-N-isopropylethan-1-
amine
2(2-(5-OH-1H-3a-azaindol-3-yl)-N-(-N-(2-(7-CF3-1H-6-azaindol-3-yl)ethyl)-N-ethyl)-N-isopropylethan-
1-amine
2(2-(6-CF3-1H-3a-azaindol-3-yl)-N-(-N-(2-(7-CF3-1H-6-azaindol-3-yl)ethyl)-N-ethyl)-N-isopropylethan-
1-amine
2(2-(7-F-1H-3a-azaindol-3-yl)-N-(-N-(2-(7-CF3-1H-6-azaindol-3-yl)ethyl)-N-ethyl)-N-isopropylethan-1-
amine
2(2-(4-D-1H-3a-azaindol-3-yl)-N-(-N-(2-(4-D-1H-6-azaindol-3-yl)ethyl)-N-ethyl)-N-allylethan-1-amine
2(2-(5-OH-1H-3a-azaindol-3-yl)-N-(-N-(2-(4-D-1H-6-azaindol-3-yl)ethyl)-N-ethyl)-N-allylethan-1-amine
2(2-(6-CF3-1H-3a-azaindol-3-yl)-N-(-N-(2-(4-D-1H-6-azaindol-3-yl)ethyl)-N-ethyl)-N-allylethan-1-amine
2(2-(7-F-1H-3a-azaindol-3-yl)-N-(-N-(2-(4-D-1H-6-azaindol-3-yl)ethyl)-N-ethyl)-N-allylethan-1-amine
2(2-(4-D-1H-3a-azaindol-3-yl)-N-(-N-(2-(5-OH-1H-6-azaindol-3-yl)ethyl)-N-ethyl)-N-allylethan-1-amine
2(2-(5-OH-1H-3a-azaindol-3-yl)-N-(-N-(2-(5-OH-1H-6-azaindol-3-yl)ethyl)-N-ethyl)-N-allylethan-1-
amine
2(2-(6-CF3-1H-3a-azaindol-3-yl)-N-(-N-(2-(5-OH-1H-6-azaindol-3-yl)ethyl)-N-ethyl)-N-allylethan-1-
amine
2(2-(7-F-1H-3a-azaindol-3-yl)-N-(-N-(2-(5-OH-1H-6-azaindol-3-yl)ethyl)-N-ethyl)-N-allylethan-1-amine
2(2-(4-D-1H-3a-azaindol-3-yl)-N-(-N-(2-(7-CF3-1H-6-azaindol-3-yl)ethyl)-N-ethyl)-N-allylethan-1-amine
2(2-(5-OH-1H-3a-azaindol-3-yl)-N-(-N-(2-(7-CF3-1H-6-azaindol-3-yl)ethyl)-N-ethyl)-N-allylethan-1-
amine
2(2-(6-CF3-1H-3a-azaindol-3-yl)-N-(-N-(2-(7-CF3-1H-6-azaindol-3-yl)ethyl)-N-ethyl)-N-allylethan-1-
amine
2(2-(7-F-1H-3a-azaindol-3-yl)-N-(-N-(2-(7-CF3-1H-6-azaindol-3-yl)ethyl)-N-ethyl)-N-allylethan-1-amine
2(2-(4-D-1H-3a-azaindol-3-yl)-N-(-N-(2-(4-D-1H-6-azaindol-3-yl)ethyl)-N-ethyl)-N-hydroethan-1-amine
2(2-(5-OH-1H-3a-azaindol-3-yl)-N-(-N-(2-(4-D-1H-6-azaindol-3-yl)ethyl)-N-ethyl)-N-hydroethan-1-
amine
2(2-(6-CF3-1H-3a-azaindol-3-yl)-N-(-N-(2-(4-D-1H-6-azaindol-3-yl)ethyl)-N-ethyl)-N-hydroethan-1-
amine
2(2-(7-F-1H-3a-azaindol-3-yl)-N-(-N-(2-(4-D-1H-6-azaindol-3-yl)ethyl)-N-ethyl)-N-hydroethan-1-amine
2(2-(4-D-1H-3a-azaindol-3-yl)-N-(-N-(2-(5-OH-1H-6-azaindol-3-yl)ethyl)-N-ethyl)-N-hydroethan-1-
amine
2(2-(5-OH-1H-3a-azaindol-3-yl)-N-(-N-(2-(5-OH-1H-6-azaindol-3-yl)ethyl)-N-ethyl)-N-hydroethan-1-
amine
2(2-(6-CF3-1H-3a-azaindol-3-yl)-N-(-N-(2-(5-OH-1H-6-azaindol-3-yl)ethyl)-N-ethyl)-N-hydroethan-1-
amine
2(2-(7-F-1H-3a-azaindol-3-yl)-N-(-N-(2-(5-OH-1H-6-azaindol-3-yl)ethyl)-N-ethyl)-N-hydroethan-1-
amine
2(2-(4-D-1H-3a-azaindol-3-yl)-N-(-N-(2-(7-CF3-1H-6-azaindol-3-yl)ethyl)-N-ethyl)-N-hydroethan-1-
amine
2(2-(5-OH-1H-3a-azaindol-3-yl)-N-(-N-(2-(7-CF3-1H-6-azaindol-3-yl)ethyl)-N-ethyl)-N-hydroethan-1-
amine
2(2-(6-CF3-1H-3a-azaindol-3-yl)-N-(-N-(2-(7-CF3-1H-6-azaindol-3-yl)ethyl)-N-ethyl)-N-hydroethan-1-
amine
2(2-(7-F-1H-3a-azaindol-3-yl)-N-(-N-(2-(7-CF3-1H-6-azaindol-3-yl)ethyl)-N-ethyl)-N-hydroethan-1-
amine
2(2-(4-D-1H-3a-azaindol-3-yl)-N-(-N-(2-(4-D-1H-6-azaindol-3-yl)ethyl)-N-ethyl)-N-(D)ethan-1-amine
2(2-(5-OH-1H-3a-azaindol-3-yl)-N-(-N-(2-(4-D-1H-6-azaindol-3-yl)ethyl)-N-ethyl)-N-(D)ethan-1-amine
2(2-(6-CF3-1H-3a-azaindol-3-yl)-N-(-N-(2-(4-D-1H-6-azaindol-3-yl)ethyl)-N-ethyl)-N-(D)ethan-1-amine
2(2-(7-F-1H-3a-azaindol-3-yl)-N-(-N-(2-(4-D-1H-6-azaindol-3-yl)ethyl)-N-ethyl)-N-(D)ethan-1-amine
2(2-(4-D-1H-3a-azaindol-3-yl)-N-(-N-(2-(5-OH-1H-6-azaindol-3-yl)ethyl)-N-ethyl)-N-(D)ethan-1-amine
2(2-(5-OH-1H-3a-azaindol-3-yl)-N-(-N-(2-(5-OH-1H-6-azaindol-3-yl)ethyl)-N-ethyl)-N-(D)ethan-1-amine
2(2-(6-CF3-1H-3a-azaindol-3-yl)-N-(-N-(2-(5-OH-1H-6-azaindol-3-yl)ethyl)-N-ethyl)-N-(D)ethan-1-
amine
2(2-(7-F-1H-3a-azaindol-3-yl)-N-(-N-(2-(5-OH-1H-6-azaindol-3-yl)ethyl)-N-ethyl)-N-(D)ethan-1-amine
2(2-(4-D-1H-3a-azaindol-3-yl)-N-(-N-(2-(7-CF3-1H-6-azaindol-3-yl)ethyl)-N-ethyl)-N-(D)ethan-1-amine
2(2-(5-OH-1H-3a-azaindol-3-yl)-N-(-N-(2-(7-CF3-1H-6-azaindol-3-yl)ethyl)-N-ethyl)-N-(D)ethan-1-
amine
2(2-(6-CF3-1H-3a-azaindol-3-yl)-N-(-N-(2-(7-CF3-1H-6-azaindol-3-yl)ethyl)-N-ethyl)-N-(D)ethan-1-
amine
2(2-(7-F-1H-3a-azaindol-3-yl)-N-(-N-(2-(7-CF3-1H-6-azaindol-3-yl)ethyl)-N-ethyl)-N-(D)ethan-1-amine
2(2-(4-D-1H-3a-azaindol-3-yl)-N-(-N-(2-(4-D-1H-6-azaindol-3-yl)ethyl)-N-ethyl)-N-propylethan-1-amine
2(2-(5-OH-1H-3a-azaindol-3-yl)-N-(-N-(2-(4-D-1H-6-azaindol-3-yl)ethyl)-N-ethyl)-N-propylethan-1-
amine
2(2-(6-CF3-1H-3a-azaindol-3-yl)-N-(-N-(2-(4-D-1H-6-azaindol-3-yl)ethyl)-N-ethyl)-N-propylethan-1-
amine
2(2-(7-F-1H-3a-azaindol-3-yl)-N-(-N-(2-(4-D-1H-6-azaindol-3-yl)ethyl)-N-ethyl)-N-propylethan-1-amine
2(2-(4-D-1H-3a-azaindol-3-yl)-N-(-N-(2-(5-OH-1H-6-azaindol-3-yl)ethyl)-N-ethyl)-N-propylethan-1-
amine
2(2-(5-OH-1H-3a-azaindol-3-yl)-N-(-N-(2-(5-OH-1H-6-azaindol-3-yl)ethyl)-N-ethyl)-N-propylethan-1-
amine
2(2-(6-CF3-1H-3a-azaindol-3-yl)-N-(-N-(2-(5-OH-1H-6-azaindol-3-yl)ethyl)-N-ethyl)-N-propylethan-1-
amine
2(2-(7-F-1H-3a-azaindol-3-yl)-N-(-N-(2-(5-OH-1H-6-azaindol-3-yl)ethyl)-N-ethyl)-N-propylethan-1-
amine
2(2-(4-D-1H-3a-azaindol-3-yl)-N-(-N-(2-(7-CF3-1H-6-azaindol-3-yl)ethyl)-N-ethyl)-N-propylethan-1-
amine
2(2-(5-OH-1H-3a-azaindol-3-yl)-N-(-N-(2-(7-CF3-1H-6-azaindol-3-yl)ethyl)-N-ethyl)-N-propylethan-1-
amine
2(2-(6-CF3-1H-3a-azaindol-3-yl)-N-(-N-(2-(7-CF3-1H-6-azaindol-3-yl)ethyl)-N-ethyl)-N-propylethan-1-
amine
2(2-(7-F-1H-3a-azaindol-3-yl)-N-(-N-(2-(7-CF3-1H-6-azaindol-3-yl)ethyl)-N-ethyl)-N-propylethan-1-
amine
2(2-(4-D-1H-3a-azaindol-3-yl)-N-(-N-(2-(4-D-1H-7-azaindol-3-yl)ethyl)-N-ethyl)-N-methylethan-1-
amine
2(2-(5-OH-1H-3a-azaindol-3-yl)-N-(-N-(2-(4-D-1H-7-azaindol-3-yl)ethyl)-N-ethyl)-N-methylethan-1-
amine
2(2-(6-CF3-1H-3a-azaindol-3-yl)-N-(-N-(2-(4-D-1H-7-azaindol-3-yl)ethyl)-N-ethyl)-N-methylethan-1-
amine
2(2-(7-F-1H-3a-azaindol-3-yl)-N-(-N-(2-(4-D-1H-7-azaindol-3-yl)ethyl)-N-ethyl)-N-methylethan-1-amine
2(2-(4-D-1H-3a-azaindol-3-yl)-N-(-N-(2-(5-OH-1H-7-azaindol-3-yl)ethyl)-N-ethyl)-N-methylethan-1-
amine
2(2-(5-OH-1H-3a-azaindol-3-yl)-N-(-N-(2-(5-OH-1H-7-azaindol-3-yl)ethyl)-N-ethyl)-N-methylethan-1-
amine
2(2-(6-CF3-1H-3a-azaindol-3-yl)-N-(-N-(2-(5-OH-1H-7-azaindol-3-yl)ethyl)-N-ethyl)-N-methylethan-1-
amine
2(2-(7-F-1H-3a-azaindol-3-yl)-N-(-N-(2-(5-OH-1H-7-azaindol-3-yl)ethyl)-N-ethyl)-N-methylethan-1-
amine
2(2-(4-D-1H-3a-azaindol-3-yl)-N-(-N-(2-(6-CF3-1H-7-azaindol-3-yl)ethyl)-N-ethyl)-N-methylethan-1-
amine
2(2-(5-OH-1H-3a-azaindol-3-yl)-N-(-N-(2-(6-CF3-1H-7-azaindol-3-yl)ethyl)-N-ethyl)-N-methylethan-1-
amine
2(2-(6-CF3-1H-3a-azaindol-3-yl)-N-(-N-(2-(6-CF3-1H-7-azaindol-3-yl)ethyl)-N-ethyl)-N-methylethan-1-
amine
2(2-(7-F-1H-3a-azaindol-3-yl)-N-(-N-(2-(6-CF3-1H-7-azaindol-3-yl)ethyl)-N-ethyl)-N-methylethan-1-
amine
2(2-(4-D-1H-3a-azaindol-3-yl)-N-(-N-(2-(4-D-1H-7-azaindol-3-yl)ethyl)-N-ethyl)-N-ethylethan-1-amine
2(2-(5-OH-1H-3a-azaindol-3-yl)-N-(-N-(2-(4-D-1H-7-azaindol-3-yl)ethyl)-N-ethyl)-N-ethylethan-1-amine
2(2-(6-CF3-1H-3a-azaindol-3-yl)-N-(-N-(2-(4-D-1H-7-azaindol-3-yl)ethyl)-N-ethyl)-N-ethylethan-1-
amine
2(2-(7-F-1H-3a-azaindol-3-yl)-N-(-N-(2-(4-D-1H-7-azaindol-3-yl)ethyl)-N-ethyl)-N-ethylethan-1-amine
2(2-(4-D-1H-3a-azaindol-3-yl)-N-(-N-(2-(5-OH-1H-7-azaindol-3-yl)ethyl)-N-ethyl)-N-ethylethan-1-amine
2(2-(5-OH-1H-3a-azaindol-3-yl)-N-(-N-(2-(5-OH-1H-7-azaindol-3-yl)ethyl)-N-ethyl)-N-ethylethan-1-
amine
2(2-(6-CF3-1H-3a-azaindol-3-yl)-N-(-N-(2-(5-OH-1H-7-azaindol-3-yl)ethyl)-N-ethyl)-N-ethylethan-1-
amine
2(2-(7-F-1H-3a-azaindol-3-yl)-N-(-N-(2-(5-OH-1H-7-azaindol-3-yl)ethyl)-N-ethyl)-N-ethylethan-1-amine
2(2-(4-D-1H-3a-azaindol-3-yl)-N-(-N-(2-(6-CF3-1H-7-azaindol-3-yl)ethyl)-N-ethyl)-N-ethylethan-1-
amine
2(2-(5-OH-1H-3a-azaindol-3-yl)-N-(-N-(2-(6-CF3-1H-7-azaindol-3-yl)ethyl)-N-ethyl)-N-ethylethan-1-
amine
2(2-(6-CF3-1H-3a-azaindol-3-yl)-N-(-N-(2-(6-CF3-1H-7-azaindol-3-yl)ethyl)-N-ethyl)-N-ethylethan-1-
amine
2(2-(7-F-1H-3a-azaindol-3-yl)-N-(-N-(2-(6-CF3-1H-7-azaindol-3-yl)ethyl)-N-ethyl)-N-ethylethan-1-
amine
2(2-(4-D-1H-3a-azaindol-3-yl)-N-(-N-(2-(4-D-1H-7-azaindol-3-yl)ethyl)-N-ethyl)-N-isopropylethan-1-
amine
2(2-(5-OH-1H-3a-azaindol-3-yl)-N-(-N-(2-(4-D-1H-7-azaindol-3-yl)ethyl)-N-ethyl)-N-isopropylethan-1-
amine
2(2-(6-CF3-1H-3a-azaindol-3-yl)-N-(-N-(2-(4-D-1H-7-azaindol-3-yl)ethyl)-N-ethyl)-N-isopropylethan-1-
amine
2(2-(7-F-1H-3a-azaindol-3-yl)-N-(-N-(2-(4-D-1H-7-azaindol-3-yl)ethyl)-N-ethyl)-N-isopropylethan-1-
amine
2(2-(4-D-1H-3a-azaindol-3-yl)-N-(-N-(2-(5-OH-1H-7-azaindol-3-yl)ethyl)-N-ethyl)-N-isopropylethan-1-
amine
2(2-(5-OH-1H-3a-azaindol-3-yl)-N-(-N-(2-(5-OH-1H-7-azaindol-3-yl)ethyl)-N-ethyl)-N-isopropylethan-1-
amine
2(2-(6-CF3-1H-3a-azaindol-3-yl)-N-(-N-(2-(5-OH-1H-7-azaindol-3-yl)ethyl)-N-ethyl)-N-isopropylethan-
1-amine
2(2-(7-F-1H-3a-azaindol-3-yl)-N-(-N-(2-(5-OH-1H-7-azaindol-3-yl)ethyl)-N-ethyl)-N-isopropylethan-1-
amine
2(2-(4-D-1H-3a-azaindol-3-yl)-N-(-N-(2-(6-CF3-1H-7-azaindol-3-yl)ethyl)-N-ethyl)-N-isopropylethan-1-
amine
2(2-(5-OH-1H-3a-azaindol-3-yl)-N-(-N-(2-(6-CF3-1H-7-azaindol-3-yl)ethyl)-N-ethyl)-N-isopropylethan-
1-amine
2(2-(6-CF3-1H-3a-azaindol-3-yl)-N-(-N-(2-(6-CF3-1H-7-azaindol-3-yl)ethyl)-N-ethyl)-N-isopropylethan-
1-amine
2(2-(7-F-1H-3a-azaindol-3-yl)-N-(-N-(2-(6-CF3-1H-7-azaindol-3-yl)ethyl)-N-ethyl)-N-isopropylethan-1-
amine
2(2-(4-D-1H-3a-azaindol-3-yl)-N-(-N-(2-(4-D-1H-7-azaindol-3-yl)ethyl)-N-ethyl)-N-allylethan-1-amine
2(2-(5-OH-1H-3a-azaindol-3-yl)-N-(-N-(2-(4-D-1H-7-azaindol-3-yl)ethyl)-N-ethyl)-N-allylethan-1-amine
2(2-(6-CF3-1H-3a-azaindol-3-yl)-N-(-N-(2-(4-D-1H-7-azaindol-3-yl)ethyl)-N-ethyl)-N-allylethan-1-amine
2(2-(7-F-1H-3a-azaindol-3-yl)-N-(-N-(2-(4-D-1H-7-azaindol-3-yl)ethyl)-N-ethyl)-N-allylethan-1-amine
2(2-(4-D-1H-3a-azaindol-3-yl)-N-(-N-(2-(5-OH-1H-7-azaindol-3-yl)ethyl)-N-ethyl)-N-allylethan-1-amine
2(2-(5-OH-1H-3a-azaindol-3-yl)-N-(-N-(2-(5-OH-1H-7-azaindol-3-yl)ethyl)-N-ethyl)-N-allylethan-1-
amine
2(2-(6-CF3-1H-3a-azaindol-3-yl)-N-(-N-(2-(5-OH-1H-7-azaindol-3-yl)ethyl)-N-ethyl)-N-allylethan-1-
amine
2(2-(7-F-1H-3a-azaindol-3-yl)-N-(-N-(2-(5-OH-1H-7-azaindol-3-yl)ethyl)-N-ethyl)-N-allylethan-1-amine
2(2-(4-D-1H-3a-azaindol-3-yl)-N-(-N-(2-(6-CF3-1H-7-azaindol-3-yl)ethyl)-N-ethyl)-N-allylethan-1-amine
2(2-(5-OH-1H-3a-azaindol-3-yl)-N-(-N-(2-(6-CF3-1H-7-azaindol-3-yl)ethyl)-N-ethyl)-N-allylethan-1-
amine
2(2-(6-CF3-1H-3a-azaindol-3-yl)-N-(-N-(2-(6-CF3-1H-7-azaindol-3-yl)ethyl)-N-ethyl)-N-allylethan-1-
amine
2(2-(7-F-1H-3a-azaindol-3-yl)-N-(-N-(2-(6-CF3-1H-7-azaindol-3-yl)ethyl)-N-ethyl)-N-allylethan-1-amine
2(2-(4-D-1H-3a-azaindol-3-yl)-N-(-N-(2-(4-D-1H-7-azaindol-3-yl)ethyl)-N-ethyl)-N-hydroethan-1-amine
2(2-(5-OH-1H-3a-azaindol-3-yl)-N-(-N-(2-(4-D-1H-7-azaindol-3-yl)ethyl)-N-ethyl)-N-hydroethan-1-
amine
2(2-(6-CF3-1H-3a-azaindol-3-yl)-N-(-N-(2-(4-D-1H-7-azaindol-3-yl)ethyl)-N-ethyl)-N-hydroethan-1-
amine
2(2-(7-F-1H-3a-azaindol-3-yl)-N-(-N-(2-(4-D-1H-7-azaindol-3-yl)ethyl)-N-ethyl)-N-hydroethan-1-amine
2(2-(4-D-1H-3a-azaindol-3-yl)-N-(-N-(2-(5-OH-1H-7-azaindol-3-yl)ethyl)-N-ethyl)-N-hydroethan-1-
amine
2(2-(5-OH-1H-3a-azaindol-3-yl)-N-(-N-(2-(5-OH-1H-7-azaindol-3-yl)ethyl)-N-ethyl)-N-hydroethan-1-
amine
2(2-(6-CF3-1H-3a-azaindol-3-yl)-N-(-N-(2-(5-OH-1H-7-azaindol-3-yl)ethyl)-N-ethyl)-N-hydroethan-1-
amine
2(2-(7-F-1H-3a-azaindol-3-yl)-N-(-N-(2-(5-OH-1H-7-azaindol-3-yl)ethyl)-N-ethyl)-N-hydroethan-1-
amine
2(2-(4-D-1H-3a-azaindol-3-yl)-N-(-N-(2-(6-CF3-1H-7-azaindol-3-yl)ethyl)-N-ethyl)-N-hydroethan-1-
amine
2(2-(5-OH-1H-3a-azaindol-3-yl)-N-(-N-(2-(6-CF3-1H-7-azaindol-3-yl)ethyl)-N-ethyl)-N-hydroethan-1-
amine
2(2-(6-CF3-1H-3a-azaindol-3-yl)-N-(-N-(2-(6-CF3-1H-7-azaindol-3-yl)ethyl)-N-ethyl)-N-hydroethan-1-
amine
2(2-(7-F-1H-3a-azaindol-3-yl)-N-(-N-(2-(6-CF3-1H-7-azaindol-3-yl)ethyl)-N-ethyl)-N-hydroethan-1-
amine
2(2-(4-D-1H-3a-azaindol-3-yl)-N-(-N-(2-(4-D-1H-7-azaindol-3-yl)ethyl)-N-ethyl)-N-(D)ethan-1-amine
2(2-(5-OH-1H-3a-azaindol-3-yl)-N-(-N-(2-(4-D-1H-7-azaindol-3-yl)ethyl)-N-ethyl)-N-(D)ethan-1-amine
2(2-(6-CF3-1H-3a-azaindol-3-yl)-N-(-N-(2-(4-D-1H-7-azaindol-3-yl)ethyl)-N-ethyl)-N-(D)ethan-1-amine
2(2-(7-F-1H-3a-azaindol-3-yl)-N-(-N-(2-(4-D-1H-7-azaindol-3-yl)ethyl)-N-ethyl)-N-(D)ethan-1-amine
2(2-(4-D-1H-3a-azaindol-3-yl)-N-(-N-(2-(5-OH-1H-7-azaindol-3-yl)ethyl)-N-ethyl)-N-(D)ethan-1-amine
2(2-(5-OH-1H-3a-azaindol-3-yl)-N-(-N-(2-(5-OH-1H-7-azaindol-3-yl)ethyl)-N-ethyl)-N-(D)ethan-1-amine
2(2-(6-CF3-1H-3a-azaindol-3-yl)-N-(-N-(2-(5-OH-1H-7-azaindol-3-yl)ethyl)-N-ethyl)-N-(D)ethan-1-
amine
2(2-(7-F-1H-3a-azaindol-3-yl)-N-(-N-(2-(5-OH-1H-7-azaindol-3-yl)ethyl)-N-ethyl)-N-(D)ethan-1-amine
2(2-(4-D-1H-3a-azaindol-3-yl)-N-(-N-(2-(6-CF3-1H-7-azaindol-3-yl)ethyl)-N-ethyl)-N-(D)ethan-1-amine
2(2-(5-OH-1H-3a-azaindol-3-yl)-N-(-N-(2-(6-CF3-1H-7-azaindol-3-yl)ethyl)-N-ethyl)-N-(D)ethan-1-
amine
2(2-(6-CF3-1H-3a-azaindol-3-yl)-N-(-N-(2-(6-CF3-1H-7-azaindol-3-yl)ethyl)-N-ethyl)-N-(D)ethan-1-
amine
2(2-(7-F-1H-3a-azaindol-3-yl)-N-(-N-(2-(6-CF3-1H-7-azaindol-3-yl)ethyl)-N-ethyl)-N-(D)ethan-1-amine
2(2-(4-D-1H-3a-azaindol-3-yl)-N-(-N-(2-(4-D-1H-7-azaindol-3-yl)ethyl)-N-ethyl)-N-propylethan-1-amine
2(2-(5-OH-1H-3a-azaindol-3-yl)-N-(-N-(2-(4-D-1H-7-azaindol-3-yl)ethyl)-N-ethyl)-N-propylethan-1-
amine
2(2-(6-CF3-1H-3a-azaindol-3-yl)-N-(-N-(2-(4-D-1H-7-azaindol-3-yl)ethyl)-N-ethyl)-N-propylethan-1-
amine
2(2-(7-F-1H-3a-azaindol-3-yl)-N-(-N-(2-(4-D-1H-7-azaindol-3-yl)ethyl)-N-ethyl)-N-propylethan-1-amine
2(2-(4-D-1H-3a-azaindol-3-yl)-N-(-N-(2-(5-OH-1H-7-azaindol-3-yl)ethyl)-N-ethyl)-N-propylethan-1-
amine
2(2-(5-OH-1H-3a-azaindol-3-yl)-N-(-N-(2-(5-OH-1H-7-azaindol-3-yl)ethyl)-N-ethyl)-N-propylethan-1-
amine
2(2-(6-CF3-1H-3a-azaindol-3-yl)-N-(-N-(2-(5-OH-1H-7-azaindol-3-yl)ethyl)-N-ethyl)-N-propylethan-1-
amine
2(2-(7-F-1H-3a-azaindol-3-yl)-N-(-N-(2-(5-OH-1H-7-azaindol-3-yl)ethyl)-N-ethyl)-N-propylethan-1-
amine
2(2-(4-D-1H-3a-azaindol-3-yl)-N-(-N-(2-(6-CF3-1H-7-azaindol-3-yl)ethyl)-N-ethyl)-N-propylethan-1-
amine
2(2-(5-OH-1H-3a-azaindol-3-yl)-N-(-N-(2-(6-CF3-1H-7-azaindol-3-yl)ethyl)-N-ethyl)-N-propylethan-1-
amine
2(2-(6-CF3-1H-3a-azaindol-3-yl)-N-(-N-(2-(6-CF3-1H-7-azaindol-3-yl)ethyl)-N-ethyl)-N-propylethan-1-
amine
2(2-(7-F-1H-3a-azaindol-3-yl)-N-(-N-(2-(6-CF3-1H-7-azaindol-3-yl)ethyl)-N-ethyl)-N-propylethan-1-
amine
2(2-(4-D-1H-3a-azaindol-3-yl)-N-(-N-(2-(4-D-1H-7a-azaindol-3-yl)ethyl)-N-ethyl)-N-methylethan-1-
amine
2(2-(5-OH-1H-3a-azaindol-3-yl)-N-(-N-(2-(4-D-1H-7a-azaindol-3-yl)ethyl)-N-ethyl)-N-methylethan-1-
amine
2(2-(6-CF3-1H-3a-azaindol-3-yl)-N-(-N-(2-(4-D-1H-7a-azaindol-3-yl)ethyl)-N-ethyl)-N-methylethan-1-
amine
2(2-(7-F-1H-3a-azaindol-3-yl)-N-(-N-(2-(4-D-1H-7a-azaindol-3-yl)ethyl)-N-ethyl)-N-methylethan-1-
amine
2(2-(4-D-1H-3a-azaindol-3-yl)-N-(-N-(2-(5-OH-1H-7a-azaindol-3-yl)ethyl)-N-ethyl)-N-methylethan-1-
amine
2(2-(5-OH-1H-3a-azaindol-3-yl)-N-(-N-(2-(5-OH-1H-7a-azaindol-3-yl)ethyl)-N-ethyl)-N-methylethan-1-
amine
2(2-(6-CF3-1H-3a-azaindol-3-yl)-N-(-N-(2-(5-OH-1H-7a-azaindol-3-yl)ethyl)-N-ethyl)-N-methylethan-1-
amine
2(2-(7-F-1H-3a-azaindol-3-yl)-N-(-N-(2-(5-OH-1H-7a-azaindol-3-yl)ethyl)-N-ethyl)-N-methylethan-1-
amine
2(2-(4-D-1H-3a-azaindol-3-yl)-N-(-N-(2-(6-CF3-1H-7a-azaindol-3-yl)ethyl)-N-ethyl)-N-methylethan-1-
amine
2(2-(5-OH-1H-3a-azaindol-3-yl)-N-(-N-(2-(6-CF3-1H-7a-azaindol-3-yl)ethyl)-N-ethyl)-N-methylethan-1-
amine
2(2-(6-CF3-1H-3a-azaindol-3-yl)-N-(-N-(2-(6-CF3-1H-7a-azaindol-3-yl)ethyl)-N-ethyl)-N-methylethan-1-
amine
2(2-(7-F-1H-3a-azaindol-3-yl)-N-(-N-(2-(6-CF3-1H-7a-azaindol-3-yl)ethyl)-N-ethyl)-N-methylethan-1-
amine
2(2-(4-D-1H-3a-azaindol-3-yl)-N-(-N-(2-(7-F-1H-7a-azaindol-3-yl)ethyl)-N-ethyl)-N-methylethan-1-
amine
2(2-(5-OH-1H-3a-azaindol-3-yl)-N-(-N-(2-(7-F-1H-7a-azaindol-3-yl)ethyl)-N-ethyl)-N-methylethan-1-
amine
2(2-(6-CF3-1H-3a-azaindol-3-yl)-N-(-N-(2-(7-F-1H-7a-azaindol-3-yl)ethyl)-N-ethyl)-N-methylethan-1-
amine
2(2-(7-F-1H-3a-azaindol-3-yl)-N-(-N-(2-(7-F-1H-7a-azaindol-3-yl)ethyl)-N-ethyl)-N-methylethan-1-
amine
2(2-(4-D-1H-3a-azaindol-3-yl)-N-(-N-(2-(4-D-1H-7a-azaindol-3-yl)ethyl)-N-ethyl)-N-ethylethan-1-amine
2(2-(5-OH-1H-3a-azaindol-3-yl)-N-(-N-(2-(4-D-1H-7a-azaindol-3-yl)ethyl)-N-ethyl)-N-ethylethan-1-
amine
2(2-(6-CF3-1H-3a-azaindol-3-yl)-N-(-N-(2-(4-D-1H-7a-azaindol-3-yl)ethyl)-N-ethyl)-N-ethylethan-1-
amine
2(2-(7-F-1H-3a-azaindol-3-yl)-N-(-N-(2-(4-D-1H-7a-azaindol-3-yl)ethyl)-N-ethyl)-N-ethylethan-1-amine
2(2-(4-D-1H-3a-azaindol-3-yl)-N-(-N-(2-(5-OH-1H-7a-azaindol-3-yl)ethyl)-N-ethyl)-N-ethylethan-1-
amine
2(2-(5-OH-1H-3a-azaindol-3-yl)-N-(-N-(2-(5-OH-1H-7a-azaindol-3-yl)ethyl)-N-ethyl)-N-ethylethan-1-
amine
2(2-(6-CF3-1H-3a-azaindol-3-yl)-N-(-N-(2-(5-OH-1H-7a-azaindol-3-yl)ethyl)-N-ethyl)-N-ethylethan-1-
amine
2(2-(7-F-1H-3a-azaindol-3-yl)-N-(-N-(2-(5-OH-1H-7a-azaindol-3-yl)ethyl)-N-ethyl)-N-ethylethan-1-
amine
2(2-(4-D-1H-3a-azaindol-3-yl)-N-(-N-(2-(6-CF3-1H-7a-azaindol-3-yl)ethyl)-N-ethyl)-N-ethylethan-1-
amine
2(2-(5-OH-1H-3a-azaindol-3-yl)-N-(-N-(2-(6-CF3-1H-7a-azaindol-3-yl)ethyl)-N-ethyl)-N-ethylethan-1-
amine
2(2-(6-CF3-1H-3a-azaindol-3-yl)-N-(-N-(2-(6-CF3-1H-7a-azaindol-3-yl)ethyl)-N-ethyl)-N-ethylethan-1-
amine
2(2-(7-F-1H-3a-azaindol-3-yl)-N-(-N-(2-(6-CF3-1H-7a-azaindol-3-yl)ethyl)-N-ethyl)-N-ethylethan-1-
amine
2(2-(4-D-1H-3a-azaindol-3-yl)-N-(-N-(2-(7-F-1H-7a-azaindol-3-yl)ethyl)-N-ethyl)-N-ethylethan-1-amine
2(2-(5-OH-1H-3a-azaindol-3-yl)-N-(-N-(2-(7-F-1H-7a-azaindol-3-yl)ethyl)-N-ethyl)-N-ethylethan-1-
amine
2(2-(6-CF3-1H-3a-azaindol-3-yl)-N-(-N-(2-(7-F-1H-7a-azaindol-3-yl)ethyl)-N-ethyl)-N-ethylethan-1-
amine
2(2-(7-F-1H-3a-azaindol-3-yl)-N-(-N-(2-(7-F-1H-7a-azaindol-3-yl)ethyl)-N-ethyl)-N-ethylethan-1-amine
2(2-(4-D-1H-3a-azaindol-3-yl)-N-(-N-(2-(4-D-1H-7a-azaindol-3-yl)ethyl)-N-ethyl)-N-isopropylethan-1-
amine
2(2-(5-OH-1H-3a-azaindol-3-yl)-N-(-N-(2-(4-D-1H-7a-azaindol-3-yl)ethyl)-N-ethyl)-N-isopropylethan-1-
amine
2(2-(6-CF3-1H-3a-azaindol-3-yl)-N-(-N-(2-(4-D-1H-7a-azaindol-3-yl)ethyl)-N-ethyl)-N-isopropylethan-1-
amine
2(2-(7-F-1H-3a-azaindol-3-yl)-N-(-N-(2-(4-D-1H-7a-azaindol-3-yl)ethyl)-N-ethyl)-N-isopropylethan-1-
amine
2(2-(4-D-1H-3a-azaindol-3-yl)-N-(-N-(2-(5-OH-1H-7a-azaindol-3-yl)ethyl)-N-ethyl)-N-isopropylethan-1-
amine
2(2-(5-OH-1H-3a-azaindol-3-yl)-N-(-N-(2-(5-OH-1H-7a-azaindol-3-yl)ethyl)-N-ethyl)-N-isopropylethan-
1-amine
2(2-(6-CF3-1H-3a-azaindol-3-yl)-N-(-N-(2-(5-OH-1H-7a-azaindol-3-yl)ethyl)-N-ethyl)-N-isopropylethan-
1-amine
2(2-(7-F-1H-3a-azaindol-3-yl)-N-(-N-(2-(5-OH-1H-7a-azaindol-3-yl)ethyl)-N-ethyl)-N-isopropylethan-1-
amine
2(2-(4-D-1H-3a-azaindol-3-yl)-N-(-N-(2-(6-CF3-1H-7a-azaindol-3-yl)ethyl)-N-ethyl)-N-isopropylethan-1-
amine
2(2-(5-OH-1H-3a-azaindol-3-yl)-N-(-N-(2-(6-CF3-1H-7a-azaindol-3-yl)ethyl)-N-ethyl)-N-isopropylethan-
1-amine
2(2-(6-CF3-1H-3a-azaindol-3-yl)-N-(-N-(2-(6-CF3-1H-7a-azaindol-3-yl)ethyl)-N-ethyl)-N-isopropylethan-
1-amine
2(2-(7-F-1H-3a-azaindol-3-yl)-N-(-N-(2-(6-CF3-1H-7a-azaindol-3-yl)ethyl)-N-ethyl)-N-isopropylethan-1-
amine
2(2-(4-D-1H-3a-azaindol-3-yl)-N-(-N-(2-(7-F-1H-7a-azaindol-3-yl)ethyl)-N-ethyl)-N-isopropylethan-1-
amine
2(2-(5-OH-1H-3a-azaindol-3-yl)-N-(-N-(2-(7-F-1H-7a-azaindol-3-yl)ethyl)-N-ethyl)-N-isopropylethan-1-
amine
2(2-(6-CF3-1H-3a-azaindol-3-yl)-N-(-N-(2-(7-F-1H-7a-azaindol-3-yl)ethyl)-N-ethyl)-N-isopropylethan-1-
amine
2(2-(7-F-1H-3a-azaindol-3-yl)-N-(-N-(2-(7-F-1H-7a-azaindol-3-yl)ethyl)-N-ethyl)-N-isopropylethan-1-
amine
2(2-(4-D-1H-3a-azaindol-3-yl)-N-(-N-(2-(4-D-1H-7a-azaindol-3-yl)ethyl)-N-ethyl)-N-allylethan-1-amine
2(2-(5-OH-1H-3a-azaindol-3-yl)-N-(-N-(2-(4-D-1H-7a-azaindol-3-yl)ethyl)-N-ethyl)-N-allylethan-1-
amine
2(2-(6-CF3-1H-3a-azaindol-3-yl)-N-(-N-(2-(4-D-1H-7a-azaindol-3-yl)ethyl)-N-ethyl)-N-allylethan-1-
amine
2(2-(7-F-1H-3a-azaindol-3-yl)-N-(-N-(2-(4-D-1H-7a-azaindol-3-yl)ethyl)-N-ethyl)-N-allylethan-1-amine
2(2-(4-D-1H-3a-azaindol-3-yl)-N-(-N-(2-(5-OH-1H-7a-azaindol-3-yl)ethyl)-N-ethyl)-N-allylethan-1-
amine
2(2-(5-OH-1H-3a-azaindol-3-yl)-N-(-N-(2-(5-OH-1H-7a-azaindol-3-yl)ethyl)-N-ethyl)-N-allylethan-1-
amine
2(2-(6-CF3-1H-3a-azaindol-3-yl)-N-(-N-(2-(5-OH-1H-7a-azaindol-3-yl)ethyl)-N-ethyl)-N-allylethan-1-
amine
2(2-(7-F-1H-3a-azaindol-3-yl)-N-(-N-(2-(5-OH-1H-7a-azaindol-3-yl)ethyl)-N-ethyl)-N-allylethan-1-amine
2(2-(4-D-1H-3a-azaindol-3-yl)-N-(-N-(2-(6-CF3-1H-7a-azaindol-3-yl)ethyl)-N-ethyl)-N-allylethan-1-
amine
2(2-(5-OH-1H-3a-azaindol-3-yl)-N-(-N-(2-(6-CF3-1H-7a-azaindol-3-yl)ethyl)-N-ethyl)-N-allylethan-1-
amine
2(2-(6-CF3-1H-3a-azaindol-3-yl)-N-(-N-(2-(6-CF3-1H-7a-azaindol-3-yl)ethyl)-N-ethyl)-N-allylethan-1-
amine
2(2-(7-F-1H-3a-azaindol-3-yl)-N-(-N-(2-(6-CF3-1H-7a-azaindol-3-yl)ethyl)-N-ethyl)-N-allylethan-1-
amine
2(2-(4-D-1H-3a-azaindol-3-yl)-N-(-N-(2-(7-F-1H-7a-azaindol-3-yl)ethyl)-N-ethyl)-N-allylethan-1-amine
2(2-(5-OH-1H-3a-azaindol-3-yl)-N-(-N-(2-(7-F-1H-7a-azaindol-3-yl)ethyl)-N-ethyl)-N-allylethan-1-amine
2(2-(6-CF3-1H-3a-azaindol-3-yl)-N-(-N-(2-(7-F-1H-7a-azaindol-3-yl)ethyl)-N-ethyl)-N-allylethan-1-
amine
2(2-(7-F-1H-3a-azaindol-3-yl)-N-(-N-(2-(7-F-1H-7a-azaindol-3-yl)ethyl)-N-ethyl)-N-allylethan-1-amine
2(2-(4-D-1H-3a-azaindol-3-yl)-N-(-N-(2-(4-D-1H-7a-azaindol-3-yl)ethyl)-N-ethyl)-N-hydroethan-1-
amine
2(2-(5-OH-1H-3a-azaindol-3-yl)-N-(-N-(2-(4-D-1H-7a-azaindol-3-yl)ethyl)-N-ethyl)-N-hydroethan-1-
amine
2(2-(6-CF3-1H-3a-azaindol-3-yl)-N-(-N-(2-(4-D-1H-7a-azaindol-3-yl)ethyl)-N-ethyl)-N-hydroethan-1-
amine
2(2-(7-F-1H-3a-azaindol-3-yl)-N-(-N-(2-(4-D-1H-7a-azaindol-3-yl)ethyl)-N-ethyl)-N-hydroethan-1-amine
2(2-(4-D-1H-3a-azaindol-3-yl)-N-(-N-(2-(5-OH-1H-7a-azaindol-3-yl)ethyl)-N-ethyl)-N-hydroethan-1-
amine
2(2-(5-OH-1H-3a-azaindol-3-yl)-N-(-N-(2-(5-OH-1H-7a-azaindol-3-yl)ethyl)-N-ethyl)-N-hydroethan-1-
amine
2(2-(6-CF3-1H-3a-azaindol-3-yl)-N-(-N-(2-(5-OH-1H-7a-azaindol-3-yl)ethyl)-N-ethyl)-N-hydroethan-1-
amine
2(2-(7-F-1H-3a-azaindol-3-yl)-N-(-N-(2-(5-OH-1H-7a-azaindol-3-yl)ethyl)-N-ethyl)-N-hydroethan-1-
amine
2(2-(4-D-1H-3a-azaindol-3-yl)-N-(-N-(2-(6-CF3-1H-7a-azaindol-3-yl)ethyl)-N-ethyl)-N-hydroethan-1-
amine
2(2-(5-OH-1H-3a-azaindol-3-yl)-N-(-N-(2-(6-CF3-1H-7a-azaindol-3-yl)ethyl)-N-ethyl)-N-hydroethan-1-
amine
2(2-(6-CF3-1H-3a-azaindol-3-yl)-N-(-N-(2-(6-CF3-1H-7a-azaindol-3-yl)ethyl)-N-ethyl)-N-hydroethan-1-
amine
2(2-(7-F-1H-3a-azaindol-3-yl)-N-(-N-(2-(6-CF3-1H-7a-azaindol-3-yl)ethyl)-N-ethyl)-N-hydroethan-1-
amine
2(2-(4-D-1H-3a-azaindol-3-yl)-N-(-N-(2-(7-F-1H-7a-azaindol-3-yl)ethyl)-N-ethyl)-N-hydroethan-1-amine
2(2-(5-OH-1H-3a-azaindol-3-yl)-N-(-N-(2-(7-F-1H-7a-azaindol-3-yl)ethyl)-N-ethyl)-N-hydroethan-1-
amine
2(2-(6-CF3-1H-3a-azaindol-3-yl)-N-(-N-(2-(7-F-1H-7a-azaindol-3-yl)ethyl)-N-ethyl)-N-hydroethan-1-
amine
2(2-(7-F-1H-3a-azaindol-3-yl)-N-(-N-(2-(7-F-1H-7a-azaindol-3-yl)ethyl)-N-ethyl)-N-hydroethan-1-amine
2(2-(4-D-1H-3a-azaindol-3-yl)-N-(-N-(2-(4-D-1H-7a-azaindol-3-yl)ethyl)-N-ethyl)-N-(D)ethan-1-amine
2(2-(5-OH-1H-3a-azaindol-3-yl)-N-(-N-(2-(4-D-1H-7a-azaindol-3-yl)ethyl)-N-ethyl)-N-(D)ethan-1-amine
2(2-(6-CF3-1H-3a-azaindol-3-yl)-N-(-N-(2-(4-D-1H-7a-azaindol-3-yl)ethyl)-N-ethyl)-N-(D)ethan-1-amine
2(2-(7-F-1H-3a-azaindol-3-yl)-N-(-N-(2-(4-D-1H-7a-azaindol-3-yl)ethyl)-N-ethyl)-N-(D)ethan-1-amine
2(2-(4-D-1H-3a-azaindol-3-yl)-N-(-N-(2-(5-OH-1H-7a-azaindol-3-yl)ethyl)-N-ethyl)-N-(D)ethan-1-amine
2(2-(5-OH-1H-3a-azaindol-3-yl)-N-(-N-(2-(5-OH-1H-7a-azaindol-3-yl)ethyl)-N-ethyl)-N-(D)ethan-1-
amine
2(2-(6-CF3-1H-3a-azaindol-3-yl)-N-(-N-(2-(5-OH-1H-7a-azaindol-3-yl)ethyl)-N-ethyl)-N-(D)ethan-1-
amine
2(2-(7-F-1H-3a-azaindol-3-yl)-N-(-N-(2-(5-OH-1H-7a-azaindol-3-yl)ethyl)-N-ethyl)-N-(D)ethan-1-amine
2(2-(4-D-1H-3a-azaindol-3-yl)-N-(-N-(2-(6-CF3-1H-7a-azaindol-3-yl)ethyl)-N-ethyl)-N-(D)ethan-1-amine
2(2-(5-OH-1H-3a-azaindol-3-yl)-N-(-N-(2-(6-CF3-1H-7a-azaindol-3-yl)ethyl)-N-ethyl)-N-(D)ethan-1-
amine
2(2-(6-CF3-1H-3a-azaindol-3-yl)-N-(-N-(2-(6-CF3-1H-7a-azaindol-3-yl)ethyl)-N-ethyl)-N-(D)ethan-1-
amine
2(2-(7-F-1H-3a-azaindol-3-yl)-N-(-N-(2-(6-CF3-1H-7a-azaindol-3-yl)ethyl)-N-ethyl)-N-(D)ethan-1-amine
2(2-(4-D-1H-3a-azaindol-3-yl)-N-(-N-(2-(7-F-1H-7a-azaindol-3-yl)ethyl)-N-ethyl)-N-(D)ethan-1-amine
2(2-(5-OH-1H-3a-azaindol-3-yl)-N-(-N-(2-(7-F-1H-7a-azaindol-3-yl)ethyl)-N-ethyl)-N-(D)ethan-1-amine
2(2-(6-CF3-1H-3a-azaindol-3-yl)-N-(-N-(2-(7-F-1H-7a-azaindol-3-yl)ethyl)-N-ethyl)-N-(D)ethan-1-amine
2(2-(7-F-1H-3a-azaindol-3-yl)-N-(-N-(2-(7-F-1H-7a-azaindol-3-yl)ethyl)-N-ethyl)-N-(D)ethan-1-amine
2(2-(4-D-1H-3a-azaindol-3-yl)-N-(-N-(2-(4-D-1H-7a-azaindol-3-yl)ethyl)-N-ethyl)-N-propylethan-1-
amine
2(2-(5-OH-1H-3a-azaindol-3-yl)-N-(-N-(2-(4-D-1H-7a-azaindol-3-yl)ethyl)-N-ethyl)-N-propylethan-1-
amine
2(2-(6-CF3-1H-3a-azaindol-3-yl)-N-(-N-(2-(4-D-1H-7a-azaindol-3-yl)ethyl)-N-ethyl)-N-propylethan-1-
amine
2(2-(7-F-1H-3a-azaindol-3-yl)-N-(-N-(2-(4-D-1H-7a-azaindol-3-yl)ethyl)-N-ethyl)-N-propylethan-1-
amine
2(2-(4-D-1H-3a-azaindol-3-yl)-N-(-N-(2-(5-OH-1H-7a-azaindol-3-yl)ethyl)-N-ethyl)-N-propylethan-1-
amine
2(2-(5-OH-1H-3a-azaindol-3-yl)-N-(-N-(2-(5-OH-1H-7a-azaindol-3-yl)ethyl)-N-ethyl)-N-propylethan-1-
amine
2(2-(6-CF3-1H-3a-azaindol-3-yl)-N-(-N-(2-(5-OH-1H-7a-azaindol-3-yl)ethyl)-N-ethyl)-N-propylethan-1-
amine
2(2-(7-F-1H-3a-azaindol-3-yl)-N-(-N-(2-(5-OH-1H-7a-azaindol-3-yl)ethyl)-N-ethyl)-N-propylethan-1-
amine
2(2-(4-D-1H-3a-azaindol-3-yl)-N-(-N-(2-(6-CF3-1H-7a-azaindol-3-yl)ethyl)-N-ethyl)-N-propylethan-1-
amine
2(2-(5-OH-1H-3a-azaindol-3-yl)-N-(-N-(2-(6-CF3-1H-7a-azaindol-3-yl)ethyl)-N-ethyl)-N-propylethan-1-
amine
2(2-(6-CF3-1H-3a-azaindol-3-yl)-N-(-N-(2-(6-CF3-1H-7a-azaindol-3-yl)ethyl)-N-ethyl)-N-propylethan-1-
amine
2(2-(7-F-1H-3a-azaindol-3-yl)-N-(-N-(2-(6-CF3-1H-7a-azaindol-3-yl)ethyl)-N-ethyl)-N-propylethan-1-
amine
2(2-(4-D-1H-3a-azaindol-3-yl)-N-(-N-(2-(7-F-1H-7a-azaindol-3-yl)ethyl)-N-ethyl)-N-propylethan-1-
amine
2(2-(5-OH-1H-3a-azaindol-3-yl)-N-(-N-(2-(7-F-1H-7a-azaindol-3-yl)ethyl)-N-ethyl)-N-propylethan-1-
amine
2(2-(6-CF3-1H-3a-azaindol-3-yl)-N-(-N-(2-(7-F-1H-7a-azaindol-3-yl)ethyl)-N-ethyl)-N-propylethan-1-
amine
2(2-(7-F-1H-3a-azaindol-3-yl)-N-(-N-(2-(7-F-1H-7a-azaindol-3-yl)ethyl)-N-ethyl)-N-propylethan-1-
amine
2(2-(4-D-1H-3a-azaindol-3-yl)-N-(-N-(2-(4-D-1H-indol-3-yl)ethyl)-N-ethyl)-N-methylethan-1-amine
2(2-(5-OH-1H-3a-azaindol-3-yl)-N-(-N-(2-(4-D-1H-indol-3-yl)ethyl)-N-ethyl)-N-methylethan-1-amine
2(2-(6-CF3-1H-3a-azaindol-3-yl)-N-(-N-(2-(4-D-1H-indol-3-yl)ethyl)-N-ethyl)-N-methylethan-1-amine
2(2-(7-F-1H-3a-azaindol-3-yl)-N-(-N-(2-(4-D-1H-indol-3-yl)ethyl)-N-ethyl)-N-methylethan-1-amine
2(2-(4-D-1H-3a-azaindol-3-yl)-N-(-N-(2-(5-OH-1H-indol-3-yl)ethyl)-N-ethyl)-N-methylethan-1-amine
2(2-(5-OH-1H-3a-azaindol-3-yl)-N-(-N-(2-(5-OH-1H-indol-3-yl)ethyl)-N-ethyl)-N-methylethan-1-amine
2(2-(6-CF3-1H-3a-azaindol-3-yl)-N-(-N-(2-(5-OH-1H-indol-3-yl)ethyl)-N-ethyl)-N-methylethan-1-amine
2(2-(7-F-1H-3a-azaindol-3-yl)-N-(-N-(2-(5-OH-1H-indol-3-yl)ethyl)-N-ethyl)-N-methylethan-1-amine
2(2-(4-D-1H-3a-azaindol-3-yl)-N-(-N-(2-(6-CF3-1H-indol-3-yl)ethyl)-N-ethyl)-N-methylethan-1-amine
2(2-(5-OH-1H-3a-azaindol-3-yl)-N-(-N-(2-(6-CF3-1H-indol-3-yl)ethyl)-N-ethyl)-N-methylethan-1-amine
2(2-(6-CF3-1H-3a-azaindol-3-yl)-N-(-N-(2-(6-CF3-1H-indol-3-yl)ethyl)-N-ethyl)-N-methylethan-1-amine
2(2-(7-F-1H-3a-azaindol-3-yl)-N-(-N-(2-(6-CF3-1H-indol-3-yl)ethyl)-N-ethyl)-N-methylethan-1-amine
2(2-(4-D-1H-3a-azaindol-3-yl)-N-(-N-(2-(7-F-1H-indol-3-yl)ethyl)-N-ethyl)-N-methylethan-1-amine
2(2-(5-OH-1H-3a-azaindol-3-yl)-N-(-N-(2-(7-F-1H-indol-3-yl)ethyl)-N-ethyl)-N-methylethan-1-amine
2(2-(6-CF3-1H-3a-azaindol-3-yl)-N-(-N-(2-(7-F-1H-indol-3-yl)ethyl)-N-ethyl)-N-methylethan-1-amine
2(2-(7-F-1H-3a-azaindol-3-yl)-N-(-N-(2-(7-F-1H-indol-3-yl)ethyl)-N-ethyl)-N-methylethan-1-amine
2(2-(4-D-1H-3a-azaindol-3-yl)-N-(-N-(2-(4-D-1H-indol-3-yl)ethyl)-N-ethyl)-N-ethylethan-1-amine
2(2-(5-OH-1H-3a-azaindol-3-yl)-N-(-N-(2-(4-D-1H-indol-3-yl)ethyl)-N-ethyl)-N-ethylethan-1-amine
2(2-(6-CF3-1H-3a-azaindol-3-yl)-N-(-N-(2-(4-D-1H-indol-3-yl)ethyl)-N-ethyl)-N-ethylethan-1-amine
2(2-(7-F-1H-3a-azaindol-3-yl)-N-(-N-(2-(4-D-1H-indol-3-yl)ethyl)-N-ethyl)-N-ethylethan-1-amine
2(2-(4-D-1H-3a-azaindol-3-yl)-N-(-N-(2-(5-OH-1H-indol-3-yl)ethyl)-N-ethyl)-N-ethylethan-1-amine
2(2-(5-OH-1H-3a-azaindol-3-yl)-N-(-N-(2-(5-OH-1H-indol-3-yl)ethyl)-N-ethyl)-N-ethylethan-1-amine
2(2-(6-CF3-1H-3a-azaindol-3-yl)-N-(-N-(2-(5-OH-1H-indol-3-yl)ethyl)-N-ethyl)-N-ethylethan-1-amine
2(2-(7-F-1H-3a-azaindol-3-yl)-N-(-N-(2-(5-OH-1H-indol-3-yl)ethyl)-N-ethyl)-N-ethylethan-1-amine
2(2-(4-D-1H-3a-azaindol-3-yl)-N-(-N-(2-(6-CF3-1H-indol-3-yl)ethyl)-N-ethyl)-N-ethylethan-1-amine
2(2-(5-OH-1H-3a-azaindol-3-yl)-N-(-N-(2-(6-CF3-1H-indol-3-yl)ethyl)-N-ethyl)-N-ethylethan-1-amine
2(2-(6-CF3-1H-3a-azaindol-3-yl)-N-(-N-(2-(6-CF3-1H-indol-3-yl)ethyl)-N-ethyl)-N-ethylethan-1-amine
2(2-(7-F-1H-3a-azaindol-3-yl)-N-(-N-(2-(6-CF3-1H-indol-3-yl)ethyl)-N-ethyl)-N-ethylethan-1-amine
2(2-(4-D-1H-3a-azaindol-3-yl)-N-(-N-(2-(7-F-1H-indol-3-yl)ethyl)-N-ethyl)-N-ethylethan-1-amine
2(2-(5-OH-1H-3a-azaindol-3-yl)-N-(-N-(2-(7-F-1H-indol-3-yl)ethyl)-N-ethyl)-N-ethylethan-1-amine
2(2-(6-CF3-1H-3a-azaindol-3-yl)-N-(-N-(2-(7-F-1H-indol-3-yl)ethyl)-N-ethyl)-N-ethylethan-1-amine
2(2-(7-F-1H-3a-azaindol-3-yl)-N-(-N-(2-(7-F-1H-indol-3-yl)ethyl)-N-ethyl)-N-ethylethan-1-amine
2(2-(4-D-1H-3a-azaindol-3-yl)-N-(-N-(2-(4-D-1H-indol-3-yl)ethyl)-N-ethyl)-N-isopropylethan-1-amine
2(2-(5-OH-1H-3a-azaindol-3-yl)-N-(-N-(2-(4-D-1H-indol-3-yl)ethyl)-N-ethyl)-N-isopropylethan-1-amine
2(2-(6-CF3-1H-3a-azaindol-3-yl)-N-(-N-(2-(4-D-1H-indol-3-yl)ethyl)-N-ethyl)-N-isopropylethan-1-amine
2(2-(7-F-1H-3a-azaindol-3-yl)-N-(-N-(2-(4-D-1H-indol-3-yl)ethyl)-N-ethyl)-N-isopropylethan-1-amine
2(2-(4-D-1H-3a-azaindol-3-yl)-N-(-N-(2-(5-OH-1H-indol-3-yl)ethyl)-N-ethyl)-N-isopropylethan-1-amine
2(2-(5-OH-1H-3a-azaindol-3-yl)-N-(-N-(2-(5-OH-1H-indol-3-yl)ethyl)-N-ethyl)-N-isopropylethan-1-
amine
2(2-(6-CF3-1H-3a-azaindol-3-yl)-N-(-N-(2-(5-OH-1H-indol-3-yl)ethyl)-N-ethyl)-N-isopropylethan-1-
amine
2(2-(7-F-1H-3a-azaindol-3-yl)-N-(-N-(2-(5-OH-1H-indol-3-yl)ethyl)-N-ethyl)-N-isopropylethan-1-amine
2(2-(4-D-1H-3a-azaindol-3-yl)-N-(-N-(2-(6-CF3-1H-indol-3-yl)ethyl)-N-ethyl)-N-isopropylethan-1-amine
2(2-(5-OH-1H-3a-azaindol-3-yl)-N-(-N-(2-(6-CF3-1H-indol-3-yl)ethyl)-N-ethyl)-N-isopropylethan-1-
amine
2(2-(6-CF3-1H-3a-azaindol-3-yl)-N-(-N-(2-(6-CF3-1H-indol-3-yl)ethyl)-N-ethyl)-N-isopropylethan-1-
amine
2(2-(7-F-1H-3a-azaindol-3-yl)-N-(-N-(2-(6-CF3-1H-indol-3-yl)ethyl)-N-ethyl)-N-isopropylethan-1-amine
2(2-(4-D-1H-3a-azaindol-3-yl)-N-(-N-(2-(7-F-1H-indol-3-yl)ethyl)-N-ethyl)-N-isopropylethan-1-amine
2(2-(5-OH-1H-3a-azaindol-3-yl)-N-(-N-(2-(7-F-1H-indol-3-yl)ethyl)-N-ethyl)-N-isopropylethan-1-amine
2(2-(6-CF3-1H-3a-azaindol-3-yl)-N-(-N-(2-(7-F-1H-indol-3-yl)ethyl)-N-ethyl)-N-isopropylethan-1-amine
2(2-(7-F-1H-3a-azaindol-3-yl)-N-(-N-(2-(7-F-1H-indol-3-yl)ethyl)-N-ethyl)-N-isopropylethan-1-amine
2(2-(4-D-1H-3a-azaindol-3-yl)-N-(-N-(2-(4-D-1H-indol-3-yl)ethyl)-N-ethyl)-N-allylethan-1-amine
2(2-(5-OH-1H-3a-azaindol-3-yl)-N-(-N-(2-(4-D-1H-indol-3-yl)ethyl)-N-ethyl)-N-allylethan-1-amine
2(2-(6-CF3-1H-3a-azaindol-3-yl)-N-(-N-(2-(4-D-1H-indol-3-yl)ethyl)-N-ethyl)-N-allylethan-1-amine
2(2-(7-F-1H-3a-azaindol-3-yl)-N-(-N-(2-(4-D-1H-indol-3-yl)ethyl)-N-ethyl)-N-allylethan-1-amine
2(2-(4-D-1H-3a-azaindol-3-yl)-N-(-N-(2-(5-OH-1H-indol-3-yl)ethyl)-N-ethyl)-N-allylethan-1-amine
2(2-(5-OH-1H-3a-azaindol-3-yl)-N-(-N-(2-(5-OH-1H-indol-3-yl)ethyl)-N-ethyl)-N-allylethan-1-amine
2(2-(6-CF3-1H-3a-azaindol-3-yl)-N-(-N-(2-(5-OH-1H-indol-3-yl)ethyl)-N-ethyl)-N-allylethan-1-amine
2(2-(7-F-1H-3a-azaindol-3-yl)-N-(-N-(2-(5-OH-1H-indol-3-yl)ethyl)-N-ethyl)-N-allylethan-1-amine
2(2-(4-D-1H-3a-azaindol-3-yl)-N-(-N-(2-(6-CF3-1H-indol-3-yl)ethyl)-N-ethyl)-N-allylethan-1-amine
2(2-(5-OH-1H-3a-azaindol-3-yl)-N-(-N-(2-(6-CF3-1H-indol-3-yl)ethyl)-N-ethyl)-N-allylethan-1-amine
2(2-(6-CF3-1H-3a-azaindol-3-yl)-N-(-N-(2-(6-CF3-1H-indol-3-yl)ethyl)-N-ethyl)-N-allylethan-1-amine
2(2-(7-F-1H-3a-azaindol-3-yl)-N-(-N-(2-(6-CF3-1H-indol-3-yl)ethyl)-N-ethyl)-N-allylethan-1-amine
2(2-(4-D-1H-3a-azaindol-3-yl)-N-(-N-(2-(7-F-1H-indol-3-yl)ethyl)-N-ethyl)-N-allylethan-1-amine
2(2-(5-OH-1H-3a-azaindol-3-yl)-N-(-N-(2-(7-F-1H-indol-3-yl)ethyl)-N-ethyl)-N-allylethan-1-amine
2(2-(6-CF3-1H-3a-azaindol-3-yl)-N-(-N-(2-(7-F-1H-indol-3-yl)ethyl)-N-ethyl)-N-allylethan-1-amine
2(2-(7-F-1H-3a-azaindol-3-yl)-N-(-N-(2-(7-F-1H-indol-3-yl)ethyl)-N-ethyl)-N-allylethan-1-amine
2(2-(4-D-1H-3a-azaindol-3-yl)-N-(-N-(2-(4-D-1H-indol-3-yl)ethyl)-N-ethyl)-N-hydroethan-1-amine
2(2-(5-OH-1H-3a-azaindol-3-yl)-N-(-N-(2-(4-D-1H-indol-3-yl)ethyl)-N-ethyl)-N-hydroethan-1-amine
2(2-(6-CF3-1H-3a-azaindol-3-yl)-N-(-N-(2-(4-D-1H-indol-3-yl)ethyl)-N-ethyl)-N-hydroethan-1-amine
2(2-(7-F-1H-3a-azaindol-3-yl)-N-(-N-(2-(4-D-1H-indol-3-yl)ethyl)-N-ethyl)-N-hydroethan-1-amine
2(2-(4-D-1H-3a-azaindol-3-yl)-N-(-N-(2-(5-OH-1H-indol-3-yl)ethyl)-N-ethyl)-N-hydroethan-1-amine
2(2-(5-OH-1H-3a-azaindol-3-yl)-N-(-N-(2-(5-OH-1H-indol-3-yl)ethyl)-N-ethyl)-N-hydroethan-1-amine
2(2-(6-CF3-1H-3a-azaindol-3-yl)-N-(-N-(2-(5-OH-1H-indol-3-yl)ethyl)-N-ethyl)-N-hydroethan-1-amine
2(2-(7-F-1H-3a-azaindol-3-yl)-N-(-N-(2-(5-OH-1H-indol-3-yl)ethyl)-N-ethyl)-N-hydroethan-1-amine
2(2-(4-D-1H-3a-azaindol-3-yl)-N-(-N-(2-(6-CF3-1H-indol-3-yl)ethyl)-N-ethyl)-N-hydroethan-1-amine
2(2-(5-OH-1H-3a-azaindol-3-yl)-N-(-N-(2-(6-CF3-1H-indol-3-yl)ethyl)-N-ethyl)-N-hydroethan-1-amine
2(2-(6-CF3-1H-3a-azaindol-3-yl)-N-(-N-(2-(6-CF3-1H-indol-3-yl)ethyl)-N-ethyl)-N-hydroethan-1-amine
2(2-(7-F-1H-3a-azaindol-3-yl)-N-(-N-(2-(6-CF3-1H-indol-3-yl)ethyl)-N-ethyl)-N-hydroethan-1-amine
2(2-(4-D-1H-3a-azaindol-3-yl)-N-(-N-(2-(7-F-1H-indol-3-yl)ethyl)-N-ethyl)-N-hydroethan-1-amine
2(2-(5-OH-1H-3a-azaindol-3-yl)-N-(-N-(2-(7-F-1H-indol-3-yl)ethyl)-N-ethyl)-N-hydroethan-1-amine
2(2-(6-CF3-1H-3a-azaindol-3-yl)-N-(-N-(2-(7-F-1H-indol-3-yl)ethyl)-N-ethyl)-N-hydroethan-1-amine
2(2-(7-F-1H-3a-azaindol-3-yl)-N-(-N-(2-(7-F-1H-indol-3-yl)ethyl)-N-ethyl)-N-hydroethan-1-amine
2(2-(4-D-1H-3a-azaindol-3-yl)-N-(-N-(2-(4-D-1H-indol-3-yl)ethyl)-N-ethyl)-N-(D)ethan-1-amine
2(2-(5-OH-1H-3a-azaindol-3-yl)-N-(-N-(2-(4-D-1H-indol-3-yl)ethyl)-N-ethyl)-N-(D)ethan-1-amine
2(2-(6-CF3-1H-3a-azaindol-3-yl)-N-(-N-(2-(4-D-1H-indol-3-yl)ethyl)-N-ethyl)-N-(D)ethan-1-amine
2(2-(7-F-1H-3a-azaindol-3-yl)-N-(-N-(2-(4-D-1H-indol-3-yl)ethyl)-N-ethyl)-N-(D)ethan-1-amine
2(2-(4-D-1H-3a-azaindol-3-yl)-N-(-N-(2-(5-OH-1H-indol-3-yl)ethyl)-N-ethyl)-N-(D)ethan-1-amine
2(2-(5-OH-1H-3a-azaindol-3-yl)-N-(-N-(2-(5-OH-1H-indol-3-yl)ethyl)-N-ethyl)-N-(D)ethan-1-amine
2(2-(6-CF3-1H-3a-azaindol-3-yl)-N-(-N-(2-(5-OH-1H-indol-3-yl)ethyl)-N-ethyl)-N-(D)ethan-1-amine
2(2-(7-F-1H-3a-azaindol-3-yl)-N-(-N-(2-(5-OH-1H-indol-3-yl)ethyl)-N-ethyl)-N-(D)ethan-1-amine
2(2-(4-D-1H-3a-azaindol-3-yl)-N-(-N-(2-(6-CF3-1H-indol-3-yl)ethyl)-N-ethyl)-N-(D)ethan-1-amine
2(2-(5-OH-1H-3a-azaindol-3-yl)-N-(-N-(2-(6-CF3-1H-indol-3-yl)ethyl)-N-ethyl)-N-(D)ethan-1-amine
2(2-(6-CF3-1H-3a-azaindol-3-yl)-N-(-N-(2-(6-CF3-1H-indol-3-yl)ethyl)-N-ethyl)-N-(D)ethan-1-amine
2(2-(7-F-1H-3a-azaindol-3-yl)-N-(-N-(2-(6-CF3-1H-indol-3-yl)ethyl)-N-ethyl)-N-(D)ethan-1-amine
2(2-(4-D-1H-3a-azaindol-3-yl)-N-(-N-(2-(7-F-1H-indol-3-yl)ethyl)-N-ethyl)-N-(D)ethan-1-amine
2(2-(5-OH-1H-3a-azaindol-3-yl)-N-(-N-(2-(7-F-1H-indol-3-yl)ethyl)-N-ethyl)-N-(D)ethan-1-amine
2(2-(6-CF3-1H-3a-azaindol-3-yl)-N-(-N-(2-(7-F-1H-indol-3-yl)ethyl)-N-ethyl)-N-(D)ethan-1-amine
2(2-(7-F-1H-3a-azaindol-3-yl)-N-(-N-(2-(7-F-1H-indol-3-yl)ethyl)-N-ethyl)-N-(D)ethan-1-amine
2(2-(4-D-1H-3a-azaindol-3-yl)-N-(-N-(2-(4-D-1H-indol-3-yl)ethyl)-N-ethyl)-N-propylethan-1-amine
2(2-(5-OH-1H-3a-azaindol-3-yl)-N-(-N-(2-(4-D-1H-indol-3-yl)ethyl)-N-ethyl)-N-propylethan-1-amine
2(2-(6-CF3-1H-3a-azaindol-3-yl)-N-(-N-(2-(4-D-1H-indol-3-yl)ethyl)-N-ethyl)-N-propylethan-1-amine
2(2-(7-F-1H-3a-azaindol-3-yl)-N-(-N-(2-(4-D-1H-indol-3-yl)ethyl)-N-ethyl)-N-propylethan-1-amine
2(2-(4-D-1H-3a-azaindol-3-yl)-N-(-N-(2-(5-OH-1H-indol-3-yl)ethyl)-N-ethyl)-N-propylethan-1-amine
2(2-(5-OH-1H-3a-azaindol-3-yl)-N-(-N-(2-(5-OH-1H-indol-3-yl)ethyl)-N-ethyl)-N-propylethan-1-amine
2(2-(6-CF3-1H-3a-azaindol-3-yl)-N-(-N-(2-(5-OH-1H-indol-3-yl)ethyl)-N-ethyl)-N-propylethan-1-amine
2(2-(7-F-1H-3a-azaindol-3-yl)-N-(-N-(2-(5-OH-1H-indol-3-yl)ethyl)-N-ethyl)-N-propylethan-1-amine
2(2-(4-D-1H-3a-azaindol-3-yl)-N-(-N-(2-(6-CF3-1H-indol-3-yl)ethyl)-N-ethyl)-N-propylethan-1-amine
2(2-(5-OH-1H-3a-azaindol-3-yl)-N-(-N-(2-(6-CF3-1H-indol-3-yl)ethyl)-N-ethyl)-N-propylethan-1-amine
2(2-(6-CF3-1H-3a-azaindol-3-yl)-N-(-N-(2-(6-CF3-1H-indol-3-yl)ethyl)-N-ethyl)-N-propylethan-1-amine
2(2-(7-F-1H-3a-azaindol-3-yl)-N-(-N-(2-(6-CF3-1H-indol-3-yl)ethyl)-N-ethyl)-N-propylethan-1-amine
2(2-(4-D-1H-3a-azaindol-3-yl)-N-(-N-(2-(7-F-1H-indol-3-yl)ethyl)-N-ethyl)-N-propylethan-1-amine
2(2-(5-OH-1H-3a-azaindol-3-yl)-N-(-N-(2-(7-F-1H-indol-3-yl)ethyl)-N-ethyl)-N-propylethan-1-amine
2(2-(6-CF3-1H-3a-azaindol-3-yl)-N-(-N-(2-(7-F-1H-indol-3-yl)ethyl)-N-ethyl)-N-propylethan-1-amine
2(2-(7-F-1H-3a-azaindol-3-yl)-N-(-N-(2-(7-F-1H-indol-3-yl)ethyl)-N-ethyl)-N-propylethan-1-amine
2(2-(4-D-1H-5-azaindol-3-yl)-N-(-N-(2-(4-D-1H-5-azaindol-3-yl)ethyl)-N-ethyl)-N-methylethan-1-amine
2(2-(6-Me-1H-5-azaindol-3-yl)-N-(-N-(2-(4-D-1H-5-azaindol-3-yl)ethyl)-N-ethyl)-N-methylethan-1-
amine
2(2-(7-CF3-1H-5-azaindol-3-yl)-N-(-N-(2-(4-D-1H-5-azaindol-3-yl)ethyl)-N-ethyl)-N-methylethan-1-
amine
2(2-(4-D-1H-5-azaindol-3-yl)-N-(-N-(2-(6-Me-1H-5-azaindol-3-yl)ethyl)-N-ethyl)-N-methylethan-1-
amine
2(2-(6-Me-1H-5-azaindol-3-yl)-N-(-N-(2-(6-Me-1H-5-azaindol-3-yl)ethyl)-N-ethyl)-N-methylethan-1-
amine
2(2-(7-CF3-1H-5-azaindol-3-yl)-N-(-N-(2-(6-Me-1H-5-azaindol-3-yl)ethyl)-N-ethyl)-N-methylethan-1-
amine
2(2-(4-D-1H-5-azaindol-3-yl)-N-(-N-(2-(7-CF3-1H-5-azaindol-3-yl)ethyl)-N-ethyl)-N-methylethan-1-
amine
2(2-(6-Me-1H-5-azaindol-3-yl)-N-(-N-(2-(7-CF3-1H-5-azaindol-3-yl)ethyl)-N-ethyl)-N-methylethan-1-
amine
2(2-(7-CF3-1H-5-azaindol-3-yl)-N-(-N-(2-(7-CF3-1H-5-azaindol-3-yl)ethyl)-N-ethyl)-N-methylethan-1-
amine
2(2-(4-D-1H-5-azaindol-3-yl)-N-(-N-(2-(4-D-1H-5-azaindol-3-yl)ethyl)-N-ethyl)-N-ethylethan-1-amine
2(2-(6-Me-1H-5-azaindol-3-yl)-N-(-N-(2-(4-D-1H-5-azaindol-3-yl)ethyl)-N-ethyl)-N-ethylethan-1-amine
2(2-(7-CF3-1H-5-azaindol-3-yl)-N-(-N-(2-(4-D-1H-5-azaindol-3-yl)ethyl)-N-ethyl)-N-ethylethan-1-amine
2(2-(4-D-1H-5-azaindol-3-yl)-N-(-N-(2-(6-Me-1H-5-azaindol-3-yl)ethyl)-N-ethyl)-N-ethylethan-1-amine
2(2-(6-Me-1H-5-azaindol-3-yl)-N-(-N-(2-(6-Me-1H-5-azaindol-3-yl)ethyl)-N-ethyl)-N-ethylethan-1-
amine
2(2-(7-CF3-1H-5-azaindol-3-yl)-N-(-N-(2-(6-Me-1H-5-azaindol-3-yl)ethyl)-N-ethyl)-N-ethylethan-1-
amine
2(2-(4-D-1H-5-azaindol-3-yl)-N-(-N-(2-(7-CF3-1H-5-azaindol-3-yl)ethyl)-N-ethyl)-N-ethylethan-1-amine
2(2-(6-Me-1H-5-azaindol-3-yl)-N-(-N-(2-(7-CF3-1H-5-azaindol-3-yl)ethyl)-N-ethyl)-N-ethylethan-1-
amine
2(2-(7-CF3-1H-5-azaindol-3-yl)-N-(-N-(2-(7-CF3-1H-5-azaindol-3-yl)ethyl)-N-ethyl)-N-ethylethan-1-
amine
2(2-(4-D-1H-5-azaindol-3-yl)-N-(-N-(2-(4-D-1H-5-azaindol-3-yl)ethyl)-N-ethyl)-N-isopropylethan-1-
amine
2(2-(6-Me-1H-5-azaindol-3-yl)-N-(-N-(2-(4-D-1H-5-azaindol-3-yl)ethyl)-N-ethyl)-N-isopropylethan-1-
amine
2(2-(7-CF3-1H-5-azaindol-3-yl)-N-(-N-(2-(4-D-1H-5-azaindol-3-yl)ethyl)-N-ethyl)-N-isopropylethan-1-
amine
2(2-(4-D-1H-5-azaindol-3-yl)-N-(-N-(2-(6-Me-1H-5-azaindol-3-yl)ethyl)-N-ethyl)-N-isopropylethan-1-
amine
2(2-(6-Me-1H-5-azaindol-3-yl)-N-(-N-(2-(6-Me-1H-5-azaindol-3-yl)ethyl)-N-ethyl)-N-isopropylethan-1-
amine
2(2-(7-CF3-1H-5-azaindol-3-yl)-N-(-N-(2-(6-Me-1H-5-azaindol-3-yl)ethyl)-N-ethyl)-N-isopropylethan-1-
amine
2(2-(4-D-1H-5-azaindol-3-yl)-N-(-N-(2-(7-CF3-1H-5-azaindol-3-yl)ethyl)-N-ethyl)-N-isopropylethan-1-
amine
2(2-(6-Me-1H-5-azaindol-3-yl)-N-(-N-(2-(7-CF3-1H-5-azaindol-3-yl)ethyl)-N-ethyl)-N-isopropylethan-1-
amine
2(2-(7-CF3-1H-5-azaindol-3-yl)-N-(-N-(2-(7-CF3-1H-5-azaindol-3-yl)ethyl)-N-ethyl)-N-isopropylethan-1-
amine
2(2-(4-D-1H-5-azaindol-3-yl)-N-(-N-(2-(4-D-1H-5-azaindol-3-yl)ethyl)-N-ethyl)-N-allylethan-1-amine
2(2-(6-Me-1H-5-azaindol-3-yl)-N-(-N-(2-(4-D-1H-5-azaindol-3-yl)ethyl)-N-ethyl)-N-allylethan-1-amine
2(2-(7-CF3-1H-5-azaindol-3-yl)-N-(-N-(2-(4-D-1H-5-azaindol-3-yl)ethyl)-N-ethyl)-N-allylethan-1-amine
2(2-(4-D-1H-5-azaindol-3-yl)-N-(-N-(2-(6-Me-1H-5-azaindol-3-yl)ethyl)-N-ethyl)-N-allylethan-1-amine
2(2-(6-Me-1H-5-azaindol-3-yl)-N-(-N-(2-(6-Me-1H-5-azaindol-3-yl)ethyl)-N-ethyl)-N-allylethan-1-amine
2(2-(7-CF3-1H-5-azaindol-3-yl)-N-(-N-(2-(6-Me-1H-5-azaindol-3-yl)ethyl)-N-ethyl)-N-allylethan-1-
amine
2(2-(4-D-1H-5-azaindol-3-yl)-N-(-N-(2-(7-CF3-1H-5-azaindol-3-yl)ethyl)-N-ethyl)-N-allylethan-1-amine
2(2-(6-Me-1H-5-azaindol-3-yl)-N-(-N-(2-(7-CF3-1H-5-azaindol-3-yl)ethyl)-N-ethyl)-N-allylethan-1-
amine
2(2-(7-CF3-1H-5-azaindol-3-yl)-N-(-N-(2-(7-CF3-1H-5-azaindol-3-yl)ethyl)-N-ethyl)-N-allylethan-1-
amine
2(2-(4-D-1H-5-azaindol-3-yl)-N-(-N-(2-(4-D-1H-5-azaindol-3-yl)ethyl)-N-ethyl)-N-hydroethan-1-amine
2(2-(6-Me-1H-5-azaindol-3-yl)-N-(-N-(2-(4-D-1H-5-azaindol-3-yl)ethyl)-N-ethyl)-N-hydroethan-1-amine
2(2-(7-CF3-1H-5-azaindol-3-yl)-N-(-N-(2-(4-D-1H-5-azaindol-3-yl)ethyl)-N-ethyl)-N-hydroethan-1-
amine
2(2-(4-D-1H-5-azaindol-3-yl)-N-(-N-(2-(6-Me-1H-5-azaindol-3-yl)ethyl)-N-ethyl)-N-hydroethan-1-amine
2(2-(6-Me-1H-5-azaindol-3-yl)-N-(-N-(2-(6-Me-1H-5-azaindol-3-yl)ethyl)-N-ethyl)-N-hydroethan-1-
amine
2(2-(7-CF3-1H-5-azaindol-3-yl)-N-(-N-(2-(6-Me-1H-5-azaindol-3-yl)ethyl)-N-ethyl)-N-hydroethan-1-
amine
2(2-(4-D-1H-5-azaindol-3-yl)-N-(-N-(2-(7-CF3-1H-5-azaindol-3-yl)ethyl)-N-ethyl)-N-hydroethan-1-
amine
2(2-(6-Me-1H-5-azaindol-3-yl)-N-(-N-(2-(7-CF3-1H-5-azaindol-3-yl)ethyl)-N-ethyl)-N-hydroethan-1-
amine
2(2-(7-CF3-1H-5-azaindol-3-yl)-N-(-N-(2-(7-CF3-1H-5-azaindol-3-yl)ethyl)-N-ethyl)-N-hydroethan-1-
amine
2(2-(4-D-1H-5-azaindol-3-yl)-N-(-N-(2-(4-D-1H-5-azaindol-3-yl)ethyl)-N-ethyl)-N-(D)ethan-1-amine
2(2-(6-Me-1H-5-azaindol-3-yl)-N-(-N-(2-(4-D-1H-5-azaindol-3-yl)ethyl)-N-ethyl)-N-(D)ethan-1-amine
2(2-(7-CF3-1H-5-azaindol-3-yl)-N-(-N-(2-(4-D-1H-5-azaindol-3-yl)ethyl)-N-ethyl)-N-(D)ethan-1-amine
2(2-(4-D-1H-5-azaindol-3-yl)-N-(-N-(2-(6-Me-1H-5-azaindol-3-yl)ethyl)-N-ethyl)-N-(D)ethan-1-amine
2(2-(6-Me-1H-5-azaindol-3-yl)-N-(-N-(2-(6-Me-1H-5-azaindol-3-yl)ethyl)-N-ethyl)-N-(D)ethan-1-amine
2(2-(7-CF3-1H-5-azaindol-3-yl)-N-(-N-(2-(6-Me-1H-5-azaindol-3-yl)ethyl)-N-ethyl)-N-(D)ethan-1-amine
2(2-(4-D-1H-5-azaindol-3-yl)-N-(-N-(2-(7-CF3-1H-5-azaindol-3-yl)ethyl)-N-ethyl)-N-(D)ethan-1-amine
2(2-(6-Me-1H-5-azaindol-3-yl)-N-(-N-(2-(7-CF3-1H-5-azaindol-3-yl)ethyl)-N-ethyl)-N-(D)ethan-1-amine
2(2-(7-CF3-1H-5-azaindol-3-yl)-N-(-N-(2-(7-CF3-1H-5-azaindol-3-yl)ethyl)-N-ethyl)-N-(D)ethan-1-amine
2(2-(4-D-1H-5-azaindol-3-yl)-N-(-N-(2-(4-D-1H-5-azaindol-3-yl)ethyl)-N-ethyl)-N-propylethan-1-amine
2(2-(6-Me-1H-5-azaindol-3-yl)-N-(-N-(2-(4-D-1H-5-azaindol-3-yl)ethyl)-N-ethyl)-N-propylethan-1-
amine
2(2-(7-CF3-1H-5-azaindol-3-yl)-N-(-N-(2-(4-D-1H-5-azaindol-3-yl)ethyl)-N-ethyl)-N-propylethan-1-
amine
2(2-(4-D-1H-5-azaindol-3-yl)-N-(-N-(2-(6-Me-1H-5-azaindol-3-yl)ethyl)-N-ethyl)-N-propylethan-1-
amine
2(2-(6-Me-1H-5-azaindol-3-yl)-N-(-N-(2-(6-Me-1H-5-azaindol-3-yl)ethyl)-N-ethyl)-N-propylethan-1-
amine
2(2-(7-CF3-1H-5-azaindol-3-yl)-N-(-N-(2-(6-Me-1H-5-azaindol-3-yl)ethyl)-N-ethyl)-N-propylethan-1-
amine
2(2-(4-D-1H-5-azaindol-3-yl)-N-(-N-(2-(7-CF3-1H-5-azaindol-3-yl)ethyl)-N-ethyl)-N-propylethan-1-
amine
2(2-(6-Me-1H-5-azaindol-3-yl)-N-(-N-(2-(7-CF3-1H-5-azaindol-3-yl)ethyl)-N-ethyl)-N-propylethan-1-
amine
2(2-(7-CF3-1H-5-azaindol-3-yl)-N-(-N-(2-(7-CF3-1H-5-azaindol-3-yl)ethyl)-N-ethyl)-N-propylethan-1-
amine
2(2-(4-D-1H-5-azaindol-3-yl)-N-(-N-(2-(4-D-1H-6-azaindol-3-yl)ethyl)-N-ethyl)-N-methylethan-1-amine
2(2-(6-Me-1H-5-azaindol-3-yl)-N-(-N-(2-(4-D-1H-6-azaindol-3-yl)ethyl)-N-ethyl)-N-methylethan-1-
amine
2(2-(7-CF3-1H-5-azaindol-3-yl)-N-(-N-(2-(4-D-1H-6-azaindol-3-yl)ethyl)-N-ethyl)-N-methylethan-1-
amine
2(2-(4-D-1H-5-azaindol-3-yl)-N-(-N-(2-(5-OH-1H-6-azaindol-3-yl)ethyl)-N-ethyl)-N-methylethan-1-
amine
2(2-(6-Me-1H-5-azaindol-3-yl)-N-(-N-(2-(5-OH-1H-6-azaindol-3-yl)ethyl)-N-ethyl)-N-methylethan-1-
amine
2(2-(7-CF3-1H-5-azaindol-3-yl)-N-(-N-(2-(5-OH-1H-6-azaindol-3-yl)ethyl)-N-ethyl)-N-methylethan-1-
amine
2(2-(4-D-1H-5-azaindol-3-yl)-N-(-N-(2-(7-CF3-1H-6-azaindol-3-yl)ethyl)-N-ethyl)-N-methylethan-1-
amine
2(2-(6-Me-1H-5-azaindol-3-yl)-N-(-N-(2-(7-CF3-1H-6-azaindol-3-yl)ethyl)-N-ethyl)-N-methylethan-1-
amine
2(2-(7-CF3-1H-5-azaindol-3-yl)-N-(-N-(2-(7-CF3-1H-6-azaindol-3-yl)ethyl)-N-ethyl)-N-methylethan-1-
amine
2(2-(4-D-1H-5-azaindol-3-yl)-N-(-N-(2-(4-D-1H-6-azaindol-3-yl)ethyl)-N-ethyl)-N-ethylethan-1-amine
2(2-(6-Me-1H-5-azaindol-3-yl)-N-(-N-(2-(4-D-1H-6-azaindol-3-yl)ethyl)-N-ethyl)-N-ethylethan-1-amine
2(2-(7-CF3-1H-5-azaindol-3-yl)-N-(-N-(2-(4-D-1H-6-azaindol-3-yl)ethyl)-N-ethyl)-N-ethylethan-1-amine
2(2-(4-D-1H-5-azaindol-3-yl)-N-(-N-(2-(5-OH-1H-6-azaindol-3-yl)ethyl)-N-ethyl)-N-ethylethan-1-amine
2(2-(6-Me-1H-5-azaindol-3-yl)-N-(-N-(2-(5-OH-1H-6-azaindol-3-yl)ethyl)-N-ethyl)-N-ethylethan-1-
amine
2(2-(7-CF3-1H-5-azaindol-3-yl)-N-(-N-(2-(5-OH-1H-6-azaindol-3-yl)ethyl)-N-ethyl)-N-ethylethan-1-
amine
2(2-(4-D-1H-5-azaindol-3-yl)-N-(-N-(2-(7-CF3-1H-6-azaindol-3-yl)ethyl)-N-ethyl)-N-ethylethan-1-amine
2(2-(6-Me-1H-5-azaindol-3-yl)-N-(-N-(2-(7-CF3-1H-6-azaindol-3-yl)ethyl)-N-ethyl)-N-ethylethan-1-
amine
2(2-(7-CF3-1H-5-azaindol-3-yl)-N-(-N-(2-(7-CF3-1H-6-azaindol-3-yl)ethyl)-N-ethyl)-N-ethylethan-1-
amine
2(2-(4-D-1H-5-azaindol-3-yl)-N-(-N-(2-(4-D-1H-6-azaindol-3-yl)ethyl)-N-ethyl)-N-isopropylethan-1-
amine
2(2-(6-Me-1H-5-azaindol-3-yl)-N-(-N-(2-(4-D-1H-6-azaindol-3-yl)ethyl)-N-ethyl)-N-isopropylethan-1-
amine
2(2-(7-CF3-1H-5-azaindol-3-yl)-N-(-N-(2-(4-D-1H-6-azaindol-3-yl)ethyl)-N-ethyl)-N-isopropylethan-1-
amine
2(2-(4-D-1H-5-azaindol-3-yl)-N-(-N-(2-(5-OH-1H-6-azaindol-3-yl)ethyl)-N-ethyl)-N-isopropylethan-1-
amine
2(2-(6-Me-1H-5-azaindol-3-yl)-N-(-N-(2-(5-OH-1H-6-azaindol-3-yl)ethyl)-N-ethyl)-N-isopropylethan-1-
amine
2(2-(7-CF3-1H-5-azaindol-3-yl)-N-(-N-(2-(5-OH-1H-6-azaindol-3-yl)ethyl)-N-ethyl)-N-isopropylethan-1-
amine
2(2-(4-D-1H-5-azaindol-3-yl)-N-(-N-(2-(7-CF3-1H-6-azaindol-3-yl)ethyl)-N-ethyl)-N-isopropylethan-1-
amine
2(2-(6-Me-1H-5-azaindol-3-yl)-N-(-N-(2-(7-CF3-1H-6-azaindol-3-yl)ethyl)-N-ethyl)-N-isopropylethan-1-
amine
2(2-(7-CF3-1H-5-azaindol-3-yl)-N-(-N-(2-(7-CF3-1H-6-azaindol-3-yl)ethyl)-N-ethyl)-N-isopropylethan-1-
amine
2(2-(4-D-1H-5-azaindol-3-yl)-N-(-N-(2-(4-D-1H-6-azaindol-3-yl)ethyl)-N-ethyl)-N-allylethan-1-amine
2(2-(6-Me-1H-5-azaindol-3-yl)-N-(-N-(2-(4-D-1H-6-azaindol-3-yl)ethyl)-N-ethyl)-N-allylethan-1-amine
2(2-(7-CF3-1H-5-azaindol-3-yl)-N-(-N-(2-(4-D-1H-6-azaindol-3-yl)ethyl)-N-ethyl)-N-allylethan-1-amine
2(2-(4-D-1H-5-azaindol-3-yl)-N-(-N-(2-(5-OH-1H-6-azaindol-3-yl)ethyl)-N-ethyl)-N-allylethan-1-amine
2(2-(6-Me-1H-5-azaindol-3-yl)-N-(-N-(2-(5-OH-1H-6-azaindol-3-yl)ethyl)-N-ethyl)-N-allylethan-1-amine
2(2-(7-CF3-1H-5-azaindol-3-yl)-N-(-N-(2-(5-OH-1H-6-azaindol-3-yl)ethyl)-N-ethyl)-N-allylethan-1-amine
2(2-(4-D-1H-5-azaindol-3-yl)-N-(-N-(2-(7-CF3-1H-6-azaindol-3-yl)ethyl)-N-ethyl)-N-allylethan-1-amine
2(2-(6-Me-1H-5-azaindol-3-yl)-N-(-N-(2-(7-CF3-1H-6-azaindol-3-yl)ethyl)-N-ethyl)-N-allylethan-1-
amine
2(2-(7-CF3-1H-5-azaindol-3-yl)-N-(-N-(2-(7-CF3-1H-6-azaindol-3-yl)ethyl)-N-ethyl)-N-allylethan-1-
amine
2(2-(4-D-1H-5-azaindol-3-yl)-N-(-N-(2-(4-D-1H-6-azaindol-3-yl)ethyl)-N-ethyl)-N-hydroethan-1-amine
2(2-(6-Me-1H-5-azaindol-3-yl)-N-(-N-(2-(4-D-1H-6-azaindol-3-yl)ethyl)-N-ethyl)-N-hydroethan-1-amine
2(2-(7-CF3-1H-5-azaindol-3-yl)-N-(-N-(2-(4-D-1H-6-azaindol-3-yl)ethyl)-N-ethyl)-N-hydroethan-1-
amine
2(2-(4-D-1H-5-azaindol-3-yl)-N-(-N-(2-(5-OH-1H-6-azaindol-3-yl)ethyl)-N-ethyl)-N-hydroethan-1-amine
2(2-(6-Me-1H-5-azaindol-3-yl)-N-(-N-(2-(5-OH-1H-6-azaindol-3-yl)ethyl)-N-ethyl)-N-hydroethan-1-
amine
2(2-(7-CF3-1H-5-azaindol-3-yl)-N-(-N-(2-(5-OH-1H-6-azaindol-3-yl)ethyl)-N-ethyl)-N-hydroethan-1-
amine
2(2-(4-D-1H-5-azaindol-3-yl)-N-(-N-(2-(7-CF3-1H-6-azaindol-3-yl)ethyl)-N-ethyl)-N-hydroethan-1-
amine
2(2-(6-Me-1H-5-azaindol-3-yl)-N-(-N-(2-(7-CF3-1H-6-azaindol-3-yl)ethyl)-N-ethyl)-N-hydroethan-1-
amine
2(2-(7-CF3-1H-5-azaindol-3-yl)-N-(-N-(2-(7-CF3-1H-6-azaindol-3-yl)ethyl)-N-ethyl)-N-hydroethan-1-
amine
2(2-(4-D-1H-5-azaindol-3-yl)-N-(-N-(2-(4-D-1H-6-azaindol-3-yl)ethyl)-N-ethyl)-N-(D)ethan-1-amine
2(2-(6-Me-1H-5-azaindol-3-yl)-N-(-N-(2-(4-D-1H-6-azaindol-3-yl)ethyl)-N-ethyl)-N-(D)ethan-1-amine
2(2-(7-CF3-1H-5-azaindol-3-yl)-N-(-N-(2-(4-D-1H-6-azaindol-3-yl)ethyl)-N-ethyl)-N-(D)ethan-1-amine
2(2-(4-D-1H-5-azaindol-3-yl)-N-(-N-(2-(5-OH-1H-6-azaindol-3-yl)ethyl)-N-ethyl)-N-(D)ethan-1-amine
2(2-(6-Me-1H-5-azaindol-3-yl)-N-(-N-(2-(5-OH-1H-6-azaindol-3-yl)ethyl)-N-ethyl)-N-(D)ethan-1-amine
2(2-(7-CF3-1H-5-azaindol-3-yl)-N-(-N-(2-(5-OH-1H-6-azaindol-3-yl)ethyl)-N-ethyl)-N-(D)ethan-1-amine
2(2-(4-D-1H-5-azaindol-3-yl)-N-(-N-(2-(7-CF3-1H-6-azaindol-3-yl)ethyl)-N-ethyl)-N-(D)ethan-1-amine
2(2-(6-Me-1H-5-azaindol-3-yl)-N-(-N-(2-(7-CF3-1H-6-azaindol-3-yl)ethyl)-N-ethyl)-N-(D)ethan-1-amine
2(2-(7-CF3-1H-5-azaindol-3-yl)-N-(-N-(2-(7-CF3-1H-6-azaindol-3-yl)ethyl)-N-ethyl)-N-(D)ethan-1-amine
2(2-(4-D-1H-5-azaindol-3-yl)-N-(-N-(2-(4-D-1H-6-azaindol-3-yl)ethyl)-N-ethyl)-N-propylethan-1-amine
2(2-(6-Me-1H-5-azaindol-3-yl)-N-(-N-(2-(4-D-1H-6-azaindol-3-yl)ethyl)-N-ethyl)-N-propylethan-1-
amine
2(2-(7-CF3-1H-5-azaindol-3-yl)-N-(-N-(2-(4-D-1H-6-azaindol-3-yl)ethyl)-N-ethyl)-N-propylethan-1-
amine
2(2-(4-D-1H-5-azaindol-3-yl)-N-(-N-(2-(5-OH-1H-6-azaindol-3-yl)ethyl)-N-ethyl)-N-propylethan-1-
amine
2(2-(6-Me-1H-5-azaindol-3-yl)-N-(-N-(2-(5-OH-1H-6-azaindol-3-yl)ethyl)-N-ethyl)-N-propylethan-1-
amine
2(2-(7-CF3-1H-5-azaindol-3-yl)-N-(-N-(2-(5-OH-1H-6-azaindol-3-yl)ethyl)-N-ethyl)-N-propylethan-1-
amine
2(2-(4-D-1H-5-azaindol-3-yl)-N-(-N-(2-(7-CF3-1H-6-azaindol-3-yl)ethyl)-N-ethyl)-N-propylethan-1-
amine
2(2-(6-Me-1H-5-azaindol-3-yl)-N-(-N-(2-(7-CF3-1H-6-azaindol-3-yl)ethyl)-N-ethyl)-N-propylethan-1-
amine
2(2-(7-CF3-1H-5-azaindol-3-yl)-N-(-N-(2-(7-CF3-1H-6-azaindol-3-yl)ethyl)-N-ethyl)-N-propylethan-1-
amine
2(2-(4-D-1H-5-azaindol-3-yl)-N-(-N-(2-(4-D-1H-7-azaindol-3-yl)ethyl)-N-ethyl)-N-methylethan-1-amine
2(2-(6-Me-1H-5-azaindol-3-yl)-N-(-N-(2-(4-D-1H-7-azaindol-3-yl)ethyl)-N-ethyl)-N-methylethan-1-
amine
2(2-(7-CF3-1H-5-azaindol-3-yl)-N-(-N-(2-(4-D-1H-7-azaindol-3-yl)ethyl)-N-ethyl)-N-methylethan-1-
amine
2(2-(4-D-1H-5-azaindol-3-yl)-N-(-N-(2-(5-OH-1H-7-azaindol-3-yl)ethyl)-N-ethyl)-N-methylethan-1-
amine
2(2-(6-Me-1H-5-azaindol-3-yl)-N-(-N-(2-(5-OH-1H-7-azaindol-3-yl)ethyl)-N-ethyl)-N-methylethan-1-
amine
2(2-(7-CF3-1H-5-azaindol-3-yl)-N-(-N-(2-(5-OH-1H-7-azaindol-3-yl)ethyl)-N-ethyl)-N-methylethan-1-
amine
2(2-(4-D-1H-5-azaindol-3-yl)-N-(-N-(2-(6-CF3-1H-7-azaindol-3-yl)ethyl)-N-ethyl)-N-methylethan-1-
amine
2(2-(6-Me-1H-5-azaindol-3-yl)-N-(-N-(2-(6-CF3-1H-7-azaindol-3-yl)ethyl)-N-ethyl)-N-methylethan-1-
amine
2(2-(7-CF3-1H-5-azaindol-3-yl)-N-(-N-(2-(6-CF3-1H-7-azaindol-3-yl)ethyl)-N-ethyl)-N-methylethan-1-
amine
2(2-(4-D-1H-5-azaindol-3-yl)-N-(-N-(2-(4-D-1H-7-azaindol-3-yl)ethyl)-N-ethyl)-N-ethylethan-1-amine
2(2-(6-Me-1H-5-azaindol-3-yl)-N-(-N-(2-(4-D-1H-7-azaindol-3-yl)ethyl)-N-ethyl)-N-ethylethan-1-amine
2(2-(7-CF3-1H-5-azaindol-3-yl)-N-(-N-(2-(4-D-1H-7-azaindol-3-yl)ethyl)-N-ethyl)-N-ethylethan-1-amine
2(2-(4-D-1H-5-azaindol-3-yl)-N-(-N-(2-(5-OH-1H-7-azaindol-3-yl)ethyl)-N-ethyl)-N-ethylethan-1-amine
2(2-(6-Me-1H-5-azaindol-3-yl)-N-(-N-(2-(5-OH-1H-7-azaindol-3-yl)ethyl)-N-ethyl)-N-ethylethan-1-
amine
2(2-(7-CF3-1H-5-azaindol-3-yl)-N-(-N-(2-(5-OH-1H-7-azaindol-3-yl)ethyl)-N-ethyl)-N-ethylethan-1-
amine
2(2-(4-D-1H-5-azaindol-3-yl)-N-(-N-(2-(6-CF3-1H-7-azaindol-3-yl)ethyl)-N-ethyl)-N-ethylethan-1-amine
2(2-(6-Me-1H-5-azaindol-3-yl)-N-(-N-(2-(6-CF3-1H-7-azaindol-3-yl)ethyl)-N-ethyl)-N-ethylethan-1-
amine
2(2-(7-CF3-1H-5-azaindol-3-yl)-N-(-N-(2-(6-CF3-1H-7-azaindol-3-yl)ethyl)-N-ethyl)-N-ethylethan-1-
amine
2(2-(4-D-1H-5-azaindol-3-yl)-N-(-N-(2-(4-D-1H-7-azaindol-3-yl)ethyl)-N-ethyl)-N-isopropylethan-1-
amine
2(2-(6-Me-1H-5-azaindol-3-yl)-N-(-N-(2-(4-D-1H-7-azaindol-3-yl)ethyl)-N-ethyl)-N-isopropylethan-1-
amine
2(2-(7-CF3-1H-5-azaindol-3-yl)-N-(-N-(2-(4-D-1H-7-azaindol-3-yl)ethyl)-N-ethyl)-N-isopropylethan-1-
amine
2(2-(4-D-1H-5-azaindol-3-yl)-N-(-N-(2-(5-OH-1H-7-azaindol-3-yl)ethyl)-N-ethyl)-N-isopropylethan-1-
amine
2(2-(6-Me-1H-5-azaindol-3-yl)-N-(-N-(2-(5-OH-1H-7-azaindol-3-yl)ethyl)-N-ethyl)-N-isopropylethan-1-
amine
2(2-(7-CF3-1H-5-azaindol-3-yl)-N-(-N-(2-(5-OH-1H-7-azaindol-3-yl)ethyl)-N-ethyl)-N-isopropylethan-1-
amine
2(2-(4-D-1H-5-azaindol-3-yl)-N-(-N-(2-(6-CF3-1H-7-azaindol-3-yl)ethyl)-N-ethyl)-N-isopropylethan-1-
amine
2(2-(6-Me-1H-5-azaindol-3-yl)-N-(-N-(2-(6-CF3-1H-7-azaindol-3-yl)ethyl)-N-ethyl)-N-isopropylethan-1-
amine
2(2-(7-CF3-1H-5-azaindol-3-yl)-N-(-N-(2-(6-CF3-1H-7-azaindol-3-yl)ethyl)-N-ethyl)-N-isopropylethan-1-
amine
2(2-(4-D-1H-5-azaindol-3-yl)-N-(-N-(2-(4-D-1H-7-azaindol-3-yl)ethyl)-N-ethyl)-N-allylethan-1-amine
2(2-(6-Me-1H-5-azaindol-3-yl)-N-(-N-(2-(4-D-1H-7-azaindol-3-yl)ethyl)-N-ethyl)-N-allylethan-1-amine
2(2-(7-CF3-1H-5-azaindol-3-yl)-N-(-N-(2-(4-D-1H-7-azaindol-3-yl)ethyl)-N-ethyl)-N-allylethan-1-amine
2(2-(4-D-1H-5-azaindol-3-yl)-N-(-N-(2-(5-OH-1H-7-azaindol-3-yl)ethyl)-N-ethyl)-N-allylethan-1-amine
2(2-(6-Me-1H-5-azaindol-3-yl)-N-(-N-(2-(5-OH-1H-7-azaindol-3-yl)ethyl)-N-ethyl)-N-allylethan-1-amine
2(2-(7-CF3-1H-5-azaindol-3-yl)-N-(-N-(2-(5-OH-1H-7-azaindol-3-yl)ethyl)-N-ethyl)-N-allylethan-1-amine
2(2-(4-D-1H-5-azaindol-3-yl)-N-(-N-(2-(6-CF3-1H-7-azaindol-3-yl)ethyl)-N-ethyl)-N-allylethan-1-amine
2(2-(6-Me-1H-5-azaindol-3-yl)-N-(-N-(2-(6-CF3-1H-7-azaindol-3-yl)ethyl)-N-ethyl)-N-allylethan-1-
amine
2(2-(7-CF3-1H-5-azaindol-3-yl)-N-(-N-(2-(6-CF3-1H-7-azaindol-3-yl)ethyl)-N-ethyl)-N-allylethan-1-
amine
2(2-(4-D-1H-5-azaindol-3-yl)-N-(-N-(2-(4-D-1H-7-azaindol-3-yl)ethyl)-N-ethyl)-N-hydroethan-1-amine
2(2-(6-Me-1H-5-azaindol-3-yl)-N-(-N-(2-(4-D-1H-7-azaindol-3-yl)ethyl)-N-ethyl)-N-hydroethan-1-amine
2(2-(7-CF3-1H-5-azaindol-3-yl)-N-(-N-(2-(4-D-1H-7-azaindol-3-yl)ethyl)-N-ethyl)-N-hydroethan-1-
amine
2(2-(4-D-1H-5-azaindol-3-yl)-N-(-N-(2-(5-OH-1H-7-azaindol-3-yl)ethyl)-N-ethyl)-N-hydroethan-1-amine
2(2-(6-Me-1H-5-azaindol-3-yl)-N-(-N-(2-(5-OH-1H-7-azaindol-3-yl)ethyl)-N-ethyl)-N-hydroethan-1-
amine
2(2-(7-CF3-1H-5-azaindol-3-yl)-N-(-N-(2-(5-OH-1H-7-azaindol-3-yl)ethyl)-N-ethyl)-N-hydroethan-1-
amine
2(2-(4-D-1H-5-azaindol-3-yl)-N-(-N-(2-(6-CF3-1H-7-azaindol-3-yl)ethyl)-N-ethyl)-N-hydroethan-1-
amine
2(2-(6-Me-1H-5-azaindol-3-yl)-N-(-N-(2-(6-CF3-1H-7-azaindol-3-yl)ethyl)-N-ethyl)-N-hydroethan-1-
amine
2(2-(7-CF3-1H-5-azaindol-3-yl)-N-(-N-(2-(6-CF3-1H-7-azaindol-3-yl)ethyl)-N-ethyl)-N-hydroethan-1-
amine
2(2-(4-D-1H-5-azaindol-3-yl)-N-(-N-(2-(4-D-1H-7-azaindol-3-yl)ethyl)-N-ethyl)-N-(D)ethan-1-amine
2(2-(6-Me-1H-5-azaindol-3-yl)-N-(-N-(2-(4-D-1H-7-azaindol-3-yl)ethyl)-N-ethyl)-N-(D)ethan-1-amine
2(2-(7-CF3-1H-5-azaindol-3-yl)-N-(-N-(2-(4-D-1H-7-azaindol-3-yl)ethyl)-N-ethyl)-N-(D)ethan-1-amine
2(2-(4-D-1H-5-azaindol-3-yl)-N-(-N-(2-(5-OH-1H-7-azaindol-3-yl)ethyl)-N-ethyl)-N-(D)ethan-1-amine
2(2-(6-Me-1H-5-azaindol-3-yl)-N-(-N-(2-(5-OH-1H-7-azaindol-3-yl)ethyl)-N-ethyl)-N-(D)ethan-1-amine
2(2-(7-CF3-1H-5-azaindol-3-yl)-N-(-N-(2-(5-OH-1H-7-azaindol-3-yl)ethyl)-N-ethyl)-N-(D)ethan-1-amine
2(2-(4-D-1H-5-azaindol-3-yl)-N-(-N-(2-(6-CF3-1H-7-azaindol-3-yl)ethyl)-N-ethyl)-N-(D)ethan-1-amine
2(2-(6-Me-1H-5-azaindol-3-yl)-N-(-N-(2-(6-CF3-1H-7-azaindol-3-yl)ethyl)-N-ethyl)-N-(D)ethan-1-amine
2(2-(7-CF3-1H-5-azaindol-3-yl)-N-(-N-(2-(6-CF3-1H-7-azaindol-3-yl)ethyl)-N-ethyl)-N-(D)ethan-1-amine
2(2-(4-D-1H-5-azaindol-3-yl)-N-(-N-(2-(4-D-1H-7-azaindol-3-yl)ethyl)-N-ethyl)-N-propylethan-1-amine
2(2-(6-Me-1H-5-azaindol-3-yl)-N-(-N-(2-(4-D-1H-7-azaindol-3-yl)ethyl)-N-ethyl)-N-propylethan-1-
amine
2(2-(7-CF3-1H-5-azaindol-3-yl)-N-(-N-(2-(4-D-1H-7-azaindol-3-yl)ethyl)-N-ethyl)-N-propylethan-1-
amine
2(2-(4-D-1H-5-azaindol-3-yl)-N-(-N-(2-(5-OH-1H-7-azaindol-3-yl)ethyl)-N-ethyl)-N-propylethan-1-
amine
2(2-(6-Me-1H-5-azaindol-3-yl)-N-(-N-(2-(5-OH-1H-7-azaindol-3-yl)ethyl)-N-ethyl)-N-propylethan-1-
amine
2(2-(7-CF3-1H-5-azaindol-3-yl)-N-(-N-(2-(5-OH-1H-7-azaindol-3-yl)ethyl)-N-ethyl)-N-propylethan-1-
amine
2(2-(4-D-1H-5-azaindol-3-yl)-N-(-N-(2-(6-CF3-1H-7-azaindol-3-yl)ethyl)-N-ethyl)-N-propylethan-1-
amine
2(2-(6-Me-1H-5-azaindol-3-yl)-N-(-N-(2-(6-CF3-1H-7-azaindol-3-yl)ethyl)-N-ethyl)-N-propylethan-1-
amine
2(2-(7-CF3-1H-5-azaindol-3-yl)-N-(-N-(2-(6-CF3-1H-7-azaindol-3-yl)ethyl)-N-ethyl)-N-propylethan-1-
amine
2(2-(4-D-1H-5-azaindol-3-yl)-N-(-N-(2-(4-D-1H-7a-azaindol-3-yl)ethyl)-N-ethyl)-N-methylethan-1-
amine
2(2-(6-Me-1H-5-azaindol-3-yl)-N-(-N-(2-(4-D-1H-7a-azaindol-3-yl)ethyl)-N-ethyl)-N-methylethan-1-
amine
2(2-(7-CF3-1H-5-azaindol-3-yl)-N-(-N-(2-(4-D-1H-7a-azaindol-3-yl)ethyl)-N-ethyl)-N-methylethan-1-
amine
2(2-(4-D-1H-5-azaindol-3-yl)-N-(-N-(2-(5-OH-1H-7a-azaindol-3-yl)ethyl)-N-ethyl)-N-methylethan-1-
amine
2(2-(6-Me-1H-5-azaindol-3-yl)-N-(-N-(2-(5-OH-1H-7a-azaindol-3-yl)ethyl)-N-ethyl)-N-methylethan-1-
amine
2(2-(7-CF3-1H-5-azaindol-3-yl)-N-(-N-(2-(5-OH-1H-7a-azaindol-3-yl)ethyl)-N-ethyl)-N-methylethan-1-
amine
2(2-(4-D-1H-5-azaindol-3-yl)-N-(-N-(2-(6-CF3-1H-7a-azaindol-3-yl)ethyl)-N-ethyl)-N-methylethan-1-
amine
2(2-(6-Me-1H-5-azaindol-3-yl)-N-(-N-(2-(6-CF3-1H-7a-azaindol-3-yl)ethyl)-N-ethyl)-N-methylethan-1-
amine
2(2-(7-CF3-1H-5-azaindol-3-yl)-N-(-N-(2-(6-CF3-1H-7a-azaindol-3-yl)ethyl)-N-ethyl)-N-methylethan-1-
amine
2(2-(4-D-1H-5-azaindol-3-yl)-N-(-N-(2-(7-F-1H-7a-azaindol-3-yl)ethyl)-N-ethyl)-N-methylethan-1-amine
2(2-(6-Me-1H-5-azaindol-3-yl)-N-(-N-(2-(7-F-1H-7a-azaindol-3-yl)ethyl)-N-ethyl)-N-methylethan-1-
amine
2(2-(7-CF3-1H-5-azaindol-3-yl)-N-(-N-(2-(7-F-1H-7a-azaindol-3-yl)ethyl)-N-ethyl)-N-methylethan-1-
amine
2(2-(4-D-1H-5-azaindol-3-yl)-N-(-N-(2-(4-D-1H-7a-azaindol-3-yl)ethyl)-N-ethyl)-N-ethylethan-1-amine
2(2-(6-Me-1H-5-azaindol-3-yl)-N-(-N-(2-(4-D-1H-7a-azaindol-3-yl)ethyl)-N-ethyl)-N-ethylethan-1-
amine
2(2-(7-CF3-1H-5-azaindol-3-yl)-N-(-N-(2-(4-D-1H-7a-azaindol-3-yl)ethyl)-N-ethyl)-N-ethylethan-1-
amine
2(2-(4-D-1H-5-azaindol-3-yl)-N-(-N-(2-(5-OH-1H-7a-azaindol-3-yl)ethyl)-N-ethyl)-N-ethylethan-1-amine
2(2-(6-Me-1H-5-azaindol-3-yl)-N-(-N-(2-(5-OH-1H-7a-azaindol-3-yl)ethyl)-N-ethyl)-N-ethylethan-1-
amine
2(2-(7-CF3-1H-5-azaindol-3-yl)-N-(-N-(2-(5-OH-1H-7a-azaindol-3-yl)ethyl)-N-ethyl)-N-ethylethan-1-
amine
2(2-(4-D-1H-5-azaindol-3-yl)-N-(-N-(2-(6-CF3-1H-7a-azaindol-3-yl)ethyl)-N-ethyl)-N-ethylethan-1-
amine
2(2-(6-Me-1H-5-azaindol-3-yl)-N-(-N-(2-(6-CF3-1H-7a-azaindol-3-yl)ethyl)-N-ethyl)-N-ethylethan-1-
amine
2(2-(7-CF3-1H-5-azaindol-3-yl)-N-(-N-(2-(6-CF3-1H-7a-azaindol-3-yl)ethyl)-N-ethyl)-N-ethylethan-1-
amine
2(2-(4-D-1H-5-azaindol-3-yl)-N-(-N-(2-(7-F-1H-7a-azaindol-3-yl)ethyl)-N-ethyl)-N-ethylethan-1-amine
2(2-(6-Me-1H-5-azaindol-3-yl)-N-(-N-(2-(7-F-1H-7a-azaindol-3-yl)ethyl)-N-ethyl)-N-ethylethan-1-amine
2(2-(7-CF3-1H-5-azaindol-3-yl)-N-(-N-(2-(7-F-1H-7a-azaindol-3-yl)ethyl)-N-ethyl)-N-ethylethan-1-
amine
2(2-(4-D-1H-5-azaindol-3-yl)-N-(-N-(2-(4-D-1H-7a-azaindol-3-yl)ethyl)-N-ethyl)-N-isopropylethan-1-
amine
2(2-(6-Me-1H-5-azaindol-3-yl)-N-(-N-(2-(4-D-1H-7a-azaindol-3-yl)ethyl)-N-ethyl)-N-isopropylethan-1-
amine
2(2-(7-CF3-1H-5-azaindol-3-yl)-N-(-N-(2-(4-D-1H-7a-azaindol-3-yl)ethyl)-N-ethyl)-N-isopropylethan-1-
amine
2(2-(4-D-1H-5-azaindol-3-yl)-N-(-N-(2-(5-OH-1H-7a-azaindol-3-yl)ethyl)-N-ethyl)-N-isopropylethan-1-
amine
2(2-(6-Me-1H-5-azaindol-3-yl)-N-(-N-(2-(5-OH-1H-7a-azaindol-3-yl)ethyl)-N-ethyl)-N-isopropylethan-1-
amine
2(2-(7-CF3-1H-5-azaindol-3-yl)-N-(-N-(2-(5-OH-1H-7a-azaindol-3-yl)ethyl)-N-ethyl)-N-isopropylethan-
1-amine
2(2-(4-D-1H-5-azaindol-3-yl)-N-(-N-(2-(6-CF3-1H-7a-azaindol-3-yl)ethyl)-N-ethyl)-N-isopropylethan-1-
amine
2(2-(6-Me-1H-5-azaindol-3-yl)-N-(-N-(2-(6-CF3-1H-7a-azaindol-3-yl)ethyl)-N-ethyl)-N-isopropylethan-
1-amine
2(2-(7-CF3-1H-5-azaindol-3-yl)-N-(-N-(2-(6-CF3-1H-7a-azaindol-3-yl)ethyl)-N-ethyl)-N-isopropylethan-
1-amine
2(2-(4-D-1H-5-azaindol-3-yl)-N-(-N-(2-(7-F-1H-7a-azaindol-3-yl)ethyl)-N-ethyl)-N-isopropylethan-1-
amine
2(2-(6-Me-1H-5-azaindol-3-yl)-N-(-N-(2-(7-F-1H-7a-azaindol-3-yl)ethyl)-N-ethyl)-N-isopropylethan-1-
amine
2(2-(7-CF3-1H-5-azaindol-3-yl)-N-(-N-(2-(7-F-1H-7a-azaindol-3-yl)ethyl)-N-ethyl)-N-isopropylethan-1-
amine
2(2-(4-D-1H-5-azaindol-3-yl)-N-(-N-(2-(4-D-1H-7a-azaindol-3-yl)ethyl)-N-ethyl)-N-allylethan-1-amine
2(2-(6-Me-1H-5-azaindol-3-yl)-N-(-N-(2-(4-D-1H-7a-azaindol-3-yl)ethyl)-N-ethyl)-N-allylethan-1-amine
2(2-(7-CF3-1H-5-azaindol-3-yl)-N-(-N-(2-(4-D-1H-7a-azaindol-3-yl)ethyl)-N-ethyl)-N-allylethan-1-amine
2(2-(4-D-1H-5-azaindol-3-yl)-N-(-N-(2-(5-OH-1H-7a-azaindol-3-yl)ethyl)-N-ethyl)-N-allylethan-1-amine
2(2-(6-Me-1H-5-azaindol-3-yl)-N-(-N-(2-(5-OH-1H-7a-azaindol-3-yl)ethyl)-N-ethyl)-N-allylethan-1-
amine
2(2-(7-CF3-1H-5-azaindol-3-yl)-N-(-N-(2-(5-OH-1H-7a-azaindol-3-yl)ethyl)-N-ethyl)-N-allylethan-1-
amine
2(2-(4-D-1H-5-azaindol-3-yl)-N-(-N-(2-(6-CF3-1H-7a-azaindol-3-yl)ethyl)-N-ethyl)-N-allylethan-1-amine
2(2-(6-Me-1H-5-azaindol-3-yl)-N-(-N-(2-(6-CF3-1H-7a-azaindol-3-yl)ethyl)-N-ethyl)-N-allylethan-1-
amine
2(2-(7-CF3-1H-5-azaindol-3-yl)-N-(-N-(2-(6-CF3-1H-7a-azaindol-3-yl)ethyl)-N-ethyl)-N-allylethan-1-
amine
2(2-(4-D-1H-5-azaindol-3-yl)-N-(-N-(2-(7-F-1H-7a-azaindol-3-yl)ethyl)-N-ethyl)-N-allylethan-1-amine
2(2-(6-Me-1H-5-azaindol-3-yl)-N-(-N-(2-(7-F-1H-7a-azaindol-3-yl)ethyl)-N-ethyl)-N-allylethan-1-amine
2(2-(7-CF3-1H-5-azaindol-3-yl)-N-(-N-(2-(7-F-1H-7a-azaindol-3-yl)ethyl)-N-ethyl)-N-allylethan-1-amine
2(2-(4-D-1H-5-azaindol-3-yl)-N-(-N-(2-(4-D-1H-7a-azaindol-3-yl)ethyl)-N-ethyl)-N-hydroethan-1-amine
2(2-(6-Me-1H-5-azaindol-3-yl)-N-(-N-(2-(4-D-1H-7a-azaindol-3-yl)ethyl)-N-ethyl)-N-hydroethan-1-
amine
2(2-(7-CF3-1H-5-azaindol-3-yl)-N-(-N-(2-(4-D-1H-7a-azaindol-3-yl)ethyl)-N-ethyl)-N-hydroethan-1-
amine
2(2-(4-D-1H-5-azaindol-3-yl)-N-(-N-(2-(5-OH-1H-7a-azaindol-3-yl)ethyl)-N-ethyl)-N-hydroethan-1-
amine
2(2-(6-Me-1H-5-azaindol-3-yl)-N-(-N-(2-(5-OH-1H-7a-azaindol-3-yl)ethyl)-N-ethyl)-N-hydroethan-1-
amine
2(2-(7-CF3-1H-5-azaindol-3-yl)-N-(-N-(2-(5-OH-1H-7a-azaindol-3-yl)ethyl)-N-ethyl)-N-hydroethan-1-
amine
2(2-(4-D-1H-5-azaindol-3-yl)-N-(-N-(2-(6-CF3-1H-7a-azaindol-3-yl)ethyl)-N-ethyl)-N-hydroethan-1-
amine
2(2-(6-Me-1H-5-azaindol-3-yl)-N-(-N-(2-(6-CF3-1H-7a-azaindol-3-yl)ethyl)-N-ethyl)-N-hydroethan-1-
amine
2(2-(7-CF3-1H-5-azaindol-3-yl)-N-(-N-(2-(6-CF3-1H-7a-azaindol-3-yl)ethyl)-N-ethyl)-N-hydroethan-1-
amine
2(2-(4-D-1H-5-azaindol-3-yl)-N-(-N-(2-(7-F-1H-7a-azaindol-3-yl)ethyl)-N-ethyl)-N-hydroethan-1-amine
2(2-(6-Me-1H-5-azaindol-3-yl)-N-(-N-(2-(7-F-1H-7a-azaindol-3-yl)ethyl)-N-ethyl)-N-hydroethan-1-
amine
2(2-(7-CF3-1H-5-azaindol-3-yl)-N-(-N-(2-(7-F-1H-7a-azaindol-3-yl)ethyl)-N-ethyl)-N-hydroethan-1-
amine
2(2-(4-D-1H-5-azaindol-3-yl)-N-(-N-(2-(4-D-1H-7a-azaindol-3-yl)ethyl)-N-ethyl)-N-(D)ethan-1-amine
2(2-(6-Me-1H-5-azaindol-3-yl)-N-(-N-(2-(4-D-1H-7a-azaindol-3-yl)ethyl)-N-ethyl)-N-(D)ethan-1-amine
2(2-(7-CF3-1H-5-azaindol-3-yl)-N-(-N-(2-(4-D-1H-7a-azaindol-3-yl)ethyl)-N-ethyl)-N-(D)ethan-1-amine
2(2-(4-D-1H-5-azaindol-3-yl)-N-(-N-(2-(5-OH-1H-7a-azaindol-3-yl)ethyl)-N-ethyl)-N-(D)ethan-1-amine
2(2-(6-Me-1H-5-azaindol-3-yl)-N-(-N-(2-(5-OH-1H-7a-azaindol-3-yl)ethyl)-N-ethyl)-N-(D)ethan-1-amine
2(2-(7-CF3-1H-5-azaindol-3-yl)-N-(-N-(2-(5-OH-1H-7a-azaindol-3-yl)ethyl)-N-ethyl)-N-(D)ethan-1-
amine
2(2-(4-D-1H-5-azaindol-3-yl)-N-(-N-(2-(6-CF3-1H-7a-azaindol-3-yl)ethyl)-N-ethyl)-N-(D)ethan-1-amine
2(2-(6-Me-1H-5-azaindol-3-yl)-N-(-N-(2-(6-CF3-1H-7a-azaindol-3-yl)ethyl)-N-ethyl)-N-(D)ethan-1-
amine
2(2-(7-CF3-1H-5-azaindol-3-yl)-N-(-N-(2-(6-CF3-1H-7a-azaindol-3-yl)ethyl)-N-ethyl)-N-(D)ethan-1-
amine
2(2-(4-D-1H-5-azaindol-3-yl)-N-(-N-(2-(7-F-1H-7a-azaindol-3-yl)ethyl)-N-ethyl)-N-(D)ethan-1-amine
2(2-(6-Me-1H-5-azaindol-3-yl)-N-(-N-(2-(7-F-1H-7a-azaindol-3-yl)ethyl)-N-ethyl)-N-(D)ethan-1-amine
2(2-(7-CF3-1H-5-azaindol-3-yl)-N-(-N-(2-(7-F-1H-7a-azaindol-3-yl)ethyl)-N-ethyl)-N-(D)ethan-1-amine
2(2-(4-D-1H-5-azaindol-3-yl)-N-(-N-(2-(4-D-1H-7a-azaindol-3-yl)ethyl)-N-ethyl)-N-propylethan-1-amine
2(2-(6-Me-1H-5-azaindol-3-yl)-N-(-N-(2-(4-D-1H-7a-azaindol-3-yl)ethyl)-N-ethyl)-N-propylethan-1-
amine
2(2-(7-CF3-1H-5-azaindol-3-yl)-N-(-N-(2-(4-D-1H-7a-azaindol-3-yl)ethyl)-N-ethyl)-N-propylethan-1-
amine
2(2-(4-D-1H-5-azaindol-3-yl)-N-(-N-(2-(5-OH-1H-7a-azaindol-3-yl)ethyl)-N-ethyl)-N-propylethan-1-
amine
2(2-(6-Me-1H-5-azaindol-3-yl)-N-(-N-(2-(5-OH-1H-7a-azaindol-3-yl)ethyl)-N-ethyl)-N-propylethan-1-
amine
2(2-(7-CF3-1H-5-azaindol-3-yl)-N-(-N-(2-(5-OH-1H-7a-azaindol-3-yl)ethyl)-N-ethyl)-N-propylethan-1-
amine
2(2-(4-D-1H-5-azaindol-3-yl)-N-(-N-(2-(6-CF3-1H-7a-azaindol-3-yl)ethyl)-N-ethyl)-N-propylethan-1-
amine
2(2-(6-Me-1H-5-azaindol-3-yl)-N-(-N-(2-(6-CF3-1H-7a-azaindol-3-yl)ethyl)-N-ethyl)-N-propylethan-1-
amine
2(2-(7-CF3-1H-5-azaindol-3-yl)-N-(-N-(2-(6-CF3-1H-7a-azaindol-3-yl)ethyl)-N-ethyl)-N-propylethan-1-
amine
2(2-(4-D-1H-5-azaindol-3-yl)-N-(-N-(2-(7-F-1H-7a-azaindol-3-yl)ethyl)-N-ethyl)-N-propylethan-1-amine
2(2-(6-Me-1H-5-azaindol-3-yl)-N-(-N-(2-(7-F-1H-7a-azaindol-3-yl)ethyl)-N-ethyl)-N-propylethan-1-
amine
2(2-(7-CF3-1H-5-azaindol-3-yl)-N-(-N-(2-(7-F-1H-7a-azaindol-3-yl)ethyl)-N-ethyl)-N-propylethan-1-
amine
2(2-(4-D-1H-5-azaindol-3-yl)-N-(-N-(2-(4-D-1H-indol-3-yl)ethyl)-N-ethyl)-N-methylethan-1-amine
2(2-(6-Me-1H-5-azaindol-3-yl)-N-(-N-(2-(4-D-1H-indol-3-yl)ethyl)-N-ethyl)-N-methylethan-1-amine
2(2-(7-CF3-1H-5-azaindol-3-yl)-N-(-N-(2-(4-D-1H-indol-3-yl)ethyl)-N-ethyl)-N-methylethan-1-amine
2(2-(4-D-1H-5-azaindol-3-yl)-N-(-N-(2-(5-OH-1H-indol-3-yl)ethyl)-N-ethyl)-N-methylethan-1-amine
2(2-(6-Me-1H-5-azaindol-3-yl)-N-(-N-(2-(5-OH-1H-indol-3-yl)ethyl)-N-ethyl)-N-methylethan-1-amine
2(2-(7-CF3-1H-5-azaindol-3-yl)-N-(-N-(2-(5-OH-1H-indol-3-yl)ethyl)-N-ethyl)-N-methylethan-1-amine
2(2-(4-D-1H-5-azaindol-3-yl)-N-(-N-(2-(6-CF3-1H-indol-3-yl)ethyl)-N-ethyl)-N-methylethan-1-amine
2(2-(6-Me-1H-5-azaindol-3-yl)-N-(-N-(2-(6-CF3-1H-indol-3-yl)ethyl)-N-ethyl)-N-methylethan-1-amine
2(2-(7-CF3-1H-5-azaindol-3-yl)-N-(-N-(2-(6-CF3-1H-indol-3-yl)ethyl)-N-ethyl)-N-methylethan-1-amine
2(2-(4-D-1H-5-azaindol-3-yl)-N-(-N-(2-(7-F-1H-indol-3-yl)ethyl)-N-ethyl)-N-methylethan-1-amine
2(2-(6-Me-1H-5-azaindol-3-yl)-N-(-N-(2-(7-F-1H-indol-3-yl)ethyl)-N-ethyl)-N-methylethan-1-amine
2(2-(7-CF3-1H-5-azaindol-3-yl)-N-(-N-(2-(7-F-1H-indol-3-yl)ethyl)-N-ethyl)-N-methylethan-1-amine
2(2-(4-D-1H-5-azaindol-3-yl)-N-(-N-(2-(4-D-1H-indol-3-yl)ethyl)-N-ethyl)-N-ethylethan-1-amine
2(2-(6-Me-1H-5-azaindol-3-yl)-N-(-N-(2-(4-D-1H-indol-3-yl)ethyl)-N-ethyl)-N-ethylethan-1-amine
2(2-(7-CF3-1H-5-azaindol-3-yl)-N-(-N-(2-(4-D-1H-indol-3-yl)ethyl)-N-ethyl)-N-ethylethan-1-amine
2(2-(4-D-1H-5-azaindol-3-yl)-N-(-N-(2-(5-OH-1H-indol-3-yl)ethyl)-N-ethyl)-N-ethylethan-1-amine
2(2-(6-Me-1H-5-azaindol-3-yl)-N-(-N-(2-(5-OH-1H-indol-3-yl)ethyl)-N-ethyl)-N-ethylethan-1-amine
2(2-(7-CF3-1H-5-azaindol-3-yl)-N-(-N-(2-(5-OH-1H-indol-3-yl)ethyl)-N-ethyl)-N-ethylethan-1-amine
2(2-(4-D-1H-5-azaindol-3-yl)-N-(-N-(2-(6-CF3-1H-indol-3-yl)ethyl)-N-ethyl)-N-ethylethan-1-amine
2(2-(6-Me-1H-5-azaindol-3-yl)-N-(-N-(2-(6-CF3-1H-indol-3-yl)ethyl)-N-ethyl)-N-ethylethan-1-amine
2(2-(7-CF3-1H-5-azaindol-3-yl)-N-(-N-(2-(6-CF3-1H-indol-3-yl)ethyl)-N-ethyl)-N-ethylethan-1-amine
2(2-(4-D-1H-5-azaindol-3-yl)-N-(-N-(2-(7-F-1H-indol-3-yl)ethyl)-N-ethyl)-N-ethylethan-1-amine
2(2-(6-Me-1H-5-azaindol-3-yl)-N-(-N-(2-(7-F-1H-indol-3-yl)ethyl)-N-ethyl)-N-ethylethan-1-amine
2(2-(7-CF3-1H-5-azaindol-3-yl)-N-(-N-(2-(7-F-1H-indol-3-yl)ethyl)-N-ethyl)-N-ethylethan-1-amine
2(2-(4-D-1H-5-azaindol-3-yl)-N-(-N-(2-(4-D-1H-indol-3-yl)ethyl)-N-ethyl)-N-isopropylethan-1-amine
2(2-(6-Me-1H-5-azaindol-3-yl)-N-(-N-(2-(4-D-1H-indol-3-yl)ethyl)-N-ethyl)-N-isopropylethan-1-amine
2(2-(7-CF3-1H-5-azaindol-3-yl)-N-(-N-(2-(4-D-1H-indol-3-yl)ethyl)-N-ethyl)-N-isopropylethan-1-amine
2(2-(4-D-1H-5-azaindol-3-yl)-N-(-N-(2-(5-OH-1H-indol-3-yl)ethyl)-N-ethyl)-N-isopropylethan-1-amine
2(2-(6-Me-1H-5-azaindol-3-yl)-N-(-N-(2-(5-OH-1H-indol-3-yl)ethyl)-N-ethyl)-N-isopropylethan-1-amine
2(2-(7-CF3-1H-5-azaindol-3-yl)-N-(-N-(2-(5-OH-1H-indol-3-yl)ethyl)-N-ethyl)-N-isopropylethan-1-amine
2(2-(4-D-1H-5-azaindol-3-yl)-N-(-N-(2-(6-CF3-1H-indol-3-yl)ethyl)-N-ethyl)-N-isopropylethan-1-amine
2(2-(6-Me-1H-5-azaindol-3-yl)-N-(-N-(2-(6-CF3-1H-indol-3-yl)ethyl)-N-ethyl)-N-isopropylethan-1-amine
2(2-(7-CF3-1H-5-azaindol-3-yl)-N-(-N-(2-(6-CF3-1H-indol-3-yl)ethyl)-N-ethyl)-N-isopropylethan-1-
amine
2(2-(4-D-1H-5-azaindol-3-yl)-N-(-N-(2-(7-F-1H-indol-3-yl)ethyl)-N-ethyl)-N-isopropylethan-1-amine
2(2-(6-Me-1H-5-azaindol-3-yl)-N-(-N-(2-(7-F-1H-indol-3-yl)ethyl)-N-ethyl)-N-isopropylethan-1-amine
2(2-(7-CF3-1H-5-azaindol-3-yl)-N-(-N-(2-(7-F-1H-indol-3-yl)ethyl)-N-ethyl)-N-isopropylethan-1-amine
2(2-(4-D-1H-5-azaindol-3-yl)-N-(-N-(2-(4-D-1H-indol-3-yl)ethyl)-N-ethyl)-N-allylethan-1-amine
2(2-(6-Me-1H-5-azaindol-3-yl)-N-(-N-(2-(4-D-1H-indol-3-yl)ethyl)-N-ethyl)-N-allylethan-1-amine
2(2-(7-CF3-1H-5-azaindol-3-yl)-N-(-N-(2-(4-D-1H-indol-3-yl)ethyl)-N-ethyl)-N-allylethan-1-amine
2(2-(4-D-1H-5-azaindol-3-yl)-N-(-N-(2-(5-OH-1H-indol-3-yl)ethyl)-N-ethyl)-N-allylethan-1-amine
2(2-(6-Me-1H-5-azaindol-3-yl)-N-(-N-(2-(5-OH-1H-indol-3-yl)ethyl)-N-ethyl)-N-allylethan-1-amine
2(2-(7-CF3-1H-5-azaindol-3-yl)-N-(-N-(2-(5-OH-1H-indol-3-yl)ethyl)-N-ethyl)-N-allylethan-1-amine
2(2-(4-D-1H-5-azaindol-3-yl)-N-(-N-(2-(6-CF3-1H-indol-3-yl)ethyl)-N-ethyl)-N-allylethan-1-amine
2(2-(6-Me-1H-5-azaindol-3-yl)-N-(-N-(2-(6-CF3-1H-indol-3-yl)ethyl)-N-ethyl)-N-allylethan-1-amine
2(2-(7-CF3-1H-5-azaindol-3-yl)-N-(-N-(2-(6-CF3-1H-indol-3-yl)ethyl)-N-ethyl)-N-allylethan-1-amine
2(2-(4-D-1H-5-azaindol-3-yl)-N-(-N-(2-(7-F-1H-indol-3-yl)ethyl)-N-ethyl)-N-allylethan-1-amine
2(2-(6-Me-1H-5-azaindol-3-yl)-N-(-N-(2-(7-F-1H-indol-3-yl)ethyl)-N-ethyl)-N-allylethan-1-amine
2(2-(7-CF3-1H-5-azaindol-3-yl)-N-(-N-(2-(7-F-1H-indol-3-yl)ethyl)-N-ethyl)-N-allylethan-1-amine
2(2-(4-D-1H-5-azaindol-3-yl)-N-(-N-(2-(4-D-1H-indol-3-yl)ethyl)-N-ethyl)-N-hydroethan-1-amine
2(2-(6-Me-1H-5-azaindol-3-yl)-N-(-N-(2-(4-D-1H-indol-3-yl)ethyl)-N-ethyl)-N-hydroethan-1-amine
2(2-(7-CF3-1H-5-azaindol-3-yl)-N-(-N-(2-(4-D-1H-indol-3-yl)ethyl)-N-ethyl)-N-hydroethan-1-amine
2(2-(4-D-1H-5-azaindol-3-yl)-N-(-N-(2-(5-OH-1H-indol-3-yl)ethyl)-N-ethyl)-N-hydroethan-1-amine
2(2-(6-Me-1H-5-azaindol-3-yl)-N-(-N-(2-(5-OH-1H-indol-3-yl)ethyl)-N-ethyl)-N-hydroethan-1-amine
2(2-(7-CF3-1H-5-azaindol-3-yl)-N-(-N-(2-(5-OH-1H-indol-3-yl)ethyl)-N-ethyl)-N-hydroethan-1-amine
2(2-(4-D-1H-5-azaindol-3-yl)-N-(-N-(2-(6-CF3-1H-indol-3-yl)ethyl)-N-ethyl)-N-hydroethan-1-amine
2(2-(6-Me-1H-5-azaindol-3-yl)-N-(-N-(2-(6-CF3-1H-indol-3-yl)ethyl)-N-ethyl)-N-hydroethan-1-amine
2(2-(7-CF3-1H-5-azaindol-3-yl)-N-(-N-(2-(6-CF3-1H-indol-3-yl)ethyl)-N-ethyl)-N-hydroethan-1-amine
2(2-(4-D-1H-5-azaindol-3-yl)-N-(-N-(2-(7-F-1H-indol-3-yl)ethyl)-N-ethyl)-N-hydroethan-1-amine
2(2-(6-Me-1H-5-azaindol-3-yl)-N-(-N-(2-(7-F-1H-indol-3-yl)ethyl)-N-ethyl)-N-hydroethan-1-amine
2(2-(7-CF3-1H-5-azaindol-3-yl)-N-(-N-(2-(7-F-1H-indol-3-yl)ethyl)-N-ethyl)-N-hydroethan-1-amine
2(2-(4-D-1H-5-azaindol-3-yl)-N-(-N-(2-(4-D-1H-indol-3-yl)ethyl)-N-ethyl)-N-(D)ethan-1-amine
2(2-(6-Me-1H-5-azaindol-3-yl)-N-(-N-(2-(4-D-1H-indol-3-yl)ethyl)-N-ethyl)-N-(D)ethan-1-amine
2(2-(7-CF3-1H-5-azaindol-3-yl)-N-(-N-(2-(4-D-1H-indol-3-yl)ethyl)-N-ethyl)-N-(D)ethan-1-amine
2(2-(4-D-1H-5-azaindol-3-yl)-N-(-N-(2-(5-OH-1H-indol-3-yl)ethyl)-N-ethyl)-N-(D)ethan-1-amine
2(2-(6-Me-1H-5-azaindol-3-yl)-N-(-N-(2-(5-OH-1H-indol-3-yl)ethyl)-N-ethyl)-N-(D)ethan-1-amine
2(2-(7-CF3-1H-5-azaindol-3-yl)-N-(-N-(2-(5-OH-1H-indol-3-yl)ethyl)-N-ethyl)-N-(D)ethan-1-amine
2(2-(4-D-1H-5-azaindol-3-yl)-N-(-N-(2-(6-CF3-1H-indol-3-yl)ethyl)-N-ethyl)-N-(D)ethan-1-amine
2(2-(6-Me-1H-5-azaindol-3-yl)-N-(-N-(2-(6-CF3-1H-indol-3-yl)ethyl)-N-ethyl)-N-(D)ethan-1-amine
2(2-(7-CF3-1H-5-azaindol-3-yl)-N-(-N-(2-(6-CF3-1H-indol-3-yl)ethyl)-N-ethyl)-N-(D)ethan-1-amine
2(2-(4-D-1H-5-azaindol-3-yl)-N-(-N-(2-(7-F-1H-indol-3-yl)ethyl)-N-ethyl)-N-(D)ethan-1-amine
2(2-(6-Me-1H-5-azaindol-3-yl)-N-(-N-(2-(7-F-1H-indol-3-yl)ethyl)-N-ethyl)-N-(D)ethan-1-amine
2(2-(7-CF3-1H-5-azaindol-3-yl)-N-(-N-(2-(7-F-1H-indol-3-yl)ethyl)-N-ethyl)-N-(D)ethan-1-amine
2(2-(4-D-1H-5-azaindol-3-yl)-N-(-N-(2-(4-D-1H-indol-3-yl)ethyl)-N-ethyl)-N-propylethan-1-amine
2(2-(6-Me-1H-5-azaindol-3-yl)-N-(-N-(2-(4-D-1H-indol-3-yl)ethyl)-N-ethyl)-N-propylethan-1-amine
2(2-(7-CF3-1H-5-azaindol-3-yl)-N-(-N-(2-(4-D-1H-indol-3-yl)ethyl)-N-ethyl)-N-propylethan-1-amine
2(2-(4-D-1H-5-azaindol-3-yl)-N-(-N-(2-(5-OH-1H-indol-3-yl)ethyl)-N-ethyl)-N-propylethan-1-amine
2(2-(6-Me-1H-5-azaindol-3-yl)-N-(-N-(2-(5-OH-1H-indol-3-yl)ethyl)-N-ethyl)-N-propylethan-1-amine
2(2-(7-CF3-1H-5-azaindol-3-yl)-N-(-N-(2-(5-OH-1H-indol-3-yl)ethyl)-N-ethyl)-N-propylethan-1-amine
2(2-(4-D-1H-5-azaindol-3-yl)-N-(-N-(2-(6-CF3-1H-indol-3-yl)ethyl)-N-ethyl)-N-propylethan-1-amine
2(2-(6-Me-1H-5-azaindol-3-yl)-N-(-N-(2-(6-CF3-1H-indol-3-yl)ethyl)-N-ethyl)-N-propylethan-1-amine
2(2-(7-CF3-1H-5-azaindol-3-yl)-N-(-N-(2-(6-CF3-1H-indol-3-yl)ethyl)-N-ethyl)-N-propylethan-1-amine
2(2-(4-D-1H-5-azaindol-3-yl)-N-(-N-(2-(7-F-1H-indol-3-yl)ethyl)-N-ethyl)-N-propylethan-1-amine
2(2-(6-Me-1H-5-azaindol-3-yl)-N-(-N-(2-(7-F-1H-indol-3-yl)ethyl)-N-ethyl)-N-propylethan-1-amine
2(2-(7-CF3-1H-5-azaindol-3-yl)-N-(-N-(2-(7-F-1H-indol-3-yl)ethyl)-N-ethyl)-N-propylethan-1-amine
2(2-(4-D-1H-6-azaindol-3-yl)-N-(-N-(2-(4-D-1H-6-azaindol-3-yl)ethyl)-N-ethyl)-N-methylethan-1-amine
2(2-(5-OH-1H-6-azaindol-3-yl)-N-(-N-(2-(4-D-1H-6-azaindol-3-yl)ethyl)-N-ethyl)-N-methylethan-1-
amine
2(2-(7-CF3-1H-6-azaindol-3-yl)-N-(-N-(2-(4-D-1H-6-azaindol-3-yl)ethyl)-N-ethyl)-N-methylethan-1-
amine
2(2-(4-D-1H-6-azaindol-3-yl)-N-(-N-(2-(5-OH-1H-6-azaindol-3-yl)ethyl)-N-ethyl)-N-methylethan-1-
amine
2(2-(5-OH-1H-6-azaindol-3-yl)-N-(-N-(2-(5-OH-1H-6-azaindol-3-yl)ethyl)-N-ethyl)-N-methylethan-1-
amine
2(2-(7-CF3-1H-6-azaindol-3-yl)-N-(-N-(2-(5-OH-1H-6-azaindol-3-yl)ethyl)-N-ethyl)-N-methylethan-1-
amine
2(2-(4-D-1H-6-azaindol-3-yl)-N-(-N-(2-(7-CF3-1H-6-azaindol-3-yl)ethyl)-N-ethyl)-N-methylethan-1-
amine
2(2-(5-OH-1H-6-azaindol-3-yl)-N-(-N-(2-(7-CF3-1H-6-azaindol-3-yl)ethyl)-N-ethyl)-N-methylethan-1-
amine
2(2-(7-CF3-1H-6-azaindol-3-yl)-N-(-N-(2-(7-CF3-1H-6-azaindol-3-yl)ethyl)-N-ethyl)-N-methylethan-1-
amine
2(2-(4-D-1H-6-azaindol-3-yl)-N-(-N-(2-(4-D-1H-6-azaindol-3-yl)ethyl)-N-ethyl)-N-ethylethan-1-amine
2(2-(5-OH-1H-6-azaindol-3-yl)-N-(-N-(2-(4-D-1H-6-azaindol-3-yl)ethyl)-N-ethyl)-N-ethylethan-1-amine
2(2-(7-CF3-1H-6-azaindol-3-yl)-N-(-N-(2-(4-D-1H-6-azaindol-3-yl)ethyl)-N-ethyl)-N-ethylethan-1-amine
2(2-(4-D-1H-6-azaindol-3-yl)-N-(-N-(2-(5-OH-1H-6-azaindol-3-yl)ethyl)-N-ethyl)-N-ethylethan-1-amine
2(2-(5-OH-1H-6-azaindol-3-yl)-N-(-N-(2-(5-OH-1H-6-azaindol-3-yl)ethyl)-N-ethyl)-N-ethylethan-1-
amine
2(2-(7-CF3-1H-6-azaindol-3-yl)-N-(-N-(2-(5-OH-1H-6-azaindol-3-yl)ethyl)-N-ethyl)-N-ethylethan-1-
amine
2(2-(4-D-1H-6-azaindol-3-yl)-N-(-N-(2-(7-CF3-1H-6-azaindol-3-yl)ethyl)-N-ethyl)-N-ethylethan-1-amine
2(2-(5-OH-1H-6-azaindol-3-yl)-N-(-N-(2-(7-CF3-1H-6-azaindol-3-yl)ethyl)-N-ethyl)-N-ethylethan-1-
amine
2(2-(7-CF3-1H-6-azaindol-3-yl)-N-(-N-(2-(7-CF3-1H-6-azaindol-3-yl)ethyl)-N-ethyl)-N-ethylethan-1-
amine
2(2-(4-D-1H-6-azaindol-3-yl)-N-(-N-(2-(4-D-1H-6-azaindol-3-yl)ethyl)-N-ethyl)-N-isopropylethan-1-
amine
2(2-(5-OH-1H-6-azaindol-3-yl)-N-(-N-(2-(4-D-1H-6-azaindol-3-yl)ethyl)-N-ethyl)-N-isopropylethan-1-
amine
2(2-(7-CF3-1H-6-azaindol-3-yl)-N-(-N-(2-(4-D-1H-6-azaindol-3-yl)ethyl)-N-ethyl)-N-isopropylethan-1-
amine
2(2-(4-D-1H-6-azaindol-3-yl)-N-(-N-(2-(5-OH-1H-6-azaindol-3-yl)ethyl)-N-ethyl)-N-isopropylethan-1-
amine
2(2-(5-OH-1H-6-azaindol-3-yl)-N-(-N-(2-(5-OH-1H-6-azaindol-3-yl)ethyl)-N-ethyl)-N-isopropylethan-1-
amine
2(2-(7-CF3-1H-6-azaindol-3-yl)-N-(-N-(2-(5-OH-1H-6-azaindol-3-yl)ethyl)-N-ethyl)-N-isopropylethan-1-
amine
2(2-(4-D-1H-6-azaindol-3-yl)-N-(-N-(2-(7-CF3-1H-6-azaindol-3-yl)ethyl)-N-ethyl)-N-isopropylethan-1-
amine
2(2-(5-OH-1H-6-azaindol-3-yl)-N-(-N-(2-(7-CF3-1H-6-azaindol-3-yl)ethyl)-N-ethyl)-N-isopropylethan-1-
amine
2(2-(7-CF3-1H-6-azaindol-3-yl)-N-(-N-(2-(7-CF3-1H-6-azaindol-3-yl)ethyl)-N-ethyl)-N-isopropylethan-1-
amine
2(2-(4-D-1H-6-azaindol-3-yl)-N-(-N-(2-(4-D-1H-6-azaindol-3-yl)ethyl)-N-ethyl)-N-allylethan-1-amine
2(2-(5-OH-1H-6-azaindol-3-yl)-N-(-N-(2-(4-D-1H-6-azaindol-3-yl)ethyl)-N-ethyl)-N-allylethan-1-amine
2(2-(7-CF3-1H-6-azaindol-3-yl)-N-(-N-(2-(4-D-1H-6-azaindol-3-yl)ethyl)-N-ethyl)-N-allylethan-1-amine
2(2-(4-D-1H-6-azaindol-3-yl)-N-(-N-(2-(5-OH-1H-6-azaindol-3-yl)ethyl)-N-ethyl)-N-allylethan-1-amine
2(2-(5-OH-1H-6-azaindol-3-yl)-N-(-N-(2-(5-OH-1H-6-azaindol-3-yl)ethyl)-N-ethyl)-N-allylethan-1-amine
2(2-(7-CF3-1H-6-azaindol-3-yl)-N-(-N-(2-(5-OH-1H-6-azaindol-3-yl)ethyl)-N-ethyl)-N-allylethan-1-amine
2(2-(4-D-1H-6-azaindol-3-yl)-N-(-N-(2-(7-CF3-1H-6-azaindol-3-yl)ethyl)-N-ethyl)-N-allylethan-1-amine
2(2-(5-OH-1H-6-azaindol-3-yl)-N-(-N-(2-(7-CF3-1H-6-azaindol-3-yl)ethyl)-N-ethyl)-N-allylethan-1-amine
2(2-(7-CF3-1H-6-azaindol-3-yl)-N-(-N-(2-(7-CF3-1H-6-azaindol-3-yl)ethyl)-N-ethyl)-N-allylethan-1-
amine
2(2-(4-D-1H-6-azaindol-3-yl)-N-(-N-(2-(4-D-1H-6-azaindol-3-yl)ethyl)-N-ethyl)-N-hydroethan-1-amine
2(2-(5-OH-1H-6-azaindol-3-yl)-N-(-N-(2-(4-D-1H-6-azaindol-3-yl)ethyl)-N-ethyl)-N-hydroethan-1-amine
2(2-(7-CF3-1H-6-azaindol-3-yl)-N-(-N-(2-(4-D-1H-6-azaindol-3-yl)ethyl)-N-ethyl)-N-hydroethan-1-
amine
2(2-(4-D-1H-6-azaindol-3-yl)-N-(-N-(2-(5-OH-1H-6-azaindol-3-yl)ethyl)-N-ethyl)-N-hydroethan-1-amine
2(2-(5-OH-1H-6-azaindol-3-yl)-N-(-N-(2-(5-OH-1H-6-azaindol-3-yl)ethyl)-N-ethyl)-N-hydroethan-1-
amine
2(2-(7-CF3-1H-6-azaindol-3-yl)-N-(-N-(2-(5-OH-1H-6-azaindol-3-yl)ethyl)-N-ethyl)-N-hydroethan-1-
amine
2(2-(4-D-1H-6-azaindol-3-yl)-N-(-N-(2-(7-CF3-1H-6-azaindol-3-yl)ethyl)-N-ethyl)-N-hydroethan-1-
amine
2(2-(5-OH-1H-6-azaindol-3-yl)-N-(-N-(2-(7-CF3-1H-6-azaindol-3-yl)ethyl)-N-ethyl)-N-hydroethan-1-
amine
2(2-(7-CF3-1H-6-azaindol-3-yl)-N-(-N-(2-(7-CF3-1H-6-azaindol-3-yl)ethyl)-N-ethyl)-N-hydroethan-1-
amine
2(2-(4-D-1H-6-azaindol-3-yl)-N-(-N-(2-(4-D-1H-6-azaindol-3-yl)ethyl)-N-ethyl)-N-(D)ethan-1-amine
2(2-(5-OH-1H-6-azaindol-3-yl)-N-(-N-(2-(4-D-1H-6-azaindol-3-yl)ethyl)-N-ethyl)-N-(D)ethan-1-amine
2(2-(7-CF3-1H-6-azaindol-3-yl)-N-(-N-(2-(4-D-1H-6-azaindol-3-yl)ethyl)-N-ethyl)-N-(D)ethan-1-amine
2(2-(4-D-1H-6-azaindol-3-yl)-N-(-N-(2-(5-OH-1H-6-azaindol-3-yl)ethyl)-N-ethyl)-N-(D)ethan-1-amine
2(2-(5-OH-1H-6-azaindol-3-yl)-N-(-N-(2-(5-OH-1H-6-azaindol-3-yl)ethyl)-N-ethyl)-N-(D)ethan-1-amine
2(2-(7-CF3-1H-6-azaindol-3-yl)-N-(-N-(2-(5-OH-1H-6-azaindol-3-yl)ethyl)-N-ethyl)-N-(D)ethan-1-amine
2(2-(4-D-1H-6-azaindol-3-yl)-N-(-N-(2-(7-CF3-1H-6-azaindol-3-yl)ethyl)-N-ethyl)-N-(D)ethan-1-amine
2(2-(5-OH-1H-6-azaindol-3-yl)-N-(-N-(2-(7-CF3-1H-6-azaindol-3-yl)ethyl)-N-ethyl)-N-(D)ethan-1-amine
2(2-(7-CF3-1H-6-azaindol-3-yl)-N-(-N-(2-(7-CF3-1H-6-azaindol-3-yl)ethyl)-N-ethyl)-N-(D)ethan-1-amine
2(2-(4-D-1H-6-azaindol-3-yl)-N-(-N-(2-(4-D-1H-6-azaindol-3-yl)ethyl)-N-ethyl)-N-propylethan-1-amine
2(2-(5-OH-1H-6-azaindol-3-yl)-N-(-N-(2-(4-D-1H-6-azaindol-3-yl)ethyl)-N-ethyl)-N-propylethan-1-
amine
2(2-(7-CF3-1H-6-azaindol-3-yl)-N-(-N-(2-(4-D-1H-6-azaindol-3-yl)ethyl)-N-ethyl)-N-propylethan-1-
amine
2(2-(4-D-1H-6-azaindol-3-yl)-N-(-N-(2-(5-OH-1H-6-azaindol-3-yl)ethyl)-N-ethyl)-N-propylethan-1-
amine
2(2-(5-OH-1H-6-azaindol-3-yl)-N-(-N-(2-(5-OH-1H-6-azaindol-3-yl)ethyl)-N-ethyl)-N-propylethan-1-
amine
2(2-(7-CF3-1H-6-azaindol-3-yl)-N-(-N-(2-(5-OH-1H-6-azaindol-3-yl)ethyl)-N-ethyl)-N-propylethan-1-
amine
2(2-(4-D-1H-6-azaindol-3-yl)-N-(-N-(2-(7-CF3-1H-6-azaindol-3-yl)ethyl)-N-ethyl)-N-propylethan-1-
amine
2(2-(5-OH-1H-6-azaindol-3-yl)-N-(-N-(2-(7-CF3-1H-6-azaindol-3-yl)ethyl)-N-ethyl)-N-propylethan-1-
amine
2(2-(7-CF3-1H-6-azaindol-3-yl)-N-(-N-(2-(7-CF3-1H-6-azaindol-3-yl)ethyl)-N-ethyl)-N-propylethan-1-
amine
2(2-(4-D-1H-6-azaindol-3-yl)-N-(-N-(2-(4-D-1H-7-azaindol-3-yl)ethyl)-N-ethyl)-N-methylethan-1-amine
2(2-(5-OH-1H-6-azaindol-3-yl)-N-(-N-(2-(4-D-1H-7-azaindol-3-yl)ethyl)-N-ethyl)-N-methylethan-1-
amine
2(2-(7-CF3-1H-6-azaindol-3-yl)-N-(-N-(2-(4-D-1H-7-azaindol-3-yl)ethyl)-N-ethyl)-N-methylethan-1-
amine
2(2-(4-D-1H-6-azaindol-3-yl)-N-(-N-(2-(5-OH-1H-7-azaindol-3-yl)ethyl)-N-ethyl)-N-methylethan-1-
amine
2(2-(5-OH-1H-6-azaindol-3-yl)-N-(-N-(2-(5-OH-1H-7-azaindol-3-yl)ethyl)-N-ethyl)-N-methylethan-1-
amine
2(2-(7-CF3-1H-6-azaindol-3-yl)-N-(-N-(2-(5-OH-1H-7-azaindol-3-yl)ethyl)-N-ethyl)-N-methylethan-1-
amine
2(2-(4-D-1H-6-azaindol-3-yl)-N-(-N-(2-(6-CF3-1H-7-azaindol-3-yl)ethyl)-N-ethyl)-N-methylethan-1-
amine
2(2-(5-OH-1H-6-azaindol-3-yl)-N-(-N-(2-(6-CF3-1H-7-azaindol-3-yl)ethyl)-N-ethyl)-N-methylethan-1-
amine
2(2-(7-CF3-1H-6-azaindol-3-yl)-N-(-N-(2-(6-CF3-1H-7-azaindol-3-yl)ethyl)-N-ethyl)-N-methylethan-1-
amine
2(2-(4-D-1H-6-azaindol-3-yl)-N-(-N-(2-(4-D-1H-7-azaindol-3-yl)ethyl)-N-ethyl)-N-ethylethan-1-amine
2(2-(5-OH-1H-6-azaindol-3-yl)-N-(-N-(2-(4-D-1H-7-azaindol-3-yl)ethyl)-N-ethyl)-N-ethylethan-1-amine
2(2-(7-CF3-1H-6-azaindol-3-yl)-N-(-N-(2-(4-D-1H-7-azaindol-3-yl)ethyl)-N-ethyl)-N-ethylethan-1-amine
2(2-(4-D-1H-6-azaindol-3-yl)-N-(-N-(2-(5-OH-1H-7-azaindol-3-yl)ethyl)-N-ethyl)-N-ethylethan-1-amine
2(2-(5-OH-1H-6-azaindol-3-yl)-N-(-N-(2-(5-OH-1H-7-azaindol-3-yl)ethyl)-N-ethyl)-N-ethylethan-1-
amine
2(2-(7-CF3-1H-6-azaindol-3-yl)-N-(-N-(2-(5-OH-1H-7-azaindol-3-yl)ethyl)-N-ethyl)-N-ethylethan-1-
amine
2(2-(4-D-1H-6-azaindol-3-yl)-N-(-N-(2-(6-CF3-1H-7-azaindol-3-yl)ethyl)-N-ethyl)-N-ethylethan-1-amine
2(2-(5-OH-1H-6-azaindol-3-yl)-N-(-N-(2-(6-CF3-1H-7-azaindol-3-yl)ethyl)-N-ethyl)-N-ethylethan-1-
amine
2(2-(7-CF3-1H-6-azaindol-3-yl)-N-(-N-(2-(6-CF3-1H-7-azaindol-3-yl)ethyl)-N-ethyl)-N-ethylethan-1-
amine
2(2-(4-D-1H-6-azaindol-3-yl)-N-(-N-(2-(4-D-1H-7-azaindol-3-yl)ethyl)-N-ethyl)-N-isopropylethan-1-
amine
2(2-(5-OH-1H-6-azaindol-3-yl)-N-(-N-(2-(4-D-1H-7-azaindol-3-yl)ethyl)-N-ethyl)-N-isopropylethan-1-
amine
2(2-(7-CF3-1H-6-azaindol-3-yl)-N-(-N-(2-(4-D-1H-7-azaindol-3-yl)ethyl)-N-ethyl)-N-isopropylethan-1-
amine
2(2-(4-D-1H-6-azaindol-3-yl)-N-(-N-(2-(5-OH-1H-7-azaindol-3-yl)ethyl)-N-ethyl)-N-isopropylethan-1-
amine
2(2-(5-OH-1H-6-azaindol-3-yl)-N-(-N-(2-(5-OH-1H-7-azaindol-3-yl)ethyl)-N-ethyl)-N-isopropylethan-1-
amine
2(2-(7-CF3-1H-6-azaindol-3-yl)-N-(-N-(2-(5-OH-1H-7-azaindol-3-yl)ethyl)-N-ethyl)-N-isopropylethan-1-
amine
2(2-(4-D-1H-6-azaindol-3-yl)-N-(-N-(2-(6-CF3-1H-7-azaindol-3-yl)ethyl)-N-ethyl)-N-isopropylethan-1-
amine
2(2-(5-OH-1H-6-azaindol-3-yl)-N-(-N-(2-(6-CF3-1H-7-azaindol-3-yl)ethyl)-N-ethyl)-N-isopropylethan-1-
amine
2(2-(7-CF3-1H-6-azaindol-3-yl)-N-(-N-(2-(6-CF3-1H-7-azaindol-3-yl)ethyl)-N-ethyl)-N-isopropylethan-1-
amine
2(2-(4-D-1H-6-azaindol-3-yl)-N-(-N-(2-(4-D-1H-7-azaindol-3-yl)ethyl)-N-ethyl)-N-allylethan-1-amine
2(2-(5-OH-1H-6-azaindol-3-yl)-N-(-N-(2-(4-D-1H-7-azaindol-3-yl)ethyl)-N-ethyl)-N-allylethan-1-amine
2(2-(7-CF3-1H-6-azaindol-3-yl)-N-(-N-(2-(4-D-1H-7-azaindol-3-yl)ethyl)-N-ethyl)-N-allylethan-1-amine
2(2-(4-D-1H-6-azaindol-3-yl)-N-(-N-(2-(5-OH-1H-7-azaindol-3-yl)ethyl)-N-ethyl)-N-allylethan-1-amine
2(2-(5-OH-1H-6-azaindol-3-yl)-N-(-N-(2-(5-OH-1H-7-azaindol-3-yl)ethyl)-N-ethyl)-N-allylethan-1-amine
2(2-(7-CF3-1H-6-azaindol-3-yl)-N-(-N-(2-(5-OH-1H-7-azaindol-3-yl)ethyl)-N-ethyl)-N-allylethan-1-amine
2(2-(4-D-1H-6-azaindol-3-yl)-N-(-N-(2-(6-CF3-1H-7-azaindol-3-yl)ethyl)-N-ethyl)-N-allylethan-1-amine
2(2-(5-OH-1H-6-azaindol-3-yl)-N-(-N-(2-(6-CF3-1H-7-azaindol-3-yl)ethyl)-N-ethyl)-N-allylethan-1-amine
2(2-(7-CF3-1H-6-azaindol-3-yl)-N-(-N-(2-(6-CF3-1H-7-azaindol-3-yl)ethyl)-N-ethyl)-N-allylethan-1-
amine
2(2-(4-D-1H-6-azaindol-3-yl)-N-(-N-(2-(4-D-1H-7-azaindol-3-yl)ethyl)-N-ethyl)-N-hydroethan-1-amine
2(2-(5-OH-1H-6-azaindol-3-yl)-N-(-N-(2-(4-D-1H-7-azaindol-3-yl)ethyl)-N-ethyl)-N-hydroethan-1-amine
2(2-(7-CF3-1H-6-azaindol-3-yl)-N-(-N-(2-(4-D-1H-7-azaindol-3-yl)ethyl)-N-ethyl)-N-hydroethan-1-
amine
2(2-(4-D-1H-6-azaindol-3-yl)-N-(-N-(2-(5-OH-1H-7-azaindol-3-yl)ethyl)-N-ethyl)-N-hydroethan-1-amine
2(2-(5-OH-1H-6-azaindol-3-yl)-N-(-N-(2-(5-OH-1H-7-azaindol-3-yl)ethyl)-N-ethyl)-N-hydroethan-1-
amine
2(2-(7-CF3-1H-6-azaindol-3-yl)-N-(-N-(2-(5-OH-1H-7-azaindol-3-yl)ethyl)-N-ethyl)-N-hydroethan-1-
amine
2(2-(4-D-1H-6-azaindol-3-yl)-N-(-N-(2-(6-CF3-1H-7-azaindol-3-yl)ethyl)-N-ethyl)-N-hydroethan-1-
amine
2(2-(5-OH-1H-6-azaindol-3-yl)-N-(-N-(2-(6-CF3-1H-7-azaindol-3-yl)ethyl)-N-ethyl)-N-hydroethan-1-
amine
2(2-(7-CF3-1H-6-azaindol-3-yl)-N-(-N-(2-(6-CF3-1H-7-azaindol-3-yl)ethyl)-N-ethyl)-N-hydroethan-1-
amine
2(2-(4-D-1H-6-azaindol-3-yl)-N-(-N-(2-(4-D-1H-7-azaindol-3-yl)ethyl)-N-ethyl)-N-(D)ethan-1-amine
2(2-(5-OH-1H-6-azaindol-3-yl)-N-(-N-(2-(4-D-1H-7-azaindol-3-yl)ethyl)-N-ethyl)-N-(D)ethan-1-amine
2(2-(7-CF3-1H-6-azaindol-3-yl)-N-(-N-(2-(4-D-1H-7-azaindol-3-yl)ethyl)-N-ethyl)-N-(D)ethan-1-amine
2(2-(4-D-1H-6-azaindol-3-yl)-N-(-N-(2-(5-OH-1H-7-azaindol-3-yl)ethyl)-N-ethyl)-N-(D)ethan-1-amine
2(2-(5-OH-1H-6-azaindol-3-yl)-N-(-N-(2-(5-OH-1H-7-azaindol-3-yl)ethyl)-N-ethyl)-N-(D)ethan-1-amine
2(2-(7-CF3-1H-6-azaindol-3-yl)-N-(-N-(2-(5-OH-1H-7-azaindol-3-yl)ethyl)-N-ethyl)-N-(D)ethan-1-amine
2(2-(4-D-1H-6-azaindol-3-yl)-N-(-N-(2-(6-CF3-1H-7-azaindol-3-yl)ethyl)-N-ethyl)-N-(D)ethan-1-amine
2(2-(5-OH-1H-6-azaindol-3-yl)-N-(-N-(2-(6-CF3-1H-7-azaindol-3-yl)ethyl)-N-ethyl)-N-(D)ethan-1-amine
2(2-(7-CF3-1H-6-azaindol-3-yl)-N-(-N-(2-(6-CF3-1H-7-azaindol-3-yl)ethyl)-N-ethyl)-N-(D)ethan-1-amine
2(2-(4-D-1H-6-azaindol-3-yl)-N-(-N-(2-(4-D-1H-7-azaindol-3-yl)ethyl)-N-ethyl)-N-propylethan-1-amine
2(2-(5-OH-1H-6-azaindol-3-yl)-N-(-N-(2-(4-D-1H-7-azaindol-3-yl)ethyl)-N-ethyl)-N-propylethan-1-
amine
2(2-(7-CF3-1H-6-azaindol-3-yl)-N-(-N-(2-(4-D-1H-7-azaindol-3-yl)ethyl)-N-ethyl)-N-propylethan-1-
amine
2(2-(4-D-1H-6-azaindol-3-yl)-N-(-N-(2-(5-OH-1H-7-azaindol-3-yl)ethyl)-N-ethyl)-N-propylethan-1-
amine
2(2-(5-OH-1H-6-azaindol-3-yl)-N-(-N-(2-(5-OH-1H-7-azaindol-3-yl)ethyl)-N-ethyl)-N-propylethan-1-
amine
2(2-(7-CF3-1H-6-azaindol-3-yl)-N-(-N-(2-(5-OH-1H-7-azaindol-3-yl)ethyl)-N-ethyl)-N-propylethan-1-
amine
2(2-(4-D-1H-6-azaindol-3-yl)-N-(-N-(2-(6-CF3-1H-7-azaindol-3-yl)ethyl)-N-ethyl)-N-propylethan-1-
amine
2(2-(5-OH-1H-6-azaindol-3-yl)-N-(-N-(2-(6-CF3-1H-7-azaindol-3-yl)ethyl)-N-ethyl)-N-propylethan-1-
amine
2(2-(7-CF3-1H-6-azaindol-3-yl)-N-(-N-(2-(6-CF3-1H-7-azaindol-3-yl)ethyl)-N-ethyl)-N-propylethan-1-
amine
2(2-(4-D-1H-6-azaindol-3-yl)-N-(-N-(2-(4-D-1H-7a-azaindol-3-yl)ethyl)-N-ethyl)-N-methylethan-1-
amine
2(2-(5-OH-1H-6-azaindol-3-yl)-N-(-N-(2-(4-D-1H-7a-azaindol-3-yl)ethyl)-N-ethyl)-N-methylethan-1-
amine
2(2-(7-CF3-1H-6-azaindol-3-yl)-N-(-N-(2-(4-D-1H-7a-azaindol-3-yl)ethyl)-N-ethyl)-N-methylethan-1-
amine
2(2-(4-D-1H-6-azaindol-3-yl)-N-(-N-(2-(5-OH-1H-7a-azaindol-3-yl)ethyl)-N-ethyl)-N-methylethan-1-
amine
2(2-(5-OH-1H-6-azaindol-3-yl)-N-(-N-(2-(5-OH-1H-7a-azaindol-3-yl)ethyl)-N-ethyl)-N-methylethan-1-
amine
2(2-(7-CF3-1H-6-azaindol-3-yl)-N-(-N-(2-(5-OH-1H-7a-azaindol-3-yl)ethyl)-N-ethyl)-N-methylethan-1-
amine
2(2-(4-D-1H-6-azaindol-3-yl)-N-(-N-(2-(6-CF3-1H-7a-azaindol-3-yl)ethyl)-N-ethyl)-N-methylethan-1-
amine
2(2-(5-OH-1H-6-azaindol-3-yl)-N-(-N-(2-(6-CF3-1H-7a-azaindol-3-yl)ethyl)-N-ethyl)-N-methylethan-1-
amine
2(2-(7-CF3-1H-6-azaindol-3-yl)-N-(-N-(2-(6-CF3-1H-7a-azaindol-3-yl)ethyl)-N-ethyl)-N-methylethan-1-
amine
2(2-(4-D-1H-6-azaindol-3-yl)-N-(-N-(2-(7-F-1H-7a-azaindol-3-yl)ethyl)-N-ethyl)-N-methylethan-1-amine
2(2-(5-OH-1H-6-azaindol-3-yl)-N-(-N-(2-(7-F-1H-7a-azaindol-3-yl)ethyl)-N-ethyl)-N-methylethan-1-
amine
2(2-(7-CF3-1H-6-azaindol-3-yl)-N-(-N-(2-(7-F-1H-7a-azaindol-3-yl)ethyl)-N-ethyl)-N-methylethan-1-
amine
2(2-(4-D-1H-6-azaindol-3-yl)-N-(-N-(2-(4-D-1H-7a-azaindol-3-yl)ethyl)-N-ethyl)-N-ethylethan-1-amine
2(2-(5-OH-1H-6-azaindol-3-yl)-N-(-N-(2-(4-D-1H-7a-azaindol-3-yl)ethyl)-N-ethyl)-N-ethylethan-1-amine
2(2-(7-CF3-1H-6-azaindol-3-yl)-N-(-N-(2-(4-D-1H-7a-azaindol-3-yl)ethyl)-N-ethyl)-N-ethylethan-1-
amine
2(2-(4-D-1H-6-azaindol-3-yl)-N-(-N-(2-(5-OH-1H-7a-azaindol-3-yl)ethyl)-N-ethyl)-N-ethylethan-1-amine
2(2-(5-OH-1H-6-azaindol-3-yl)-N-(-N-(2-(5-OH-1H-7a-azaindol-3-yl)ethyl)-N-ethyl)-N-ethylethan-1-
amine
2(2-(7-CF3-1H-6-azaindol-3-yl)-N-(-N-(2-(5-OH-1H-7a-azaindol-3-yl)ethyl)-N-ethyl)-N-ethylethan-1-
amine
2(2-(4-D-1H-6-azaindol-3-yl)-N-(-N-(2-(6-CF3-1H-7a-azaindol-3-yl)ethyl)-N-ethyl)-N-ethylethan-1-
amine
2(2-(5-OH-1H-6-azaindol-3-yl)-N-(-N-(2-(6-CF3-1H-7a-azaindol-3-yl)ethyl)-N-ethyl)-N-ethylethan-1-
amine
2(2-(7-CF3-1H-6-azaindol-3-yl)-N-(-N-(2-(6-CF3-1H-7a-azaindol-3-yl)ethyl)-N-ethyl)-N-ethylethan-1-
amine
2(2-(4-D-1H-6-azaindol-3-yl)-N-(-N-(2-(7-F-1H-7a-azaindol-3-yl)ethyl)-N-ethyl)-N-ethylethan-1-amine
2(2-(5-OH-1H-6-azaindol-3-yl)-N-(-N-(2-(7-F-1H-7a-azaindol-3-yl)ethyl)-N-ethyl)-N-ethylethan-1-amine
2(2-(7-CF3-1H-6-azaindol-3-yl)-N-(-N-(2-(7-F-1H-7a-azaindol-3-yl)ethyl)-N-ethyl)-N-ethylethan-1-
amine
2(2-(4-D-1H-6-azaindol-3-yl)-N-(-N-(2-(4-D-1H-7a-azaindol-3-yl)ethyl)-N-ethyl)-N-isopropylethan-1-
amine
2(2-(5-OH-1H-6-azaindol-3-yl)-N-(-N-(2-(4-D-1H-7a-azaindol-3-yl)ethyl)-N-ethyl)-N-isopropylethan-1-
amine
2(2-(7-CF3-1H-6-azaindol-3-yl)-N-(-N-(2-(4-D-1H-7a-azaindol-3-yl)ethyl)-N-ethyl)-N-isopropylethan-1-
amine
2(2-(4-D-1H-6-azaindol-3-yl)-N-(-N-(2-(5-OH-1H-7a-azaindol-3-yl)ethyl)-N-ethyl)-N-isopropylethan-1-
amine
2(2-(5-OH-1H-6-azaindol-3-yl)-N-(-N-(2-(5-OH-1H-7a-azaindol-3-yl)ethyl)-N-ethyl)-N-isopropylethan-1-
amine
2(2-(7-CF3-1H-6-azaindol-3-yl)-N-(-N-(2-(5-OH-1H-7a-azaindol-3-yl)ethyl)-N-ethyl)-N-isopropylethan-
1-amine
2(2-(4-D-1H-6-azaindol-3-yl)-N-(-N-(2-(6-CF3-1H-7a-azaindol-3-yl)ethyl)-N-ethyl)-N-isopropylethan-1-
amine
2(2-(5-OH-1H-6-azaindol-3-yl)-N-(-N-(2-(6-CF3-1H-7a-azaindol-3-yl)ethyl)-N-ethyl)-N-isopropylethan-
1-amine
2(2-(7-CF3-1H-6-azaindol-3-yl)-N-(-N-(2-(6-CF3-1H-7a-azaindol-3-yl)ethyl)-N-ethyl)-N-isopropylethan-
1-amine
2(2-(4-D-1H-6-azaindol-3-yl)-N-(-N-(2-(7-F-1H-7a-azaindol-3-yl)ethyl)-N-ethyl)-N-isopropylethan-1-
amine
2(2-(5-OH-1H-6-azaindol-3-yl)-N-(-N-(2-(7-F-1H-7a-azaindol-3-yl)ethyl)-N-ethyl)-N-isopropylethan-1-
amine
2(2-(7-CF3-1H-6-azaindol-3-yl)-N-(-N-(2-(7-F-1H-7a-azaindol-3-yl)ethyl)-N-ethyl)-N-isopropylethan-1-
amine
2(2-(4-D-1H-6-azaindol-3-yl)-N-(-N-(2-(4-D-1H-7a-azaindol-3-yl)ethyl)-N-ethyl)-N-allylethan-1-amine
2(2-(5-OH-1H-6-azaindol-3-yl)-N-(-N-(2-(4-D-1H-7a-azaindol-3-yl)ethyl)-N-ethyl)-N-allylethan-1-amine
2(2-(7-CF3-1H-6-azaindol-3-yl)-N-(-N-(2-(4-D-1H-7a-azaindol-3-yl)ethyl)-N-ethyl)-N-allylethan-1-amine
2(2-(4-D-1H-6-azaindol-3-yl)-N-(-N-(2-(5-OH-1H-7a-azaindol-3-yl)ethyl)-N-ethyl)-N-allylethan-1-amine
2(2-(5-OH-1H-6-azaindol-3-yl)-N-(-N-(2-(5-OH-1H-7a-azaindol-3-yl)ethyl)-N-ethyl)-N-allylethan-1-
amine
2(2-(7-CF3-1H-6-azaindol-3-yl)-N-(-N-(2-(5-OH-1H-7a-azaindol-3-yl)ethyl)-N-ethyl)-N-allylethan-1-
amine
2(2-(4-D-1H-6-azaindol-3-yl)-N-(-N-(2-(6-CF3-1H-7a-azaindol-3-yl)ethyl)-N-ethyl)-N-allylethan-1-amine
2(2-(5-OH-1H-6-azaindol-3-yl)-N-(-N-(2-(6-CF3-1H-7a-azaindol-3-yl)ethyl)-N-ethyl)-N-allylethan-1-
amine
2(2-(7-CF3-1H-6-azaindol-3-yl)-N-(-N-(2-(6-CF3-1H-7a-azaindol-3-yl)ethyl)-N-ethyl)-N-allylethan-1-
amine
2(2-(4-D-1H-6-azaindol-3-yl)-N-(-N-(2-(7-F-1H-7a-azaindol-3-yl)ethyl)-N-ethyl)-N-allylethan-1-amine
2(2-(5-OH-1H-6-azaindol-3-yl)-N-(-N-(2-(7-F-1H-7a-azaindol-3-yl)ethyl)-N-ethyl)-N-allylethan-1-amine
2(2-(7-CF3-1H-6-azaindol-3-yl)-N-(-N-(2-(7-F-1H-7a-azaindol-3-yl)ethyl)-N-ethyl)-N-allylethan-1-amine
2(2-(4-D-1H-6-azaindol-3-yl)-N-(-N-(2-(4-D-1H-7a-azaindol-3-yl)ethyl)-N-ethyl)-N-hydroethan-1-amine
2(2-(5-OH-1H-6-azaindol-3-yl)-N-(-N-(2-(4-D-1H-7a-azaindol-3-yl)ethyl)-N-ethyl)-N-hydroethan-1-
amine
2(2-(7-CF3-1H-6-azaindol-3-yl)-N-(-N-(2-(4-D-1H-7a-azaindol-3-yl)ethyl)-N-ethyl)-N-hydroethan-1-
amine
2(2-(4-D-1H-6-azaindol-3-yl)-N-(-N-(2-(5-OH-1H-7a-azaindol-3-yl)ethyl)-N-ethyl)-N-hydroethan-1-
amine
2(2-(5-OH-1H-6-azaindol-3-yl)-N-(-N-(2-(5-OH-1H-7a-azaindol-3-yl)ethyl)-N-ethyl)-N-hydroethan-1-
amine
2(2-(7-CF3-1H-6-azaindol-3-yl)-N-(-N-(2-(5-OH-1H-7a-azaindol-3-yl)ethyl)-N-ethyl)-N-hydroethan-1-
amine
2(2-(4-D-1H-6-azaindol-3-yl)-N-(-N-(2-(6-CF3-1H-7a-azaindol-3-yl)ethyl)-N-ethyl)-N-hydroethan-1-
amine
2(2-(5-OH-1H-6-azaindol-3-yl)-N-(-N-(2-(6-CF3-1H-7a-azaindol-3-yl)ethyl)-N-ethyl)-N-hydroethan-1-
amine
2(2-(7-CF3-1H-6-azaindol-3-yl)-N-(-N-(2-(6-CF3-1H-7a-azaindol-3-yl)ethyl)-N-ethyl)-N-hydroethan-1-
amine
2(2-(4-D-1H-6-azaindol-3-yl)-N-(-N-(2-(7-F-1H-7a-azaindol-3-yl)ethyl)-N-ethyl)-N-hydroethan-1-amine
2(2-(5-OH-1H-6-azaindol-3-yl)-N-(-N-(2-(7-F-1H-7a-azaindol-3-yl)ethyl)-N-ethyl)-N-hydroethan-1-
amine
2(2-(7-CF3-1H-6-azaindol-3-yl)-N-(-N-(2-(7-F-1H-7a-azaindol-3-yl)ethyl)-N-ethyl)-N-hydroethan-1-
amine
2(2-(4-D-1H-6-azaindol-3-yl)-N-(-N-(2-(4-D-1H-7a-azaindol-3-yl)ethyl)-N-ethyl)-N-(D)ethan-1-amine
2(2-(5-OH-1H-6-azaindol-3-yl)-N-(-N-(2-(4-D-1H-7a-azaindol-3-yl)ethyl)-N-ethyl)-N-(D)ethan-1-amine
2(2-(7-CF3-1H-6-azaindol-3-yl)-N-(-N-(2-(4-D-1H-7a-azaindol-3-yl)ethyl)-N-ethyl)-N-(D)ethan-1-amine
2(2-(4-D-1H-6-azaindol-3-yl)-N-(-N-(2-(5-OH-1H-7a-azaindol-3-yl)ethyl)-N-ethyl)-N-(D)ethan-1-amine
2(2-(5-OH-1H-6-azaindol-3-yl)-N-(-N-(2-(5-OH-1H-7a-azaindol-3-yl)ethyl)-N-ethyl)-N-(D)ethan-1-amine
2(2-(7-CF3-1H-6-azaindol-3-yl)-N-(-N-(2-(5-OH-1H-7a-azaindol-3-yl)ethyl)-N-ethyl)-N-(D)ethan-1-
amine
2(2-(4-D-1H-6-azaindol-3-yl)-N-(-N-(2-(6-CF3-1H-7a-azaindol-3-yl)ethyl)-N-ethyl)-N-(D)ethan-1-amine
2(2-(5-OH-1H-6-azaindol-3-yl)-N-(-N-(2-(6-CF3-1H-7a-azaindol-3-yl)ethyl)-N-ethyl)-N-(D)ethan-1-
amine
2(2-(7-CF3-1H-6-azaindol-3-yl)-N-(-N-(2-(6-CF3-1H-7a-azaindol-3-yl)ethyl)-N-ethyl)-N-(D)ethan-1-
amine
2(2-(4-D-1H-6-azaindol-3-yl)-N-(-N-(2-(7-F-1H-7a-azaindol-3-yl)ethyl)-N-ethyl)-N-(D)ethan-1-amine
2(2-(5-OH-1H-6-azaindol-3-yl)-N-(-N-(2-(7-F-1H-7a-azaindol-3-yl)ethyl)-N-ethyl)-N-(D)ethan-1-amine
2(2-(7-CF3-1H-6-azaindol-3-yl)-N-(-N-(2-(7-F-1H-7a-azaindol-3-yl)ethyl)-N-ethyl)-N-(D)ethan-1-amine
2(2-(4-D-1H-6-azaindol-3-yl)-N-(-N-(2-(4-D-1H-7a-azaindol-3-yl)ethyl)-N-ethyl)-N-propylethan-1-amine
2(2-(5-OH-1H-6-azaindol-3-yl)-N-(-N-(2-(4-D-1H-7a-azaindol-3-yl)ethyl)-N-ethyl)-N-propylethan-1-
amine
2(2-(7-CF3-1H-6-azaindol-3-yl)-N-(-N-(2-(4-D-1H-7a-azaindol-3-yl)ethyl)-N-ethyl)-N-propylethan-1-
amine
2(2-(4-D-1H-6-azaindol-3-yl)-N-(-N-(2-(5-OH-1H-7a-azaindol-3-yl)ethyl)-N-ethyl)-N-propylethan-1-
amine
2(2-(5-OH-1H-6-azaindol-3-yl)-N-(-N-(2-(5-OH-1H-7a-azaindol-3-yl)ethyl)-N-ethyl)-N-propylethan-1-
amine
2(2-(7-CF3-1H-6-azaindol-3-yl)-N-(-N-(2-(5-OH-1H-7a-azaindol-3-yl)ethyl)-N-ethyl)-N-propylethan-1-
amine
2(2-(4-D-1H-6-azaindol-3-yl)-N-(-N-(2-(6-CF3-1H-7a-azaindol-3-yl)ethyl)-N-ethyl)-N-propylethan-1-
amine
2(2-(5-OH-1H-6-azaindol-3-yl)-N-(-N-(2-(6-CF3-1H-7a-azaindol-3-yl)ethyl)-N-ethyl)-N-propylethan-1-
amine
2(2-(7-CF3-1H-6-azaindol-3-yl)-N-(-N-(2-(6-CF3-1H-7a-azaindol-3-yl)ethyl)-N-ethyl)-N-propylethan-1-
amine
2(2-(4-D-1H-6-azaindol-3-yl)-N-(-N-(2-(7-F-1H-7a-azaindol-3-yl)ethyl)-N-ethyl)-N-propylethan-1-amine
2(2-(5-OH-1H-6-azaindol-3-yl)-N-(-N-(2-(7-F-1H-7a-azaindol-3-yl)ethyl)-N-ethyl)-N-propylethan-1-
amine
2(2-(7-CF3-1H-6-azaindol-3-yl)-N-(-N-(2-(7-F-1H-7a-azaindol-3-yl)ethyl)-N-ethyl)-N-propylethan-1-
amine
2(2-(4-D-1H-6-azaindol-3-yl)-N-(-N-(2-(4-D-1H-indol-3-yl)ethyl)-N-ethyl)-N-methylethan-1-amine
2(2-(5-OH-1H-6-azaindol-3-yl)-N-(-N-(2-(4-D-1H-indol-3-yl)ethyl)-N-ethyl)-N-methylethan-1-amine
2(2-(7-CF3-1H-6-azaindol-3-yl)-N-(-N-(2-(4-D-1H-indol-3-yl)ethyl)-N-ethyl)-N-methylethan-1-amine
2(2-(4-D-1H-6-azaindol-3-yl)-N-(-N-(2-(5-OH-1H-indol-3-yl)ethyl)-N-ethyl)-N-methylethan-1-amine
2(2-(5-OH-1H-6-azaindol-3-yl)-N-(-N-(2-(5-OH-1H-indol-3-yl)ethyl)-N-ethyl)-N-methylethan-1-amine
2(2-(7-CF3-1H-6-azaindol-3-yl)-N-(-N-(2-(5-OH-1H-indol-3-yl)ethyl)-N-ethyl)-N-methylethan-1-amine
2(2-(4-D-1H-6-azaindol-3-yl)-N-(-N-(2-(6-CF3-1H-indol-3-yl)ethyl)-N-ethyl)-N-methylethan-1-amine
2(2-(5-OH-1H-6-azaindol-3-yl)-N-(-N-(2-(6-CF3-1H-indol-3-yl)ethyl)-N-ethyl)-N-methylethan-1-amine
2(2-(7-CF3-1H-6-azaindol-3-yl)-N-(-N-(2-(6-CF3-1H-indol-3-yl)ethyl)-N-ethyl)-N-methylethan-1-amine
2(2-(4-D-1H-6-azaindol-3-yl)-N-(-N-(2-(7-F-1H-indol-3-yl)ethyl)-N-ethyl)-N-methylethan-1-amine
2(2-(5-OH-1H-6-azaindol-3-yl)-N-(-N-(2-(7-F-1H-indol-3-yl)ethyl)-N-ethyl)-N-methylethan-1-amine
2(2-(7-CF3-1H-6-azaindol-3-yl)-N-(-N-(2-(7-F-1H-indol-3-yl)ethyl)-N-ethyl)-N-methylethan-1-amine
2(2-(4-D-1H-6-azaindol-3-yl)-N-(-N-(2-(4-D-1H-indol-3-yl)ethyl)-N-ethyl)-N-ethylethan-1-amine
2(2-(5-OH-1H-6-azaindol-3-yl)-N-(-N-(2-(4-D-1H-indol-3-yl)ethyl)-N-ethyl)-N-ethylethan-1-amine
2(2-(7-CF3-1H-6-azaindol-3-yl)-N-(-N-(2-(4-D-1H-indol-3-yl)ethyl)-N-ethyl)-N-ethylethan-1-amine
2(2-(4-D-1H-6-azaindol-3-yl)-N-(-N-(2-(5-OH-1H-indol-3-yl)ethyl)-N-ethyl)-N-ethylethan-1-amine
2(2-(5-OH-1H-6-azaindol-3-yl)-N-(-N-(2-(5-OH-1H-indol-3-yl)ethyl)-N-ethyl)-N-ethylethan-1-amine
2(2-(7-CF3-1H-6-azaindol-3-yl)-N-(-N-(2-(5-OH-1H-indol-3-yl)ethyl)-N-ethyl)-N-ethylethan-1-amine
2(2-(4-D-1H-6-azaindol-3-yl)-N-(-N-(2-(6-CF3-1H-indol-3-yl)ethyl)-N-ethyl)-N-ethylethan-1-amine
2(2-(5-OH-1H-6-azaindol-3-yl)-N-(-N-(2-(6-CF3-1H-indol-3-yl)ethyl)-N-ethyl)-N-ethylethan-1-amine
2(2-(7-CF3-1H-6-azaindol-3-yl)-N-(-N-(2-(6-CF3-1H-indol-3-yl)ethyl)-N-ethyl)-N-ethylethan-1-amine
2(2-(4-D-1H-6-azaindol-3-yl)-N-(-N-(2-(7-F-1H-indol-3-yl)ethyl)-N-ethyl)-N-ethylethan-1-amine
2(2-(5-OH-1H-6-azaindol-3-yl)-N-(-N-(2-(7-F-1H-indol-3-yl)ethyl)-N-ethyl)-N-ethylethan-1-amine
2(2-(7-CF3-1H-6-azaindol-3-yl)-N-(-N-(2-(7-F-1H-indol-3-yl)ethyl)-N-ethyl)-N-ethylethan-1-amine
2(2-(4-D-1H-6-azaindol-3-yl)-N-(-N-(2-(4-D-1H-indol-3-yl)ethyl)-N-ethyl)-N-isopropylethan-1-amine
2(2-(5-OH-1H-6-azaindol-3-yl)-N-(-N-(2-(4-D-1H-indol-3-yl)ethyl)-N-ethyl)-N-isopropylethan-1-amine
2(2-(7-CF3-1H-6-azaindol-3-yl)-N-(-N-(2-(4-D-1H-indol-3-yl)ethyl)-N-ethyl)-N-isopropylethan-1-amine
2(2-(4-D-1H-6-azaindol-3-yl)-N-(-N-(2-(5-OH-1H-indol-3-yl)ethyl)-N-ethyl)-N-isopropylethan-1-amine
2(2-(5-OH-1H-6-azaindol-3-yl)-N-(-N-(2-(5-OH-1H-indol-3-yl)ethyl)-N-ethyl)-N-isopropylethan-1-amine
2(2-(7-CF3-1H-6-azaindol-3-yl)-N-(-N-(2-(5-OH-1H-indol-3-yl)ethyl)-N-ethyl)-N-isopropylethan-1-amine
2(2-(4-D-1H-6-azaindol-3-yl)-N-(-N-(2-(6-CF3-1H-indol-3-yl)ethyl)-N-ethyl)-N-isopropylethan-1-amine
2(2-(5-OH-1H-6-azaindol-3-yl)-N-(-N-(2-(6-CF3-1H-indol-3-yl)ethyl)-N-ethyl)-N-isopropylethan-1-amine
2(2-(7-CF3-1H-6-azaindol-3-yl)-N-(-N-(2-(6-CF3-1H-indol-3-yl)ethyl)-N-ethyl)-N-isopropylethan-1-
amine
2(2-(4-D-1H-6-azaindol-3-yl)-N-(-N-(2-(7-F-1H-indol-3-yl)ethyl)-N-ethyl)-N-isopropylethan-1-amine
2(2-(5-OH-1H-6-azaindol-3-yl)-N-(-N-(2-(7-F-1H-indol-3-yl)ethyl)-N-ethyl)-N-isopropylethan-1-amine
2(2-(7-CF3-1H-6-azaindol-3-yl)-N-(-N-(2-(7-F-1H-indol-3-yl)ethyl)-N-ethyl)-N-isopropylethan-1-amine
2(2-(4-D-1H-6-azaindol-3-yl)-N-(-N-(2-(4-D-1H-indol-3-yl)ethyl)-N-ethyl)-N-allylethan-1-amine
2(2-(5-OH-1H-6-azaindol-3-yl)-N-(-N-(2-(4-D-1H-indol-3-yl)ethyl)-N-ethyl)-N-allylethan-1-amine
2(2-(7-CF3-1H-6-azaindol-3-yl)-N-(-N-(2-(4-D-1H-indol-3-yl)ethyl)-N-ethyl)-N-allylethan-1-amine
2(2-(4-D-1H-6-azaindol-3-yl)-N-(-N-(2-(5-OH-1H-indol-3-yl)ethyl)-N-ethyl)-N-allylethan-1-amine
2(2-(5-OH-1H-6-azaindol-3-yl)-N-(-N-(2-(5-OH-1H-indol-3-yl)ethyl)-N-ethyl)-N-allylethan-1-amine
2(2-(7-CF3-1H-6-azaindol-3-yl)-N-(-N-(2-(5-OH-1H-indol-3-yl)ethyl)-N-ethyl)-N-allylethan-1-amine
2(2-(4-D-1H-6-azaindol-3-yl)-N-(-N-(2-(6-CF3-1H-indol-3-yl)ethyl)-N-ethyl)-N-allylethan-1-amine
2(2-(5-OH-1H-6-azaindol-3-yl)-N-(-N-(2-(6-CF3-1H-indol-3-yl)ethyl)-N-ethyl)-N-allylethan-1-amine
2(2-(7-CF3-1H-6-azaindol-3-yl)-N-(-N-(2-(6-CF3-1H-indol-3-yl)ethyl)-N-ethyl)-N-allylethan-1-amine
2(2-(4-D-1H-6-azaindol-3-yl)-N-(-N-(2-(7-F-1H-indol-3-yl)ethyl)-N-ethyl)-N-allylethan-1-amine
2(2-(5-OH-1H-6-azaindol-3-yl)-N-(-N-(2-(7-F-1H-indol-3-yl)ethyl)-N-ethyl)-N-allylethan-1-amine
2(2-(7-CF3-1H-6-azaindol-3-yl)-N-(-N-(2-(7-F-1H-indol-3-yl)ethyl)-N-ethyl)-N-allylethan-1-amine
2(2-(4-D-1H-6-azaindol-3-yl)-N-(-N-(2-(4-D-1H-indol-3-yl)ethyl)-N-ethyl)-N-hydroethan-1-amine
2(2-(5-OH-1H-6-azaindol-3-yl)-N-(-N-(2-(4-D-1H-indol-3-yl)ethyl)-N-ethyl)-N-hydroethan-1-amine
2(2-(7-CF3-1H-6-azaindol-3-yl)-N-(-N-(2-(4-D-1H-indol-3-yl)ethyl)-N-ethyl)-N-hydroethan-1-amine
2(2-(4-D-1H-6-azaindol-3-yl)-N-(-N-(2-(5-OH-1H-indol-3-yl)ethyl)-N-ethyl)-N-hydroethan-1-amine
2(2-(5-OH-1H-6-azaindol-3-yl)-N-(-N-(2-(5-OH-1H-indol-3-yl)ethyl)-N-ethyl)-N-hydroethan-1-amine
2(2-(7-CF3-1H-6-azaindol-3-yl)-N-(-N-(2-(5-OH-1H-indol-3-yl)ethyl)-N-ethyl)-N-hydroethan-1-amine
2(2-(4-D-1H-6-azaindol-3-yl)-N-(-N-(2-(6-CF3-1H-indol-3-yl)ethyl)-N-ethyl)-N-hydroethan-1-amine
2(2-(5-OH-1H-6-azaindol-3-yl)-N-(-N-(2-(6-CF3-1H-indol-3-yl)ethyl)-N-ethyl)-N-hydroethan-1-amine
2(2-(7-CF3-1H-6-azaindol-3-yl)-N-(-N-(2-(6-CF3-1H-indol-3-yl)ethyl)-N-ethyl)-N-hydroethan-1-amine
2(2-(4-D-1H-6-azaindol-3-yl)-N-(-N-(2-(7-F-1H-indol-3-yl)ethyl)-N-ethyl)-N-hydroethan-1-amine
2(2-(5-OH-1H-6-azaindol-3-yl)-N-(-N-(2-(7-F-1H-indol-3-yl)ethyl)-N-ethyl)-N-hydroethan-1-amine
2(2-(7-CF3-1H-6-azaindol-3-yl)-N-(-N-(2-(7-F-1H-indol-3-yl)ethyl)-N-ethyl)-N-hydroethan-1-amine
2(2-(4-D-1H-6-azaindol-3-yl)-N-(-N-(2-(4-D-1H-indol-3-yl)ethyl)-N-ethyl)-N-(D)ethan-1-amine
2(2-(5-OH-1H-6-azaindol-3-yl)-N-(-N-(2-(4-D-1H-indol-3-yl)ethyl)-N-ethyl)-N-(D)ethan-1-amine
2(2-(7-CF3-1H-6-azaindol-3-yl)-N-(-N-(2-(4-D-1H-indol-3-yl)ethyl)-N-ethyl)-N-(D)ethan-1-amine
2(2-(4-D-1H-6-azaindol-3-yl)-N-(-N-(2-(5-OH-1H-indol-3-yl)ethyl)-N-ethyl)-N-(D)ethan-1-amine
2(2-(5-OH-1H-6-azaindol-3-yl)-N-(-N-(2-(5-OH-1H-indol-3-yl)ethyl)-N-ethyl)-N-(D)ethan-1-amine
2(2-(7-CF3-1H-6-azaindol-3-yl)-N-(-N-(2-(5-OH-1H-indol-3-yl)ethyl)-N-ethyl)-N-(D)ethan-1-amine
2(2-(4-D-1H-6-azaindol-3-yl)-N-(-N-(2-(6-CF3-1H-indol-3-yl)ethyl)-N-ethyl)-N-(D)ethan-1-amine
2(2-(5-OH-1H-6-azaindol-3-yl)-N-(-N-(2-(6-CF3-1H-indol-3-yl)ethyl)-N-ethyl)-N-(D)ethan-1-amine
2(2-(7-CF3-1H-6-azaindol-3-yl)-N-(-N-(2-(6-CF3-1H-indol-3-yl)ethyl)-N-ethyl)-N-(D)ethan-1-amine
2(2-(4-D-1H-6-azaindol-3-yl)-N-(-N-(2-(7-F-1H-indol-3-yl)ethyl)-N-ethyl)-N-(D)ethan-1-amine
2(2-(5-OH-1H-6-azaindol-3-yl)-N-(-N-(2-(7-F-1H-indol-3-yl)ethyl)-N-ethyl)-N-(D)ethan-1-amine
2(2-(7-CF3-1H-6-azaindol-3-yl)-N-(-N-(2-(7-F-1H-indol-3-yl)ethyl)-N-ethyl)-N-(D)ethan-1-amine
2(2-(4-D-1H-6-azaindol-3-yl)-N-(-N-(2-(4-D-1H-indol-3-yl)ethyl)-N-ethyl)-N-propylethan-1-amine
2(2-(5-OH-1H-6-azaindol-3-yl)-N-(-N-(2-(4-D-1H-indol-3-yl)ethyl)-N-ethyl)-N-propylethan-1-amine
2(2-(7-CF3-1H-6-azaindol-3-yl)-N-(-N-(2-(4-D-1H-indol-3-yl)ethyl)-N-ethyl)-N-propylethan-1-amine
2(2-(4-D-1H-6-azaindol-3-yl)-N-(-N-(2-(5-OH-1H-indol-3-yl)ethyl)-N-ethyl)-N-propylethan-1-amine
2(2-(5-OH-1H-6-azaindol-3-yl)-N-(-N-(2-(5-OH-1H-indol-3-yl)ethyl)-N-ethyl)-N-propylethan-1-amine
2(2-(7-CF3-1H-6-azaindol-3-yl)-N-(-N-(2-(5-OH-1H-indol-3-yl)ethyl)-N-ethyl)-N-propylethan-1-amine
2(2-(4-D-1H-6-azaindol-3-yl)-N-(-N-(2-(6-CF3-1H-indol-3-yl)ethyl)-N-ethyl)-N-propylethan-1-amine
2(2-(5-OH-1H-6-azaindol-3-yl)-N-(-N-(2-(6-CF3-1H-indol-3-yl)ethyl)-N-ethyl)-N-propylethan-1-amine
2(2-(7-CF3-1H-6-azaindol-3-yl)-N-(-N-(2-(6-CF3-1H-indol-3-yl)ethyl)-N-ethyl)-N-propylethan-1-amine
2(2-(4-D-1H-6-azaindol-3-yl)-N-(-N-(2-(7-F-1H-indol-3-yl)ethyl)-N-ethyl)-N-propylethan-1-amine
2(2-(5-OH-1H-6-azaindol-3-yl)-N-(-N-(2-(7-F-1H-indol-3-yl)ethyl)-N-ethyl)-N-propylethan-1-amine
2(2-(7-CF3-1H-6-azaindol-3-yl)-N-(-N-(2-(7-F-1H-indol-3-yl)ethyl)-N-ethyl)-N-propylethan-1-amine
2(2-(4-D-1H-7-azaindol-3-yl)-N-(-N-(2-(4-D-1H-7-azaindol-3-yl)ethyl)-N-ethyl)-N-methylethan-1-amine
2(2-(5-OH-1H-7-azaindol-3-yl)-N-(-N-(2-(4-D-1H-7-azaindol-3-yl)ethyl)-N-ethyl)-N-methylethan-1-
amine
2(2-(6-CF3-1H-7-azaindol-3-yl)-N-(-N-(2-(4-D-1H-7-azaindol-3-yl)ethyl)-N-ethyl)-N-methylethan-1-
amine
2(2-(4-D-1H-7-azaindol-3-yl)-N-(-N-(2-(5-OH-1H-7-azaindol-3-yl)ethyl)-N-ethyl)-N-methylethan-1-
amine
2(2-(5-OH-1H-7-azaindol-3-yl)-N-(-N-(2-(5-OH-1H-7-azaindol-3-yl)ethyl)-N-ethyl)-N-methylethan-1-
amine
2(2-(6-CF3-1H-7-azaindol-3-yl)-N-(-N-(2-(5-OH-1H-7-azaindol-3-yl)ethyl)-N-ethyl)-N-methylethan-1-
amine
2(2-(4-D-1H-7-azaindol-3-yl)-N-(-N-(2-(6-CF3-1H-7-azaindol-3-yl)ethyl)-N-ethyl)-N-methylethan-1-
amine
2(2-(5-OH-1H-7-azaindol-3-yl)-N-(-N-(2-(6-CF3-1H-7-azaindol-3-yl)ethyl)-N-ethyl)-N-methylethan-1-
amine
2(2-(6-CF3-1H-7-azaindol-3-yl)-N-(-N-(2-(6-CF3-1H-7-azaindol-3-yl)ethyl)-N-ethyl)-N-methylethan-1-
amine
2(2-(4-D-1H-7-azaindol-3-yl)-N-(-N-(2-(4-D-1H-7-azaindol-3-yl)ethyl)-N-ethyl)-N-ethylethan-1-amine
2(2-(5-OH-1H-7-azaindol-3-yl)-N-(-N-(2-(4-D-1H-7-azaindol-3-yl)ethyl)-N-ethyl)-N-ethylethan-1-amine
2(2-(6-CF3-1H-7-azaindol-3-yl)-N-(-N-(2-(4-D-1H-7-azaindol-3-yl)ethyl)-N-ethyl)-N-ethylethan-1-amine
2(2-(4-D-1H-7-azaindol-3-yl)-N-(-N-(2-(5-OH-1H-7-azaindol-3-yl)ethyl)-N-ethyl)-N-ethylethan-1-amine
2(2-(5-OH-1H-7-azaindol-3-yl)-N-(-N-(2-(5-OH-1H-7-azaindol-3-yl)ethyl)-N-ethyl)-N-ethylethan-1-
amine
2(2-(6-CF3-1H-7-azaindol-3-yl)-N-(-N-(2-(5-OH-1H-7-azaindol-3-yl)ethyl)-N-ethyl)-N-ethylethan-1-
amine
2(2-(4-D-1H-7-azaindol-3-yl)-N-(-N-(2-(6-CF3-1H-7-azaindol-3-yl)ethyl)-N-ethyl)-N-ethylethan-1-amine
2(2-(5-OH-1H-7-azaindol-3-yl)-N-(-N-(2-(6-CF3-1H-7-azaindol-3-yl)ethyl)-N-ethyl)-N-ethylethan-1-
amine
2(2-(6-CF3-1H-7-azaindol-3-yl)-N-(-N-(2-(6-CF3-1H-7-azaindol-3-yl)ethyl)-N-ethyl)-N-ethylethan-1-
amine
2(2-(4-D-1H-7-azaindol-3-yl)-N-(-N-(2-(4-D-1H-7-azaindol-3-yl)ethyl)-N-ethyl)-N-isopropylethan-1-
amine
2(2-(5-OH-1H-7-azaindol-3-yl)-N-(-N-(2-(4-D-1H-7-azaindol-3-yl)ethyl)-N-ethyl)-N-isopropylethan-1-
amine
2(2-(6-CF3-1H-7-azaindol-3-yl)-N-(-N-(2-(4-D-1H-7-azaindol-3-yl)ethyl)-N-ethyl)-N-isopropylethan-1-
amine
2(2-(4-D-1H-7-azaindol-3-yl)-N-(-N-(2-(5-OH-1H-7-azaindol-3-yl)ethyl)-N-ethyl)-N-isopropylethan-1-
amine
2(2-(5-OH-1H-7-azaindol-3-yl)-N-(-N-(2-(5-OH-1H-7-azaindol-3-yl)ethyl)-N-ethyl)-N-isopropylethan-1-
amine
2(2-(6-CF3-1H-7-azaindol-3-yl)-N-(-N-(2-(5-OH-1H-7-azaindol-3-yl)ethyl)-N-ethyl)-N-isopropylethan-1-
amine
2(2-(4-D-1H-7-azaindol-3-yl)-N-(-N-(2-(6-CF3-1H-7-azaindol-3-yl)ethyl)-N-ethyl)-N-isopropylethan-1-
amine
2(2-(5-OH-1H-7-azaindol-3-yl)-N-(-N-(2-(6-CF3-1H-7-azaindol-3-yl)ethyl)-N-ethyl)-N-isopropylethan-1-
amine
2(2-(6-CF3-1H-7-azaindol-3-yl)-N-(-N-(2-(6-CF3-1H-7-azaindol-3-yl)ethyl)-N-ethyl)-N-isopropylethan-1-
amine
2(2-(4-D-1H-7-azaindol-3-yl)-N-(-N-(2-(4-D-1H-7-azaindol-3-yl)ethyl)-N-ethyl)-N-allylethan-1-amine
2(2-(5-OH-1H-7-azaindol-3-yl)-N-(-N-(2-(4-D-1H-7-azaindol-3-yl)ethyl)-N-ethyl)-N-allylethan-1-amine
2(2-(6-CF3-1H-7-azaindol-3-yl)-N-(-N-(2-(4-D-1H-7-azaindol-3-yl)ethyl)-N-ethyl)-N-allylethan-1-amine
2(2-(4-D-1H-7-azaindol-3-yl)-N-(-N-(2-(5-OH-1H-7-azaindol-3-yl)ethyl)-N-ethyl)-N-allylethan-1-amine
2(2-(5-OH-1H-7-azaindol-3-yl)-N-(-N-(2-(5-OH-1H-7-azaindol-3-yl)ethyl)-N-ethyl)-N-allylethan-1-amine
2(2-(6-CF3-1H-7-azaindol-3-yl)-N-(-N-(2-(5-OH-1H-7-azaindol-3-yl)ethyl)-N-ethyl)-N-allylethan-1-amine
2(2-(4-D-1H-7-azaindol-3-yl)-N-(-N-(2-(6-CF3-1H-7-azaindol-3-yl)ethyl)-N-ethyl)-N-allylethan-1-amine
2(2-(5-OH-1H-7-azaindol-3-yl)-N-(-N-(2-(6-CF3-1H-7-azaindol-3-yl)ethyl)-N-ethyl)-N-allylethan-1-amine
2(2-(6-CF3-1H-7-azaindol-3-yl)-N-(-N-(2-(6-CF3-1H-7-azaindol-3-yl)ethyl)-N-ethyl)-N-allylethan-1-
amine
2(2-(4-D-1H-7-azaindol-3-yl)-N-(-N-(2-(4-D-1H-7-azaindol-3-yl)ethyl)-N-ethyl)-N-hydroethan-1-amine
2(2-(5-OH-1H-7-azaindol-3-yl)-N-(-N-(2-(4-D-1H-7-azaindol-3-yl)ethyl)-N-ethyl)-N-hydroethan-1-amine
2(2-(6-CF3-1H-7-azaindol-3-yl)-N-(-N-(2-(4-D-1H-7-azaindol-3-yl)ethyl)-N-ethyl)-N-hydroethan-1-
amine
2(2-(4-D-1H-7-azaindol-3-yl)-N-(-N-(2-(5-OH-1H-7-azaindol-3-yl)ethyl)-N-ethyl)-N-hydroethan-1-amine
2(2-(5-OH-1H-7-azaindol-3-yl)-N-(-N-(2-(5-OH-1H-7-azaindol-3-yl)ethyl)-N-ethyl)-N-hydroethan-1-
amine
2(2-(6-CF3-1H-7-azaindol-3-yl)-N-(-N-(2-(5-OH-1H-7-azaindol-3-yl)ethyl)-N-ethyl)-N-hydroethan-1-
amine
2(2-(4-D-1H-7-azaindol-3-yl)-N-(-N-(2-(6-CF3-1H-7-azaindol-3-yl)ethyl)-N-ethyl)-N-hydroethan-1-
amine
2(2-(5-OH-1H-7-azaindol-3-yl)-N-(-N-(2-(6-CF3-1H-7-azaindol-3-yl)ethyl)-N-ethyl)-N-hydroethan-1-
amine
2(2-(6-CF3-1H-7-azaindol-3-yl)-N-(-N-(2-(6-CF3-1H-7-azaindol-3-yl)ethyl)-N-ethyl)-N-hydroethan-1-
amine
2(2-(4-D-1H-7-azaindol-3-yl)-N-(-N-(2-(4-D-1H-7-azaindol-3-yl)ethyl)-N-ethyl)-N-(D)ethan-1-amine
2(2-(5-OH-1H-7-azaindol-3-yl)-N-(-N-(2-(4-D-1H-7-azaindol-3-yl)ethyl)-N-ethyl)-N-(D)ethan-1-amine
2(2-(6-CF3-1H-7-azaindol-3-yl)-N-(-N-(2-(4-D-1H-7-azaindol-3-yl)ethyl)-N-ethyl)-N-(D)ethan-1-amine
2(2-(4-D-1H-7-azaindol-3-yl)-N-(-N-(2-(5-OH-1H-7-azaindol-3-yl)ethyl)-N-ethyl)-N-(D)ethan-1-amine
2(2-(5-OH-1H-7-azaindol-3-yl)-N-(-N-(2-(5-OH-1H-7-azaindol-3-yl)ethyl)-N-ethyl)-N-(D)ethan-1-amine
2(2-(6-CF3-1H-7-azaindol-3-yl)-N-(-N-(2-(5-OH-1H-7-azaindol-3-yl)ethyl)-N-ethyl)-N-(D)ethan-1-amine
2(2-(4-D-1H-7-azaindol-3-yl)-N-(-N-(2-(6-CF3-1H-7-azaindol-3-yl)ethyl)-N-ethyl)-N-(D)ethan-1-amine
2(2-(5-OH-1H-7-azaindol-3-yl)-N-(-N-(2-(6-CF3-1H-7-azaindol-3-yl)ethyl)-N-ethyl)-N-(D)ethan-1-amine
2(2-(6-CF3-1H-7-azaindol-3-yl)-N-(-N-(2-(6-CF3-1H-7-azaindol-3-yl)ethyl)-N-ethyl)-N-(D)ethan-1-amine
2(2-(4-D-1H-7-azaindol-3-yl)-N-(-N-(2-(4-D-1H-7-azaindol-3-yl)ethyl)-N-ethyl)-N-propylethan-1-amine
2(2-(5-OH-1H-7-azaindol-3-yl)-N-(-N-(2-(4-D-1H-7-azaindol-3-yl)ethyl)-N-ethyl)-N-propylethan-1-
amine
2(2-(6-CF3-1H-7-azaindol-3-yl)-N-(-N-(2-(4-D-1H-7-azaindol-3-yl)ethyl)-N-ethyl)-N-propylethan-1-
amine
2(2-(4-D-1H-7-azaindol-3-yl)-N-(-N-(2-(5-OH-1H-7-azaindol-3-yl)ethyl)-N-ethyl)-N-propylethan-1-
amine
2(2-(5-OH-1H-7-azaindol-3-yl)-N-(-N-(2-(5-OH-1H-7-azaindol-3-yl)ethyl)-N-ethyl)-N-propylethan-1-
amine
2(2-(6-CF3-1H-7-azaindol-3-yl)-N-(-N-(2-(5-OH-1H-7-azaindol-3-yl)ethyl)-N-ethyl)-N-propylethan-1-
amine
2(2-(4-D-1H-7-azaindol-3-yl)-N-(-N-(2-(6-CF3-1H-7-azaindol-3-yl)ethyl)-N-ethyl)-N-propylethan-1-
amine
2(2-(5-OH-1H-7-azaindol-3-yl)-N-(-N-(2-(6-CF3-1H-7-azaindol-3-yl)ethyl)-N-ethyl)-N-propylethan-1-
amine
2(2-(6-CF3-1H-7-azaindol-3-yl)-N-(-N-(2-(6-CF3-1H-7-azaindol-3-yl)ethyl)-N-ethyl)-N-propylethan-1-
amine
2(2-(4-D-1H-7-azaindol-3-yl)-N-(-N-(2-(4-D-1H-7a-azaindol-3-yl)ethyl)-N-ethyl)-N-methylethan-1-
amine
2(2-(5-OH-1H-7-azaindol-3-yl)-N-(-N-(2-(4-D-1H-7a-azaindol-3-yl)ethyl)-N-ethyl)-N-methylethan-1-
amine
2(2-(6-CF3-1H-7-azaindol-3-yl)-N-(-N-(2-(4-D-1H-7a-azaindol-3-yl)ethyl)-N-ethyl)-N-methylethan-1-
amine
2(2-(4-D-1H-7-azaindol-3-yl)-N-(-N-(2-(5-OH-1H-7a-azaindol-3-yl)ethyl)-N-ethyl)-N-methylethan-1-
amine
2(2-(5-OH-1H-7-azaindol-3-yl)-N-(-N-(2-(5-OH-1H-7a-azaindol-3-yl)ethyl)-N-ethyl)-N-methylethan-1-
amine
2(2-(6-CF3-1H-7-azaindol-3-yl)-N-(-N-(2-(5-OH-1H-7a-azaindol-3-yl)ethyl)-N-ethyl)-N-methylethan-1-
amine
2(2-(4-D-1H-7-azaindol-3-yl)-N-(-N-(2-(6-CF3-1H-7a-azaindol-3-yl)ethyl)-N-ethyl)-N-methylethan-1-
amine
2(2-(5-OH-1H-7-azaindol-3-yl)-N-(-N-(2-(6-CF3-1H-7a-azaindol-3-yl)ethyl)-N-ethyl)-N-methylethan-1-
amine
2(2-(6-CF3-1H-7-azaindol-3-yl)-N-(-N-(2-(6-CF3-1H-7a-azaindol-3-yl)ethyl)-N-ethyl)-N-methylethan-1-
amine
2(2-(4-D-1H-7-azaindol-3-yl)-N-(-N-(2-(7-F-1H-7a-azaindol-3-yl)ethyl)-N-ethyl)-N-methylethan-1-amine
2(2-(5-OH-1H-7-azaindol-3-yl)-N-(-N-(2-(7-F-1H-7a-azaindol-3-yl)ethyl)-N-ethyl)-N-methylethan-1-
amine
2(2-(6-CF3-1H-7-azaindol-3-yl)-N-(-N-(2-(7-F-1H-7a-azaindol-3-yl)ethyl)-N-ethyl)-N-methylethan-1-
amine
2(2-(4-D-1H-7-azaindol-3-yl)-N-(-N-(2-(4-D-1H-7a-azaindol-3-yl)ethyl)-N-ethyl)-N-ethylethan-1-amine
2(2-(5-OH-1H-7-azaindol-3-yl)-N-(-N-(2-(4-D-1H-7a-azaindol-3-yl)ethyl)-N-ethyl)-N-ethylethan-1-amine
2(2-(6-CF3-1H-7-azaindol-3-yl)-N-(-N-(2-(4-D-1H-7a-azaindol-3-yl)ethyl)-N-ethyl)-N-ethylethan-1-
amine
2(2-(4-D-1H-7-azaindol-3-yl)-N-(-N-(2-(5-OH-1H-7a-azaindol-3-yl)ethyl)-N-ethyl)-N-ethylethan-1-amine
2(2-(5-OH-1H-7-azaindol-3-yl)-N-(-N-(2-(5-OH-1H-7a-azaindol-3-yl)ethyl)-N-ethyl)-N-ethylethan-1-
amine
2(2-(6-CF3-1H-7-azaindol-3-yl)-N-(-N-(2-(5-OH-1H-7a-azaindol-3-yl)ethyl)-N-ethyl)-N-ethylethan-1-
amine
2(2-(4-D-1H-7-azaindol-3-yl)-N-(-N-(2-(6-CF3-1H-7a-azaindol-3-yl)ethyl)-N-ethyl)-N-ethylethan-1-
amine
2(2-(5-OH-1H-7-azaindol-3-yl)-N-(-N-(2-(6-CF3-1H-7a-azaindol-3-yl)ethyl)-N-ethyl)-N-ethylethan-1-
amine
2(2-(6-CF3-1H-7-azaindol-3-yl)-N-(-N-(2-(6-CF3-1H-7a-azaindol-3-yl)ethyl)-N-ethyl)-N-ethylethan-1-
amine
2(2-(4-D-1H-7-azaindol-3-yl)-N-(-N-(2-(7-F-1H-7a-azaindol-3-yl)ethyl)-N-ethyl)-N-ethylethan-1-amine
2(2-(5-OH-1H-7-azaindol-3-yl)-N-(-N-(2-(7-F-1H-7a-azaindol-3-yl)ethyl)-N-ethyl)-N-ethylethan-1-amine
2(2-(6-CF3-1H-7-azaindol-3-yl)-N-(-N-(2-(7-F-1H-7a-azaindol-3-yl)ethyl)-N-ethyl)-N-ethylethan-1-
amine
2(2-(4-D-1H-7-azaindol-3-yl)-N-(-N-(2-(4-D-1H-7a-azaindol-3-yl)ethyl)-N-ethyl)-N-isopropylethan-1-
amine
2(2-(5-OH-1H-7-azaindol-3-yl)-N-(-N-(2-(4-D-1H-7a-azaindol-3-yl)ethyl)-N-ethyl)-N-isopropylethan-1-
amine
2(2-(6-CF3-1H-7-azaindol-3-yl)-N-(-N-(2-(4-D-1H-7a-azaindol-3-yl)ethyl)-N-ethyl)-N-isopropylethan-1-
amine
2(2-(4-D-1H-7-azaindol-3-yl)-N-(-N-(2-(5-OH-1H-7a-azaindol-3-yl)ethyl)-N-ethyl)-N-isopropylethan-1-
amine
2(2-(5-OH-1H-7-azaindol-3-yl)-N-(-N-(2-(5-OH-1H-7a-azaindol-3-yl)ethyl)-N-ethyl)-N-isopropylethan-1-
amine
2(2-(6-CF3-1H-7-azaindol-3-yl)-N-(-N-(2-(5-OH-1H-7a-azaindol-3-yl)ethyl)-N-ethyl)-N-isopropylethan-
1-amine
2(2-(4-D-1H-7-azaindol-3-yl)-N-(-N-(2-(6-CF3-1H-7a-azaindol-3-yl)ethyl)-N-ethyl)-N-isopropylethan-1-
amine
2(2-(5-OH-1H-7-azaindol-3-yl)-N-(-N-(2-(6-CF3-1H-7a-azaindol-3-yl)ethyl)-N-ethyl)-N-isopropylethan-
1-amine
2(2-(6-CF3-1H-7-azaindol-3-yl)-N-(-N-(2-(6-CF3-1H-7a-azaindol-3-yl)ethyl)-N-ethyl)-N-isopropylethan-
1-amine
2(2-(4-D-1H-7-azaindol-3-yl)-N-(-N-(2-(7-F-1H-7a-azaindol-3-yl)ethyl)-N-ethyl)-N-isopropylethan-1-
amine
2(2-(5-OH-1H-7-azaindol-3-yl)-N-(-N-(2-(7-F-1H-7a-azaindol-3-yl)ethyl)-N-ethyl)-N-isopropylethan-1-
amine
2(2-(6-CF3-1H-7-azaindol-3-yl)-N-(-N-(2-(7-F-1H-7a-azaindol-3-yl)ethyl)-N-ethyl)-N-isopropylethan-1-
amine
2(2-(4-D-1H-7-azaindol-3-yl)-N-(-N-(2-(4-D-1H-7a-azaindol-3-yl)ethyl)-N-ethyl)-N-allylethan-1-amine
2(2-(5-OH-1H-7-azaindol-3-yl)-N-(-N-(2-(4-D-1H-7a-azaindol-3-yl)ethyl)-N-ethyl)-N-allylethan-1-amine
2(2-(6-CF3-1H-7-azaindol-3-yl)-N-(-N-(2-(4-D-1H-7a-azaindol-3-yl)ethyl)-N-ethyl)-N-allylethan-1-amine
2(2-(4-D-1H-7-azaindol-3-yl)-N-(-N-(2-(5-OH-1H-7a-azaindol-3-yl)ethyl)-N-ethyl)-N-allylethan-1-amine
2(2-(5-OH-1H-7-azaindol-3-yl)-N-(-N-(2-(5-OH-1H-7a-azaindol-3-yl)ethyl)-N-ethyl)-N-allylethan-1-
amine
2(2-(6-CF3-1H-7-azaindol-3-yl)-N-(-N-(2-(5-OH-1H-7a-azaindol-3-yl)ethyl)-N-ethyl)-N-allylethan-1-
amine
2(2-(4-D-1H-7-azaindol-3-yl)-N-(-N-(2-(6-CF3-1H-7a-azaindol-3-yl)ethyl)-N-ethyl)-N-allylethan-1-amine
2(2-(5-OH-1H-7-azaindol-3-yl)-N-(-N-(2-(6-CF3-1H-7a-azaindol-3-yl)ethyl)-N-ethyl)-N-allylethan-1-
amine
2(2-(6-CF3-1H-7-azaindol-3-yl)-N-(-N-(2-(6-CF3-1H-7a-azaindol-3-yl)ethyl)-N-ethyl)-N-allylethan-1-
amine
2(2-(4-D-1H-7-azaindol-3-yl)-N-(-N-(2-(7-F-1H-7a-azaindol-3-yl)ethyl)-N-ethyl)-N-allylethan-1-amine
2(2-(5-OH-1H-7-azaindol-3-yl)-N-(-N-(2-(7-F-1H-7a-azaindol-3-yl)ethyl)-N-ethyl)-N-allylethan-1-amine
2(2-(6-CF3-1H-7-azaindol-3-yl)-N-(-N-(2-(7-F-1H-7a-azaindol-3-yl)ethyl)-N-ethyl)-N-allylethan-1-amine
2(2-(4-D-1H-7-azaindol-3-yl)-N-(-N-(2-(4-D-1H-7a-azaindol-3-yl)ethyl)-N-ethyl)-N-hydroethan-1-amine
2(2-(5-OH-1H-7-azaindol-3-yl)-N-(-N-(2-(4-D-1H-7a-azaindol-3-yl)ethyl)-N-ethyl)-N-hydroethan-1-
amine
2(2-(6-CF3-1H-7-azaindol-3-yl)-N-(-N-(2-(4-D-1H-7a-azaindol-3-yl)ethyl)-N-ethyl)-N-hydroethan-1-
amine
2(2-(4-D-1H-7-azaindol-3-yl)-N-(-N-(2-(5-OH-1H-7a-azaindol-3-yl)ethyl)-N-ethyl)-N-hydroethan-1-
amine
2(2-(5-OH-1H-7-azaindol-3-yl)-N-(-N-(2-(5-OH-1H-7a-azaindol-3-yl)ethyl)-N-ethyl)-N-hydroethan-1-
amine
2(2-(6-CF3-1H-7-azaindol-3-yl)-N-(-N-(2-(5-OH-1H-7a-azaindol-3-yl)ethyl)-N-ethyl)-N-hydroethan-1-
amine
2(2-(4-D-1H-7-azaindol-3-yl)-N-(-N-(2-(6-CF3-1H-7a-azaindol-3-yl)ethyl)-N-ethyl)-N-hydroethan-1-
amine
2(2-(5-OH-1H-7-azaindol-3-yl)-N-(-N-(2-(6-CF3-1H-7a-azaindol-3-yl)ethyl)-N-ethyl)-N-hydroethan-1-
amine
2(2-(6-CF3-1H-7-azaindol-3-yl)-N-(-N-(2-(6-CF3-1H-7a-azaindol-3-yl)ethyl)-N-ethyl)-N-hydroethan-1-
amine
2(2-(4-D-1H-7-azaindol-3-yl)-N-(-N-(2-(7-F-1H-7a-azaindol-3-yl)ethyl)-N-ethyl)-N-hydroethan-1-amine
2(2-(5-OH-1H-7-azaindol-3-yl)-N-(-N-(2-(7-F-1H-7a-azaindol-3-yl)ethyl)-N-ethyl)-N-hydroethan-1-
amine
2(2-(6-CF3-1H-7-azaindol-3-yl)-N-(-N-(2-(7-F-1H-7a-azaindol-3-yl)ethyl)-N-ethyl)-N-hydroethan-1-
amine
2(2-(4-D-1H-7-azaindol-3-yl)-N-(-N-(2-(4-D-1H-7a-azaindol-3-yl)ethyl)-N-ethyl)-N-(D)ethan-1-amine
2(2-(5-OH-1H-7-azaindol-3-yl)-N-(-N-(2-(4-D-1H-7a-azaindol-3-yl)ethyl)-N-ethyl)-N-(D)ethan-1-amine
2(2-(6-CF3-1H-7-azaindol-3-yl)-N-(-N-(2-(4-D-1H-7a-azaindol-3-yl)ethyl)-N-ethyl)-N-(D)ethan-1-amine
2(2-(4-D-1H-7-azaindol-3-yl)-N-(-N-(2-(5-OH-1H-7a-azaindol-3-yl)ethyl)-N-ethyl)-N-(D)ethan-1-amine
2(2-(5-OH-1H-7-azaindol-3-yl)-N-(-N-(2-(5-OH-1H-7a-azaindol-3-yl)ethyl)-N-ethyl)-N-(D)ethan-1-amine
2(2-(6-CF3-1H-7-azaindol-3-yl)-N-(-N-(2-(5-OH-1H-7a-azaindol-3-yl)ethyl)-N-ethyl)-N-(D)ethan-1-
amine
2(2-(4-D-1H-7-azaindol-3-yl)-N-(-N-(2-(6-CF3-1H-7a-azaindol-3-yl)ethyl)-N-ethyl)-N-(D)ethan-1-amine
2(2-(5-OH-1H-7-azaindol-3-yl)-N-(-N-(2-(6-CF3-1H-7a-azaindol-3-yl)ethyl)-N-ethyl)-N-(D)ethan-1-
amine
2(2-(6-CF3-1H-7-azaindol-3-yl)-N-(-N-(2-(6-CF3-1H-7a-azaindol-3-yl)ethyl)-N-ethyl)-N-(D)ethan-1-
amine
2(2-(4-D-1H-7-azaindol-3-yl)-N-(-N-(2-(7-F-1H-7a-azaindol-3-yl)ethyl)-N-ethyl)-N-(D)ethan-1-amine
2(2-(5-OH-1H-7-azaindol-3-yl)-N-(-N-(2-(7-F-1H-7a-azaindol-3-yl)ethyl)-N-ethyl)-N-(D)ethan-1-amine
2(2-(6-CF3-1H-7-azaindol-3-yl)-N-(-N-(2-(7-F-1H-7a-azaindol-3-yl)ethyl)-N-ethyl)-N-(D)ethan-1-amine
2(2-(4-D-1H-7-azaindol-3-yl)-N-(-N-(2-(4-D-1H-7a-azaindol-3-yl)ethyl)-N-ethyl)-N-propylethan-1-amine
2(2-(5-OH-1H-7-azaindol-3-yl)-N-(-N-(2-(4-D-1H-7a-azaindol-3-yl)ethyl)-N-ethyl)-N-propylethan-1-
amine
2(2-(6-CF3-1H-7-azaindol-3-yl)-N-(-N-(2-(4-D-1H-7a-azaindol-3-yl)ethyl)-N-ethyl)-N-propylethan-1-
amine
2(2-(4-D-1H-7-azaindol-3-yl)-N-(-N-(2-(5-OH-1H-7a-azaindol-3-yl)ethyl)-N-ethyl)-N-propylethan-1-
amine
2(2-(5-OH-1H-7-azaindol-3-yl)-N-(-N-(2-(5-OH-1H-7a-azaindol-3-yl)ethyl)-N-ethyl)-N-propylethan-1-
amine
2(2-(6-CF3-1H-7-azaindol-3-yl)-N-(-N-(2-(5-OH-1H-7a-azaindol-3-yl)ethyl)-N-ethyl)-N-propylethan-1-
amine
2(2-(4-D-1H-7-azaindol-3-yl)-N-(-N-(2-(6-CF3-1H-7a-azaindol-3-yl)ethyl)-N-ethyl)-N-propylethan-1-
amine
2(2-(5-OH-1H-7-azaindol-3-yl)-N-(-N-(2-(6-CF3-1H-7a-azaindol-3-yl)ethyl)-N-ethyl)-N-propylethan-1-
amine
2(2-(6-CF3-1H-7-azaindol-3-yl)-N-(-N-(2-(6-CF3-1H-7a-azaindol-3-yl)ethyl)-N-ethyl)-N-propylethan-1-
amine
2(2-(4-D-1H-7-azaindol-3-yl)-N-(-N-(2-(7-F-1H-7a-azaindol-3-yl)ethyl)-N-ethyl)-N-propylethan-1-amine
2(2-(5-OH-1H-7-azaindol-3-yl)-N-(-N-(2-(7-F-1H-7a-azaindol-3-yl)ethyl)-N-ethyl)-N-propylethan-1-
amine
2(2-(6-CF3-1H-7-azaindol-3-yl)-N-(-N-(2-(7-F-1H-7a-azaindol-3-yl)ethyl)-N-ethyl)-N-propylethan-1-
amine
2(2-(4-D-1H-7-azaindol-3-yl)-N-(-N-(2-(4-D-1H-indol-3-yl)ethyl)-N-ethyl)-N-methylethan-1-amine
2(2-(5-OH-1H-7-azaindol-3-yl)-N-(-N-(2-(4-D-1H-indol-3-yl)ethyl)-N-ethyl)-N-methylethan-1-amine
2(2-(6-CF3-1H-7-azaindol-3-yl)-N-(-N-(2-(4-D-1H-indol-3-yl)ethyl)-N-ethyl)-N-methylethan-1-amine
2(2-(4-D-1H-7-azaindol-3-yl)-N-(-N-(2-(5-OH-1H-indol-3-yl)ethyl)-N-ethyl)-N-methylethan-1-amine
2(2-(5-OH-1H-7-azaindol-3-yl)-N-(-N-(2-(5-OH-1H-indol-3-yl)ethyl)-N-ethyl)-N-methylethan-1-amine
2(2-(6-CF3-1H-7-azaindol-3-yl)-N-(-N-(2-(5-OH-1H-indol-3-yl)ethyl)-N-ethyl)-N-methylethan-1-amine
2(2-(4-D-1H-7-azaindol-3-yl)-N-(-N-(2-(6-CF3-1H-indol-3-yl)ethyl)-N-ethyl)-N-methylethan-1-amine
2(2-(5-OH-1H-7-azaindol-3-yl)-N-(-N-(2-(6-CF3-1H-indol-3-yl)ethyl)-N-ethyl)-N-methylethan-1-amine
2(2-(6-CF3-1H-7-azaindol-3-yl)-N-(-N-(2-(6-CF3-1H-indol-3-yl)ethyl)-N-ethyl)-N-methylethan-1-amine
2(2-(4-D-1H-7-azaindol-3-yl)-N-(-N-(2-(7-F-1H-indol-3-yl)ethyl)-N-ethyl)-N-methylethan-1-amine
2(2-(5-OH-1H-7-azaindol-3-yl)-N-(-N-(2-(7-F-1H-indol-3-yl)ethyl)-N-ethyl)-N-methylethan-1-amine
2(2-(6-CF3-1H-7-azaindol-3-yl)-N-(-N-(2-(7-F-1H-indol-3-yl)ethyl)-N-ethyl)-N-methylethan-1-amine
2(2-(4-D-1H-7-azaindol-3-yl)-N-(-N-(2-(4-D-1H-indol-3-yl)ethyl)-N-ethyl)-N-ethylethan-1-amine
2(2-(5-OH-1H-7-azaindol-3-yl)-N-(-N-(2-(4-D-1H-indol-3-yl)ethyl)-N-ethyl)-N-ethylethan-1-amine
2(2-(6-CF3-1H-7-azaindol-3-yl)-N-(-N-(2-(4-D-1H-indol-3-yl)ethyl)-N-ethyl)-N-ethylethan-1-amine
2(2-(4-D-1H-7-azaindol-3-yl)-N-(-N-(2-(5-OH-1H-indol-3-yl)ethyl)-N-ethyl)-N-ethylethan-1-amine
2(2-(5-OH-1H-7-azaindol-3-yl)-N-(-N-(2-(5-OH-1H-indol-3-yl)ethyl)-N-ethyl)-N-ethylethan-1-amine
2(2-(6-CF3-1H-7-azaindol-3-yl)-N-(-N-(2-(5-OH-1H-indol-3-yl)ethyl)-N-ethyl)-N-ethylethan-1-amine
2(2-(4-D-1H-7-azaindol-3-yl)-N-(-N-(2-(6-CF3-1H-indol-3-yl)ethyl)-N-ethyl)-N-ethylethan-1-amine
2(2-(5-OH-1H-7-azaindol-3-yl)-N-(-N-(2-(6-CF3-1H-indol-3-yl)ethyl)-N-ethyl)-N-ethylethan-1-amine
2(2-(6-CF3-1H-7-azaindol-3-yl)-N-(-N-(2-(6-CF3-1H-indol-3-yl)ethyl)-N-ethyl)-N-ethylethan-1-amine
2(2-(4-D-1H-7-azaindol-3-yl)-N-(-N-(2-(7-F-1H-indol-3-yl)ethyl)-N-ethyl)-N-ethylethan-1-amine
2(2-(5-OH-1H-7-azaindol-3-yl)-N-(-N-(2-(7-F-1H-indol-3-yl)ethyl)-N-ethyl)-N-ethylethan-1-amine
2(2-(6-CF3-1H-7-azaindol-3-yl)-N-(-N-(2-(7-F-1H-indol-3-yl)ethyl)-N-ethyl)-N-ethylethan-1-amine
2(2-(4-D-1H-7-azaindol-3-yl)-N-(-N-(2-(4-D-1H-indol-3-yl)ethyl)-N-ethyl)-N-isopropylethan-1-amine
2(2-(5-OH-1H-7-azaindol-3-yl)-N-(-N-(2-(4-D-1H-indol-3-yl)ethyl)-N-ethyl)-N-isopropylethan-1-amine
2(2-(6-CF3-1H-7-azaindol-3-yl)-N-(-N-(2-(4-D-1H-indol-3-yl)ethyl)-N-ethyl)-N-isopropylethan-1-amine
2(2-(4-D-1H-7-azaindol-3-yl)-N-(-N-(2-(5-OH-1H-indol-3-yl)ethyl)-N-ethyl)-N-isopropylethan-1-amine
2(2-(5-OH-1H-7-azaindol-3-yl)-N-(-N-(2-(5-OH-1H-indol-3-yl)ethyl)-N-ethyl)-N-isopropylethan-1-amine
2(2-(6-CF3-1H-7-azaindol-3-yl)-N-(-N-(2-(5-OH-1H-indol-3-yl)ethyl)-N-ethyl)-N-isopropylethan-1-amine
2(2-(4-D-1H-7-azaindol-3-yl)-N-(-N-(2-(6-CF3-1H-indol-3-yl)ethyl)-N-ethyl)-N-isopropylethan-1-amine
2(2-(5-OH-1H-7-azaindol-3-yl)-N-(-N-(2-(6-CF3-1H-indol-3-yl)ethyl)-N-ethyl)-N-isopropylethan-1-amine
2(2-(6-CF3-1H-7-azaindol-3-yl)-N-(-N-(2-(6-CF3-1H-indol-3-yl)ethyl)-N-ethyl)-N-isopropylethan-1-
amine
2(2-(4-D-1H-7-azaindol-3-yl)-N-(-N-(2-(7-F-1H-indol-3-yl)ethyl)-N-ethyl)-N-isopropylethan-1-amine
2(2-(5-OH-1H-7-azaindol-3-yl)-N-(-N-(2-(7-F-1H-indol-3-yl)ethyl)-N-ethyl)-N-isopropylethan-1-amine
2(2-(6-CF3-1H-7-azaindol-3-yl)-N-(-N-(2-(7-F-1H-indol-3-yl)ethyl)-N-ethyl)-N-isopropylethan-1-amine
2(2-(4-D-1H-7-azaindol-3-yl)-N-(-N-(2-(4-D-1H-indol-3-yl)ethyl)-N-ethyl)-N-allylethan-1-amine
2(2-(5-OH-1H-7-azaindol-3-yl)-N-(-N-(2-(4-D-1H-indol-3-yl)ethyl)-N-ethyl)-N-allylethan-1-amine
2(2-(6-CF3-1H-7-azaindol-3-yl)-N-(-N-(2-(4-D-1H-indol-3-yl)ethyl)-N-ethyl)-N-allylethan-1-amine
2(2-(4-D-1H-7-azaindol-3-yl)-N-(-N-(2-(5-OH-1H-indol-3-yl)ethyl)-N-ethyl)-N-allylethan-1-amine
2(2-(5-OH-1H-7-azaindol-3-yl)-N-(-N-(2-(5-OH-1H-indol-3-yl)ethyl)-N-ethyl)-N-allylethan-1-amine
2(2-(6-CF3-1H-7-azaindol-3-yl)-N-(-N-(2-(5-OH-1H-indol-3-yl)ethyl)-N-ethyl)-N-allylethan-1-amine
2(2-(4-D-1H-7-azaindol-3-yl)-N-(-N-(2-(6-CF3-1H-indol-3-yl)ethyl)-N-ethyl)-N-allylethan-1-amine
2(2-(5-OH-1H-7-azaindol-3-yl)-N-(-N-(2-(6-CF3-1H-indol-3-yl)ethyl)-N-ethyl)-N-allylethan-1-amine
2(2-(6-CF3-1H-7-azaindol-3-yl)-N-(-N-(2-(6-CF3-1H-indol-3-yl)ethyl)-N-ethyl)-N-allylethan-1-amine
2(2-(4-D-1H-7-azaindol-3-yl)-N-(-N-(2-(7-F-1H-indol-3-yl)ethyl)-N-ethyl)-N-allylethan-1-amine
2(2-(5-OH-1H-7-azaindol-3-yl)-N-(-N-(2-(7-F-1H-indol-3-yl)ethyl)-N-ethyl)-N-allylethan-1-amine
2(2-(6-CF3-1H-7-azaindol-3-yl)-N-(-N-(2-(7-F-1H-indol-3-yl)ethyl)-N-ethyl)-N-allylethan-1-amine
2(2-(4-D-1H-7-azaindol-3-yl)-N-(-N-(2-(4-D-1H-indol-3-yl)ethyl)-N-ethyl)-N-hydroethan-1-amine
2(2-(5-OH-1H-7-azaindol-3-yl)-N-(-N-(2-(4-D-1H-indol-3-yl)ethyl)-N-ethyl)-N-hydroethan-1-amine
2(2-(6-CF3-1H-7-azaindol-3-yl)-N-(-N-(2-(4-D-1H-indol-3-yl)ethyl)-N-ethyl)-N-hydroethan-1-amine
2(2-(4-D-1H-7-azaindol-3-yl)-N-(-N-(2-(5-OH-1H-indol-3-yl)ethyl)-N-ethyl)-N-hydroethan-1-amine
2(2-(5-OH-1H-7-azaindol-3-yl)-N-(-N-(2-(5-OH-1H-indol-3-yl)ethyl)-N-ethyl)-N-hydroethan-1-amine
2(2-(6-CF3-1H-7-azaindol-3-yl)-N-(-N-(2-(5-OH-1H-indol-3-yl)ethyl)-N-ethyl)-N-hydroethan-1-amine
2(2-(4-D-1H-7-azaindol-3-yl)-N-(-N-(2-(6-CF3-1H-indol-3-yl)ethyl)-N-ethyl)-N-hydroethan-1-amine
2(2-(5-OH-1H-7-azaindol-3-yl)-N-(-N-(2-(6-CF3-1H-indol-3-yl)ethyl)-N-ethyl)-N-hydroethan-1-amine
2(2-(6-CF3-1H-7-azaindol-3-yl)-N-(-N-(2-(6-CF3-1H-indol-3-yl)ethyl)-N-ethyl)-N-hydroethan-1-amine
2(2-(4-D-1H-7-azaindol-3-yl)-N-(-N-(2-(7-F-1H-indol-3-yl)ethyl)-N-ethyl)-N-hydroethan-1-amine
2(2-(5-OH-1H-7-azaindol-3-yl)-N-(-N-(2-(7-F-1H-indol-3-yl)ethyl)-N-ethyl)-N-hydroethan-1-amine
2(2-(6-CF3-1H-7-azaindol-3-yl)-N-(-N-(2-(7-F-1H-indol-3-yl)ethyl)-N-ethyl)-N-hydroethan-1-amine
2(2-(4-D-1H-7-azaindol-3-yl)-N-(-N-(2-(4-D-1H-indol-3-yl)ethyl)-N-ethyl)-N-(D)ethan-1-amine
2(2-(5-OH-1H-7-azaindol-3-yl)-N-(-N-(2-(4-D-1H-indol-3-yl)ethyl)-N-ethyl)-N-(D)ethan-1-amine
2(2-(6-CF3-1H-7-azaindol-3-yl)-N-(-N-(2-(4-D-1H-indol-3-yl)ethyl)-N-ethyl)-N-(D)ethan-1-amine
2(2-(4-D-1H-7-azaindol-3-yl)-N-(-N-(2-(5-OH-1H-indol-3-yl)ethyl)-N-ethyl)-N-(D)ethan-1-amine
2(2-(5-OH-1H-7-azaindol-3-yl)-N-(-N-(2-(5-OH-1H-indol-3-yl)ethyl)-N-ethyl)-N-(D)ethan-1-amine
2(2-(6-CF3-1H-7-azaindol-3-yl)-N-(-N-(2-(5-OH-1H-indol-3-yl)ethyl)-N-ethyl)-N-(D)ethan-1-amine
2(2-(4-D-1H-7-azaindol-3-yl)-N-(-N-(2-(6-CF3-1H-indol-3-yl)ethyl)-N-ethyl)-N-(D)ethan-1-amine
2(2-(5-OH-1H-7-azaindol-3-yl)-N-(-N-(2-(6-CF3-1H-indol-3-yl)ethyl)-N-ethyl)-N-(D)ethan-1-amine
2(2-(6-CF3-1H-7-azaindol-3-yl)-N-(-N-(2-(6-CF3-1H-indol-3-yl)ethyl)-N-ethyl)-N-(D)ethan-1-amine
2(2-(4-D-1H-7-azaindol-3-yl)-N-(-N-(2-(7-F-1H-indol-3-yl)ethyl)-N-ethyl)-N-(D)ethan-1-amine
2(2-(5-OH-1H-7-azaindol-3-yl)-N-(-N-(2-(7-F-1H-indol-3-yl)ethyl)-N-ethyl)-N-(D)ethan-1-amine
2(2-(6-CF3-1H-7-azaindol-3-yl)-N-(-N-(2-(7-F-1H-indol-3-yl)ethyl)-N-ethyl)-N-(D)ethan-1-amine
2(2-(4-D-1H-7-azaindol-3-yl)-N-(-N-(2-(4-D-1H-indol-3-yl)ethyl)-N-ethyl)-N-propylethan-1-amine
2(2-(5-OH-1H-7-azaindol-3-yl)-N-(-N-(2-(4-D-1H-indol-3-yl)ethyl)-N-ethyl)-N-propylethan-1-amine
2(2-(6-CF3-1H-7-azaindol-3-yl)-N-(-N-(2-(4-D-1H-indol-3-yl)ethyl)-N-ethyl)-N-propylethan-1-amine
2(2-(4-D-1H-7-azaindol-3-yl)-N-(-N-(2-(5-OH-1H-indol-3-yl)ethyl)-N-ethyl)-N-propylethan-1-amine
2(2-(5-OH-1H-7-azaindol-3-yl)-N-(-N-(2-(5-OH-1H-indol-3-yl)ethyl)-N-ethyl)-N-propylethan-1-amine
2(2-(6-CF3-1H-7-azaindol-3-yl)-N-(-N-(2-(5-OH-1H-indol-3-yl)ethyl)-N-ethyl)-N-propylethan-1-amine
2(2-(4-D-1H-7-azaindol-3-yl)-N-(-N-(2-(6-CF3-1H-indol-3-yl)ethyl)-N-ethyl)-N-propylethan-1-amine
2(2-(5-OH-1H-7-azaindol-3-yl)-N-(-N-(2-(6-CF3-1H-indol-3-yl)ethyl)-N-ethyl)-N-propylethan-1-amine
2(2-(6-CF3-1H-7-azaindol-3-yl)-N-(-N-(2-(6-CF3-1H-indol-3-yl)ethyl)-N-ethyl)-N-propylethan-1-amine
2(2-(4-D-1H-7-azaindol-3-yl)-N-(-N-(2-(7-F-1H-indol-3-yl)ethyl)-N-ethyl)-N-propylethan-1-amine
2(2-(5-OH-1H-7-azaindol-3-yl)-N-(-N-(2-(7-F-1H-indol-3-yl)ethyl)-N-ethyl)-N-propylethan-1-amine
2(2-(6-CF3-1H-7-azaindol-3-yl)-N-(-N-(2-(7-F-1H-indol-3-yl)ethyl)-N-ethyl)-N-propylethan-1-amine
2(2-(4-D-1H-7a-azaindol-3-yl)-N-(-N-(2-(4-D-1H-7a-azaindol-3-yl)ethyl)-N-ethyl)-N-methylethan-1-
amine
2(2-(5-OH-1H-7a-azaindol-3-yl)-N-(-N-(2-(4-D-1H-7a-azaindol-3-yl)ethyl)-N-ethyl)-N-methylethan-1-
amine
2(2-(6-CF3-1H-7a-azaindol-3-yl)-N-(-N-(2-(4-D-1H-7a-azaindol-3-yl)ethyl)-N-ethyl)-N-methylethan-1-
amine
2(2-(7-F-1H-7a-azaindol-3-yl)-N-(-N-(2-(4-D-1H-7a-azaindol-3-yl)ethyl)-N-ethyl)-N-methylethan-1-
amine
2(2-(4-D-1H-7a-azaindol-3-yl)-N-(-N-(2-(5-OH-1H-7a-azaindol-3-yl)ethyl)-N-ethyl)-N-methylethan-1-
amine
2(2-(5-OH-1H-7a-azaindol-3-yl)-N-(-N-(2-(5-OH-1H-7a-azaindol-3-yl)ethyl)-N-ethyl)-N-methylethan-1-
amine
2(2-(6-CF3-1H-7a-azaindol-3-yl)-N-(-N-(2-(5-OH-1H-7a-azaindol-3-yl)ethyl)-N-ethyl)-N-methylethan-1-
amine
2(2-(7-F-1H-7a-azaindol-3-yl)-N-(-N-(2-(5-OH-1H-7a-azaindol-3-yl)ethyl)-N-ethyl)-N-methylethan-1-
amine
2(2-(4-D-1H-7a-azaindol-3-yl)-N-(-N-(2-(6-CF3-1H-7a-azaindol-3-yl)ethyl)-N-ethyl)-N-methylethan-1-
amine
2(2-(5-OH-1H-7a-azaindol-3-yl)-N-(-N-(2-(6-CF3-1H-7a-azaindol-3-yl)ethyl)-N-ethyl)-N-methylethan-1-
amine
2(2-(6-CF3-1H-7a-azaindol-3-yl)-N-(-N-(2-(6-CF3-1H-7a-azaindol-3-yl)ethyl)-N-ethyl)-N-methylethan-1-
amine
2(2-(7-F-1H-7a-azaindol-3-yl)-N-(-N-(2-(6-CF3-1H-7a-azaindol-3-yl)ethyl)-N-ethyl)-N-methylethan-1-
amine
2(2-(4-D-1H-7a-azaindol-3-yl)-N-(-N-(2-(7-F-1H-7a-azaindol-3-yl)ethyl)-N-ethyl)-N-methylethan-1-
amine
2(2-(5-OH-1H-7a-azaindol-3-yl)-N-(-N-(2-(7-F-1H-7a-azaindol-3-yl)ethyl)-N-ethyl)-N-methylethan-1-
amine
2(2-(6-CF3-1H-7a-azaindol-3-yl)-N-(-N-(2-(7-F-1H-7a-azaindol-3-yl)ethyl)-N-ethyl)-N-methylethan-1-
amine
2(2-(7-F-1H-7a-azaindol-3-yl)-N-(-N-(2-(7-F-1H-7a-azaindol-3-yl)ethyl)-N-ethyl)-N-methylethan-1-
amine
2(2-(4-D-1H-7a-azaindol-3-yl)-N-(-N-(2-(4-D-1H-7a-azaindol-3-yl)ethyl)-N-ethyl)-N-ethylethan-1-amine
2(2-(5-OH-1H-7a-azaindol-3-yl)-N-(-N-(2-(4-D-1H-7a-azaindol-3-yl)ethyl)-N-ethyl)-N-ethylethan-1-
amine
2(2-(6-CF3-1H-7a-azaindol-3-yl)-N-(-N-(2-(4-D-1H-7a-azaindol-3-yl)ethyl)-N-ethyl)-N-ethylethan-1-
amine
2(2-(7-F-1H-7a-azaindol-3-yl)-N-(-N-(2-(4-D-1H-7a-azaindol-3-yl)ethyl)-N-ethyl)-N-ethylethan-1-amine
2(2-(4-D-1H-7a-azaindol-3-yl)-N-(-N-(2-(5-OH-1H-7a-azaindol-3-yl)ethyl)-N-ethyl)-N-ethylethan-1-
amine
2(2-(5-OH-1H-7a-azaindol-3-yl)-N-(-N-(2-(5-OH-1H-7a-azaindol-3-yl)ethyl)-N-ethyl)-N-ethylethan-1-
amine
2(2-(6-CF3-1H-7a-azaindol-3-yl)-N-(-N-(2-(5-OH-1H-7a-azaindol-3-yl)ethyl)-N-ethyl)-N-ethylethan-1-
amine
2(2-(7-F-1H-7a-azaindol-3-yl)-N-(-N-(2-(5-OH-1H-7a-azaindol-3-yl)ethyl)-N-ethyl)-N-ethylethan-1-
amine
2(2-(4-D-1H-7a-azaindol-3-yl)-N-(-N-(2-(6-CF3-1H-7a-azaindol-3-yl)ethyl)-N-ethyl)-N-ethylethan-1-
amine
2(2-(5-OH-1H-7a-azaindol-3-yl)-N-(-N-(2-(6-CF3-1H-7a-azaindol-3-yl)ethyl)-N-ethyl)-N-ethylethan-1-
amine
2(2-(6-CF3-1H-7a-azaindol-3-yl)-N-(-N-(2-(6-CF3-1H-7a-azaindol-3-yl)ethyl)-N-ethyl)-N-ethylethan-1-
amine
2(2-(7-F-1H-7a-azaindol-3-yl)-N-(-N-(2-(6-CF3-1H-7a-azaindol-3-yl)ethyl)-N-ethyl)-N-ethylethan-1-
amine
2(2-(4-D-1H-7a-azaindol-3-yl)-N-(-N-(2-(7-F-1H-7a-azaindol-3-yl)ethyl)-N-ethyl)-N-ethylethan-1-amine
2(2-(5-OH-1H-7a-azaindol-3-yl)-N-(-N-(2-(7-F-1H-7a-azaindol-3-yl)ethyl)-N-ethyl)-N-ethylethan-1-
amine
2(2-(6-CF3-1H-7a-azaindol-3-yl)-N-(-N-(2-(7-F-1H-7a-azaindol-3-yl)ethyl)-N-ethyl)-N-ethylethan-1-
amine
2(2-(7-F-1H-7a-azaindol-3-yl)-N-(-N-(2-(7-F-1H-7a-azaindol-3-yl)ethyl)-N-ethyl)-N-ethylethan-1-amine
2(2-(4-D-1H-7a-azaindol-3-yl)-N-(-N-(2-(4-D-1H-7a-azaindol-3-yl)ethyl)-N-ethyl)-N-isopropylethan-1-
amine
2(2-(5-OH-1H-7a-azaindol-3-yl)-N-(-N-(2-(4-D-1H-7a-azaindol-3-yl)ethyl)-N-ethyl)-N-isopropylethan-1-
amine
2(2-(6-CF3-1H-7a-azaindol-3-yl)-N-(-N-(2-(4-D-1H-7a-azaindol-3-yl)ethyl)-N-ethyl)-N-isopropylethan-1-
amine
2(2-(7-F-1H-7a-azaindol-3-yl)-N-(-N-(2-(4-D-1H-7a-azaindol-3-yl)ethyl)-N-ethyl)-N-isopropylethan-1-
amine
2(2-(4-D-1H-7a-azaindol-3-yl)-N-(-N-(2-(5-OH-1H-7a-azaindol-3-yl)ethyl)-N-ethyl)-N-isopropylethan-1-
amine
2(2-(5-OH-1H-7a-azaindol-3-yl)-N-(-N-(2-(5-OH-1H-7a-azaindol-3-yl)ethyl)-N-ethyl)-N-isopropylethan-
1-amine
2(2-(6-CF3-1H-7a-azaindol-3-yl)-N-(-N-(2-(5-OH-1H-7a-azaindol-3-yl)ethyl)-N-ethyl)-N-isopropylethan-
1-amine
2(2-(7-F-1H-7a-azaindol-3-yl)-N-(-N-(2-(5-OH-1H-7a-azaindol-3-yl)ethyl)-N-ethyl)-N-isopropylethan-1-
amine
2(2-(4-D-1H-7a-azaindol-3-yl)-N-(-N-(2-(6-CF3-1H-7a-azaindol-3-yl)ethyl)-N-ethyl)-N-isopropylethan-1-
amine
2(2-(5-OH-1H-7a-azaindol-3-yl)-N-(-N-(2-(6-CF3-1H-7a-azaindol-3-yl)ethyl)-N-ethyl)-N-isopropylethan-
1-amine
2(2-(6-CF3-1H-7a-azaindol-3-yl)-N-(-N-(2-(6-CF3-1H-7a-azaindol-3-yl)ethyl)-N-ethyl)-N-isopropylethan-
1-amine
2(2-(7-F-1H-7a-azaindol-3-yl)-N-(-N-(2-(6-CF3-1H-7a-azaindol-3-yl)ethyl)-N-ethyl)-N-isopropylethan-1-
amine
2(2-(4-D-1H-7a-azaindol-3-yl)-N-(-N-(2-(7-F-1H-7a-azaindol-3-yl)ethyl)-N-ethyl)-N-isopropylethan-1-
amine
2(2-(5-OH-1H-7a-azaindol-3-yl)-N-(-N-(2-(7-F-1H-7a-azaindol-3-yl)ethyl)-N-ethyl)-N-isopropylethan-1-
amine
2(2-(6-CF3-1H-7a-azaindol-3-yl)-N-(-N-(2-(7-F-1H-7a-azaindol-3-yl)ethyl)-N-ethyl)-N-isopropylethan-1-
amine
2(2-(7-F-1H-7a-azaindol-3-yl)-N-(-N-(2-(7-F-1H-7a-azaindol-3-yl)ethyl)-N-ethyl)-N-isopropylethan-1-
amine
2(2-(4-D-1H-7a-azaindol-3-yl)-N-(-N-(2-(4-D-1H-7a-azaindol-3-yl)ethyl)-N-ethyl)-N-allylethan-1-amine
2(2-(5-OH-1H-7a-azaindol-3-yl)-N-(-N-(2-(4-D-1H-7a-azaindol-3-yl)ethyl)-N-ethyl)-N-allylethan-1-
amine
2(2-(6-CF3-1H-7a-azaindol-3-yl)-N-(-N-(2-(4-D-1H-7a-azaindol-3-yl)ethyl)-N-ethyl)-N-allylethan-1-
amine
2(2-(7-F-1H-7a-azaindol-3-yl)-N-(-N-(2-(4-D-1H-7a-azaindol-3-yl)ethyl)-N-ethyl)-N-allylethan-1-amine
2(2-(4-D-1H-7a-azaindol-3-yl)-N-(-N-(2-(5-OH-1H-7a-azaindol-3-yl)ethyl)-N-ethyl)-N-allylethan-1-
amine
2(2-(5-OH-1H-7a-azaindol-3-yl)-N-(-N-(2-(5-OH-1H-7a-azaindol-3-yl)ethyl)-N-ethyl)-N-allylethan-1-
amine
2(2-(6-CF3-1H-7a-azaindol-3-yl)-N-(-N-(2-(5-OH-1H-7a-azaindol-3-yl)ethyl)-N-ethyl)-N-allylethan-1-
amine
2(2-(7-F-1H-7a-azaindol-3-yl)-N-(-N-(2-(5-OH-1H-7a-azaindol-3-yl)ethyl)-N-ethyl)-N-allylethan-1-amine
2(2-(4-D-1H-7a-azaindol-3-yl)-N-(-N-(2-(6-CF3-1H-7a-azaindol-3-yl)ethyl)-N-ethyl)-N-allylethan-1-
amine
2(2-(5-OH-1H-7a-azaindol-3-yl)-N-(-N-(2-(6-CF3-1H-7a-azaindol-3-yl)ethyl)-N-ethyl)-N-allylethan-1-
amine
2(2-(6-CF3-1H-7a-azaindol-3-yl)-N-(-N-(2-(6-CF3-1H-7a-azaindol-3-yl)ethyl)-N-ethyl)-N-allylethan-1-
amine
2(2-(7-F-1H-7a-azaindol-3-yl)-N-(-N-(2-(6-CF3-1H-7a-azaindol-3-yl)ethyl)-N-ethyl)-N-allylethan-1-
amine
2(2-(4-D-1H-7a-azaindol-3-yl)-N-(-N-(2-(7-F-1H-7a-azaindol-3-yl)ethyl)-N-ethyl)-N-allylethan-1-amine
2(2-(5-OH-1H-7a-azaindol-3-yl)-N-(-N-(2-(7-F-1H-7a-azaindol-3-yl)ethyl)-N-ethyl)-N-allylethan-1-amine
2(2-(6-CF3-1H-7a-azaindol-3-yl)-N-(-N-(2-(7-F-1H-7a-azaindol-3-yl)ethyl)-N-ethyl)-N-allylethan-1-
amine
2(2-(7-F-1H-7a-azaindol-3-yl)-N-(-N-(2-(7-F-1H-7a-azaindol-3-yl)ethyl)-N-ethyl)-N-allylethan-1-amine
2(2-(4-D-1H-7a-azaindol-3-yl)-N-(-N-(2-(4-D-1H-7a-azaindol-3-yl)ethyl)-N-ethyl)-N-hydroethan-1-
amine
2(2-(5-OH-1H-7a-azaindol-3-yl)-N-(-N-(2-(4-D-1H-7a-azaindol-3-yl)ethyl)-N-ethyl)-N-hydroethan-1-
amine
2(2-(6-CF3-1H-7a-azaindol-3-yl)-N-(-N-(2-(4-D-1H-7a-azaindol-3-yl)ethyl)-N-ethyl)-N-hydroethan-1-
amine
2(2-(7-F-1H-7a-azaindol-3-yl)-N-(-N-(2-(4-D-1H-7a-azaindol-3-yl)ethyl)-N-ethyl)-N-hydroethan-1-amine
2(2-(4-D-1H-7a-azaindol-3-yl)-N-(-N-(2-(5-OH-1H-7a-azaindol-3-yl)ethyl)-N-ethyl)-N-hydroethan-1-
amine
2(2-(5-OH-1H-7a-azaindol-3-yl)-N-(-N-(2-(5-OH-1H-7a-azaindol-3-yl)ethyl)-N-ethyl)-N-hydroethan-1-
amine
2(2-(6-CF3-1H-7a-azaindol-3-yl)-N-(-N-(2-(5-OH-1H-7a-azaindol-3-yl)ethyl)-N-ethyl)-N-hydroethan-1-
amine
2(2-(7-F-1H-7a-azaindol-3-yl)-N-(-N-(2-(5-OH-1H-7a-azaindol-3-yl)ethyl)-N-ethyl)-N-hydroethan-1-
amine
2(2-(4-D-1H-7a-azaindol-3-yl)-N-(-N-(2-(6-CF3-1H-7a-azaindol-3-yl)ethyl)-N-ethyl)-N-hydroethan-1-
amine
2(2-(5-OH-1H-7a-azaindol-3-yl)-N-(-N-(2-(6-CF3-1H-7a-azaindol-3-yl)ethyl)-N-ethyl)-N-hydroethan-1-
amine
2(2-(6-CF3-1H-7a-azaindol-3-yl)-N-(-N-(2-(6-CF3-1H-7a-azaindol-3-yl)ethyl)-N-ethyl)-N-hydroethan-1-
amine
2(2-(7-F-1H-7a-azaindol-3-yl)-N-(-N-(2-(6-CF3-1H-7a-azaindol-3-yl)ethyl)-N-ethyl)-N-hydroethan-1-
amine
2(2-(4-D-1H-7a-azaindol-3-yl)-N-(-N-(2-(7-F-1H-7a-azaindol-3-yl)ethyl)-N-ethyl)-N-hydroethan-1-amine
2(2-(5-OH-1H-7a-azaindol-3-yl)-N-(-N-(2-(7-F-1H-7a-azaindol-3-yl)ethyl)-N-ethyl)-N-hydroethan-1-
amine
2(2-(6-CF3-1H-7a-azaindol-3-yl)-N-(-N-(2-(7-F-1H-7a-azaindol-3-yl)ethyl)-N-ethyl)-N-hydroethan-1-
amine
2(2-(7-F-1H-7a-azaindol-3-yl)-N-(-N-(2-(7-F-1H-7a-azaindol-3-yl)ethyl)-N-ethyl)-N-hydroethan-1-amine
2(2-(4-D-1H-7a-azaindol-3-yl)-N-(-N-(2-(4-D-1H-7a-azaindol-3-yl)ethyl)-N-ethyl)-N-(D)ethan-1-amine
2(2-(5-OH-1H-7a-azaindol-3-yl)-N-(-N-(2-(4-D-1H-7a-azaindol-3-yl)ethyl)-N-ethyl)-N-(D)ethan-1-amine
2(2-(6-CF3-1H-7a-azaindol-3-yl)-N-(-N-(2-(4-D-1H-7a-azaindol-3-yl)ethyl)-N-ethyl)-N-(D)ethan-1-amine
2(2-(7-F-1H-7a-azaindol-3-yl)-N-(-N-(2-(4-D-1H-7a-azaindol-3-yl)ethyl)-N-ethyl)-N-(D)ethan-1-amine
2(2-(4-D-1H-7a-azaindol-3-yl)-N-(-N-(2-(5-OH-1H-7a-azaindol-3-yl)ethyl)-N-ethyl)-N-(D)ethan-1-amine
2(2-(5-OH-1H-7a-azaindol-3-yl)-N-(-N-(2-(5-OH-1H-7a-azaindol-3-yl)ethyl)-N-ethyl)-N-(D)ethan-1-
amine
2(2-(6-CF3-1H-7a-azaindol-3-yl)-N-(-N-(2-(5-OH-1H-7a-azaindol-3-yl)ethyl)-N-ethyl)-N-(D)ethan-1-
amine
2(2-(7-F-1H-7a-azaindol-3-yl)-N-(-N-(2-(5-OH-1H-7a-azaindol-3-yl)ethyl)-N-ethyl)-N-(D)ethan-1-amine
2(2-(4-D-1H-7a-azaindol-3-yl)-N-(-N-(2-(6-CF3-1H-7a-azaindol-3-yl)ethyl)-N-ethyl)-N-(D)ethan-1-amine
2(2-(5-OH-1H-7a-azaindol-3-yl)-N-(-N-(2-(6-CF3-1H-7a-azaindol-3-yl)ethyl)-N-ethyl)-N-(D)ethan-1-
amine
2(2-(6-CF3-1H-7a-azaindol-3-yl)-N-(-N-(2-(6-CF3-1H-7a-azaindol-3-yl)ethyl)-N-ethyl)-N-(D)ethan-1-
amine
2(2-(7-F-1H-7a-azaindol-3-yl)-N-(-N-(2-(6-CF3-1H-7a-azaindol-3-yl)ethyl)-N-ethyl)-N-(D)ethan-1-amine
2(2-(4-D-1H-7a-azaindol-3-yl)-N-(-N-(2-(7-F-1H-7a-azaindol-3-yl)ethyl)-N-ethyl)-N-(D)ethan-1-amine
2(2-(5-OH-1H-7a-azaindol-3-yl)-N-(-N-(2-(7-F-1H-7a-azaindol-3-yl)ethyl)-N-ethyl)-N-(D)ethan-1-amine
2(2-(6-CF3-1H-7a-azaindol-3-yl)-N-(-N-(2-(7-F-1H-7a-azaindol-3-yl)ethyl)-N-ethyl)-N-(D)ethan-1-amine
2(2-(7-F-1H-7a-azaindol-3-yl)-N-(-N-(2-(7-F-1H-7a-azaindol-3-yl)ethyl)-N-ethyl)-N-(D)ethan-1-amine
2(2-(4-D-1H-7a-azaindol-3-yl)-N-(-N-(2-(4-D-1H-7a-azaindol-3-yl)ethyl)-N-ethyl)-N-propylethan-1-
amine
2(2-(5-OH-1H-7a-azaindol-3-yl)-N-(-N-(2-(4-D-1H-7a-azaindol-3-yl)ethyl)-N-ethyl)-N-propylethan-1-
amine
2(2-(6-CF3-1H-7a-azaindol-3-yl)-N-(-N-(2-(4-D-1H-7a-azaindol-3-yl)ethyl)-N-ethyl)-N-propylethan-1-
amine
2(2-(7-F-1H-7a-azaindol-3-yl)-N-(-N-(2-(4-D-1H-7a-azaindol-3-yl)ethyl)-N-ethyl)-N-propylethan-1-
amine
2(2-(4-D-1H-7a-azaindol-3-yl)-N-(-N-(2-(5-OH-1H-7a-azaindol-3-yl)ethyl)-N-ethyl)-N-propylethan-1-
amine
2(2-(5-OH-1H-7a-azaindol-3-yl)-N-(-N-(2-(5-OH-1H-7a-azaindol-3-yl)ethyl)-N-ethyl)-N-propylethan-1-
amine
2(2-(6-CF3-1H-7a-azaindol-3-yl)-N-(-N-(2-(5-OH-1H-7a-azaindol-3-yl)ethyl)-N-ethyl)-N-propylethan-1-
amine
2(2-(7-F-1H-7a-azaindol-3-yl)-N-(-N-(2-(5-OH-1H-7a-azaindol-3-yl)ethyl)-N-ethyl)-N-propylethan-1-
amine
2(2-(4-D-1H-7a-azaindol-3-yl)-N-(-N-(2-(6-CF3-1H-7a-azaindol-3-yl)ethyl)-N-ethyl)-N-propylethan-1-
amine
2(2-(5-OH-1H-7a-azaindol-3-yl)-N-(-N-(2-(6-CF3-1H-7a-azaindol-3-yl)ethyl)-N-ethyl)-N-propylethan-1-
amine
2(2-(6-CF3-1H-7a-azaindol-3-yl)-N-(-N-(2-(6-CF3-1H-7a-azaindol-3-yl)ethyl)-N-ethyl)-N-propylethan-1-
amine
2(2-(7-F-1H-7a-azaindol-3-yl)-N-(-N-(2-(6-CF3-1H-7a-azaindol-3-yl)ethyl)-N-ethyl)-N-propylethan-1-
amine
2(2-(4-D-1H-7a-azaindol-3-yl)-N-(-N-(2-(7-F-1H-7a-azaindol-3-yl)ethyl)-N-ethyl)-N-propylethan-1-
amine
2(2-(5-OH-1H-7a-azaindol-3-yl)-N-(-N-(2-(7-F-1H-7a-azaindol-3-yl)ethyl)-N-ethyl)-N-propylethan-1-
amine
2(2-(6-CF3-1H-7a-azaindol-3-yl)-N-(-N-(2-(7-F-1H-7a-azaindol-3-yl)ethyl)-N-ethyl)-N-propylethan-1-
amine
2(2-(7-F-1H-7a-azaindol-3-yl)-N-(-N-(2-(7-F-1H-7a-azaindol-3-yl)ethyl)-N-ethyl)-N-propylethan-1-
amine
2(2-(4-D-1H-7a-azaindol-3-yl)-N-(-N-(2-(4-D-1H-indol-3-yl)ethyl)-N-ethyl)-N-methylethan-1-amine
2(2-(5-OH-1H-7a-azaindol-3-yl)-N-(-N-(2-(4-D-1H-indol-3-yl)ethyl)-N-ethyl)-N-methylethan-1-amine
2(2-(6-CF3-1H-7a-azaindol-3-yl)-N-(-N-(2-(4-D-1H-indol-3-yl)ethyl)-N-ethyl)-N-methylethan-1-amine
2(2-(7-F-1H-7a-azaindol-3-yl)-N-(-N-(2-(4-D-1H-indol-3-yl)ethyl)-N-ethyl)-N-methylethan-1-amine
2(2-(4-D-1H-7a-azaindol-3-yl)-N-(-N-(2-(5-OH-1H-indol-3-yl)ethyl)-N-ethyl)-N-methylethan-1-amine
2(2-(5-OH-1H-7a-azaindol-3-yl)-N-(-N-(2-(5-OH-1H-indol-3-yl)ethyl)-N-ethyl)-N-methylethan-1-amine
2(2-(6-CF3-1H-7a-azaindol-3-yl)-N-(-N-(2-(5-OH-1H-indol-3-yl)ethyl)-N-ethyl)-N-methylethan-1-amine
2(2-(7-F-1H-7a-azaindol-3-yl)-N-(-N-(2-(5-OH-1H-indol-3-yl)ethyl)-N-ethyl)-N-methylethan-1-amine
2(2-(4-D-1H-7a-azaindol-3-yl)-N-(-N-(2-(6-CF3-1H-indol-3-yl)ethyl)-N-ethyl)-N-methylethan-1-amine
2(2-(5-OH-1H-7a-azaindol-3-yl)-N-(-N-(2-(6-CF3-1H-indol-3-yl)ethyl)-N-ethyl)-N-methylethan-1-amine
2(2-(6-CF3-1H-7a-azaindol-3-yl)-N-(-N-(2-(6-CF3-1H-indol-3-yl)ethyl)-N-ethyl)-N-methylethan-1-amine
2(2-(7-F-1H-7a-azaindol-3-yl)-N-(-N-(2-(6-CF3-1H-indol-3-yl)ethyl)-N-ethyl)-N-methylethan-1-amine
2(2-(4-D-1H-7a-azaindol-3-yl)-N-(-N-(2-(7-F-1H-indol-3-yl)ethyl)-N-ethyl)-N-methylethan-1-amine
2(2-(5-OH-1H-7a-azaindol-3-yl)-N-(-N-(2-(7-F-1H-indol-3-yl)ethyl)-N-ethyl)-N-methylethan-1-amine
2(2-(6-CF3-1H-7a-azaindol-3-yl)-N-(-N-(2-(7-F-1H-indol-3-yl)ethyl)-N-ethyl)-N-methylethan-1-amine
2(2-(7-F-1H-7a-azaindol-3-yl)-N-(-N-(2-(7-F-1H-indol-3-yl)ethyl)-N-ethyl)-N-methylethan-1-amine
2(2-(4-D-1H-7a-azaindol-3-yl)-N-(-N-(2-(4-D-1H-indol-3-yl)ethyl)-N-ethyl)-N-ethylethan-1-amine
2(2-(5-OH-1H-7a-azaindol-3-yl)-N-(-N-(2-(4-D-1H-indol-3-yl)ethyl)-N-ethyl)-N-ethylethan-1-amine
2(2-(6-CF3-1H-7a-azaindol-3-yl)-N-(-N-(2-(4-D-1H-indol-3-yl)ethyl)-N-ethyl)-N-ethylethan-1-amine
2(2-(7-F-1H-7a-azaindol-3-yl)-N-(-N-(2-(4-D-1H-indol-3-yl)ethyl)-N-ethyl)-N-ethylethan-1-amine
2(2-(4-D-1H-7a-azaindol-3-yl)-N-(-N-(2-(5-OH-1H-indol-3-yl)ethyl)-N-ethyl)-N-ethylethan-1-amine
2(2-(5-OH-1H-7a-azaindol-3-yl)-N-(-N-(2-(5-OH-1H-indol-3-yl)ethyl)-N-ethyl)-N-ethylethan-1-amine
2(2-(6-CF3-1H-7a-azaindol-3-yl)-N-(-N-(2-(5-OH-1H-indol-3-yl)ethyl)-N-ethyl)-N-ethylethan-1-amine
2(2-(7-F-1H-7a-azaindol-3-yl)-N-(-N-(2-(5-OH-1H-indol-3-yl)ethyl)-N-ethyl)-N-ethylethan-1-amine
2(2-(4-D-1H-7a-azaindol-3-yl)-N-(-N-(2-(6-CF3-1H-indol-3-yl)ethyl)-N-ethyl)-N-ethylethan-1-amine
2(2-(5-OH-1H-7a-azaindol-3-yl)-N-(-N-(2-(6-CF3-1H-indol-3-yl)ethyl)-N-ethyl)-N-ethylethan-1-amine
2(2-(6-CF3-1H-7a-azaindol-3-yl)-N-(-N-(2-(6-CF3-1H-indol-3-yl)ethyl)-N-ethyl)-N-ethylethan-1-amine
2(2-(7-F-1H-7a-azaindol-3-yl)-N-(-N-(2-(6-CF3-1H-indol-3-yl)ethyl)-N-ethyl)-N-ethylethan-1-amine
2(2-(4-D-1H-7a-azaindol-3-yl)-N-(-N-(2-(7-F-1H-indol-3-yl)ethyl)-N-ethyl)-N-ethylethan-1-amine
2(2-(5-OH-1H-7a-azaindol-3-yl)-N-(-N-(2-(7-F-1H-indol-3-yl)ethyl)-N-ethyl)-N-ethylethan-1-amine
2(2-(6-CF3-1H-7a-azaindol-3-yl)-N-(-N-(2-(7-F-1H-indol-3-yl)ethyl)-N-ethyl)-N-ethylethan-1-amine
2(2-(7-F-1H-7a-azaindol-3-yl)-N-(-N-(2-(7-F-1H-indol-3-yl)ethyl)-N-ethyl)-N-ethylethan-1-amine
2(2-(4-D-1H-7a-azaindol-3-yl)-N-(-N-(2-(4-D-1H-indol-3-yl)ethyl)-N-ethyl)-N-isopropylethan-1-amine
2(2-(5-OH-1H-7a-azaindol-3-yl)-N-(-N-(2-(4-D-1H-indol-3-yl)ethyl)-N-ethyl)-N-isopropylethan-1-amine
2(2-(6-CF3-1H-7a-azaindol-3-yl)-N-(-N-(2-(4-D-1H-indol-3-yl)ethyl)-N-ethyl)-N-isopropylethan-1-amine
2(2-(7-F-1H-7a-azaindol-3-yl)-N-(-N-(2-(4-D-1H-indol-3-yl)ethyl)-N-ethyl)-N-isopropylethan-1-amine
2(2-(4-D-1H-7a-azaindol-3-yl)-N-(-N-(2-(5-OH-1H-indol-3-yl)ethyl)-N-ethyl)-N-isopropylethan-1-amine
2(2-(5-OH-1H-7a-azaindol-3-yl)-N-(-N-(2-(5-OH-1H-indol-3-yl)ethyl)-N-ethyl)-N-isopropylethan-1-
amine
2(2-(6-CF3-1H-7a-azaindol-3-yl)-N-(-N-(2-(5-OH-1H-indol-3-yl)ethyl)-N-ethyl)-N-isopropylethan-1-
amine
2(2-(7-F-1H-7a-azaindol-3-yl)-N-(-N-(2-(5-OH-1H-indol-3-yl)ethyl)-N-ethyl)-N-isopropylethan-1-amine
2(2-(4-D-1H-7a-azaindol-3-yl)-N-(-N-(2-(6-CF3-1H-indol-3-yl)ethyl)-N-ethyl)-N-isopropylethan-1-amine
2(2-(5-OH-1H-7a-azaindol-3-yl)-N-(-N-(2-(6-CF3-1H-indol-3-yl)ethyl)-N-ethyl)-N-isopropylethan-1-
amine
2(2-(6-CF3-1H-7a-azaindol-3-yl)-N-(-N-(2-(6-CF3-1H-indol-3-yl)ethyl)-N-ethyl)-N-isopropylethan-1-
amine
2(2-(7-F-1H-7a-azaindol-3-yl)-N-(-N-(2-(6-CF3-1H-indol-3-yl)ethyl)-N-ethyl)-N-isopropylethan-1-amine
2(2-(4-D-1H-7a-azaindol-3-yl)-N-(-N-(2-(7-F-1H-indol-3-yl)ethyl)-N-ethyl)-N-isopropylethan-1-amine
2(2-(5-OH-1H-7a-azaindol-3-yl)-N-(-N-(2-(7-F-1H-indol-3-yl)ethyl)-N-ethyl)-N-isopropylethan-1-amine
2(2-(6-CF3-1H-7a-azaindol-3-yl)-N-(-N-(2-(7-F-1H-indol-3-yl)ethyl)-N-ethyl)-N-isopropylethan-1-amine
2(2-(7-F-1H-7a-azaindol-3-yl)-N-(-N-(2-(7-F-1H-indol-3-yl)ethyl)-N-ethyl)-N-isopropylethan-1-amine
2(2-(4-D-1H-7a-azaindol-3-yl)-N-(-N-(2-(4-D-1H-indol-3-yl)ethyl)-N-ethyl)-N-allylethan-1-amine
2(2-(5-OH-1H-7a-azaindol-3-yl)-N-(-N-(2-(4-D-1H-indol-3-yl)ethyl)-N-ethyl)-N-allylethan-1-amine
2(2-(6-CF3-1H-7a-azaindol-3-yl)-N-(-N-(2-(4-D-1H-indol-3-yl)ethyl)-N-ethyl)-N-allylethan-1-amine
2(2-(7-F-1H-7a-azaindol-3-yl)-N-(-N-(2-(4-D-1H-indol-3-yl)ethyl)-N-ethyl)-N-allylethan-1-amine
2(2-(4-D-1H-7a-azaindol-3-yl)-N-(-N-(2-(5-OH-1H-indol-3-yl)ethyl)-N-ethyl)-N-allylethan-1-amine
2(2-(5-OH-1H-7a-azaindol-3-yl)-N-(-N-(2-(5-OH-1H-indol-3-yl)ethyl)-N-ethyl)-N-allylethan-1-amine
2(2-(6-CF3-1H-7a-azaindol-3-yl)-N-(-N-(2-(5-OH-1H-indol-3-yl)ethyl)-N-ethyl)-N-allylethan-1-amine
2(2-(7-F-1H-7a-azaindol-3-yl)-N-(-N-(2-(5-OH-1H-indol-3-yl)ethyl)-N-ethyl)-N-allylethan-1-amine
2(2-(4-D-1H-7a-azaindol-3-yl)-N-(-N-(2-(6-CF3-1H-indol-3-yl)ethyl)-N-ethyl)-N-allylethan-1-amine
2(2-(5-OH-1H-7a-azaindol-3-yl)-N-(-N-(2-(6-CF3-1H-indol-3-yl)ethyl)-N-ethyl)-N-allylethan-1-amine
2(2-(6-CF3-1H-7a-azaindol-3-yl)-N-(-N-(2-(6-CF3-1H-indol-3-yl)ethyl)-N-ethyl)-N-allylethan-1-amine
2(2-(7-F-1H-7a-azaindol-3-yl)-N-(-N-(2-(6-CF3-1H-indol-3-yl)ethyl)-N-ethyl)-N-allylethan-1-amine
2(2-(4-D-1H-7a-azaindol-3-yl)-N-(-N-(2-(7-F-1H-indol-3-yl)ethyl)-N-ethyl)-N-allylethan-1-amine
2(2-(5-OH-1H-7a-azaindol-3-yl)-N-(-N-(2-(7-F-1H-indol-3-yl)ethyl)-N-ethyl)-N-allylethan-1-amine
2(2-(6-CF3-1H-7a-azaindol-3-yl)-N-(-N-(2-(7-F-1H-indol-3-yl)ethyl)-N-ethyl)-N-allylethan-1-amine
2(2-(7-F-1H-7a-azaindol-3-yl)-N-(-N-(2-(7-F-1H-indol-3-yl)ethyl)-N-ethyl)-N-allylethan-1-amine
2(2-(4-D-1H-7a-azaindol-3-yl)-N-(-N-(2-(4-D-1H-indol-3-yl)ethyl)-N-ethyl)-N-hydroethan-1-amine
2(2-(5-OH-1H-7a-azaindol-3-yl)-N-(-N-(2-(4-D-1H-indol-3-yl)ethyl)-N-ethyl)-N-hydroethan-1-amine
2(2-(6-CF3-1H-7a-azaindol-3-yl)-N-(-N-(2-(4-D-1H-indol-3-yl)ethyl)-N-ethyl)-N-hydroethan-1-amine
2(2-(7-F-1H-7a-azaindol-3-yl)-N-(-N-(2-(4-D-1H-indol-3-yl)ethyl)-N-ethyl)-N-hydroethan-1-amine
2(2-(4-D-1H-7a-azaindol-3-yl)-N-(-N-(2-(5-OH-1H-indol-3-yl)ethyl)-N-ethyl)-N-hydroethan-1-amine
2(2-(5-OH-1H-7a-azaindol-3-yl)-N-(-N-(2-(5-OH-1H-indol-3-yl)ethyl)-N-ethyl)-N-hydroethan-1-amine
2(2-(6-CF3-1H-7a-azaindol-3-yl)-N-(-N-(2-(5-OH-1H-indol-3-yl)ethyl)-N-ethyl)-N-hydroethan-1-amine
2(2-(7-F-1H-7a-azaindol-3-yl)-N-(-N-(2-(5-OH-1H-indol-3-yl)ethyl)-N-ethyl)-N-hydroethan-1-amine
2(2-(4-D-1H-7a-azaindol-3-yl)-N-(-N-(2-(6-CF3-1H-indol-3-yl)ethyl)-N-ethyl)-N-hydroethan-1-amine
2(2-(5-OH-1H-7a-azaindol-3-yl)-N-(-N-(2-(6-CF3-1H-indol-3-yl)ethyl)-N-ethyl)-N-hydroethan-1-amine
2(2-(6-CF3-1H-7a-azaindol-3-yl)-N-(-N-(2-(6-CF3-1H-indol-3-yl)ethyl)-N-ethyl)-N-hydroethan-1-amine
2(2-(7-F-1H-7a-azaindol-3-yl)-N-(-N-(2-(6-CF3-1H-indol-3-yl)ethyl)-N-ethyl)-N-hydroethan-1-amine
2(2-(4-D-1H-7a-azaindol-3-yl)-N-(-N-(2-(7-F-1H-indol-3-yl)ethyl)-N-ethyl)-N-hydroethan-1-amine
2(2-(5-OH-1H-7a-azaindol-3-yl)-N-(-N-(2-(7-F-1H-indol-3-yl)ethyl)-N-ethyl)-N-hydroethan-1-amine
2(2-(6-CF3-1H-7a-azaindol-3-yl)-N-(-N-(2-(7-F-1H-indol-3-yl)ethyl)-N-ethyl)-N-hydroethan-1-amine
2(2-(7-F-1H-7a-azaindol-3-yl)-N-(-N-(2-(7-F-1H-indol-3-yl)ethyl)-N-ethyl)-N-hydroethan-1-amine
2(2-(4-D-1H-7a-azaindol-3-yl)-N-(-N-(2-(4-D-1H-indol-3-yl)ethyl)-N-ethyl)-N-(D)ethan-1-amine
2(2-(5-OH-1H-7a-azaindol-3-yl)-N-(-N-(2-(4-D-1H-indol-3-yl)ethyl)-N-ethyl)-N-(D)ethan-1-amine
2(2-(6-CF3-1H-7a-azaindol-3-yl)-N-(-N-(2-(4-D-1H-indol-3-yl)ethyl)-N-ethyl)-N-(D)ethan-1-amine
2(2-(7-F-1H-7a-azaindol-3-yl)-N-(-N-(2-(4-D-1H-indol-3-yl)ethyl)-N-ethyl)-N-(D)ethan-1-amine
2(2-(4-D-1H-7a-azaindol-3-yl)-N-(-N-(2-(5-OH-1H-indol-3-yl)ethyl)-N-ethyl)-N-(D)ethan-1-amine
2(2-(5-OH-1H-7a-azaindol-3-yl)-N-(-N-(2-(5-OH-1H-indol-3-yl)ethyl)-N-ethyl)-N-(D)ethan-1-amine
2(2-(6-CF3-1H-7a-azaindol-3-yl)-N-(-N-(2-(5-OH-1H-indol-3-yl)ethyl)-N-ethyl)-N-(D)ethan-1-amine
2(2-(7-F-1H-7a-azaindol-3-yl)-N-(-N-(2-(5-OH-1H-indol-3-yl)ethyl)-N-ethyl)-N-(D)ethan-1-amine
2(2-(4-D-1H-7a-azaindol-3-yl)-N-(-N-(2-(6-CF3-1H-indol-3-yl)ethyl)-N-ethyl)-N-(D)ethan-1-amine
2(2-(5-OH-1H-7a-azaindol-3-yl)-N-(-N-(2-(6-CF3-1H-indol-3-yl)ethyl)-N-ethyl)-N-(D)ethan-1-amine
2(2-(6-CF3-1H-7a-azaindol-3-yl)-N-(-N-(2-(6-CF3-1H-indol-3-yl)ethyl)-N-ethyl)-N-(D)ethan-1-amine
2(2-(7-F-1H-7a-azaindol-3-yl)-N-(-N-(2-(6-CF3-1H-indol-3-yl)ethyl)-N-ethyl)-N-(D)ethan-1-amine
2(2-(4-D-1H-7a-azaindol-3-yl)-N-(-N-(2-(7-F-1H-indol-3-yl)ethyl)-N-ethyl)-N-(D)ethan-1-amine
2(2-(5-OH-1H-7a-azaindol-3-yl)-N-(-N-(2-(7-F-1H-indol-3-yl)ethyl)-N-ethyl)-N-(D)ethan-1-amine
2(2-(6-CF3-1H-7a-azaindol-3-yl)-N-(-N-(2-(7-F-1H-indol-3-yl)ethyl)-N-ethyl)-N-(D)ethan-1-amine
2(2-(7-F-1H-7a-azaindol-3-yl)-N-(-N-(2-(7-F-1H-indol-3-yl)ethyl)-N-ethyl)-N-(D)ethan-1-amine
2(2-(4-D-1H-7a-azaindol-3-yl)-N-(-N-(2-(4-D-1H-indol-3-yl)ethyl)-N-ethyl)-N-propylethan-1-amine
2(2-(5-OH-1H-7a-azaindol-3-yl)-N-(-N-(2-(4-D-1H-indol-3-yl)ethyl)-N-ethyl)-N-propylethan-1-amine
2(2-(6-CF3-1H-7a-azaindol-3-yl)-N-(-N-(2-(4-D-1H-indol-3-yl)ethyl)-N-ethyl)-N-propylethan-1-amine
2(2-(7-F-1H-7a-azaindol-3-yl)-N-(-N-(2-(4-D-1H-indol-3-yl)ethyl)-N-ethyl)-N-propylethan-1-amine
2(2-(4-D-1H-7a-azaindol-3-yl)-N-(-N-(2-(5-OH-1H-indol-3-yl)ethyl)-N-ethyl)-N-propylethan-1-amine
2(2-(5-OH-1H-7a-azaindol-3-yl)-N-(-N-(2-(5-OH-1H-indol-3-yl)ethyl)-N-ethyl)-N-propylethan-1-amine
2(2-(6-CF3-1H-7a-azaindol-3-yl)-N-(-N-(2-(5-OH-1H-indol-3-yl)ethyl)-N-ethyl)-N-propylethan-1-amine
2(2-(7-F-1H-7a-azaindol-3-yl)-N-(-N-(2-(5-OH-1H-indol-3-yl)ethyl)-N-ethyl)-N-propylethan-1-amine
2(2-(4-D-1H-7a-azaindol-3-yl)-N-(-N-(2-(6-CF3-1H-indol-3-yl)ethyl)-N-ethyl)-N-propylethan-1-amine
2(2-(5-OH-1H-7a-azaindol-3-yl)-N-(-N-(2-(6-CF3-1H-indol-3-yl)ethyl)-N-ethyl)-N-propylethan-1-amine
2(2-(6-CF3-1H-7a-azaindol-3-yl)-N-(-N-(2-(6-CF3-1H-indol-3-yl)ethyl)-N-ethyl)-N-propylethan-1-amine
2(2-(7-F-1H-7a-azaindol-3-yl)-N-(-N-(2-(6-CF3-1H-indol-3-yl)ethyl)-N-ethyl)-N-propylethan-1-amine
2(2-(4-D-1H-7a-azaindol-3-yl)-N-(-N-(2-(7-F-1H-indol-3-yl)ethyl)-N-ethyl)-N-propylethan-1-amine
2(2-(5-OH-1H-7a-azaindol-3-yl)-N-(-N-(2-(7-F-1H-indol-3-yl)ethyl)-N-ethyl)-N-propylethan-1-amine
2(2-(6-CF3-1H-7a-azaindol-3-yl)-N-(-N-(2-(7-F-1H-indol-3-yl)ethyl)-N-ethyl)-N-propylethan-1-amine
2(2-(7-F-1H-7a-azaindol-3-yl)-N-(-N-(2-(7-F-1H-indol-3-yl)ethyl)-N-ethyl)-N-propylethan-1-amine
2(2-(4-D-1H-indol-3-yl)-N-(-N-(2-(4-D-1H-indol-3-yl)ethyl)-N-ethyl)-N-methylethan-1-amine
2(2-(5-OH-1H-indol-3-yl)-N-(-N-(2-(4-D-1H-indol-3-yl)ethyl)-N-ethyl)-N-methylethan-1-amine
2(2-(6-CF3-1H-indol-3-yl)-N-(-N-(2-(4-D-1H-indol-3-yl)ethyl)-N-ethyl)-N-methylethan-1-amine
2(2-(7-F-1H-indol-3-yl)-N-(-N-(2-(4-D-1H-indol-3-yl)ethyl)-N-ethyl)-N-methylethan-1-amine
2(2-(4-D-1H-indol-3-yl)-N-(-N-(2-(5-OH-1H-indol-3-yl)ethyl)-N-ethyl)-N-methylethan-1-amine
2(2-(5-OH-1H-indol-3-yl)-N-(-N-(2-(5-OH-1H-indol-3-yl)ethyl)-N-ethyl)-N-methylethan-1-amine
2(2-(6-CF3-1H-indol-3-yl)-N-(-N-(2-(5-OH-1H-indol-3-yl)ethyl)-N-ethyl)-N-methylethan-1-amine
2(2-(7-F-1H-indol-3-yl)-N-(-N-(2-(5-OH-1H-indol-3-yl)ethyl)-N-ethyl)-N-methylethan-1-amine
2(2-(4-D-1H-indol-3-yl)-N-(-N-(2-(6-CF3-1H-indol-3-yl)ethyl)-N-ethyl)-N-methylethan-1-amine
2(2-(5-OH-1H-indol-3-yl)-N-(-N-(2-(6-CF3-1H-indol-3-yl)ethyl)-N-ethyl)-N-methylethan-1-amine
2(2-(6-CF3-1H-indol-3-yl)-N-(-N-(2-(6-CF3-1H-indol-3-yl)ethyl)-N-ethyl)-N-methylethan-1-amine
2(2-(7-F-1H-indol-3-yl)-N-(-N-(2-(6-CF3-1H-indol-3-yl)ethyl)-N-ethyl)-N-methylethan-1-amine
2(2-(4-D-1H-indol-3-yl)-N-(-N-(2-(7-F-1H-indol-3-yl)ethyl)-N-ethyl)-N-methylethan-1-amine
2(2-(5-OH-1H-indol-3-yl)-N-(-N-(2-(7-F-1H-indol-3-yl)ethyl)-N-ethyl)-N-methylethan-1-amine
2(2-(6-CF3-1H-indol-3-yl)-N-(-N-(2-(7-F-1H-indol-3-yl)ethyl)-N-ethyl)-N-methylethan-1-amine
2(2-(7-F-1H-indol-3-yl)-N-(-N-(2-(7-F-1H-indol-3-yl)ethyl)-N-ethyl)-N-methylethan-1-amine
2(2-(4-D-1H-indol-3-yl)-N-(-N-(2-(4-D-1H-indol-3-yl)ethyl)-N-ethyl)-N-ethylethan-1-amine
2(2-(5-OH-1H-indol-3-yl)-N-(-N-(2-(4-D-1H-indol-3-yl)ethyl)-N-ethyl)-N-ethylethan-1-amine
2(2-(6-CF3-1H-indol-3-yl)-N-(-N-(2-(4-D-1H-indol-3-yl)ethyl)-N-ethyl)-N-ethylethan-1-amine
2(2-(7-F-1H-indol-3-yl)-N-(-N-(2-(4-D-1H-indol-3-yl)ethyl)-N-ethyl)-N-ethylethan-1-amine
2(2-(4-D-1H-indol-3-yl)-N-(-N-(2-(5-OH-1H-indol-3-yl)ethyl)-N-ethyl)-N-ethylethan-1-amine
2(2-(5-OH-1H-indol-3-yl)-N-(-N-(2-(5-OH-1H-indol-3-yl)ethyl)-N-ethyl)-N-ethylethan-1-amine
2(2-(6-CF3-1H-indol-3-yl)-N-(-N-(2-(5-OH-1H-indol-3-yl)ethyl)-N-ethyl)-N-ethylethan-1-amine
2(2-(7-F-1H-indol-3-yl)-N-(-N-(2-(5-OH-1H-indol-3-yl)ethyl)-N-ethyl)-N-ethylethan-1-amine
2(2-(4-D-1H-indol-3-yl)-N-(-N-(2-(6-CF3-1H-indol-3-yl)ethyl)-N-ethyl)-N-ethylethan-1-amine
2(2-(5-OH-1H-indol-3-yl)-N-(-N-(2-(6-CF3-1H-indol-3-yl)ethyl)-N-ethyl)-N-ethylethan-1-amine
2(2-(6-CF3-1H-indol-3-yl)-N-(-N-(2-(6-CF3-1H-indol-3-yl)ethyl)-N-ethyl)-N-ethylethan-1-amine
2(2-(7-F-1H-indol-3-yl)-N-(-N-(2-(6-CF3-1H-indol-3-yl)ethyl)-N-ethyl)-N-ethylethan-1-amine
2(2-(4-D-1H-indol-3-yl)-N-(-N-(2-(7-F-1H-indol-3-yl)ethyl)-N-ethyl)-N-ethylethan-1-amine
2(2-(5-OH-1H-indol-3-yl)-N-(-N-(2-(7-F-1H-indol-3-yl)ethyl)-N-ethyl)-N-ethylethan-1-amine
2(2-(6-CF3-1H-indol-3-yl)-N-(-N-(2-(7-F-1H-indol-3-yl)ethyl)-N-ethyl)-N-ethylethan-1-amine
2(2-(7-F-1H-indol-3-yl)-N-(-N-(2-(7-F-1H-indol-3-yl)ethyl)-N-ethyl)-N-ethylethan-1-amine
2(2-(4-D-1H-indol-3-yl)-N-(-N-(2-(4-D-1H-indol-3-yl)ethyl)-N-ethyl)-N-isopropylethan-1-amine
2(2-(5-OH-1H-indol-3-yl)-N-(-N-(2-(4-D-1H-indol-3-yl)ethyl)-N-ethyl)-N-isopropylethan-1-amine
2(2-(6-CF3-1H-indol-3-yl)-N-(-N-(2-(4-D-1H-indol-3-yl)ethyl)-N-ethyl)-N-isopropylethan-1-amine
2(2-(7-F-1H-indol-3-yl)-N-(-N-(2-(4-D-1H-indol-3-yl)ethyl)-N-ethyl)-N-isopropylethan-1-amine
2(2-(4-D-1H-indol-3-yl)-N-(-N-(2-(5-OH-1H-indol-3-yl)ethyl)-N-ethyl)-N-isopropylethan-1-amine
2(2-(5-OH-1H-indol-3-yl)-N-(-N-(2-(5-OH-1H-indol-3-yl)ethyl)-N-ethyl)-N-isopropylethan-1-amine
2(2-(6-CF3-1H-indol-3-yl)-N-(-N-(2-(5-OH-1H-indol-3-yl)ethyl)-N-ethyl)-N-isopropylethan-1-amine
2(2-(7-F-1H-indol-3-yl)-N-(-N-(2-(5-OH-1H-indol-3-yl)ethyl)-N-ethyl)-N-isopropylethan-1-amine
2(2-(4-D-1H-indol-3-yl)-N-(-N-(2-(6-CF3-1H-indol-3-yl)ethyl)-N-ethyl)-N-isopropylethan-1-amine
2(2-(5-OH-1H-indol-3-yl)-N-(-N-(2-(6-CF3-1H-indol-3-yl)ethyl)-N-ethyl)-N-isopropylethan-1-amine
2(2-(6-CF3-1H-indol-3-yl)-N-(-N-(2-(6-CF3-1H-indol-3-yl)ethyl)-N-ethyl)-N-isopropylethan-1-amine
2(2-(7-F-1H-indol-3-yl)-N-(-N-(2-(6-CF3-1H-indol-3-yl)ethyl)-N-ethyl)-N-isopropylethan-1-amine
2(2-(4-D-1H-indol-3-yl)-N-(-N-(2-(7-F-1H-indol-3-yl)ethyl)-N-ethyl)-N-isopropylethan-1-amine
2(2-(5-OH-1H-indol-3-yl)-N-(-N-(2-(7-F-1H-indol-3-yl)ethyl)-N-ethyl)-N-isopropylethan-1-amine
2(2-(6-CF3-1H-indol-3-yl)-N-(-N-(2-(7-F-1H-indol-3-yl)ethyl)-N-ethyl)-N-isopropylethan-1-amine
2(2-(7-F-1H-indol-3-yl)-N-(-N-(2-(7-F-1H-indol-3-yl)ethyl)-N-ethyl)-N-isopropylethan-1-amine
2(2-(4-D-1H-indol-3-yl)-N-(-N-(2-(4-D-1H-indol-3-yl)ethyl)-N-ethyl)-N-allylethan-1-amine
2(2-(5-OH-1H-indol-3-yl)-N-(-N-(2-(4-D-1H-indol-3-yl)ethyl)-N-ethyl)-N-allylethan-1-amine
2(2-(6-CF3-1H-indol-3-yl)-N-(-N-(2-(4-D-1H-indol-3-yl)ethyl)-N-ethyl)-N-allylethan-1-amine
2(2-(7-F-1H-indol-3-yl)-N-(-N-(2-(4-D-1H-indol-3-yl)ethyl)-N-ethyl)-N-allylethan-1-amine
2(2-(4-D-1H-indol-3-yl)-N-(-N-(2-(5-OH-1H-indol-3-yl)ethyl)-N-ethyl)-N-allylethan-1-amine
2(2-(5-OH-1H-indol-3-yl)-N-(-N-(2-(5-OH-1H-indol-3-yl)ethyl)-N-ethyl)-N-allylethan-1-amine
2(2-(6-CF3-1H-indol-3-yl)-N-(-N-(2-(5-OH-1H-indol-3-yl)ethyl)-N-ethyl)-N-allylethan-1-amine
2(2-(7-F-1H-indol-3-yl)-N-(-N-(2-(5-OH-1H-indol-3-yl)ethyl)-N-ethyl)-N-allylethan-1-amine
2(2-(4-D-1H-indol-3-yl)-N-(-N-(2-(6-CF3-1H-indol-3-yl)ethyl)-N-ethyl)-N-allylethan-1-amine
2(2-(5-OH-1H-indol-3-yl)-N-(-N-(2-(6-CF3-1H-indol-3-yl)ethyl)-N-ethyl)-N-allylethan-1-amine
2(2-(6-CF3-1H-indol-3-yl)-N-(-N-(2-(6-CF3-1H-indol-3-yl)ethyl)-N-ethyl)-N-allylethan-1-amine
2(2-(7-F-1H-indol-3-yl)-N-(-N-(2-(6-CF3-1H-indol-3-yl)ethyl)-N-ethyl)-N-allylethan-1-amine
2(2-(4-D-1H-indol-3-yl)-N-(-N-(2-(7-F-1H-indol-3-yl)ethyl)-N-ethyl)-N-allylethan-1-amine
2(2-(5-OH-1H-indol-3-yl)-N-(-N-(2-(7-F-1H-indol-3-yl)ethyl)-N-ethyl)-N-allylethan-1-amine
2(2-(6-CF3-1H-indol-3-yl)-N-(-N-(2-(7-F-1H-indol-3-yl)ethyl)-N-ethyl)-N-allylethan-1-amine
2(2-(7-F-1H-indol-3-yl)-N-(-N-(2-(7-F-1H-indol-3-yl)ethyl)-N-ethyl)-N-allylethan-1-amine
2(2-(4-D-1H-indol-3-yl)-N-(-N-(2-(4-D-1H-indol-3-yl)ethyl)-N-ethyl)-N-hydroethan-1-amine
2(2-(5-OH-1H-indol-3-yl)-N-(-N-(2-(4-D-1H-indol-3-yl)ethyl)-N-ethyl)-N-hydroethan-1-amine
2(2-(6-CF3-1H-indol-3-yl)-N-(-N-(2-(4-D-1H-indol-3-yl)ethyl)-N-ethyl)-N-hydroethan-1-amine
2(2-(7-F-1H-indol-3-yl)-N-(-N-(2-(4-D-1H-indol-3-yl)ethyl)-N-ethyl)-N-hydroethan-1-amine
2(2-(4-D-1H-indol-3-yl)-N-(-N-(2-(5-OH-1H-indol-3-yl)ethyl)-N-ethyl)-N-hydroethan-1-amine
2(2-(5-OH-1H-indol-3-yl)-N-(-N-(2-(5-OH-1H-indol-3-yl)ethyl)-N-ethyl)-N-hydroethan-1-amine
2(2-(6-CF3-1H-indol-3-yl)-N-(-N-(2-(5-OH-1H-indol-3-yl)ethyl)-N-ethyl)-N-hydroethan-1-amine
2(2-(7-F-1H-indol-3-yl)-N-(-N-(2-(5-OH-1H-indol-3-yl)ethyl)-N-ethyl)-N-hydroethan-1-amine
2(2-(4-D-1H-indol-3-yl)-N-(-N-(2-(6-CF3-1H-indol-3-yl)ethyl)-N-ethyl)-N-hydroethan-1-amine
2(2-(5-OH-1H-indol-3-yl)-N-(-N-(2-(6-CF3-1H-indol-3-yl)ethyl)-N-ethyl)-N-hydroethan-1-amine
2(2-(6-CF3-1H-indol-3-yl)-N-(-N-(2-(6-CF3-1H-indol-3-yl)ethyl)-N-ethyl)-N-hydroethan-1-amine
2(2-(7-F-1H-indol-3-yl)-N-(-N-(2-(6-CF3-1H-indol-3-yl)ethyl)-N-ethyl)-N-hydroethan-1-amine
2(2-(4-D-1H-indol-3-yl)-N-(-N-(2-(7-F-1H-indol-3-yl)ethyl)-N-ethyl)-N-hydroethan-1-amine
2(2-(5-OH-1H-indol-3-yl)-N-(-N-(2-(7-F-1H-indol-3-yl)ethyl)-N-ethyl)-N-hydroethan-1-amine
2(2-(6-CF3-1H-indol-3-yl)-N-(-N-(2-(7-F-1H-indol-3-yl)ethyl)-N-ethyl)-N-hydroethan-1-amine
2(2-(7-F-1H-indol-3-yl)-N-(-N-(2-(7-F-1H-indol-3-yl)ethyl)-N-ethyl)-N-hydroethan-1-amine
2(2-(4-D-1H-indol-3-yl)-N-(-N-(2-(4-D-1H-indol-3-yl)ethyl)-N-ethyl)-N-(D)ethan-1-amine
2(2-(5-OH-1H-indol-3-yl)-N-(-N-(2-(4-D-1H-indol-3-yl)ethyl)-N-ethyl)-N-(D)ethan-1-amine
2(2-(6-CF3-1H-indol-3-yl)-N-(-N-(2-(4-D-1H-indol-3-yl)ethyl)-N-ethyl)-N-(D)ethan-1-amine
2(2-(7-F-1H-indol-3-yl)-N-(-N-(2-(4-D-1H-indol-3-yl)ethyl)-N-ethyl)-N-(D)ethan-1-amine
2(2-(4-D-1H-indol-3-yl)-N-(-N-(2-(5-OH-1H-indol-3-yl)ethyl)-N-ethyl)-N-(D)ethan-1-amine
2(2-(5-OH-1H-indol-3-yl)-N-(-N-(2-(5-OH-1H-indol-3-yl)ethyl)-N-ethyl)-N-(D)ethan-1-amine
2(2-(6-CF3-1H-indol-3-yl)-N-(-N-(2-(5-OH-1H-indol-3-yl)ethyl)-N-ethyl)-N-(D)ethan-1-amine
2(2-(7-F-1H-indol-3-yl)-N-(-N-(2-(5-OH-1H-indol-3-yl)ethyl)-N-ethyl)-N-(D)ethan-1-amine
2(2-(4-D-1H-indol-3-yl)-N-(-N-(2-(6-CF3-1H-indol-3-yl)ethyl)-N-ethyl)-N-(D)ethan-1-amine
2(2-(5-OH-1H-indol-3-yl)-N-(-N-(2-(6-CF3-1H-indol-3-yl)ethyl)-N-ethyl)-N-(D)ethan-1-amine
2(2-(6-CF3-1H-indol-3-yl)-N-(-N-(2-(6-CF3-1H-indol-3-yl)ethyl)-N-ethyl)-N-(D)ethan-1-amine
2(2-(7-F-1H-indol-3-yl)-N-(-N-(2-(6-CF3-1H-indol-3-yl)ethyl)-N-ethyl)-N-(D)ethan-1-amine
2(2-(4-D-1H-indol-3-yl)-N-(-N-(2-(7-F-1H-indol-3-yl)ethyl)-N-ethyl)-N-(D)ethan-1-amine
2(2-(5-OH-1H-indol-3-yl)-N-(-N-(2-(7-F-1H-indol-3-yl)ethyl)-N-ethyl)-N-(D)ethan-1-amine
2(2-(6-CF3-1H-indol-3-yl)-N-(-N-(2-(7-F-1H-indol-3-yl)ethyl)-N-ethyl)-N-(D)ethan-1-amine
2(2-(7-F-1H-indol-3-yl)-N-(-N-(2-(7-F-1H-indol-3-yl)ethyl)-N-ethyl)-N-(D)ethan-1-amine
2(2-(4-D-1H-indol-3-yl)-N-(-N-(2-(4-D-1H-indol-3-yl)ethyl)-N-ethyl)-N-propylethan-1-amine
2(2-(5-OH-1H-indol-3-yl)-N-(-N-(2-(4-D-1H-indol-3-yl)ethyl)-N-ethyl)-N-propylethan-1-amine
2(2-(6-CF3-1H-indol-3-yl)-N-(-N-(2-(4-D-1H-indol-3-yl)ethyl)-N-ethyl)-N-propylethan-1-amine
2(2-(7-F-1H-indol-3-yl)-N-(-N-(2-(4-D-1H-indol-3-yl)ethyl)-N-ethyl)-N-propylethan-1-amine
2(2-(4-D-1H-indol-3-yl)-N-(-N-(2-(5-OH-1H-indol-3-yl)ethyl)-N-ethyl)-N-propylethan-1-amine
2(2-(5-OH-1H-indol-3-yl)-N-(-N-(2-(5-OH-1H-indol-3-yl)ethyl)-N-ethyl)-N-propylethan-1-amine
2(2-(6-CF3-1H-indol-3-yl)-N-(-N-(2-(5-OH-1H-indol-3-yl)ethyl)-N-ethyl)-N-propylethan-1-amine
2(2-(7-F-1H-indol-3-yl)-N-(-N-(2-(5-OH-1H-indol-3-yl)ethyl)-N-ethyl)-N-propylethan-1-amine
2(2-(4-D-1H-indol-3-yl)-N-(-N-(2-(6-CF3-1H-indol-3-yl)ethyl)-N-ethyl)-N-propylethan-1-amine
2(2-(5-OH-1H-indol-3-yl)-N-(-N-(2-(6-CF3-1H-indol-3-yl)ethyl)-N-ethyl)-N-propylethan-1-amine
2(2-(6-CF3-1H-indol-3-yl)-N-(-N-(2-(6-CF3-1H-indol-3-yl)ethyl)-N-ethyl)-N-propylethan-1-amine
2(2-(7-F-1H-indol-3-yl)-N-(-N-(2-(6-CF3-1H-indol-3-yl)ethyl)-N-ethyl)-N-propylethan-1-amine
2(2-(4-D-1H-indol-3-yl)-N-(-N-(2-(7-F-1H-indol-3-yl)ethyl)-N-ethyl)-N-propylethan-1-amine
2(2-(5-OH-1H-indol-3-yl)-N-(-N-(2-(7-F-1H-indol-3-yl)ethyl)-N-ethyl)-N-propylethan-1-amine
2(2-(6-CF3-1H-indol-3-yl)-N-(-N-(2-(7-F-1H-indol-3-yl)ethyl)-N-ethyl)-N-propylethan-1-amine and
2(2-(7-F-1H-indol-3-yl)-N-(-N-(2-(7-F-1H-indol-3-yl)ethyl)-N-ethyl)-N-propylethan-1-amine.

### EXAMPLES

### Example 1

The following outlines the steps that were used in order to make the methylated derivative of tryptamine dimer N-(2-(1H-indol-3-yl)ethyl)-2-(1H-indol-3-yl)-N-methylethan-1-amine:

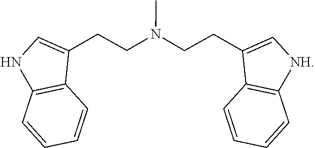 [SMILES: CN(CCc1c[nH]c2ccccc12)CCc1c[nH]c2ccccc12]

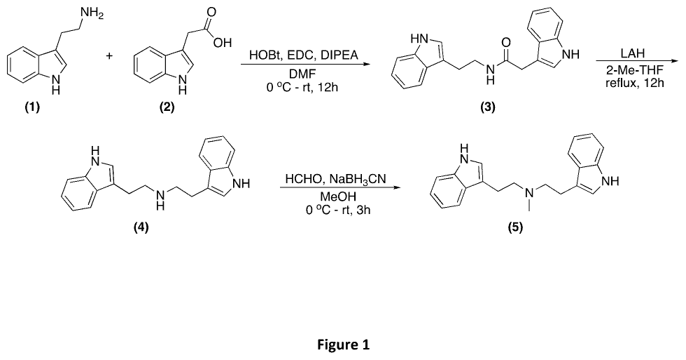

Indole-3-acetic acid was coupled to tryptamine via a peptide coupling reagent. The amide was reduced with lithium aluminum hydride. Finally, the R alkyl group was installed via a reductive amination with the appropriate aldehyde. This synthetic pathway is shown in FIG. 1.

In vitro pharmacological assessment of this compound (N-(2-(1H-indol-3-yl)ethyl)-2-(1H-indol-3-yl)-N-methylethan-1-amine) was performed using radioligand binding at various serotonergic and sigma receptors. The results appear in table 2 below

TABLE 2

| | % | % | % | % | |
|---|---|---|---|---|---|
| | Inhibition | Inhibition | Inhibition | Inhibition | |
| Receptor | at 50 nM | at 500 nM | at 1 mM | at 2 mM | Radioligand |
| 5-HT1A | 70.9% | NT | 98.0% | NT | [³H]8-OH-DPAT |
| 5-HT2A | 82.7% | NT | 95.5% | NT | [³H]ketanserin |
| 5-HT2B | 96.8% | NT | 96.9% | NT | [³H]mesulergine |
| 5-HT2C | 72.8% | NT | 97.8% | NT | [³H]mesulergine |
| 5-HT6 | 26.3% | NT | 90.4% | NT | [³H]LSD |
| 5-HT7 | 79.7% | NT | 99.5% | NT | [³H]LSD |
| Sigma 1 | NT | 21.7% | NT | 55.6% | [³H](+)pentazocine |
| Sigma 2 | NT | 26.1% | NT | 60.9% | [³H]DTG + |
| | | | | | (+)pentazocine |

### Example 2 (Prophetic)

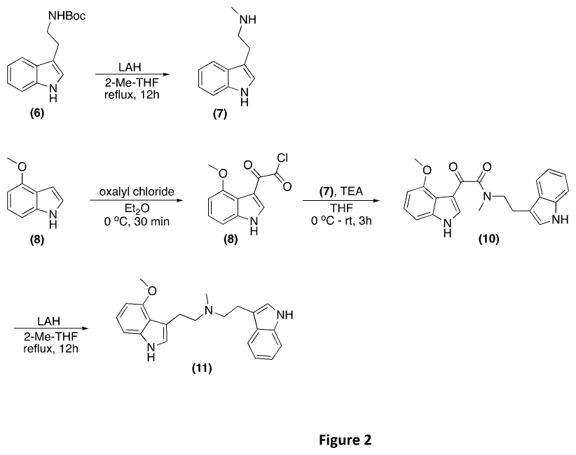

N-Boc-Tryptamine may be reduced with lithium aluminum hydride to give N-methyltryptamine. Friedel crafts acylation of 4-Methoxyindole using oxalyl chloride may be used to give the corresponding acid chloride, which will then be treated with NMT and triethylamine. The alpha ketoamide may be reduced using LAH under refluxing conditions to give the final product. This synthetic pathway is shown in FIG. 2.

### Example 3 (Prophetic)

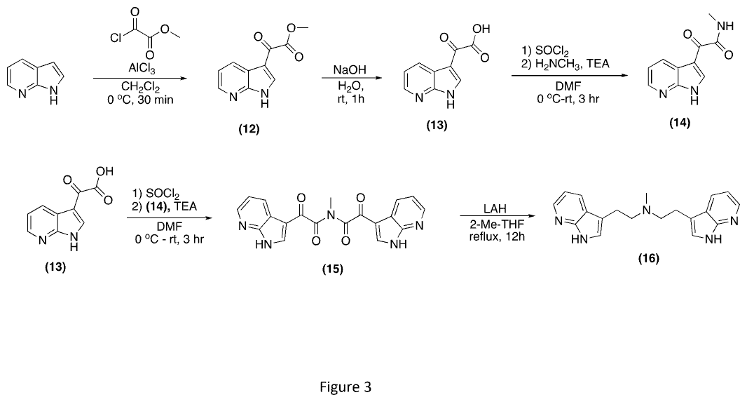

7-azaindole may be treated with methyl chlorooxoacetate and aluminum chloride in dichloromethane. The methyl ester may be saponified to form the carboxylic acid (13). The acid chloride, formed in the presence of thionyl chloride, may be treated with methylamine to give an amide (14). Carboxylic acid intermediate (13) may be converted to the acid chloride and treated with methyl amide (14) to form an imide (15), which may then be reduced with lithium aluminum hydride under reflux to give azatryptamine dimer (16). This synthetic pathway is shown in FIG. 3.

### Example 4

For the four compounds identified in Table 3 (below), the serotonin 5-HT2A receptor activity was assessed and predicted using virtual docking of the RCSB Protein Data Bank (PDB) crystal structure 6WGT with (+)-LSD bound. The overall workflow was performed using various aspects of the Schrödinger software suite. Each ligand was uploaded following literature recommended protonation and/or charge for tryptamines and various conformers were generated and minimized using Schrödinger LigPrep. The final list of compounds was screened with glide scores being generated by Schrödinger GLIDE including the structures listed in Table 3. For reference, (+)-LSD was docked giving a score of-9.953. All of the proposed 5 tryptamine dimers are of equal or greater (lower) values than (+)-LSD in the 5-HT2A receptor with two structures being predicted as the most potent compounds seen to date in our virtual screenings of −13.767 and −13.559.

TABLE 3
Virtual Docking Scores of Dimeric Tryptamine Compounds

| | Docking Score |
|---|---|
| Compound Structure | 6WGT (5-HT2_{A}) |
| 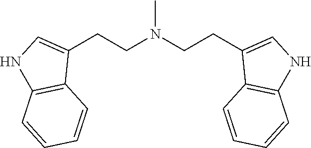 [SMILES: CN(CCc1c[nH]c2ccccc12)CCc1c[nH]c2ccccc12] | −13.767 |
| 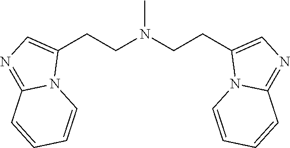 [SMILES: CN(CCc1cnc2ccccn12)CCc1cnc2ccccn12] | −9.136 |
| 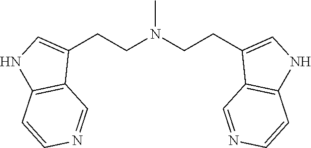 [SMILES: CN(CCc1c[nH]c2ccncc12)CCc1c[nH]c2ccncc12] | −10.534 |
| 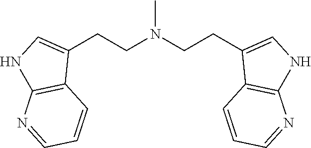 [SMILES: CN(CCc1c[nH]c2ncccc12)CCc1c[nH]c2ncccc12] | −13.559 |
| (+)-Lisergic Acid Diethylamide ((+)-LSD) | −9.953 |

### Example 5

The proposed scaffolds were predicted to produce unique serotonin 5 activity, especially at the serotonin 2A receptor (5-HT2AR). Using proprietary computational modeling of this serotonergic receptor (PDB 6WGT, Kim et al., Structure of a Hallucinogen-Activated Gq-Coupled 5-HT2A Serotonin Receptor, Cell 2020 Sep. 17; 182(6):1574-1588.e19), a detailed comparison of the tryptamine and azaindole dimers revealed interactions within the binding pocket of 5-HT2AR not triggered by common psychedelics like (+)-LSD. This relative residue contact analysis for N-(2-(1H-indol-3-yl)ethyl)-2-(1H-indol-3-yl)-N-methylethan-1-amine (sDMT; Formula I) shows favored residue Van der Waals (VDW) contacts with ILE 135 and 139, which indicates that this molecule may span into the “handlebar” region of 5-HT2A as occupied by (+)-LSD with its ethyl groups. ASN 343, GLY 238, and SER 239, which are on the benzyl side of the indole occupied region (by both compounds), also have significant VDW contacts for sDMT as compared to (+)-LSD. For hydrogen bond interactions, the most notable are SER 242 and ASN 343, which are significantly favored by both compounds. Collectively, these modeled results yield evidence of a shift in interactions away from SER 242 and closer to the benzyl side of the indole region, while maintaining ASP 155 interactions. This shift is important as it mimics less hallucinogenic psychedelic mimics like lisuride and (+)-2-Br-LSD (not presented), therefore predicting dimeric tryptamine and azaindole scaffolds are candidates for sub-hallucinogenic characterization. In addition, the modeled docked pose of sDMT spans more of the 5HT2A active site than (+)-LSD predicting high selectivity for this specific serotonergic receptor.

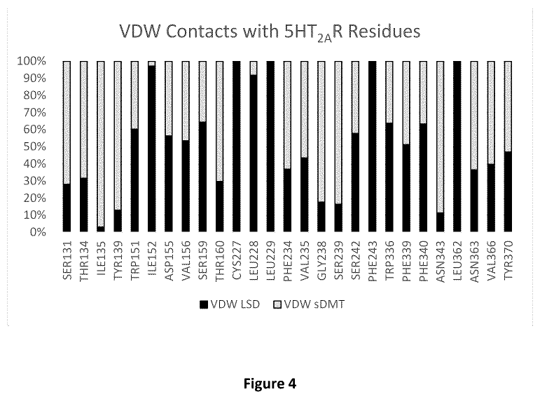

FIG. 4 shows the normalized atomic Van de Waals (“VDW”) contacts between sDMT (gray) and (+)-LSD (black) modeled in the active site of Serotonin 2A Receptor. In this representation, a significant comparison for any one residue is a 60% difference where the division of the represented ligands is either in the top or lower 20%.

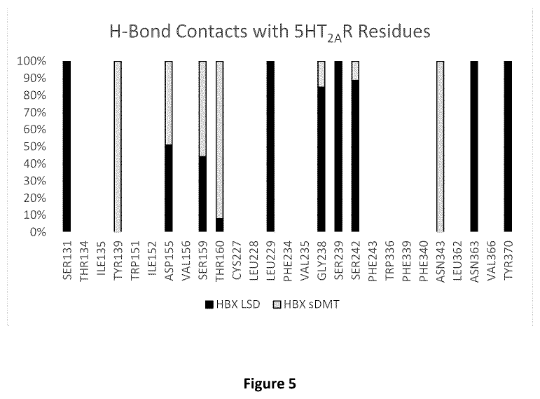

FIG. 5 shows the normalized atomic hydrogen bond contacts (“HBX”) between sDMT (gray) and (+)-LSD (black) modeled in the active site of Serotonin 2A Receptor. In this representation, a significant comparison for any one residue is a 60% difference where the division of the represented ligands is either in the top or lower 20%.

### Examples 6-12

Table 4 provide both a structure and IUPAC name for products synthesized in examples 6-12.

In examples 6-12, the following procedures were used:

### Coupling Procedure

Carboxylic acid (1.00 eq., 2.85 mmol) was dissolved in 10.6 mL of DMF. Amine (1.50 eq., 4.28 mmol), HOBt (1.50 eq., 4.28 mmol), EDC (1.50 eq., 4.28 mmol), and triethylamine (6.00 eq., 17.2 mmol) were added to the solution. The solution was stirred overnight at room temperature. The reaction was quenched with cold water and extracted with ethyl acetate. The organic layer was dried over Na₂SO₄, filtered, and concentrated under reduced pressure. The resultant residue was loaded onto silica and purified by column chromatography using EtOAc: Hexanes or MeOH: DCM to give the appropriate amide.

Reduction Procedure A with BH₃

In a flame dried flask was an amide (1.00 eq., 2.06 mmol). The flask was equipped with a magnetic stir bar and reflux condenser under argon. Anhydrous 2-Me-THF (17.6 mL) was added to the flask, and the reaction was cooled down to 0° C. 0.900 M BH₃ in THF (6.00 eq., 12.3 mmol) was added dropwise. The reaction was refluxed overnight. Upon completion, the excess BH₃ was quenched with MeOH and the reaction was concentrated under reduced pressure. An oil was obtained and purified by column chromatography using MeOH/NH₃ in DCM.

Reduction Procedure B with LiAlH₄

In a flame dried flask charged with a magnetic stir bar was an amide (1.00 eq., 0.122 mmol) in 0.407 mL anhydrous 2-Me-THF under argon. The solution was heated up to 64° C., and 2.40 M LiAlH₄ (3.75 eq., 0.458 mmol) was added dropwise. Once the addition was complete, the reaction was refluxed overnight. The reaction was quenched with 3.20 eq. of Glauber's Salt, filtered through celite, and concentrated under reduced pressure to give crude amine.

### Reductive Amination Procedure

Into a round-bottom flask was an amine (1.00 eq., 0.214 mmol) in MeOH (1.43 mL). The solution was cooled to 0° C. NaCNBH₄ (1.10 eq., 0.236 mmol) and AcOH (2.50 eq., 0.540 mmol) were added, and the solution stirred at 0° C. for 5 minutes. The carbon source (1.30 eq., 0.280 mmol) was added dropwise, and the solution was stirred at room temperature until completion. NaOH was added to the reaction, and the reaction was concentrated under reduced pressure to remove the MeOH. The water was extracted with CHCl₃, dried over Na₂SO₄, and concentrated under reduced pressure. The crude product was loaded onto silica and purified by column chromatography using EtOAc: Hexanes with 2% triethylamine to give the product.

### Speeter-Anthony Procedure

To a flame dried round bottom flask, the appropriate indole (1.00 eq, 13.6 mmol) was dissolved in anhydrous diethyl ether (14.3 mL) under argon and cooled to 0° C. A solution of oxalyl chloride (2.00 eq, 27.2 mmol) in 16.0 mL of anhydrous diethyl ether was added dropwise. The solution was allowed to stir for 30 minutes at 0° C., after which heptanes (30.0 mL) were added. The flask was stored in the freezer overnight.

The precipitate was filtered and washed with 4:1 heptanes: MTBE. The acid chloride was dissolved in anhydrous 2-methyl-THF (6.01 mL and cooled to 0° C. A solution of amine (1.01 eq, 2.12 mmol) in 2-methyl-THF (5.00 mL) was added dropwise followed by a solution of triethylamine (1.23 eq, 2.59 mmol) in 1.00 mL of 2-Me-THF. The reaction was allowed to warm to room temperature and stirred for 3 hours. Heptanes (14.0 mL) were added. The precipitate was collected by filtration and washed with ice cold water. The product was dried under vacuum overnight.

### Example 6

Using the coupling procedure described in the introduction to examples 6-12, indole-3-acetic acid (0.500 g 2.85 mmol) and tryptamine (0.686 g, 4.28 mmol) were used to synthesize an amide as a clear oil (0.653 g, 72.1%). %). 1H NMR (METHANOL-d₄, 600 MHz) δ 7.60 (s, 1H), 7.4-7.4 (m, 2H), 7.29 (d, 1H, J=8.4 Hz), 7.24 (d, 1H, J=8.0 Hz), 7.0-7.1 (m, 1H), 7.0-7.0 (m, 1H), 6.9-7.0 (m, 2H), 6.9-6.9 (m, 1H), 6.62 (s, 1H), 3.52 (s, 2H), 3.3-3.4 (m, 2H), 2.7-2.8 (m, 2H) ppm. ¹³C NMR (METHANOL-d₄, 600 MHz) δ 174.8, 138.2, 138.2, 128.8, 128.6, 125.3, 123.7, 122.9, 122.6, 120.3, 119.9, 119.6, 119.5, 113.1, 112.7, 112.5, 109.4, 41.5, 34.2, 31.7 ppm. LRMS m/z: [M+H]⁺=318.200. The amide has the following structure:

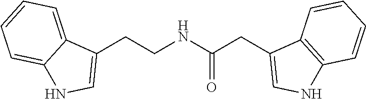 [SMILES: O=C(Cc1c[nH]c2ccccc12)NCCc1c[nH]c2ccccc12]

Using reduction procedure A described in the introduction to examples 6-12, this amide (0.653 g, 2.06 mmol) was converted to an amine that was obtained as a clear oil (0.387 g, 62.1%). The amine had the following structure:

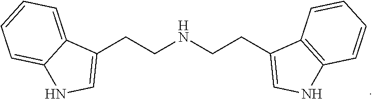 [SMILES: c1ccc2c(CCNCCc3c[nH]c4ccccc34)c[nH]c2c1]

Using the reductive amination procedure described in the introduction to examples 6-12, the amine (0.0470 g, 0.155 mmol):

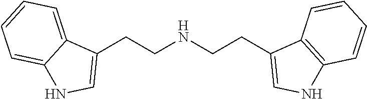 [SMILES: c1ccc2c(CCNCCc3c[nH]c4ccccc34)c[nH]c2c1]

and formaldehyde (0.0200 g, 0.280 mmol) were used to produce the following tertiary amine (0.0310, 63.3%):

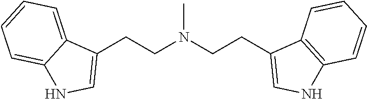 [SMILES: CN(CCc1c[nH]c2ccccc12)CCc1c[nH]c2ccccc12]

as a pale yellow oil. HRMS calculated for C₂₁H₂₃N₃ ([M+H]⁺): 317.1892; found, 317.1889.1H NMR (CHLOROFORM-d, 400 MHz) δ 7.97 (br s, 2H), 7.63 (d, 2H, J=8.0 Hz), 7.37 (d, 2H, J=8.3 Hz), 7.20 (t, 2H, J=7.5 Hz), 7.13 (t, 2H, J=1.0 Hz), 7.04 (s, 2H), 3.04 (t, 4H, J=1.0 Hz), 2.89 (t, 4H, J=1.0 Hz), 2.54 (s, 3H) ppm. ¹³C NMR (CHLOROFORM-d, 400 MHz) δ 136.2, 127.5, 121.9, 121.6, 119.2, 118.8, 114.3, 111.1, 58.1, 42.1, 23.1 ppm. HRMS calculated for C₂₁H₂₃N₃ ([M+H]⁺): 317.1892; found, 317.1889.

### Example 7

Using the reductive amination procedure provided above, an amine (0.276 g, 0.909 mmol),

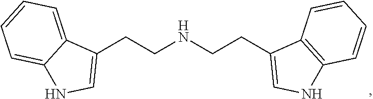 [SMILES: c1ccc2c(CCNCCc3c[nH]c4ccccc34)c[nH]c2c1]

and acetone (0.190 g, 0.118 mmol) were used to produce a tertiary amine (0.229 g, 72.9% BRSM) as a pale yellow oil. 1H NMR (CHLOROFORM-d, 400 MHz) δ 7.91 (br s, 2H), 7.54 (br d, 2H, J-7.5 Hz), 7.24 (br d, 2H, J=8.0 Hz), 7.1-7.1 (m, 2H), 7.0-7.1 (m, 2H), 6.90 (br s, 2H), 3.0-3.2 (m, 1H), 2.8-2.9 (m, 4H), 2.8-2.8 (m, 4H), 1.00 (br d, 6H, J=6.5 Hz) ppm. ¹³C NMR (CHLOROFORM-d, 400 MHz) δ 135.9, 127.3, 121.5, 121.2, 118.8, 118.6, 114.5, 110.8, 51.2, 50.8, 25.1, 18.3 ppm. HRMS calculated for C₂₃H₂₇N₃ ([M+H]⁺): 345.2205; found, 345.2206. This tertiary amine had the following structure:

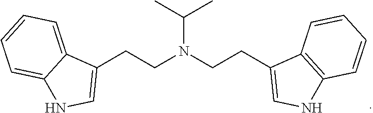 [SMILES: CC(C)N(CCc1c[nH]c2ccccc12)CCc1c[nH]c2ccccc12]

### Example 8

To a stirred suspension of aluminum chloride (5.64 g, 43.3 mmol) in anhydrous DCM (201 mL), azaindole (1.00 g, 8.46 mmol) was added and stirred at room temperature for one hour. Methyl chlorooxoacetate (3.89 mL, 43.3 eq) was added dropwise and left to stir for 8 hours. The reaction was carefully quenched with methanol. The solvents were removed under reduced pressure and purified by column chromatography, eluting with 0-15% methanol in ethyl acetate. The product was a pale-yellow solid (0.648 g, 37.5%). The product was methyl glyoxylate azaindole, which had the following structure:

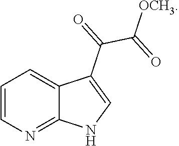 [SMILES: COC(=O)C(=O)c1c[nH]c2ncccc12]

Methyl glyoxylate azaindole (0.648 g, 3.17 mmol) was dissolved in 6.16 mL of 1:1 MeOH: water. Potassium carbonate (1.73 g, 1.73 mmol) was added and the reaction was heated to reflux overnight. The solvent was removed under reduced pressure. Aqueous hydrochloric acid was added to adjust the pH of the solution to pH 4 and the product was extracted with dichloromethane (3×50.0 mL). The combined organic layers were washed with brine, dried over sodium sulfate, and filtered. The solvent was removed under reduced pressure to give a glyoxylic acid. The product was collected as an off-white solid (0.221 g, 30.5%). The glyoxylic acid had the following structure:

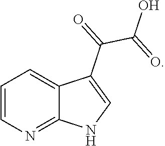 [SMILES: O=C(O)C(=O)c1c[nH]c2ncccc12]

Using the coupling procedure described in the introduction to examples 6-12 with 7-azaindole-3-glyoxylic acid (0.0712 g, 0.374 mmol) and N-methyltryptamine (0.0590 g, 0.374 mmol), a glyoxyl amide (0.05400 g, 41.6%) as a pale-yellow solid was produced. The glyoxyl amide had the following structure:

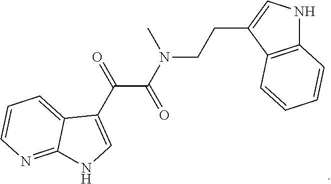 [SMILES: CN(CCc1c[nH]c2ccccc12)C(=O)C(=O)c1c[nH]c2ncccc12]

Using reduction procedure B with LiAlH₄ described in the introduction to examples 6-12, the glyoxyl amide

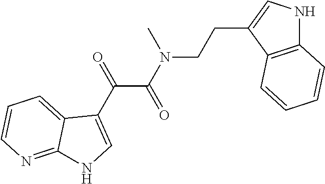 [SMILES: CN(CCc1c[nH]c2ccccc12)C(=O)C(=O)c1c[nH]c2ncccc12]

(0.0540 g, 0.155 mmol) was reduced to form an amine (0.0100 g, 20.2%) as an off-white solid. HRMS calculated for C₂₀H₂₂N₄ ([M+H]⁺): 318.1844; found: 318.1844. This amine had the following structure:

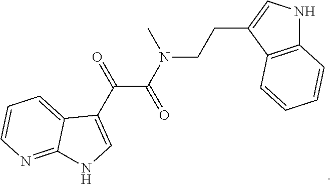 [SMILES: CN(CCc1c[nH]c2ccccc12)C(=O)C(=O)c1c[nH]c2ncccc12]

### Example 9

The Speeter-Anthony procedure described in the introduction to examples 6-12 was used to add the N-methyltryptamine (0.370 g, 2.123 mmol) into the 4-methoxyindole-3-acid chloride (0.500 g, 2.10 mmol) to give a yellow solid (0.658 g, 83.4%), the methoxyglyoxyl amide:

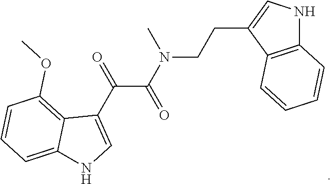 [SMILES: COc1cccc2[nH]cc(C(=O)C(=O)N(C)CCc3c[nH]c4ccccc34)c12]

Reduction procedure B with LiAlH₄ (2.74 mL, 6.57 mmol) described in the introduction to examples 6-12 was used to reduce the methoxyglyoxyl amide (0.658 g, 1.75 mmol) to give the product

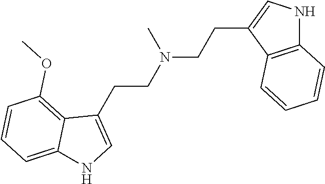 [SMILES: COc1cccc2[nH]cc(CCN(C)CCc3c[nH]c4ccccc34)c12]

as an off-white oil. 1H NMR (400 MHz, CHLOROFORM-d) δ 8.29 (br s, 2H), 7.60 (br d, 1H), 7.27 (m, 1H), 7.20-6.99 (m, 3H), 6.91-6.89 (m, 2H), 6.75 (s, 1H), 6.43 (br d, 1H), 3.82 (s, 3H), 3.10 (br t, 2H), 3.00 (br t, 2H), 2.86 (m, 4H), 2.49 (s, 3H). 13C NMR (400 MHz, CHLOROFORM-d) δ 154.79, 138.21, 136.41, 127.62, 122.71, 121.89, 121.81, 120.78, 120.78, 119.18, 118.92, 117.47, 114.61, 114.37, 111.31, 104.70, 99.28, 59.52, 58.27, 55.05, 42.41, 24.57, 23.14. HRMS calculated for C₂₂H₂₅N₃O ([M+H]⁺): 347.1998; found: 347.2010.

### Example 10

Beginning with 4-cyanoindole (2.00 g, 14.1 mmol), the procedure for Speeter-Anthony described in the introduction to examples 6-12 was used to add tryptamine (1.79 g, 11.2 mmol) into the acid chloride of 4-cyanoindole (2.57 g, 11.0 mmol) to give the following amide

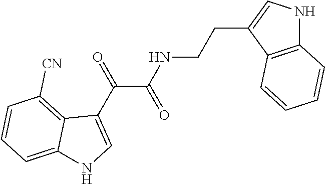 [SMILES: N#Cc1cccc2[nH]cc(C(=O)C(=O)NCCc3c[nH]c4ccccc34)c12]

as a yellow solid (3.79 g, 73.6%).

The following amine

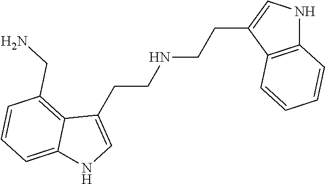 [SMILES: NCc1cccc2[nH]cc(CCNCCc3c[nH]c4ccccc34)c12]

was synthesized via the procedure using LiAlH₄ (6.78 mL, 16.3 mmol) described in the introduction to examples 6-12 by reducing the amide

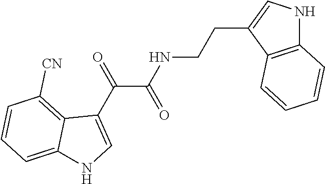 [SMILES: N#Cc1cccc2[nH]cc(C(=O)C(=O)NCCc3c[nH]c4ccccc34)c12]

(1.00 g, 2.81 mmol). The final product was a dark orange oil (0.903 g, 97.1%).

The amine (0.295 g, 0.887 mmol),

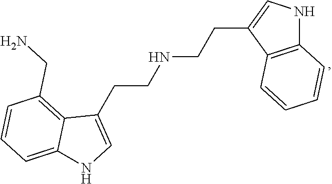 [SMILES: NCc1cccc2[nH]cc(CCNCCc3c[nH]c4ccccc34)c12]

was used in the reductive amination procedure described in the introduction to examples 6-12 with excess formaldehyde as the carbon source (0.290 mL, 3.55 mmol) to give the product as an off-white oil. LRMS m/z: [M+H]⁺=375.20. This product had the following structure

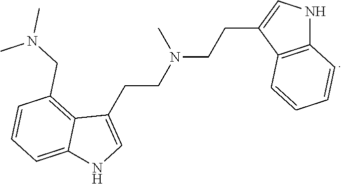 [SMILES: CN(C)Cc1cccc2[nH]cc(CCN(C)CCc3c[nH]c4ccccc34)c12]

### Example 11

4-Br-Indole (0.500 mL, 3.99 mmol, 1.00 eq.) was dissolved in 1.78 mL trifluoroacetic acid (TFA) in a round-bottom flask. 1-Dimethylamino-2-nitroethylene (0.462 g, 3.99 mmol, 1.00 eq.) was added to the reaction and stirred for 30 minutes as a deep red solution. The reaction was quenched by adding it dropwise to a rapidly stirring solution of saturated NaHCO₃. The resulting slurry was extracted with EtOAc, dried over Na₂SO₄, filtered, and concentrated under reduced pressure to give a crude orange product containing:

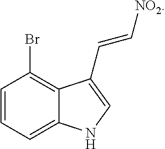 [SMILES: O=[N+]([O-])/C=C/c1c[nH]c2cccc(Br)c12]

The crude product was purified by silica-gel chromatography using hexanes: EtOAc to give the product (1.03 g, 97.2% as an orange solid). 1H NMR (DMSO-d₆, 600 MHz) δ 12.61 (br s, 1H), 9.13 (d, 1H, J=13.4 Hz), 8.56 (d, 1H, J=2.9 Hz), 8.13 (d, 1H, J=13.1 Hz), 7.55 (d, 1H, J-8.0 Hz), 7.43 (d, 1H, J=7.6 Hz), 7.16 (t, 1H, J=8.0 Hz) ppm.

(E)-4-bromo-3-(2-nitrovinyl)-1H-indole (0.100 g, 0.374 mmol) was put in a flame dried flask charged with a magnetic stir bar under argon. 2-Me-THF (2.45 mL) was added to the flask at room temperature. The reaction was cooled to 0° C. and 1.00 M LAH (2.25 mL, 2.25 mmol) was carefully added dropwise. The reaction was allowed to warm to room temperature and then refluxed for an hour (or until the reaction went to completion). Upon completion, the reaction was quenched with Glauber's Salt at 0° C. The slurry was filtered through a pad of celite and washed with an extensive amount of EtOAc. The filtrate was concentrated under reduced pressure to give a pale red oil that solidified into a white solid spontaneously (0.0803 g, 89.7%). The crude product, which contained a compound with the following structure

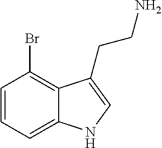 [SMILES: NCCc1c[nH]c2cccc(Br)c12]

was used in the next step without purification. LRMS m/z: [M+H]⁺=240.800.

Using the coupling procedure described in the introduction to examples 6-12, indole-3-acetic acid (0.250 g, 1.43 mmol) and 4-bromo-tryptamine (0.348 g, 1.46 mmol) were used to synthesize an amide (0.341 g, 60.3%) as a white solid. LRMS m/z: [M+H]⁺=396.100. The amide had the following structure:

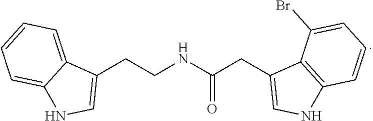 [SMILES: O=C(Cc1c[nH]c2cccc(Br)c12)NCCc1c[nH]c2ccccc12]

Using the reduction procedure A described in the introduction to examples 6-12, this amide (0.197 g, 0.497 mmol) was converted to the appropriate amine and obtained as a clear oil (0.190 g, 100%). This amine, which had the following structure:

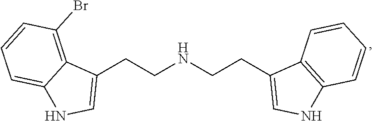 [SMILES: Brc1cccc2[nH]cc(CCNCCc3c[nH]c4ccccc34)c12]

was used in the next step without further purification. LRMS m/z: [M+H]⁺=382.100.

Using the reductive amination procedure described in the introduction to examples 6-12, the amine (0.190 g, 0.497 mmol) and formaldehyde (0.0500 g, 0.650 mmol) were converted to a tertiary amine as a white solid. LRMS m/z: [M+H]⁺=396.100. The tertiary amine had the following structure:

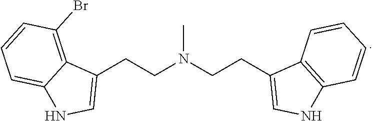 [SMILES: CN(CCc1c[nH]c2ccccc12)CCc1c[nH]c2cccc(Br)c12]

### Example 12

Using the coupling procedure described in the introduction to examples 6-12, 2-(1H-indol-3-yl)-2-oxoacetic acid (0,100 g, 0.405 mmol) and N-methyl-tryptamine (0.106 g, 0.607 mmol) were used to synthesize an amide as a tan oil (0.0493 g, 30.2%). The amide, which had the following structure:

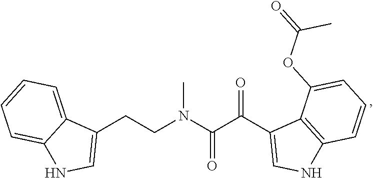 [SMILES: CC(=O)Oc1cccc2[nH]cc(C(=O)C(=O)N(C)CCc3c[nH]c4ccccc34)c12]

was used in the next step without further purification. LRMS m/z: [M+H]⁺=404.200.

Using the reduction procedure B described in the introduction to examples 6-12, the amide (0.0490 g, 0.122 mmol) was converted to the corresponding amine (0.0400 g, 100%) as a tan oil. The amine, which had the following structure:

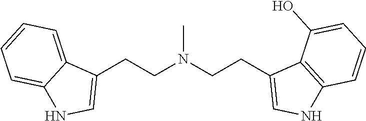 [SMILES: CN(CCc1c[nH]c2ccccc12)CCc1c[nH]c2cccc(O)c12]

was used in the next step without further purification. LRMS m/z: [M+H]⁺=334.200.

The amine with the free phenol (0.0400 g, 0.120 mmol)

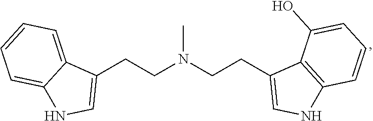 [SMILES: CN(CCc1c[nH]c2ccccc12)CCc1c[nH]c2cccc(O)c12]

was dissolved in 0.600 mL of anhydrous DCM under argon. The reaction was cooled on ice and acetyl chloride (0.00942 g, 0.132 mmol) followed by triethylamine (0.0184 mL, 0.132 mmol) were added dropwise. The reaction was stirred at room temperature until completion. The reaction was washed with water, dried over Na₂SO₄, and dried down under reduced pressure to give the product as a brown oil. LRMS m/z: [M+H]⁺=376.200. The product had the following structure:

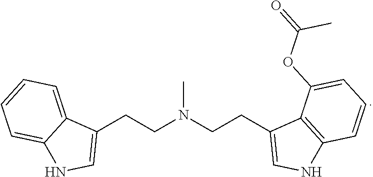 [SMILES: CC(=O)Oc1cccc2[nH]cc(CCN(C)CCc3c[nH]c4ccccc34)c12]

TABLE 4

| Ex. | | |
|---|---|---|
| No. | Structure | IUPAC Name |
| 6 | 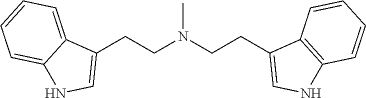 [SMILES: CN(CCc1c[nH]c2ccccc12)CCc1c[nH]c2ccccc12] | N-(2-(1H-indol-3-yl)ethyl)-2-(1H-indol-3-yl)-N- methylethan-1-amine |
| 7 | 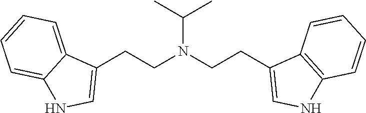 [SMILES: CC(C)N(CCc1c[nH]c2ccccc12)CCc1c[nH]c2ccccc12] | N,N-bis(2-(1H-indol-3-yl)ethyl)propan-2-amine |
| 8 | 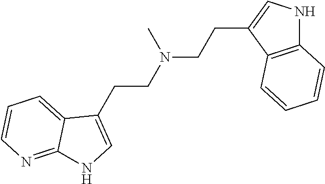 [SMILES: CN(CCc1c[nH]c2ccccc12)CCc1c[nH]c2ncccc12] | N-(2-(1H-indol-3-yl)ethyl)-N-methyl-2-(1H-pyrrolo[2,3- b]pyridin-3-yl)ethan-1-amine |
| 9 | 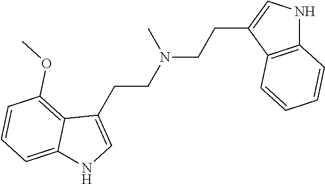 [SMILES: COc1cccc2[nH]cc(CCN(C)CCc3c[nH]c4ccccc34)c12] | N-(2-(1H-indol-3-yl)ethyl)-2-(4-methoxy-1H-indol-3-yl)-N- methylethan-1-amine |
| 10 | 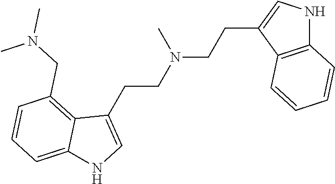 [SMILES: CN(C)Cc1cccc2[nH]cc(CCN(C)CCc3c[nH]c4ccccc34)c12] | N-(2-(1H-indol-3-yl)ethyl)-2-(4-((dimethylamino)methyl)- 1H-indol-3-yl)-N-methylethan-1-amine |
| 11 | 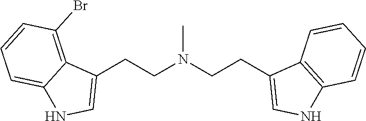 [SMILES: CN(CCc1c[nH]c2ccccc12)CCc1c[nH]c2cccc(Br)c12] | N-(2-(1H-indol-3-yl)ethyl)-2-(4-bromo-1H-indol-3-yl)-N- methylethan-1-amine |
| 12 | 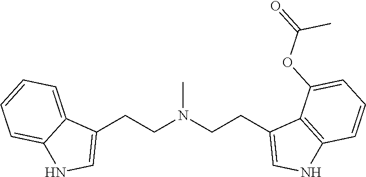 [SMILES: CC(=O)Oc1cccc2[nH]cc(CCN(C)CCc3c[nH]c4ccccc34)c12] | 3-(2-((2-(1H-indol-3-yl)ethyl)(methyl)amino)ethyl)-1H- indol-4-yl acetate |

1.-58. (canceled)

59. A compound of the formula: 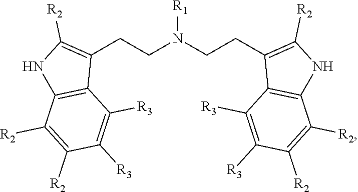
formula I, wherein
R₁ is H, D or an aliphatic substituent with a primary, secondary, tertiary, or quaternary amine;
R₂ is H, D, CH₃, CF₃ or a halogen; and
R₃ is H, D, or an oxidized substituent with an alcohol, ester, ether, aldehyde, acid, amide, thiol, sulfone, sulfonamide, reversed sulfonamide, urea or halogen.

60. The compound of claim 59, wherein R₁ is H.

61. The compound of claim 59, wherein R₁ is a primary, secondary, tertiary, or quaternary amine.

62. The compound of claim 59, wherein R₂ is H.

63. The compound of claim 59, wherein R₂ is CH₃.

64. The compound of claim 59, wherein R₂ is CF₃.

65. The compound of claim 59, wherein R₂ is a halogen.

66. The compound of claim 60, wherein R₂ is H.

67. The compound of claim 60, wherein R₂ is CH₃.

68. The compound of claim 60, wherein R₂ is CF₃.

69. The compound of claim 60, wherein R₂ is a halogen.

70. The compound of claim 61, wherein R₂ is H.

71. The compound of claim 61, wherein R₂ is CH₃.

72. The compound of claim 61, wherein R₂ is CF₃.

73. The compound of claim 61, wherein R₂ is a halogen.

74. The compound of claim 59, wherein R₃ is H.

75. The compound of claim 62, wherein R₃ is H.

76. The compound of claim 59, wherein R₃ is an oxidized substituent with an alcohol, ester, ether, aldehyde, acid, amide, thiol, sulfone, sulfonamide, reversed sulfonamide, urea or halogen.

77. The compound of claim 62, wherein R₃ is an oxidized substituent with an alcohol, ester, ether, aldehyde, acid, amide, thiol, sulfone, sulfonamide, reversed sulfonamide, urea or halogen.

78. The compound of claim 59, wherein the compound is 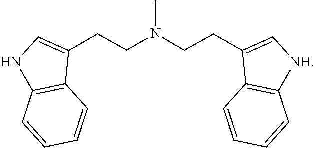 [SMILES: CN(CCc1c[nH]c2ccccc12)CCc1c[nH]c2ccccc12]
